(19) United States
(12) Patent Application Publication
(10) Pub. No.: US 2024/0228595 A1
(43) Pub. Date: Jul. 11, 2024

(54) Methods for Monoclonal Antibody Generation

(71) Applicant: YALE UNIVERSITY, New Haven, CT (US)

(72) Inventors: Sidi Chen, Milford, CT (US); Lei Peng, New Haven, CT (US); Ping Ren, New Haven, CT (US)

(21) Appl. No.: 18/559,559

(22) PCT Filed: May 10, 2022
(86) PCT No.: PCT/US22/72227

§ 371 Date: Nov. 7, 2023

Related U.S. Application Data
(60) Provisional application No. 63/187,081, filed on May 11, 2021.
(60) Provisional application No. 63/319,905, filed on Mar. 15, 2022.

(51) Int. Cl. C07K 16/10; A61K 39/215; A61P 31/14; A61P 37/04; C12Q 1/6876; G01N 33/569; A61K 39/00

(52) U.S. Cl. CPC C07K 16/1003; A61K 39/215; A61P 31/14; A61P 37/04; C12Q 1/6876; G01N 33/56983; A61K 2039/505; A61K 2039/53; A61K 2039/575; C07K 2317/31; C07K 2317/76; C12N 2770/20034; G01N 2333/165; G01N 2469/10

(57) ABSTRACT

The present disclosure provides a set of high-throughput methods for rapid and efficient identification and generation of monoclonal antibodies. Also provided are highly potent human and humanized monoclonal antibodies and antigen-binding fragments and bispecific antibodies which are capable of binding to a wild-type or variant SARS-COV-2 spike protein receptor-binding domain (RBD), isolated nucleic acids and expression vectors encoding the antibodies and antigen-binding fragments and bispecific antibodies, cells comprising the nucleic acids and/or expression vectors, and methods for detecting, diagnosing, and/or neutralizing SARS-COV-2 and for treating at least one sign or symptom of a condition, disorder, or disease caused by SARS-COV-2 infection, including COVID-19.

### CROSS-REFERENCE TO RELATED APPLICATIONS

This application claims priority under 35 U.S.C. § 119(e) to U.S. Provisional Patent Application No. 63/187,081, filed May 11, 2021, and to U.S. Provisional Patent Application No. 63/319,905, filed Mar. 15, 2022, which are incorporated herein by reference in their entireties.

### STATEMENT REGARDING FEDERALLY SPONSORED RESEARCH OR DEVELOPMENT

This invention was made with government support under W81XWH-21-1-0019 awarded by the United States Department of Defense. The government has certain rights in the invention.

### BACKGROUND

Severe acute respiratory syndrome coronavirus 2 (SARS-COV-2), one of a family of human coronaviruses, causes a disease presentation termed coronavirus disease 2019 (COVID-19). Initially identified in Wuhan, China in December 2019, the virus has subsequently spread throughout the world and was declared a pandemic by the World Health Organization on Mar. 11, 2020. To date, SARS-COV-2 has infected nearly 500 million confirmed individuals and caused over 6 million deaths.

SARS-COV-2 uses the host angiotensin converting enzyme II (ACE2) as its cell entry receptor protein to access and infect human cells via binding of ACE2 by the viral spike (S) protein receptor-binding domain (RBD), located on the surface of coronaviruses. Viral entry further requires priming of the S protein by host cellular proteases.

Recently, fast emergence of several different SARS-COV-2 variants (i.e., mutation strains) urgently demands therapeutic and prophylactic interventions, such as neutralization antibody with multi-neutralization ability. Pre-clinical data and clinical studies indicate that monoclonal antibodies (mAbs) can be effectively deployed for prevention or treatment of the COVID-19 disease. However, new viral mutants have continually emerged (and may continue to emerge), and some significantly affect the rate of spread and/or the ability to evade immune recognition, and dampen the efficacy of existing antibody therapy or of vaccines (Tegally, Wilkinson et al. 2020, Tegally, Wilkinson et al. 2021, Wang, Liu et al. 2021). In particular, a new lineage, Omicron (B.1.1.529), initially reported in South Africa in late 2021, rapidly became the predominant variant circulating in many countries and led the fourth pandemic wave globally. As compared with the genome sequences of previous variants of concerns (VOCs), the recent Omicron variant has harbored a high number of genomic mutations, especially in the spike (S) glycoprotein and clustered in the receptor-binding domain (RBD). These mutations drastically decrease the efficacy of currently available vaccines and FDA-approved or emergency use authorized (EUA) monoclonal antibody-based therapies (Liu, et al., 2022, Nature 602, 676-681; Planas, et al., 2022, Nature 602, 671-675; Rossler, et al., 2022, N Engl J Med 386, 698-700; Cao, et al., 2022, Nature 602, 657-663; Cele, et al., 2022, Nature 602, 654-656; Zhou, et al., 2022, Science, eabn8897; Iketani, et al, 2022, Nature). Several major sublineages of Omicron evolved, including BA.1, BA.1.1, BA.2, BA.3, BA.4, and BA.5. The rapidly rising BA.2 sublineage is leading the current pandemic waves globally, which has more severe resistance profile to the majority of existing COVID-19 therapeutic antibodies, leaving few effective antibodies.

Thus, there is a need in the art not only for specific monoclonal antibodies capable of neutralizing newly emerging SARS-COV-2 variants, but also for strategies which can rapidly produce highly-specific monoclonal antibodies against specific antigens, including but not limited to viral antigens. The current invention addresses these needs.

### SUMMARY OF THE INVENTION

In some aspects, the invention provides a method of identifying antigen-specific antibodies, the method comprising:
a. vaccinating a subject with an effective amount of a lipid nanoparticle comprising at least one mRNA encoding an antigen;
b. boosting the subject with at least one additional vaccination with an effective amount of the lipid nanoparticle comprising at least one mRNA encoding the antigen;
c. isolating B cells from the subject after the vaccination of step a;
d. identifying and cloning BCR heavy and light chain pairs expressed by enriched B cells; and
e. screening the BCR heavy and light chains for antigen-specificity thereby identifying antigen-specific antibodies.

In some embodiments, isolating B cells from the subject is performed at least about 14 days after the vaccination of step a.

In some embodiments, boosting the subject comprises four additional vaccinations.

In some embodiments, the additional vaccinations occur about two days, four days, seven days, and eleven days after the initial vaccination.

In some embodiments, identifying BCR heavy and light chain pairs is performed by single-cell BCR sequencing (scBCRseq).

In some embodiments, the subject is a mammal.

In some embodiments, the subject is a mouse.

In some embodiments, the subject is a transgenic mouse.

In some embodiments, the subject is a humanized mouse.

In some embodiments, the humanized mouse comprises human IgG and IgK transgenes.

In some embodiments, the vaccination is administered intramuscularly.

In some embodiments, the antigen is a severe acute respiratory syndrome coronavirus 2 (SARS-COV-2) antigen.

In some embodiments, the SARS-COV-2 antigen is a SARS-COV-2 spike protein receptor binding domain (RBD).

In some aspects, the invention provides an antibody produced by a method disclosed herein.

In some aspects, the invention provides a method for rapid identification of a monoclonal antibody or antigen-binding fragment which is capable of binding a spike protein receptor binding domain (RBD) of a severe acute respiratory syndrome coronavirus 2 (SARS-COV-2), the method comprising:
a. immunizing an animal with a polypeptide comprising the spike protein RBD or with a nucleic acid comprising a nucleotide sequence encoding the spike protein RBD;
b. sequencing fully recombined VDJ sequences of B cell receptor (BCR) repertoires from multiple individual B cells of the animal, thereby generating sequencing data;
c. identifying candidate antibody clones from the sequencing data;
d. producing a recombinant monoclonal antibody or antigen-binding fragment comprising paired heavy and light chain variable regions from one or more candidate antibody clones; and
e. testing the produced antibody or antigen-binding fragment for an ability to bind the spike protein RBD.

In some embodiments, the sequencing is high-throughput single-cell B-cell receptor sequencing (scBCR-seq).

In some embodiments, the animal is a mouse.

In some embodiments, the animal is a C57BL/6J mouse or a BALB/c mouse.

In some embodiments, the multiple individual B cells are isolated progenitor B cells and/or plasma B cells from spleen, lymph node, and/or bone marrow of the animal.

In some embodiments, the SARS-COV-2 is wild-type SARS-COV-2 or a variant SARS-CoV-2.

In some embodiments, the produced antibody or antigen-binding fragment binds the RBD with nanomolar affinity.

In some embodiments, the produced antibody or antigen-binding fragment binds the RBD with picomolar avidity.

In some embodiments, the produced antibody or antigen-binding fragment has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 PM, less than 20 pM, less than 10 pM, less than 5 pM, or less than 2.5 pM.

In some embodiments, the produced antibody or antigen-binding fragment specifically binds the SARS-COV-2 spike protein RBD.

In some embodiments, the antibody or antigen-binding fragment binds an epitope of the RBD which partially or completely overlaps with the angiotensin converting enzyme II (ACE2) binding site of RBD, thereby preventing RBD binding to ACE2.

In some embodiments, the produced antibody or antigen-binding fragment is a neutralizing antibody or antigen-binding fragment.

In some embodiments, the produced antibody or antigen-binding fragment is effective against lethal challenge of SARS-COV-2 in vivo.

In some embodiments, the produced antibody or antigen-binding fragment is selected from the group consisting of a monospecific antibody, a bispecific antibody, a Fab, an Fab′, an F(ab′)₂, an Fv, an scFv, a linear antibody, and a single domain antibody (sdAb).

In some embodiments, the produced antibody or antigen-binding fragment is a bispecific antibody selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb).

In some embodiments, the bispecific antibody is an asymmetric IgG-like bispecific antibody which binds two distinct epitopes of the RBD.

In some embodiments, the produced antibody or antigen-binding fragment is conjugated or recombinantly fused to at least one other moiety or molecule selected from the group consisting of a diagnostic agent, a detectable agent, a therapeutic agent, a purification tag, a molecule affecting one or more biological or molecular properties of the antibody or antigen-binding domain, and any combination thereof, wherein the biological or molecular properties comprise serum stability, half-life, solubility, and antigenicity.

In some aspects, the invention provides an antibody produced by a method disclosed herein.

In some aspects, the invention provides monoclonal antibody or antigen-binding fragment, wherein the antibody or antigen-binding fragment is capable of binding a severe acute respiratory syndrome coronavirus 2 (SARS-COV-2) spike protein receptor binding domain (RBD), wherein the antibody or antigen-binding fragment comprises three heavy chain complementarity determining regions (HCDR1, HCDR2, and HCDR3) and three light chain complementarity determining regions (LCDR1, LCDR2, and LCDR3) selected from the group consisting of:
a. an HCDR1 comprising SEQ ID NO: 1, an HCDR2 comprising SEQ ID NO: 2, an HCDR3 comprising SEQ ID NO: 3, an LCDR1 comprising SEQ ID NO: 4, an LCDR2 comprising the amino acid sequence AAS, and an LCDR3 comprising SEQ ID NO: 6;
b. an HCDR1 comprising SEQ ID NO: 11, an HCDR2 comprising SEQ ID NO: 12, an HCDR3 comprising SEQ ID NO: 13, an LCDR1 comprising SEQ ID NO: 14, an LCDR2 comprising the amino acid sequence GSS, and an LCDR3 comprising SEQ ID NO: 16;
c. an HCDR1 comprising SEQ ID NO: 21, an HCDR2 comprising SEQ ID NO: 22, an HCDR3 comprising SEQ ID NO: 23, an LCDR1 comprising SEQ ID NO: 24, an LCDR2 comprising the amino acid sequence AAS, and an LCDR3 comprising SEQ ID NO: 26;
d. an HCDR1 comprising SEQ ID NO: 31, an HCDR2 comprising SEQ ID NO: 32, an HCDR3 comprising SEQ ID NO: 33, an LCDR1 comprising SEQ ID NO: 34, an LCDR2 comprising the amino acid sequence AAS, and an LCDR3 comprising SEQ ID NO: 36;
e. an HCDR1 comprising SEQ ID NO: 107, an HCDR2 comprising SEQ ID NO: 108, an HCDR3 comprising SEQ ID NO: 109, an LCDR1 comprising SEQ ID NO: 116, an LCDR2 comprising the amino acid sequence GAS, and an LCDR3 comprising SEQ ID NO: 118;
f. an HCDR1 comprising SEQ ID NO: 110, an HCDR2 comprising SEQ ID NO: 111, an HCDR3 comprising SEQ ID NO: 112, an LCDR1 comprising SEQ ID NO: 119, an LCDR2 comprising the amino acid sequence WAS, and an LCDR3 comprising SEQ ID NO: 121; and
g. an HCDR1 comprising SEQ ID NO: 113, an HCDR2 comprising SEQ ID NO: 114, an HCDR3 comprising SEQ ID NO: 115, an LCDR1 comprising SEQ ID NO: 122, an LCDR2 comprising the amino acid sequence DAS, and an LCDR3 comprising SEQ ID NO: 124.

In some embodiments, the SARS—Co-V-2 is wild-type SARS-COV-2 or a variant SARS-CoV-2.

In some embodiments, the antibody or antigen-binding fragment binds the RBD with nanomolar affinity.

In some embodiments, the antibody or antigen-binding fragment binds the RBD with picomolar avidity.

In some embodiments, the antibody or antigen-binding fragment has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 pM, less than 20 pM, less than 10 pM, less than 5 pM, or less than 2.5 pM.

In some embodiments, the antibody or antigen-binding fragment specifically binds the SARS-COV-2 spike protein RBD.

In some embodiments, the antibody or antigen-binding fragment binds an epitope of the RBD which partially or completely overlaps with the angiotensin converting enzyme II (ACE2) binding site of RBD, thereby preventing RBD binding to ACE2.

In some embodiments, the antibody or antigen-binding fragment is a neutralizing antibody or antigen-binding fragment.

In some embodiments, the antibody or antigen-binding fragment is effective against lethal challenge of SARS-COV-2 in vivo.

In some embodiments, the antibody or antigen-binding fragment is selected from the group consisting of a monospecific antibody, a bispecific antibody, an Fab, an Fab′, an F(ab′)₂, an Fv, an scFv, a linear antibody, and a single domain antibody (sdAb).

In some embodiments, the antibody or antigen-binding fragment is a bispecific antibody selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb).

In some embodiments, the bispecific antibody is an asymmetric IgG-like bispecific antibody which binds two distinct epitopes of the RBD.

In some embodiments, the antibody or antigen-binding fragment comprises at least one heavy chain variable region (VH) and at least one light chain variable region (VL) selected from the group consisting of:
a. a VH comprising an amino acid sequence at least 80%, 85%, 90%, 95%, 96%, 97%, 98%, 99%, or 100% identical to SEQ ID NO: 7, and a VL comprising an amino acid sequence at least 80%, 85%, 90%, 95%, 96%, 97%, 98%, 99%, or 100% identical to SEQ ID NO: 8;
b. a VH comprising an amino acid sequence at least 80%, 85%, 90%, 95%, 96%, 97%, 98%, 99%, or 100% identical to SEQ ID NO: 17, and a VL comprising an amino acid sequence at least 80%, 85%, 90%, 95%, 96%, 97%, 98%, 99%, or
c. a VH comprising an amino acid sequence at least 80%, 85%, 90%, 95%, 96%, 97%, 98%, 99%, or 100% identical to SEQ ID NO: 27, and a VL comprising an amino acid sequence at least 80%, 85%, 90%, 95%, 96%, 97%, 98%, 99%, or 100% identical to SEQ ID NO: 28;
d. a VH comprising an amino acid sequence at least 80%, 85%, 90%, 95%, 96%, 97%, 98%, 99%, or 100% identical to SEQ ID NO: 37, and a VL comprising an amino acid sequence at least 80%, 85%, 90%, 95%, 96%, 97%, 98%, 99%, or 100% identical to SEQ ID NO: 38;
e a VH comprising an amino acid sequence at least 80%, 85%, 90%, 95%, 96%, 97%, 98%, 99%, or 100% identical to SEQ ID NO: 101, and a VL comprising an amino acid sequence at least 80%, 85%, 90%, 95%, 96%, 97%, 98%, 99%, or 100% identical to SEQ ID NO: 104;
f. a VH comprising an amino acid sequence at least 80%, 85%, 90%, 95%, 96%, 97%, 98%, 99%, or 100% identical to SEQ ID NO: 102, and a VL comprising an amino acid sequence at least 80%, 85%, 90%, 95%, 96%, 97%, 98%, 99%, or 100% identical to SEQ ID NO: 105; and
g. a VH comprising an amino acid sequence at least 80%, 85%, 90%, 95%, 96%, 97%, 98%, 99%, or 100% identical to SEQ ID NO: 103, and a VL comprising an amino acid sequence at least 80%, 85%, 90%, 95%, 96%, 97%, 98%, 99%, or 100% identical to SEQ ID NO: 106.

In some embodiments, the antibody or antigen-binding fragment comprises at least one heavy chain and at least one light chain selected from the group consisting of:
a. a heavy chain comprising an amino acid sequence at least 80%, 85%, 90%, 95%, 96%, 97%, 98%, 99%, or 100% identical to SEQ ID NO: 9, and a light chain comprising an amino acid sequence at least 80%, 85%, 90%, 95%, 96%, 97%, 98%, 99%, or 100% identical to SEQ ID NO: 10;
b. a heavy chain comprising an amino acid sequence at least 80%, 85%, 90%, 95%, 96%, 97%, 98%, 99%, or 100% identical to SEQ ID NO: 19, and a light chain comprising an amino acid sequence at least 80%, 85%, 90%, 95%, 96%, 97%, 98%, 99%, or 100% identical to SEQ ID NO: 20;
c. a heavy chain comprising an amino acid sequence at least 80%, 85%, 90%, 95%, 96%, 97%, 98%, 99%, or 100% identical to SEQ ID NO: 29, and a light chain comprising an amino acid sequence at least 80%, 85%, 90%, 95%, 96%, 97%, 98%, 99%, or 100% identical to SEQ ID NO: 30;
d. a heavy chain comprising an amino acid sequence at least 80%, 85%, 90%, 95%, 96%, 97%, 98%, 99%, or 100% identical to SEQ ID NO: 39, and a light chain comprising an amino acid sequence at least 80%, 85%, 90%, 95%, 96%, 97%, 98%, 99%, or 100% identical to SEQ ID NO: 40;
e. a heavy chain comprising an amino acid sequence at least 80%, 85%, 90%, 95%, 96%, 97%, 98%, 99%, or 100% identical to SEQ ID NO: 133, and a light chain comprising an amino acid sequence at least 80%, 85%, 90%, 95%, 96%, 97%, 98%, 99%, or 100% identical to SEQ ID NO: 134;
f. a heavy chain comprising an amino acid sequence at least 80%, 85%, 90%, 95%, 96%, 97%, 98%, 99%, or 100% identical to SEQ ID NO: 135, and a light chain comprising an amino acid sequence at least 80%, 85%, 90%, 95%, 96%, 97%, 98%, 99%, or 100% identical to SEQ ID NO: 136; and
g. a heavy chain comprising an amino acid sequence at least 80%, 85%, 90%, 95%, 96%, 97%, 98%, 99%, or 100% identical to SEQ ID NO: 137, and a light chain comprising an amino acid sequence at least 80%, 85%, 90%, 95%, 96%, 97%, 98%, 99%, or 100% identical to SEQ ID NO: 138.

In some embodiments, the antibody or antigen-binding fragment is conjugated or recombinantly fused to at least one other moiety or molecule selected from the group consisting of a diagnostic agent, a detectable agent, a therapeutic agent, a purification tag, a molecule affecting one or more biological or molecular properties of the antibody or antigen-binding domain, or any combination thereof, wherein the biological or molecular properties comprise serum stability, half-life, solubility, and antigenicity.

In some aspects, the invention provides a bispecific antibody capable of binding a severe acute respiratory syndrome coronavirus 2 (SARS-COV-2) spike protein receptor binding domain (RBD), wherein the bispecific antibody comprises (i) a first heavy chain comprising a first heavy chain variable region (VH1), (ii) a first light chain comprising a first light chain variable region (VL1), (iii) a second heavy chain comprising a second heavy chain variable region (VH2), and (iv) a second light chain comprising a second light chain variable region (VL2); wherein the VH1 and the VH2 each comprises three heavy chain complementarity determining regions (HCDR1, HCDR2, and HCDR3) and the VL1 and the VL2 each comprises three light chain complementarity determining regions (LCDR1, LCDR2, and LCDR3); wherein the complementarity determining regions are selected from the group consisting of:
a. a VH1 HCDR1 comprising SEQ ID NO: 41, a VH1 HCDR2 comprising SEQ ID NO: 42, a VH1 HCDR3 comprising SEQ ID NO: 43, a VL1 LCDR1 comprising SEQ ID NO: 47, a VL1 LCDR2 comprising the amino acid sequence GSS, a VL1 LCDR3 comprising SEQ ID NO: 49, a VH2 HCDR1 comprising SEQ ID NO: 44, a VH2 HCDR2 comprising SEQ ID NO: 45, a VH2 HCDR3 comprising SEQ ID NO: 46, a VL2 LCDR1 comprising SEQ ID NO: 50, a VL2 LCDR2 comprising the amino acid sequence AAS, and a VL2 LCDR3 comprising SEQ ID NO: 52;
b. a VH1 HCDR1 comprising SEQ ID NO: 151, a VH1 HCDR2 comprising SEQ ID NO: 152, a VH1 HCDR3 comprising SEQ ID NO: 153, a VL1 LCDR1 comprising SEQ ID NO: 165, a VL1 LCDR2 comprising the amino acid sequence GAS, a VL1 LCDR3 comprising SEQ ID NO: 166, a VH2 HCDR1 comprising SEQ ID NO: 158, a VH2 HCDR2 comprising SEQ ID NO: 159, a VH2 HCDR3 comprising SEQ ID NO: 160, a VL2 LCDR1 comprising SEQ ID NO: 171, a VL2 LCDR2 comprising the amino acid sequence WAS, and a VL2 LCDR3 comprising SEQ ID NO: 172;
c. a VH1 HCDR1 comprising SEQ ID NO: 177, a VH1 HCDR2 comprising SEQ ID NO: 178, a VH1 HCDR3 comprising SEQ ID NO: 179, a VL1 LCDR1 comprising SEQ ID NO: 191, a VL1 LCDR2 comprising the amino acid sequence DAS, a VL1 LCDR3 comprising SEQ ID NO: 192, a VH2 HCDR1 comprising SEQ ID NO: 184, a VH2 HCDR2 comprising SEQ ID NO: 185, a VH2 HCDR3 comprising SEQ ID NO: 186, a VL2 LCDR1 comprising SEQ ID NO: 197, a VL2 LCDR2 comprising the amino acid sequence GAS, and a VL2 LCDR3 comprising SEQ ID NO: 198;
d. a VH1 HCDR1 comprising SEQ ID NO: 203, a VH1 HCDR2 comprising SEQ ID NO: 204, a VH1 HCDR3 comprising SEQ ID NO: 205, a VL1 LCDR1 comprising SEQ ID NO: 217, a VL1 LCDR2 comprising the amino acid sequence DAS, a VL1 LCDR3 comprising SEQ ID NO: 218, a VH2 HCDR1 comprising SEQ ID NO: 210, a VH2 HCDR2 comprising SEQ ID NO: 211, a VH2 HCDR3 comprising SEQ ID NO: 212, a VL2 LCDR1 comprising SEQ ID NO: 223, a VL2 LCDR2 comprising the amino acid sequence WAS, and a VL2 LCDR3 comprising SEQ ID NO: 224;
e. a VH1 HCDR1 comprising SEQ ID NO: 229, a VH1 HCDR2 comprising SEQ ID NO: 230, a VH1 HCDR3 comprising SEQ ID NO: 231, a VL1 LCDR1 comprising SEQ ID NO: 243, a VL1 LCDR2 comprising the amino acid sequence AAS, a VL1 LCDR3 comprising SEQ ID NO: 244, a VH2 HCDR1 comprising SEQ ID NO: 236, a VH2 HCDR2 comprising SEQ ID NO: 237, a VH2 HCDR3 comprising SEQ ID NO: 238, a VL2 LCDR1 comprising SEQ ID NO: 249, a VL2 LCDR2 comprising the amino acid sequence GAS, and a VL2 LCDR3 comprising SEQ ID NO: 250; and
f. a VH1 HCDR1 comprising SEQ ID NO: 255, a VH1 HCDR2 comprising SEQ ID NO: 256, a VH1 HCDR3 comprising SEQ ID NO: 257, a VL1 LCDR1 comprising SEQ ID NO: 269, a VL1 LCDR2 comprising the amino acid sequence AAS, a VL1 LCDR3 comprising SEQ ID NO: 270, a VH2 HCDR1 comprising SEQ ID NO: 262, a VH2 HCDR2 comprising SEQ ID NO: 263, a VH2 HCDR3 comprising SEQ ID NO: 264, a VL2 LCDR1 comprising SEQ ID NO: 275, a VL2 LCDR2 comprising the amino acid sequence WAS, and a VL2 LCDR3 comprising SEQ ID NO: 276.

In some embodiments, the SARS—Co-V-2 is wild-type SARS-COV-2 or a variant SARS-CoV-2.

In some embodiments, the bispecific antibody binds the RBD with nanomolar affinity.

In some embodiments, the bispecific antibody binds the RBD with picomolar avidity.

In some embodiments, the bispecific antibody has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 PM, less than 20 pM, less than 10 PM, less than 5 pM, or less than 2.5 pM.

In some embodiments, the bispecific antibody specifically binds the SARS-COV-2 spike protein RBD.

In some embodiments, the bispecific antibody binds an epitope of the RBD which partially or completely overlaps with the angiotensin converting enzyme II (ACE2) binding site of RBD, thereby preventing RBD binding to ACE2.

In some embodiments, the bispecific antibody is a neutralizing bispecific antibody.

In some embodiments, the bispecific antibody is effective against lethal challenge of SARS-COV-2 in vivo.

In some embodiments, the bispecific antibody is selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb).

In some embodiments, the bispecific antibody is an asymmetric IgG-like bispecific antibody which binds two distinct epitopes of the RBD.

In some embodiments, the VH1, VL1, VH2, and VL2 are selected from the group consisting of:
a. a VH1 comprising an amino acid sequence at least 80%, 85%, 90%, 95%, 96%, 97%, 98%, 99%, or 100% identical to SEQ ID NO: 53, a VL1 comprising an amino acid sequence at least 80%, 85%, 90%, 95%, 96%, 97%, 98%, 99%, or 100% identical to SEQ ID NO: 55, a VH2 comprising an amino acid sequence at least 80%, 85%, 90%, 95%, 96%, 97%, 98%, 99%, or 100% identical to SEQ ID NO: 54, and a VL2 comprising an amino acid sequence at least 80%, 85%, 90%, 95%, 96%, 97%, 98%, 99%, or 100% identical to SEQ ID NO: 56;
b. a VH1 comprising an amino acid sequence at least 80%, 85%, 90%, 95%, 96%, 97%, 98%, 99%, or 100% identical to SEQ ID NO: 154, a VL1 comprising an amino acid sequence at least 80%, 85%, 90%, 95%, 96%, 97%, 98%, 99%, or 100% identical to SEQ ID NO: 167, a VH2 comprising an amino acid sequence at least 80%, 85%, 90%, 95%, 96%, 97%, 98%, 99%, or 100% identical to SEQ ID NO: 161, and a VL2 comprising an amino acid sequence at least 80%, 85%, 90%, 95%, 96%, 97%, 98%, 99%, or 100% identical to SEQ ID NO: 173;
c. a VH1 comprising an amino acid sequence at least 80%, 85%, 90%, 95%, 96%, 97%, 98%, 99%, or 100% identical to SEQ ID NO: 180, a VL1 comprising an amino acid sequence at least 80%, 85%, 90%, 95%, 96%, 97%, 98%, 99%, or 100% identical to SEQ ID NO: 193, a VH2 comprising an amino acid sequence at least 80%, 85%, 90%, 95%, 96%, 97%, 98%, 99%, or 100% identical to SEQ ID NO: 187, and a VL2 comprising an amino acid sequence at least 80%, 85%, 90%, 95%, 96%, 97%, 98%, 99%, or 100% identical to SEQ ID NO: 199;
d. a VH1 comprising an amino acid sequence at least 80%, 85%, 90%, 95%, 96%, 97%, 98%, 99%, or 100% identical to SEQ ID NO: 206, a VL1 comprising an amino acid sequence at least 80%, 85%, 90%, 95%, 96%, 97%, 98%, 99%, or 100% identical to SEQ ID NO: 219, a VH2 comprising an amino acid sequence at least 80%, 85%, 90%, 95%, 96%, 97%, 98%, 99%, or 100% identical to SEQ ID NO: 213, and a VL2 comprising an amino acid sequence at least 80%, 85%, 90%, 95%, 96%, 97%, 98%, 99%, or 100% identical to SEQ ID NO: 225;
e. a VH1 comprising an amino acid sequence at least 80%, 85%, 90%, 95%, 96%, 97%, 98%, 99%, or 100% identical to SEQ ID NO: 232, a VL1 comprising an amino acid sequence at least 80%, 85%, 90%, 95%, 96%, 97%, 98%, 99%, or 100% identical to SEQ ID NO: 245, a VH2 comprising an amino acid sequence at least 80%, 85%, 90%, 95%, 96%, 97%, 98%, 99%, or 100% identical to SEQ ID NO: 239, and a VL2 comprising an amino acid sequence at least 80%, 85%, 90%, 95%, 96%, 97%, 98%, 99%, or 100% identical to SEQ ID NO: 251; and
f. a VH1 comprising an amino acid sequence at least 80%, 85%, 90%, 95%, 96%, 97%, 98%, 99%, or 100% identical to SEQ ID NO: 258, a VL1 comprising an amino acid sequence at least 80%, 85%, 90%, 95%, 96%, 97%, 98%, 99%, or 100% identical to SEQ ID NO: 271, a VH2 comprising an amino acid sequence at least 80%, 85%, 90%, 95%, 96%, 97%, 98%, 99%, or 100% identical to SEQ ID NO: 265, and a VL2 comprising an amino acid sequence at least 80%, 85%, 90%, 95%, 96%, 97%, 98%, 99%, or 100% identical to SEQ ID NO: 277.

In some embodiments, the first heavy chain, the first light chain, the second heavy chain, and the second light chain are selected from the group consisting of:
a. a first heavy chain comprising an amino acid sequence at least 80%, 85%, 90%, 95%, 96%, 97%, 98%, 99%, or 100% identical to SEQ ID NO: 57, a first light chain comprising an amino acid sequence at least 80%, 85%, 90%, 95%, 96%, 97%, 98%, 99%, or 100% identical to SEQ ID NO: 59, a second heavy chain comprising an amino acid sequence at least 80%, 85%, 90%, 95%, 96%, 97%, 98%, 99%, or 100% identical to SEQ ID NO: 58, and a second light chain comprising an amino acid sequence at least 80%, 85%, 90%, 95%, 96%, 97%, 98%, 99%, or 100% identical to SEQ ID NO: 60;
b. a first heavy chain comprising an amino acid sequence at least 80%, 85%, 90%, 95%, 96%, 97%, 98%, 99%, or 100% identical to SEQ ID NO: 155, a first light chain comprising an amino acid sequence at least 80%, 85%, 90%, 95%, 96%, 97%, 98%, 99%, or 100% identical to SEQ ID NO: 168, a second heavy chain comprising an amino acid sequence at least 80%, 85%, 90%, 95%, 96%, 97%, 98%, 99%, or 100% identical to SEQ ID NO: 162, and a second light chain comprising an amino acid sequence at least 80%, 85%, 90%, 95%, 96%, 97%, 98%, 99%, or 100% identical to SEQ ID NO: 174;
c. a first heavy chain comprising an amino acid sequence at least 80%, 85%, 90%, 95%, 96%, 97%, 98%, 99%, or 100% identical to SEQ ID NO: 181, a first light chain comprising an amino acid sequence at least 80%, 85%, 90%, 95%, 96%, 97%, 98%, 99%, or 100% identical to SEQ ID NO: 194, a second heavy chain comprising an amino acid sequence at least 80%, 85%, 90%, 95%, 96%, 97%, 98%, 99%, or 100% identical to SEQ ID NO: 188, and a second light chain comprising an amino acid sequence at least 80%, 85%, 90%, 95%, 96%, 97%, 98%, 99%, or 100% identical to SEQ ID NO: 200;
d. a first heavy chain comprising an amino acid sequence at least 80%, 85%, 90%, 95%, 96%, 97%, 98%, 99%, or 100% identical to SEQ ID NO: 207, a first light chain comprising an amino acid sequence at least 80%, 85%, 90%, 95%, 96%, 97%, 98%, 99%, or 100% identical to SEQ ID NO: 220, a second heavy chain comprising an amino acid sequence at least 80%, 85%, 90%, 95%, 96%, 97%, 98%, 99%, or 100% identical to SEQ ID NO: 214, and a second light chain comprising an amino acid sequence at least 80%, 85%, 90%, 95%, 96%, 97%, 98%, 99%, or 100% identical to SEQ ID NO: 226;
e. a first heavy chain comprising an amino acid sequence at least 80%, 85%, 90%, 95%, 96%, 97%, 98%, 99%, or 100% identical to SEQ ID NO: 233, a first light chain comprising an amino acid sequence at least 80%, 85%, 90%, 95%, 96%, 97%, 98%, 99%, or 100% identical to SEQ ID NO: 246, a second heavy chain comprising an amino acid sequence at least 80%, 85%, 90%, 95%, 96%, 97%, 98%, 99%, or 100% identical to SEQ ID NO: 240, and a second light chain comprising an amino acid sequence at least 80%, 85%, 90%, 95%, 96%, 97%, 98%, 99%, or 100% identical to SEQ ID NO: 252; and
f. a first heavy chain comprising an amino acid sequence at least 80%, 85%, 90%, 95%, 96%, 97%, 98%, 99%, or 100% identical to SEQ ID NO: 259, a first light chain comprising an amino acid sequence at least 80%, 85%, 90%, 95%, 96%, 97%, 98%, 99%, or 100% identical to SEQ ID NO: 272, a second heavy chain comprising an amino acid sequence at least 80%, 85%, 90%, 95%, 96%, 97%, 98%, 99%, or 100% identical to SEQ ID NO: 266, and a second light chain comprising an amino acid sequence at least 80%, 85%, 90%, 95%, 96%, 97%, 98%, 99%, or 100% identical to SEQ ID NO: 278.

In some embodiments, the bispecific antibody is conjugated or recombinantly fused to at least one other moiety or molecule selected from the group consisting of a diagnostic agent, a detectable agent, a therapeutic agent, a purification tag, a molecule affecting one or more biological or molecular properties of the antibody or antigen-binding domain, or any combination thereof, wherein the biological or molecular properties comprise serum stability, half-life, solubility, and antigenicity.

In some aspects, the invention provides an isolated nucleic acid comprising at least one polynucleotide sequence encoding the antibody or antigen-binding fragment disclosed herein or the bispecific antibody disclosed herein.

In some embodiments, the nucleic acid is DNA.

In some aspects, the invention provides a vector comprising the isolated nucleic acid disclosed herein.

In some embodiments, the vector is a cloning vector or an expression vector.

In some aspects, the invention provides a host cell comprising the isolated nucleic acid disclosed herein and/or the vector disclosed herein.

In some aspects, the invention provides a pharmaceutical composition comprising the monoclonal antibody or antigen-binding fragment disclosed herein or the bispecific antibody disclosed herein, and at least one pharmaceutical carrier, vehicle or diluent.

In some aspects, the invention provides a method of detecting a severe acute respiratory syndrome coronavirus 2 (SARS-COV-2), wherein the method comprises contacting a sample with the monoclonal antibody or antigen-binding fragment disclosed herein or the bispecific antibody disclosed herein.

In some aspects, the invention provides a method of diagnosing a severe acute respiratory syndrome coronavirus 2 (SARS-COV-2) infection in a subject, wherein the method comprises contacting a sample obtained from the subject with the monoclonal antibody or antigen-binding fragment disclosed herein or the bispecific antibody disclosed herein.

In some aspects, the invention provides a method of neutralizing a severe acute respiratory syndrome coronavirus 2 (SARS-COV-2) in a subject, wherein the method comprises administering to the subject the monoclonal antibody or antigen-binding fragment disclosed herein or the bispecific antibody disclosed herein.

In some aspects, the invention provides a method of treating at least one sign or symptom of a severe acute respiratory syndrome coronavirus 2 (SARS-COV-2) infection in a subject, wherein the method comprises administering to the subject the monoclonal antibody or antigen-binding fragment disclosed herein or the bispecific antibody disclosed herein.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A-FIG. 1G illustrate the finding that immunization-single B cell sequencing leads to development of potent monoclonal antibodies in both monospecific and bispecific forms. FIG. 1A shows a schematic timeline of mouse immunization for antibody development. FIG. 1B shows a schematic which illustrates neutralizing antibody identification process through RBD-his tag protein mouse immunization-single B cell sequencing. The paired heavy chain and light chain sequence of the antibody was obtained using 10× Genomics VDJ sequencing. Antibodies were reconstructed by cloning of IgG heavy and light chains into human IgG1 backbone and expressed as recombinant monospecific or bispecific mAbs. Lead antibody clones were subjected to characterizations including neutralization assays, BL3 level anti-authentic SARS-COV-2 efficacy testing, and structural analyses by Cryo-EM. FIG. 1C presents two charts providing data related to frequency distribution of immunoglobulin isotypes in SARS-COV-2 Spike RBD immunized mice. (Left panel) Analysis of isotypes in both bone marrow and spleen in RBD immunized C57BL/6J mice. (Right panel) Analysis of isotypes in RBD immunized C57BL/6J and BALB/c mice. FIG. 1D presents two tables providing CDR3 sequences of heavy and light chains of the top enriched antibody clones from RBD-his tag immunized C57BL/6J mice (Top) and BALB/c mice (Bottom). FIG. 1E presents two charts providing data related to SARS-COV-2 RBD reactivity ELISA result of recombinant monospecific mAb clones identified from single BCR sequencing of RBD immunized C57BL/6J (top) and BALB/c (bottom) mice. FIG. 1F is a schematic of construct design and antibody structure of bispecific antibodies used in this study. FIG. 1G is a chart providing data related to SARS-COV-2 RBD reactivity ELISA result of top monospecific mAb clones (clones 2 and 6) and a bispecific mAb clone (clone 16).

FIG. 2A-FIG. 2H illustrate the finding that anti-spike RBD monoclonal antibodies have potent activity against SARS-COV-2. FIG. 2A presents two graphs providing data related to BLI measurement of binding strengths for top SARS-COV-2 RBD-specific monospecific mAb clones (Clones 2 and 6). FIG. 2B presents two graphs providing data from SPR measurement of binding strengths top SARS-COV-2 RBD-specific monospecific mAb clones (Clones 2 and 6), using a NTA chip where the antigen (RBD) was fixed on the chip. FIG. 2C presents four graphs providing data from SPR measurement of binding strengths top SARS-COV-2 RBD-specific monospecific mAb clones (Clones 2 and 6), a humanized mAb clone (Clone 13), and a bispecific mAb (Clone 16), using a Fc chip where antibodies were fixed on the chip. FIG. 2D presents two graphs providing data from neutralization assay on top mAb clones using WT (left) and South African variant (right) SARS-COV-2 Spike pseudotyped HIV1-lentivirus carrying a EGFP reporter. FIG. 2E is a schematic of in vivo efficacy testing of top mAb clones against lethal challenges of authentic SARS-COV-2 in hACE2 transgenic mice at BL3 level. FIG. 2F presents three graphs providing data related to body weight curves of antibody and placebo (DPBS) treated hACE2 transgenic mice under lethal challenges of authentic SARS-COV-2. The graphs show spider curves of body weight changes of individual mouse plotted separated by group. Clone 2, n=8; Clone 6, n=7, and DPBS, n=8. FIG. 2G presents a graph providing data related to body weight curves of antibody and placebo (DPBS) treated hACE2 transgenic mice under lethal challenges of authentic SARS-COV-2. The graph shows mean+/−SEM curves of body weight changes. Clone 2, n=8; Clone 6, n=7, and DPBS, n=8. FIG. 2H presents a graph showing survival curves of antibody and placebo (DPBS) treated hACE2 transgenic mice under lethal challenges of authentic SARS-CoV-2 (the same experiment as in FIG. 2F and FIG. 2G). FIG. 2I is a schematic of in vivo efficacy testing of Clone 2 and Clone 6 against replication-competent SARS-COV-2 virus, using a therapeutic setting where the animals were treated post-infection. FIG. 2J-FIG. 2L presents the efficacy data of Clone 2 and Clone 6 against replication-competent SARS-COV-2 virus in vivo, using a therapeutic setting where the animals were treated post infection. FIG. 2M presents Clone 2 and Clone 6, and the bispecific Clone 16's potency against replication-competent SARS-COV-2 (USA-WA1/2020). The authentic virus neutralization assay was performed in a biosafety level 3 (BSL3) setting, Clone 2, Clone 6 and the bispecific Clone 16 inhibited the viral propagation of the fully infectious SARS-COV-2 WA1 (low-mid ng/ml level IC50s). For FIG. 2A-FIG. 2M: Data are shown as mean±s.e.m. plus individual data points in dot plots. Statistics: Two-way ANOVA was used to assess statistical significance for multi-group curve comparisons; Log-Rank test was used to assess statistical significance for survival curve comparisons, unless otherwise noted. The p-values are indicated in the plots.

FIG. 3A-FIG. 3B illustrate the finding that cryo-EM structures of monoclonal antibodies reveal combinations of open and closed conformations of trimeric spike. FIG. 3A provides side views of three cryo-EM structures of Clone 6 monoclonal antibody bound to trimeric S showing binding epitopes and combinations of open and close conformations. FIG. 3B provides top views of three cryo-EM structures of Clone 6 monoclonal antibody bound to trimeric S showing binding epitopes and combinations of open and close conformations.

FIG. 4A-FIG. 4B provide data from the measurement of mouse serum titers in SARS-CoV-2 Spike RBD immunized mice in this study. FIG. 4A presents five graphs showing SARS-CoV-2 RBD reactivity ELISA result of serum samples from different RBD immunized C57BL/6J mice. FIG. 4B presents three graphs showing SARS-COV-2 RBD reactivity ELISA result of serum samples from different RBD immunized BALB/c mice.

FIG. 5A-FIG. 5D provide data for neutralization capability testing of antibody clones with HIV1-based and VSV-G-based WT SARS-COV-2 spike pseudotyped virus. FIG. 5A is a graph showing data from neutralization assay on Clone 2, and Clone 3 using WT SARS-COV-2 Spike pseudotyped lentivirus carrying a luciferase reporter. FIG. 5B is a graph showing data from validation neutralization assay on Clone 2 using WT SARS-COV-2 Spike pseudotyped lentivirus carrying a luciferase reporter. FIG. 5C is a graph showing data from neutralization assay on Clone 2, Clone 6 and Clone 5 and Clone 11 using WT SARS-COV-2 Spike pseudotyped VSV-virus carrying a luciferase reporter. FIG. 5D is a graph showing data from neutralization assay on Clone 2, Clone 5, Clone 6, two humanized antibodies of Clone 2 (Clone 12 and Clone 13, a commercial anti-SARS-COV-2 spike RBD protein antibody and a IgG1 isotype control using WT SARS-COV-2 Spike pseudotyped HIV-lentivirus carrying a luciferase reporter.

FIG. 6A-FIG. 6B provide chromatograph data for CryoEM studies. FIG. 6A provides an FPLC chromatogram and an SDS-PAGE gel showing purification of Clone 2 Fab and formation of antibody-spike protein complexes for CryoEM studies. FIG. 6B provides an FPLC chromatogram and an SDS-PAGE gel showing purification of Clone 6 Fab and formation of antibody-spike protein complexes for CryoEM studies.

FIG. 7A-FIG. 7F illustrate the development of RAMIHM for rapid discovery of fully human monoclonal antibodies and application with Omicron mRNA immunization. FIG. 7A, Schematic showing the domain arrangement of the SARS-COV-2 WT spike and its variant SARS CoV-2 B.1.1.529 (Omicron). Mutations present in Omicron spike protein are labeled. Full-length of Omicron spike gene was synthesized to construct Omicron-specific mRNA-lipid nanoparticle and Omicron-specific pseudo-virus. FIG. 7B, Footprint of SARS-COV-2 RBD-directed antibodies. The SARS-COV-2 Omicron RBD/hACE2 structure was downloaded from PDB 7T9K, approved or authorized antibodies are labeled. FIG. 7C, Schematic illustration of immunization and blood sample collection. Three humanized mice were repetitively immunized with Omicron LNP-mRNA as immunogen. 10 μg of Omicron LNP-mRNA were given for each mouse on day 0, day 2 and day 4 and day 7, and followed by 20 μg of Omicron LNP-mRNA were injected on day 11. Retro-orbital blood was collected on day 0, day 7 and day 14. Plasma was isolated from blood for downstream experiments. FIG. 7D, Anti-plasma titer determination. Upper panel, all plasma samples were serially 5-fold diluted from 1:1000 and assayed by a direct coating ELISA with Omicron RBD protein coated plate. Error bars represent mean±SEM of triplicates with individual data points in plots. Lower panel, all post-immunized plasma samples (2nd blood) were serially 5-fold diluted from 1:1000 and assayed by a direct coating ELISA with selected pan-CoV-RBD proteins coated plate, respectively. Error bars represent mean±SEM of triplicates with individual data points in plots. FIG. 7E, B cell characterization by customized scBCR-seq profiling. Top panel, Distribution of heavy chain complementarity-determining region 3 (HCDR3) length in each B cell group (Memory B, Plasma B and PBMC) from Omicron-RAMIHM mice. Bottom panel, distributions of heavy chain V-segment in each B cell group (Memory B, Plasma B and PBMC) from Omicron-RAMIHM mice. Total number of single cells sequenced with BCRs (Memory B library, n=2,646; Plasma B library, n=617; PBMC library, n=239; Total n=3,502). FIG. 1F, Ig class distributions of Omicron-RAMIHM mice's clonotypes. Distribution and frequency analysis of immunoglobulin isotypes usage in spleen, bone marrow and PBMC from Omicron-RAMIHM mice. Source data and additional statistics for experiments are in supplemental excel file(s).

FIG. 8A-FIG. 8C illustrate the identification of fully human Omicron-specific monoclonal antibodies against Omicron. FIG. 8A, Distribution of top 10 heavy- and light-chain V/J segment recombination. Chord diagrams (circos plots) showing the distribution of top 10 heavy- and light-chain V and J gene-segment recombination obtained in each representative library. Interconnecting lines indicate the relationship between antibodies that share V and J gene-segment at both IGH and IGL. Top to bottom: Memory B library, PBMC library, and Plasma B library. FIG. 8B, Single B cell variable chains for antibody cloning. Variable (V) genes and CDR3 lengths for paired heavy- and light-chains of top-enriched clones to SARS-CoV-2 Omicron from single BCR sequencing. FIG. 8C, ELISA of mAbs supernatant binding specificity against Omicron RBD protein. All full length mAb clones from single BCR sequencing and control were evaluated against Omicron RBD protein coated on the ELISA plate and binding activity was recorded at an optical density (OD) of 450 nm. Triplicate datapoints (n=3 each). Data are shown as mean±s.e.m. plus individual data points in dot plots. Statistics: One-way ANOVA was used to assess statistical significance. Each mAb clone was compared to control. Multiple testing correction was made to correct the p values. Two-sided tests were performed. The p-values are indicated in the plots. Statistical significance labels: *p<0.05; **p<0.01: ***p<0.001; ****p<0.0001. Non-significant comparisons are not shown, unless otherwise noted as n.s., not significant.

FIG. 9A-FIG. 9E illustrate the biophysical and functional characterization of lead clones of Omicron-specific antibodies. FIG. 9A, Schematic of human IgG1 mAb production.

FIG. 9B, SDS-PAGE analysis of purified mAbs under nonreducing and reducing (10 mM DTT) conditions. Four micrograms of purified protein were analyzed using a Novex WedgeWell 4-20% (wt/vol) Tirs-Glycine gel. FIG. 9C, Graph shows leading Omicron mAbs reactivity. The ELISA EC50 values were calculated by Prism V8.0 software using a four-parameter logistic curve fitting approach. Error bars represent mean±SEM of triplicates with individual data points in plots. FIG. 9D, Binding characteristics of the neutralizing mAbs determined by using BLI. Upper panel, recombinant SARS-COV-2 Omicron RBD were covalently immobilized onto a HIS1K sensor, all measurements were performed by using a serial 2-fold dilution of purified mAbs, starting from 50 nM (Top) to 1.56 nM (Bottom). Lower panel, purified mAbs were immobilized onto an AHC sensor, all measurements were performed by using a serial 2-fold dilution of soluble SARS-COV-2 Omicron RBD, starting from 50 nM (Top) to 1.56 nM (Bottom). Global fit curves are shown as red dashed lines, The vertical black dotted dashed lines indicate the transition between association and disassociation phases. FIG. 9E, Summary data of BLI results shown in FIG. 9D.

FIG. 10A-FIG. 10C illustrate additional functional characterization of lead clones of Omicron-specific antibodies. FIG. 10A, Neutralization assay of SARS-COV-2 Omicron pseudovirus by WA-1/Delta mAbs. Graph shows the normalized relative GFP signals for detection of 293T cells expressing hACE2, 24 h after infection with SARS-COV-2 Omicron pseudovirus, in the presence of increasing concentration of indicated WA-1/Delta mAbs. FIG. 10B, Neutralization assay of SARS-COV-2 Omicron pseudovirus by leading Omicron mAbs. Graph shows the normalized relative GFP signals for detection of 293T cells expressing hACE2, 24 h after infection with SARS-COV-2 Omicron pseudovirus, in the presence of increasing concentration of indicated Omicron mAbs. FIG. 10C, Neutralization assay of SARS-COV-2 Omicron pseudovirus by leading Omicron mAb combinations. Graph shows the normalized relative GFP signals for detection of 293T cells expressing hACE2, 24 h after infection with SARS-COV-2 Omicron pseudovirus, in the presence of increasing concentration of indicated Omicron mAb combinations (MB.02-MB.08, MB.08+PC.03, MB.02+PC.03). For FIG. 10A-FIG. 10C, the IC₅₀ values were calculated by Prism V8.0 software using a four-parameter logistic curve fitting approach. Dashed line indicated 50% reduction in viral infectivity. Error bars represent mean±SEM of triplicates with individual data points in plots.

FIG. 11A-FIG. 11C illustrate the characterization of Omicron-spike specific LNP-mRNA. FIG. 11A, Omicron LNP-mRNA image collected on transmission electron microscope. FIG. 11B, Dynamic light scattering derived histogram depicting the particle radius distribution of Omicron spike LNP-mRNA. FIG. 11C, Human ACE2 receptor binding of Omicron spike expressed in 293T cells as detected by human ACE2-Fc fusion protein and PE-anti-human Fc antibody on Flow cytometry.

FIG. 12A-FIG. 12C illustrate heatmaps for non-stochastic pair BCR repertoire. FIG. 12A, Memory B library heatmap, FIG. 12B, Plasma B library heatmap, and FIG. 12C, PBMC library heatmap, each showing the paired of immunoglobin heavy chains and light chains gene variable region segment of clonotypes in Omicron-RAMIHM mice. The reader color means the higher usage of specific VH-VL gene pairs.

FIG. 13A-FIG. 13C illustrate distribution of heavy- and light-chain V/J segment recombination. Chord diagrams (circos plots) showing the distribution of all heavy- and light-chain V and J gene-segment recombination obtained in Memory B library (FIG. 13A), PBMC library (FIG. 13B), and Plasma B library (FIG. 13C). Interconnecting lines indicate the relationship between antibodies that share V and J gene-segment at both IGH and IGL.

FIG. 14A-FIG. 14B illustrate clone screening for mAbs neutralization activity against Omicron pseudovirus. FIG. 14A, Gating strategy used for GFP-based neutralization analysis. FIG. 14B, mAbs supernatant neutralization curves in clone screening. Serial dilutions of all full length mAb clones from single BCR sequencing and control were added with Omicron pseudovirus-GFP to hACE2-O/E cells, and GFP expression was monitored and measured 24 hours after infection as a readout for virus infectivity. Data are graphed as percentage neutralization relative to virus-only infection control.

FIG. 15A-FIG. 15C illustrate epitope mapping through competitive binding measured by BLI. FIG. 15A, Schematic of epitope binning experiment. FIG. 15B, Summary data of BLI results shown in FIG. 15C. The matrix presents the concluded epitope specificity for each competition experiments. The columns indicate the primary loading antibody, and the rows indicate the secondary antibody combinations. FIG. 15C, Epitope binning of the three potent neutralizing mAbs. Sensorgrams showing distinct binding patterns when pairs of testing antibodies were sequentially applied to the recombinant SARS-COV2 Omicron RBD covalently immobilized onto a HIS1K sensor. The level of increment in response unit comparing with or without prior antibody incubation is the key criteria for determining the two mAbs recognize the separate or closely situated epitopes.

FIG. 16A-FIG. 16D illustrate ACE2 competition for binding to SARS-COV-2 Omicron RBD measured by ELISA. FIG. 16A-FIG. 16D, Curves show distinct binding patterns of ACE2 to SARS-COV-2 Omicron RBD with or without prior antibody incubation with each testing antibody (i.e., MB.02 clone (FIG. 16A), MB.08 clone (FIG. 16B), PB.03 clone (FIG. 16C), CR3022 control mAb (FIG. 16D)). The competition capacity of each antibody is indicated by the level of reduction in response unit of ACE comparing with or without prior antibody incubation. A commercial mAb CR3022 that binds to conserved region of spike was used as a control (FIG. 16D).

FIG. 17A-FIG. 17B illustrate a neutralization assay of leading Omicron mAbs with pseudotyped SARS-COV-2 variants. FIG. 17A, Individual neutralization curves for leading Omicron mAbs against Omicron pseudovirus. FIG. 17B, Individual neutralization curves for leading Omicron mAbs against Delta pseudovirus.

FIG. 18A-FIG. 18E illustrate the design, characterization and neutralization of bispecific Abs against pseudovirus of SARS-COV-2 variants of concern. FIG. 18A is a schematic overview and domain organization of the S proteins of SARS-COV-2 Omicron sublineages. Full-length mutated spike genes were used for production of pseudovirus. Specific mutations are colored compared with the sequences of SARS-COV-2 Omicron B.1.1529 lineage (purple, Omicron BA. 1 specific mutations; red, Omicron BA.1.1 specific mutations; orange, Omicron BA.2 specific mutations). FIG. 18B is a scheme of bispecific Ab design. Antibody domain are colored according to their architecture (purple, variable heavy chain; blue, variable light chain; red, variable heavy chain; yellow, variable light chain; deep grey, constant domain of the light chain; light gray, constant domain of the heavy chain). FIG. 18C shows SDS-PAGE analysis of affinity purified bispecific Abs. The antibody samples are analyzed under reducing conditions (+DTT). FIG. 18D shows ELISA binding curves of five human bispecific Abs against SARS-COV-2 WT, Delta, Omicron BA. 1 and BA.2 RBD proteins. The EC50 was determined by log (agonist) response of nonlinear regression and is displayed as the mean±s.e.m. FIG. 18E shows neutralization potency of lead bispecific Abs against SARS-COV-2 Delta and Omicron sublineages pseudovirus. The IC50 was determined by log (inhibitor) response of nonlinear regression and is displayed as the mean±s.e.m.

FIG. 19 illustrates Omicron BA.2 spike resistance against two out of three Omicron BA.1 RBD-directed monoclonal antibodies. ELISA binding curves of individual monoclonal Abs to Omicron sublineages RBD domains. The EC50 was determined by log (agonist) response of nonlinear regression and is displayed as the mean±s.e.m.

FIG. 20 shows ELISA binding curves of lead bispecific Abs compared with S309 against SARS-COV-2 WA-1, Delta, Omicron BA.1 and BA.2 RBD proteins.

FIG. 21 illustrates ACE2/RBD binding inhibition for potent lead bispecific Abs. Competition ELISA of lead bispecific Abs with human ACE2 for epitope identification to SARS-COV-2 WT, Delta, Omicron BA.1 and BA.2 RBD proteins. The data were obtained from a representative experiment with three replicates. Data are represented as mean±s.e.m. IC50 values were calculated from ACE2 competition ELISA assays by fitting a log (inhibitor) response of a nonlinear regression model.

FIG. 22 illustrates neutralization potency of three bispecific Abs against SARS-COV-2 Delta and Omicron sublineages pseudoviruses.

FIG. 23 shows binding affinity analysis of leading bispecific antibody to SARS-COV-2 Omicron BA.1 and BA.2 RBD proteins measured by BLI. His-tagged RBDs were immobilized on HIS1K biosensor and tested for real-time association and dissociation of the bsAbs. The colored lines are the actual operation curves, and the dashed lines are the fitted curves.

FIG. 24 shows representative flow gating plots.

### DETAILED DESCRIPTION OF THE INVENTION

The present invention is based in one non-limiting aspect on the discovery that monoclonal antibodies can be generated via the rapid mRNA immunization of a mammalian host followed by high-throughput RNA sequencing and screening to clone and identify antigen-specific antibody clones. Thus, in one aspect, the present invention provides a method of rapid antigen-specific antibody production, the method comprising (a) vaccinating a subject with an effective amount of a lipid nanoparticle comprising at least one mRNA encoding an antigen; (b) boosting the subject with at least one additional vaccination with an effective amount of the lipid nanoparticle comprising at least one mRNA encoding the antigen; (c) isolating B cells from the subject at least 14 days after the vaccination of step (a); (d) identifying and cloning BCR heavy and light chain pairs expressed by enriched B cells; and (e) screening the BCR heavy and light chains for antigen-specificity thereby identifying antigen-specific antibodies.

In another aspect, provided herein is a method for rapid identification of anti-RBD antibodies. In certain embodiments, the method comprises high-throughput single-cell B-cell receptor (BCR) sequencing from an animal (e.g., a mouse) immunized with SARS-COV-2 spike protein RBD. In some aspects, the SARS—Co-V-2 is wild-type SARS-COV-2. In some aspects, the SARS—Co-V-2 is a variant SARS-COV-2.

The invention also includes monoclonal antibodies or antigen-binding fragments or bispecific antibodies which are capable of binding a spike protein receptor binding domain (RBD) of a severe acute respiratory syndrome coronavirus 2 (SARS-COV-2)

In other aspects, provided herein are methods and uses of the anti-RBD antibodies or antigen-binding fragments or bispecific antibodies for neutralizing SARS-COV-2, for detecting or diagnosing SARS-COV-2, and/or for treating at least one sign and/or symptom of a SARS-CoV-2 infection (e.g., COVID-19). Related compositions (e.g., pharmaceutical compositions), kits, and diagnostic methods are also provided.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting. Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “about” as used herein can allow for a degree of variability in a value or range, for example, within 10%, within 5%, or within 1% of a stated value or of a stated limit of a range, and includes the exact stated value or range.

The term “antibody” or “Ab” or “immunoglobulin” are terms of art and can be used interchangeably and refer to a protein, or polypeptide sequence which is or is derived from an immunoglobulin molecule having at least one antigen binding site which specifically binds to a specific epitope on an antigen (See, e.g., Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, hetero-conjugate antibodies, single domain antibodies, monovalent antibodies, single chain antibodies or single chain Fv (scFv), nanobodies, intracellular antibodies, intrabodies, camelized antibodies, camelid antibodies, IgNAR antibodies, affybodies, Fab fragments, F(ab′) fragments, F(ab)₂, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. Full-length antibodies are typically tetramers comprising two heavy chain and two light chain immunoglobulin molecules.

As used herein, the term “monoclonal antibody” refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., a hybridoma or host cell producing a recombinant antibody), wherein the antibody binds to a coronavirus spike protein epitope (e.g., an epitope of a SARS-COV-2 spike protein receptor binding domain (RBD)) as determined, e.g., by ELISA or other antigen-binding or competitive binding assay known in the art or in the Examples provided herein. In particular embodiments, a monoclonal antibody can be a chimeric antibody, a human antibody, or a humanized antibody. Methods for generating a humanized antibody are known in the art. In certain embodiments, a monoclonal antibody is a monovalent antibody or multivalent (e.g., bivalent) antibody. In particular embodiments, a monoclonal antibody is a monospecific or multi-specific antibody (e.g., bispecific antibody). Monoclonal antibodies described herein can, for example, can be made by the hybridoma method as described in Kohler et al.; Nature, 256:495 (1975) or can be isolated from phage libraries, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Monoclonal antibodies may be identified by high-throughput direct sequencing of fully recombined VDJ sequences of B cell receptor (BCR) repertoires from single cells of animals immunized with an antigen for which the desired monoclonal antibody will specifically bind as described herein. See, e.g. Goldstein et al., Communications Biology (2019) 2:304; Horns et al., Cell Reports (2020) 30:905-913). The identified monoclonal antibodies are then produced recombinantly.

The terms “antibody fragment,” “antigen-binding fragment,” and “antigen-binding domain” of an antibody and similar terms are used interchangeably and refer to at least one portion of an intact antibody, or recombinant variants thereof, and comprising or consisting of the antigen-binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), VHH domains, and multi-specific (e.g., bispecific) antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (K) and lambda (2) light chains refer to the two major antibody light chain isotypes.

As used herein, the term “constant region” or “constant domain” refers to an antibody portion, e.g., a carboxyl terminal portion of a light and/or heavy chain which is not directly involved in binding of an antibody to antigen but which can exhibit various effector functions, such as interaction with the Fe receptor. The terms refer to a portion of an immunoglobulin molecule having a generally more conserved amino acid sequence relative to an immunoglobulin variable domain.

As used herein, the terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs).

By the term “recombinant antibody” as used herein, is meant an antibody which is generated using recombinant DNA technology, such as, for example, an antibody expressed by a bacteriophage, insect, or yeast expression system or by a human cell line expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA or mRNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized, or can be derived from a biological sample. Such a biological sample can include, but is not limited to a tissue sample, a tumor sample, a cell, a virus, or a biological fluid.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope can be determined by structural methods, e.g., X-ray diffraction crystallography, nuclear magnetic resonance (NMR), or electron microscopy (e.g., negative stain or cryo-EM), ELISA assays, hydrogen/deuterium exchange coupled with mass spectrometry (e.g., MALDI mass spectrometry), array-based oligo-peptide scanning assays, and/or mutagenesis mapping (e.g., site-directed mutagenesis mapping). In a specific embodiment, the epitope of an antibody or antigen-binding fragment is determined using cryo-EM studies, such as described herein.

As used herein, the term “conservative sequence modifications” is intended to refer to nucleotide or amino acid modifications that do not change the amino acid sequence or significantly affect or alter the binding characteristics of the antibody containing the amino acid sequence, respectively. Amino acid conservative modifications include amino acid substitutions, additions, and deletions. Modifications can be introduced into an antibody of the disclosure by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine), and aromatic side chains (e.g., tyrosine, phenylalanine, tryptophan, histidine). Thus, one or more amino acid residues within the complementarity-determining regions (CDRs) of an antibody can be replaced with other amino acid residues from the same side chain family and the altered antibody can be tested for the ability to bind antigens using the functional assays described herein.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}). Affinity can be measured and/or expressed in a number of ways known in the art, including, but not limited to, equilibrium dissociation constant (K_{D}), equilibrium association constant (K_{A}), and IC50. The K_{D} is calculated from the quotient of k_{off}/kₒₙ, whereas K_{A} is calculated from the quotient of kₒₙ/k_{off}, where kₒₙ refers to the association rate constant of, e.g., an antibody to an antigen, and k_{off} refers to the dissociation rate constant of, e.g., an antibody to an antigen.

The term “avidity” as used herein refers to the total binding strength of an antibody for an antigen at every binding site.

As used herein, a “neutralizing” antibody or antigen-binding fragment is an antibody that defends a cell from a pathogen or infectious particle such as a virus by neutralizing any effect it has biologically. Neutralization renders the particle no longer infectious or pathogenic. By binding specifically to surface structures (antigen) on an infectious particle, neutralizing antibodies prevent the particle from interacting with its host cells it might otherwise infect and destroy. Neutralizing antibodies can inhibit the infectivity of the pathogen by binding to the pathogen and blocking the molecules needed for cell entry. This can be due to the antibodies sterically interfering with the pathogens or toxins attaching to host cell receptors. In case of a virus infection, neutralizing antibodies can bind, e.g., to glycoproteins of enveloped viruses or capsid proteins of non-enveloped viruses. Furthermore, neutralizing antibodies can act by preventing particles from undergoing structural changes often needed for successful cell entry. For example, neutralizing antibodies can prevent conformational changes of viral proteins that mediate the membrane fusion needed for entry into the host cell. In some cases, the virus is unable to infect even after the antibody dissociates. The pathogen-antibody complex is eventually taken up and degraded by host macrophages.

The term “complementary” as used herein refers to the broad concept of subunit sequence complementarity between two nucleic acids, e.g., two DNA molecules. When a nucleotide position in both of the molecules is occupied by nucleotides normally capable of base pairing with each other, the nucleic acids are considered to be complementary to each other at this position. Thus, two nucleic acids are substantially complementary to each other when at least 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A “condition” is a state of health, whether well or ill, and may include, for example, the state of having an infection (e.g., a viral, bacterial, or fungal infection) whether or not the animal has noticeable or detectable symptoms of the infection.

A disease, disorder, or condition is “alleviated” or “ameliorated” if the severity or frequency of at least one sign or symptom of the disease or disorder experienced by a subject is reduced and/or eliminated.

The terms “treat,” “treating,” and “treatment,” refer to one or more therapeutic or palliative measures described herein. The methods of “treatment” employ administration of a composition to a subject in need of such treatment, for example, a subject afflicted with a disease or disorder or condition, or a subject who has one or more signs or symptoms of such a disease or disorder or condition, in order to cure, delay, reduce the severity of, alleviate, or ameliorate one or more signs or symptoms of the disease, disorder or condition, or in order to prolong the survival of a subject beyond that expected in the absence of such treatment.

The term “inhibit” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists. As used herein, the term “metabolite” refers to an intermediate or end product of a metabolic process, including, but not limited to, hydrolysis, esterification, conjugation, oxidation, and reduction.

As used herein, the term “mutation” is a change in a DNA sequence resulting in an alteration from its natural, original, or previous state. The mutation can comprise deletion and/or insertion and/or duplication and/or substitution of at least one deoxyribonucleic acid base such as a purine (adenine and/or guanine) and/or a pyrimidine (thymine and/or cytosine). Mutations may or may not produce discernible changes in the observable characteristics (phenotype) of an organism.

As used herein, the term “immunogenicity” refers to the ability of a particular substance, such as an antigen or epitope, to provoke an immune response in the body of a mammal. This immune response could be humoral and/or cell-mediated.

As used herein, the terms “peptide,” “polypeptide,” and “protein” are used interchangeably, and refer to a compound comprised of amino acid residues covalently linked by peptide bonds, having an N-terminus and a C-terminus. A protein or peptide must contain at least two amino acids, and no limitation is placed on the maximum number of amino acids that may comprise a protein or peptide's sequence. Polypeptides include any peptide comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. Proteins may comprise more than one polypeptide chain. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

The terms “pharmaceutically effective amount” and “effective amount” refer to a non-toxic but sufficient amount of an agent to provide the desired biological result. That result can be reduction and/or alleviation of the signs, symptoms, or causes of a disease or disorder, or any other desired alteration of a biological system.

As used herein, the terms “RT-PCR” or “reverse transcription polymerase chain reaction” refer to a laboratory technique combining reverse transcription of the RNA present in a sample to DNA, with amplification of specific DNA targets using the polymerase chain reaction. These terms may also refer to real time PCR, wherein the amplification of the DNA target is monitored and quantified by at least one of several detection methods, such methods comprising nonspecific fluorescent dye intercalation with DNA and sequence-specific DNA probes consisting of oligonucleotides labeled with a fluorescent reporter, wherein fluorescence is detected only upon hybridization of the probe with its complementary sequence.

The term “SARS-COV-2” refers to the severe acute respiratory syndrome coronavirus 2, one of a family of human coronaviruses, which was initially identified in Wuhan, China in December, 2019. This initially-identified SARS-COV-2 virus is referred to herein as “wild-type” or “WT” SARS-COV-2. Numerous genetic variants of SARS-COV-2 have since emerged and have been identified through sequence-based surveillance, laboratory studies, and epidemiological investigations. These SARS-COV-2 genetic variants include, but are not limited to, the B.1.1.7 variant (also termed the “UK variant” or “Alpha variant”), the B.1.351 variant (also termed the “South African variant” or “Beta variant”), the P.1 variant (also termed “Gamma variant”), the B. 1.427 variant, the B. 1.429 variant, the B.1.526 variant, the B. 1.526.1 variant, the B.1.525 variant, the P.2 variant, the B.1.617 variant (“Indian variant”), the cluster 5/ΔFVI-spike variant (“Denmark Mink variant”), the B.1.526 variant (“New York variant”), the B.1.427 and B.1.429 variants (“California variants”), the BV-1 variant (“Texas variant”), the B.1.617.2 variant (“Delta variant”), B.1.1.529 (“Omicron variant”) and sublineages thereof (e.g., BA.1, BA.1.1, BA.2, BA.3, BA.4, and BA.5). SARS-COV-2 variants contain mutations relative to wild-type SARS-COV-2. For example, the L452R spike protein mutation is present in the B.1.526.1, B.1.427, and B.1.429 variants and the E484K spike protein mutation is present in B.1.525, P.2, P.1, and B.1.351 variants, but only in some strains of B.1.526 and B.1.1.7 variants.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “subject” or “patient” or “individual” for the purposes of the present disclosure includes humans and other animals, particularly mammals, and other organisms. Non-human mammals include, for example, livestock and pets, non-human simian, ovine, bovine, porcine, canine, feline and murine mammals. Preferably, the subject is human. Thus the methods are applicable to both human therapy and veterinary applications.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Rapid Generation of Monoclonal Antibodies by mRNA Vaccination

The rapid evolution of the SARS-COV-2 coronavirus has highlighted a pressing need in biotechnology and clinical medicine for methods of rapidly generating monoclonal antibodies specific for novel antigens capable of being used for testing and therapeutic use. While the first generation of SARS-COV-2 therapies included monoclonal antibodies generated from patent-derived antisera or more traditional methods have been useful for treating patients, each successive wave of SARS-COV-2 variant evolution and spread (e.g. Delta and Omicron) has resulted in lower efficacy for these products. As such, the current invention includes a method of rapidly producing highly-specific monoclonal antibodies. This method takes advantage of immunizing humanized hosts with multiple vaccinations of relatively high-dose mRNA encoding the antigen of interest, e.g. the RBD domain of the SARS-COV-2 spike protein.

As such, in one aspect, the invention provides a method of identifying antigen-specific antibodies (i.e., a method of rapid antigen-specific antibody production). In certain embodiments, the method comprises providing a lipid nanoparticle comprising at least one mRNA encoding an antigen. In certain embodiments, the method comprises vaccinating a subject with an effective amount of the nanoparticle. In certain embodiments, the method comprises boosting the subject with at least one additional vaccination with an effective amount of the nanoparticle. In certain embodiments, the method comprises isolating B cells from the subject after vaccination. In certain embodiments, the method comprises identifying BCR heavy and light chain pairs expressed by the B cells which are enriched. In certain embodiments, the method comprises cloning the BCR heavy and light chains. In certain embodiments, the method comprises screening the BCR heavy and light chains for antigen-specificity thereby identifying antigen-specific antibodies. In some embodiments, isolating B cells from the subject is performed at least about 14 days after the vaccination of step a. A person of skill in the art will realize that this method is not specific to the antigen and that the method is applicable to any antigen which can be encoded by mRNA.

In certain embodiments, the vaccines used to immunize and the subjects are comprised of lipid nanoparticles formulated with mRNA encoding the antigen of interest (e.g. the SARS-COV-2 spike protein). Lipid nanoparticles (LNPs), as used herein, refer to lipid vesicles that possess a homogenous lipid core. LNPs have proved useful for the delivery of small molecules and labile molecules such as RNA which are otherwise difficult to deliver across the plasma membrane into cells. Notably, the two most successful mRNA-based SARS-COV-2 vaccines, mRNA-1273 and BNT126b are formulated within lipid nanoparticles. In certain embodiments, the lipid nanoparticles used in the invention can comprise more than one type of RNA, encoding more than one epitope or protein. In this way, one or more antigens can be used to immunize the subject simultaneously.

In certain embodiments, the additional or booster vaccinations occur two days, three days, four days, five days, six days, seven days, eight days, nine days, ten days, and eleven days after the initial vaccination. In one specific embodiment, the booster vaccinations occur two days, four days, seven days, and eleven days after the initial vaccination.

In certain embodiments, the B cells from the vaccinated subject are isolated at least 14 days after the initial priming vaccination. This time is sufficient for B cell priming, class switching, and affinity maturation to result in the generation of antigen-specific IgG, IgA, and IgE isotype antibodies. Isolation and purification of various B cell populations from the host can be accomplished by a number of different techniques known in the art including density gradient centrifugation and methods using antibodies against B cell surface antigens such as CD45/B220, CD138, and the like. These methods include but are not limited to labeling B cells with antibodies conjugated to magnetic beads and passed over a column and magnet to achieve positive selection, or antibodies conjugated to fluorochromes which allow for sorting by flow cytometry. It is contemplated that any method of sorting or purifying B cells or subpopulations of B cells known in the art could be used in methods of the current invention.

In certain embodiments, mRNA is isolated from the B cells and sequenced in order to identify BCR heavy and light chain pairs is by single-cell BCR sequencing (scBCRseq). In certain embodiments, this mRNA is converted to cDNA which is then amplified for BCR sequences before subjected to high-throughput sequencing to identify heavy and light chain pairs for further cloning and screening. Single-cell BCR sequencing is described, for example, in Goldstein et al., 2019; Horns et al., 2020, Corti et al., 2016; and Setliff et al., 2019.

In order to provide antibodies suitable for immediate use in human patients, certain embodiments of the invention use humanized mammal subjects including but not limited to mice, rats, or other rodents. These subjects are transgenic for human immune genes, including those that encode the immunoglobulin loci. In certain embodiments, the subject is a humanized or transgenic mouse which comprises human IgG and IgK transgenics. In this way, immunization of these mice results in antigen-specific human antibodies. The generation of human or “fully-human” antibodies directly is preferable to the use of non-human antibody isotypes and the “humanization” of non-human antibodies, which involves the cloning of CDRs or variable regions onto human constant region or framework region backbone. Non-human and humanized antibodies are more likely to result in anti-isotype or anti-idiotype antibodies generated by treated patients immune systems. Thus in certain embodiments, the immunized subject is a transgenic mouse.

The method may be used for rapid identification and production of a monoclonal antibody or antigen-binding fragment which is capable of binding a spike protein receptor binding domain (RBD) of a severe acute respiratory syndrome coronavirus 2 (SARS-COV-2), such as the antibodies and antigen-binding fragments and bispecific antibodies described herein

### Method for Identifying Monoclonal Antibodies

Further provided herein is a method for rapid identification of a monoclonal antibody or antigen-binding fragment which is capable of binding a spike protein receptor binding domain (RBD) of a severe acute respiratory syndrome coronavirus 2 (SARS-COV-2), such as the antibodies and antigen-binding fragments and bispecific antibodies described herein.

In certain embodiments, the method comprises a. immunizing an animal with a polypeptide comprising a SARS-COV-2 spike protein RBD or with a nucleic acid comprising a nucleotide sequence encoding the spike protein RBD; b. sequencing fully recombined VDJ sequences of B cell receptor (BCR) repertoires from multiple individual B cells of the animal, thereby generating sequencing data; c. identifying candidate antibody clones from the sequencing data; d. producing a recombinant monoclonal antibody or antigen-binding fragment comprising paired heavy and light chain variable regions from one or more candidate antibody clones; and e. testing the produced antibody or antigen-binding fragment for an ability to bind the spike protein RBD.

In certain embodiments, the sequencing is high-throughput single-cell B-cell receptor sequencing (scBCR-seq) as described, for example, in Goldstein et al., 2019; Horns et al., 2020, Corti et al., 2016; and Setliff et al., 2019.

Any mammalian animal may be immunized, such as a mouse, a rat, a rabbit, a hamster, or a monkey. In some embodiments, the animal is a mouse. In some embodiments, the animal is a C57BL/6J mouse or a BALB/c mouse.

In some embodiments, the multiple individual B cells are isolated progenitor B cells and/or plasma B cells from spleen, lymph node, and/or bone marrow of the animal.

In certain embodiments, the SARS-COV-2 is wild type SARS-COV-2. In certain embodiments, the SARS-COV-2 is a variant SARS-COV-2. In certain embodiments, the SARS-CoV-2 is B.1.351 SARS-COV-2 variant.

In some embodiments, the produced antibody or antigen-binding fragment binds the RBD with nanomolar affinity. In some embodiments, the produced antibody or antigen-binding fragment binds the RBD with picomolar avidity. In some embodiments, the produced antibody or antigen-binding fragment has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 pM, less than 20 pM, less than 10 pM, less than 5 pM, or less than 2.5 pM. In some embodiments, the produced antibody or antigen-binding fragment specifically binds the SARS-COV-2 spike protein RBD.

In certain embodiments, the produced antibody or antigen-binding fragment binds an epitope of the RBD which partially or completely overlaps with the angiotensin converting enzyme II (ACE2) binding site of RBD, thereby preventing RBD binding to ACE2.

In certain embodiments, the produced antibody or antigen-binding fragment is a neutralizing antibody or antigen-binding fragment.

In certain embodiments, the produced antibody or antigen-binding fragment is effective against lethal challenge of SARS-COV-2 in vivo.

In certain embodiments, the produced antibody or antigen-binding fragment is selected from the group consisting of a monospecific antibody, a bispecific antibody, an Fab, an Fab′, an F(ab′)₂, an Fv, an scFv, a linear antibody, and a single domain antibody (sdAb).

In some embodiments, the antibody or antigen-binding fragment is a bispecific antibody selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb). In some embodiments, the bispecific antibody is an asymmetric IgG-like bispecific antibody which binds two distinct epitopes of the RBD.

In some embodiments, the produced antibody or antigen-binding fragment is conjugated or recombinantly fused to at least one other moiety or molecule selected from the group consisting of a diagnostic agent, a detectable agent, a therapeutic agent, a purification tag, a molecule affecting one or more biological or molecular properties of the antibody or antigen-binding domain, and any combination thereof, wherein the biological or molecular properties comprise serum stability, half-life, solubility, and antigenicity

### Antibody Treatment of SARS-COV-2

The emergence of the highly pathogenic coronavirus SARS-COV-2 in 2019 and its rapid international spread has posed a serious on-going global public-health emergency. In late 2021, the Omicron variant (B.1.1.529) of SARS-COV-2 emerged to take over the viral landscape in US and rapidly spread across Europe and many other countries of the world. The speed with which new variants of SARS-COV-2 emerge and spread poses an ever growing threat to efforts to contain and minimize the impact of the virus. Therefore, it is critical to rapidly develop countermeasures such as new monoclonal antibodies.

The Omicron variant has a total of 60 mutations compared to the reference virus (ancestral variant, or “wild type”), of which most are new and not observed in any other variants such as Alpha, Beta, Kappa, or Delta. Importantly, 32 of those mutations are in the spike protein, of which 15 are located in the receptor-binding domain (RBD). These mutations bring multiple mechanisms of immune evasion of the virus, negating the effect of virtually all existing antibodies and vaccines that have been developed against the ancestral WT virus. Over the past two months, since the report of the first incidence in South Africa, the scientific community moved rapidly to study this new variant. A number of recent publications have highlighted a set of critical findings: (1) Omicron dampens all existing vaccines, including mRNA vaccine from Pfizer/BioTech and Moderna, inactivated vaccines and protein subunit vaccines, and (2) Omicron renders the vast majority of existing antibody therapies ineffective. These antibodies either being completely lost their neutralization activity, or have drastic drops of efficacy (100 to 10,000 fold). In fact, such levels of activity drop will fail to provide treatment benefit, including those antibodies approved or under emergency use authorization for clinical usage against Omicron infected COVID-19 patients.

Coronaviruses, including SARS-COV-2, use the homotrimeric spike protein on the outer surface of the virion envelope to bind to their cellular receptors, which for SARS-COV-2 in humans is angiotensin converting enzyme 2 or ACE2. The binding of spike protein to receptor triggers a cascade of events that leads to the fusion between cell and viral membranes and entry of viral proteins and nucleic acids into the host cell. Therefore, binding to the ACE2 receptor is a critical initial step for SARS-COV to enter into target cells. The receptor binding domain or RBD of the spike protein is the domain which is responsible for directly interacting with receptor proteins (e.g. ACE2). As such, the RBD has been the target of vaccine and monoclonal antibody development, as antibody blockade of this domain has proven effective in reducing or preventing viral cell entry. This strategy is informed and reinforced by the observation that most natural, productive antibody responses observed in human patients are against RBD epitopes. Unfortunately, the spike protein and the RBD in particular are capable of considerable mutational plasticity without losing ACE2 binding function, resulting in rapid viral antigenic drift which contributes to the relatively quick loss of efficacy of both endogenous immune responses and commercially developed vaccine and monoclonal antibody treatments.

In one aspect, the invention provides a monoclonal antibody or antigen-binding fragment which is capable of binding a severe acute respiratory syndrome coronavirus 2 (SARS-COV-2) spike protein receptor binding domain (RBD). In another aspect, the invention provides a bispecific antibody which is capable of binding a severe acute respiratory syndrome coronavirus 2 (SARS-COV-2) spike protein receptor binding domain (RBD). In some embodiments, the SARS—Co-V-2 is wild-type SARS-COV-2. In some embodiments, the SARS—Co-V-2 is a variant SARS-COV-2.

Numerous genetic variants of SARS-COV-2 have emerged in various locations worldwide and have been identified by the scientific and medical communities through sequence-based surveillance, laboratory studies, and epidemiological investigations. These SARS-COV-2 genetic variants include, but are not limited to, the B.1.1.7 variant (also termed the “UK variant” or “Alpha variant”), the B.1.351 variant (also termed the “South African variant” or “Beta variant”), the P.1 variant (also termed “Gamma variant”), the B.1.427 variant, the B.1.429 variant, the B.1.526 variant, the B.1.526. 1 variant, the B. 1.525 variant, the P.2 variant, the B.1.617 variant (“Indian variant”), the cluster 5/ΔFVI-spike variant (“Denmark Mink variant”), the B.1.526 variant (“New York variant”), the B.1.427 and B. 1.429 variants (“California variants”), the BV-1 variant (“Texas variant”), the B.1.617.2 variant (“Delta variant”), B.1.1.529 (“Omicron variant”) and sublineages thereof (e.g., BA.1, BA.1.1, BA.2, BA.3, BA.4, and BA.5). SARS-COV-2 variants contain mutations relative to wild-type SARS-CoV-2. For example, the L452R spike protein mutation is present in the B.1.526.1, B.1.427, and B.1.429 variants and the E484K spike protein mutation is present in B.1.525, P.2, P.1, and B.1.351 variants, but only in some strains of B.1.526 and B. 1.1.7 variants. In certain embodiments, the SARS-COV-2 is selected from a beta variant, a delta variant, an omicron variant, or any sublineage thereof. In certain embodiments, the SARS-COV-2 is selected from the group consisting of Omicron BA.1, BA1.1 and BA.2.

In certain embodiments, the antibody or antigen-binding fragment or bispecific antibody binds the RBD with nanomolar affinity. In certain embodiments, the antibody or antigen-binding fragment or bispecific antibody binds the RBD with picomolar avidity. In certain embodiments, the antibody or antigen-binding fragment or bispecific antibody has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 pM, less than 20 pM, less than 10 pM, less than 5 pM, or less than 2.5 pM. In certain embodiments, the antibody or antigen-binding fragment or bispecific antibody specifically binds the SARS-COV-2 spike protein RBD. Any method known in the art may be used to determine affinity, avidity, dissociation constant, and specific binding of the antibody or antigen-binding fragment for the SARS-COV-2 spike protein RBD. Various known methods, assays, and instrumentation include, but are not limited to, surface plasmon resonance (SPR) (e.g., BioCore SPR, GE Healthcare), bio-layer interferometry (BLI) (e.g., Octet BLI), ELISA, EMSA, co-purification, fluorescence polarization, and competition assays.

In certain embodiments, the antibody or antigen-binding fragment or bispecific antibody binds an epitope of the RBD which partially or completely overlaps with the angiotensin converting enzyme II (ACE2) binding site of RBD, thereby preventing RBD binding to ACE2.

In certain embodiments, the antibody or antigen-binding fragment or bispecific antibody is a neutralizing antibody or antigen-binding fragment or bispecific antibody.

In certain embodiments, the antibody or antigen-binding fragment or bispecific antibody is effective against lethal challenge of SARS-COV-2 in vivo, such as in a mouse or a human infected with SARS-COV-2.

The antibody or antigen-binding fragment or bispecific antibody of the invention may be any type of full-length antibody or antigen-binding fragment known in the art. Various examples of antibodies and antigen-binding fragments include, but are not limited to, monospecific antibodies, multi-specific antibodies (e.g., bispecific antibodies), Fab, Fab′, F(ab′)₂, Fv, scFv, linear antibodies, and single domain antibodies (sdAb). In certain embodiments, the antibody is a monospecific antibody. In certain embodiments, the antibody is a bispecific antibody. In certain embodiments, the bispecific antibody is selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb). In certain embodiments, the bispecific antibody is an asymmetric IgG-like bispecific antibody which binds two distinct epitopes of the RBD.

Various bispecific antibody configurations are known in the art and include, for example, IgG-like asymmetric bispecific antibodies, bispecific T cell engagers (BITEs), Fc-fused BITEs (BITE-Fc), dual-affinity re-targeting proteins (DARTs), and Fc-fused DARTs (DART-Fc) (see, e.g., Wang, et al., Antibodies (2019), 8:43). In certain embodiments, the bispecific antibody is an IgG-like asymmetric bispecific antibody. There are mainly two problems that must be solved to produce the IgG-like asymmetric bispecific antibody—the heterodimerization of two different heavy chains and the discrimination between the two light-chain/heavy-chain interactions. Judicious genetic and cellular engineering strategies, such as quadroma technology, common heavy chain and common light-chain strategies, knobs-into-holes, CrossMab, and co-culture methods, are known in the art and have been implemented to produce optimized Y-shape IgG-like bispecific antibodies. These, or any other known method can be used to generate the IgG-like bispecific antibodies disclosed herein. In certain aspects, a method which produces a monoclonal bispecific antibody, such as knobs-into-holes with CrossMab technology, is used.

In certain embodiments, the antibody or antigen-binding fragment comprises at least one heavy chain variable region (VH) and at least one light chain variable region (VL). For example, a monospecific antibody generally comprises two copies of VH and two copies of VL. Each VH and each VL comprises three complementarity determining regions (CDRs). The three VH CDRs are referred to herein as HCDR1, HCDR2, and HCDR3 whereas the three VL CDRs are referred to herein as LCDR1, LCDR2, and LCDR3. Various methods are known in the art for determining CDR amino acid sequences of an antibody.

In certain aspects, the CDRs of an antibody can be determined according to the Kabat numbering system (see, e.g., Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

In certain aspects, the CDRs of an antibody can be determined according to the Chothia numbering scheme, which refers to the location of immunoglobulin structural loops (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDRLI loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, the CDRs of an antibody can be determined according to the IMGT numbering system as described in Lefranc, M.-P., 1999, The Immunologist, 7:132-136 and Lefranc, M.-P. et al., 1999, Nucleic Acids Res., 27:209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In certain aspects, the CDRs of an antibody can be determined according to MacCallum et al., 1996, J. Mol. Biol., 262:732-745. See also, e.g., Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001).

In certain aspects, the CDRs of an antibody can be determined according to the AbM numbering scheme, which refers AbM hypervariable regions which represent a compromise between the Kabat CDRs and Chothia structural loops, and are used by Oxford Molecular's AbM antibody modeling software.

In certain embodiments, the antibody or antigen-binding fragment comprises three heavy chain complementarity determining regions (HCDR1, HCDR2, and HCDR3) and three light chain complementarity determining regions (LCDR1, LCDR2, and LCDR3) selected from the group consisting of:
a. an HCDR1 comprising SEQ ID NO: 1, an HCDR2 comprising SEQ ID NO: 2, an HCDR3 comprising SEQ ID NO: 3, an LCDR1 comprising SEQ ID NO: 4, an LCDR2 comprising the amino acid sequence AAS, and an LCDR3 comprising SEQ ID NO: 6;
b. an HCDR1 comprising SEQ ID NO: 11, an HCDR2 comprising SEQ ID NO: 12, an HCDR3 comprising SEQ ID NO: 13, an LCDR1 comprising SEQ ID NO: 14, an LCDR2 comprising the amino acid sequence GSS, and an LCDR3 comprising SEQ ID NO: 16;
c. an HCDR1 comprising SEQ ID NO: 21, an HCDR2 comprising SEQ ID NO: 22, an HCDR3 comprising SEQ ID NO: 23, an LCDR1 comprising SEQ ID NO: 24, an LCDR2 comprising the amino acid sequence AAS, and an LCDR3 comprising SEQ ID NO: 26;
d. an HCDR1 comprising SEQ ID NO: 31, an HCDR2 comprising SEQ ID NO: 32, an HCDR3 comprising SEQ ID NO: 33, an LCDR1 comprising SEQ ID NO: 34, an LCDR2 comprising the amino acid sequence AAS, and an LCDR3 comprising SEQ ID NO: 36;
e. an HCDR1 comprising SEQ ID NO: 107, an HCDR2 comprising SEQ ID NO: 108, an HCDR3 comprising SEQ ID NO: 109, an LCDR1 comprising SEQ ID NO: 116, an LCDR2 comprising the amino acid sequence GAS, and an LCDR3 comprising SEQ ID NO: 118;
f. an HCDR1 comprising SEQ ID NO: 110, an HCDR2 comprising SEQ ID NO: 111, an HCDR3 comprising SEQ ID NO: 112, an LCDR1 comprising SEQ ID NO: 119, an LCDR2 comprising the amino acid sequence WAS, and an LCDR3 comprising SEQ ID NO: 121; and
g. an HCDR1 comprising SEQ ID NO: 113, an HCDR2 comprising SEQ ID NO: 114, an HCDR3 comprising SEQ ID NO: 115, an LCDR1 comprising SEQ ID NO: 122, an LCDR2 comprising the amino acid sequence DAS, and an LCDR3 comprising SEQ ID NO: 124.

In certain embodiments, the antibody or antigen-binding fragment comprises at least one heavy chain variable region (VH) and at least one light chain variable region (VL) selected from the group consisting of:
a. a VH comprising an amino acid sequence at least 80%, 85%, 90%, 95%, 96%, 97%, 98%, 99%, or 100% identical to SEQ ID NO: 7, and a VL comprising an amino acid sequence at least 80%, 85%, 90%, 95%, 96%, 97%, 98%, 99%, or 100% identical to SEQ ID NO: 8;
b. a VH comprising an amino acid sequence at least 80%, 85%, 90%, 95%, 96%, 97%, 98%, 99%, or 100% identical to SEQ ID NO: 17, and a VL comprising an amino acid sequence at least 80%, 85%, 90%, 95%, 96%, 97%, 98%, 99%, or 100% identical to SEQ ID NO: 18;
c. a VH comprising an amino acid sequence at least 80%, 85%, 90%, 95%, 96%, 97%, 98%, 99%, or 100% identical to SEQ ID NO: 27, and a VL comprising an amino acid sequence at least 80%, 85%, 90%, 95%, 96%, 97%, 98%, 99%, or 100% identical to SEQ ID NO: 28;
d. a VH comprising an amino acid sequence at least 80%, 85%, 90%, 95%, 96%, 97%, 98%, 99%, or 100% identical to SEQ ID NO: 37, and a VL comprising an amino acid sequence at least 80%, 85%, 90%, 95%, 96%, 97%, 98%, 99%, or 100% identical to SEQ ID NO: 38;
e. a VH comprising an amino acid sequence at least 80%, 85%, 90%, 95%, 96%, 97%, 98%, 99%, or 100% identical to SEQ ID NO: 101, and a VL comprising an amino acid sequence at least 80%, 85%, 90%, 95%, 96%, 97%, 98%, 99%, or 100% identical to SEQ ID NO: 104;
f. a VH comprising an amino acid sequence at least 80%, 85%, 90%, 95%, 96%, 97%, 98%, 99%, or 100% identical to SEQ ID NO: 102, and a VL comprising an amino acid sequence at least 80%, 85%, 90%, 95%, 96%, 97%, 98%, 99%, or 100% identical to SEQ ID NO: 105; and
g. a VH comprising an amino acid sequence at least 80%, 85%, 90%, 95%, 96%, 97%, 98%, 99%, or 100% identical to SEQ ID NO: 103, and a VL comprising an amino acid sequence at least 80%, 85%, 90%, 95%, 96%, 97%, 98%, 99%, or 100% identical to SEQ ID NO: 106.

In certain embodiments, the antibody comprises at least one heavy chain and at least one light chain selected from the group consisting of:
a. a heavy chain comprising an amino acid sequence at least 80%, 85%, 90%, 95%, 96%, 97%, 98%, 99%, or 100% identical to SEQ ID NO: 9, and a light chain comprising an amino acid sequence at least 80%, 85%, 90%, 95%, 96%, 97%, 98%, 99%, or 100% identical to SEQ ID NO: 10;
b. a heavy chain comprising an amino acid sequence at least 80%, 85%, 90%, 95%, 96%, 97%, 98%, 99%, or 100% identical to SEQ ID NO: 19, and a light chain comprising an amino acid sequence at least 80%, 85%, 90%, 95%, 96%, 97%, 98%, 99%, or 100% identical to SEQ ID NO: 20;
c. a heavy chain comprising an amino acid sequence at least 80%, 85%, 90%, 95%, 96%, 97%, 98%, 99%, or 100% identical to SEQ ID NO: 29, and a light chain comprising an amino acid sequence at least 80%, 85%, 90%, 95%, 96%, 97%, 98%, 99%, or 100% identical to SEQ ID NO: 30;
d. a heavy chain comprising an amino acid sequence at least 80%, 85%, 90%, 95%, 96%, 97%, 98%, 99%, or 100% identical to SEQ ID NO: 39, and a light chain comprising an amino acid sequence at least 80%, 85%, 90%, 95%, 96%, 97%, 98%, 99%, or 100% identical to SEQ ID NO: 40;
e. a heavy chain comprising an amino acid sequence at least 80%, 85%, 90%, 95%, 96%, 97%, 98%, 99%, or 100% identical to SEQ ID NO: 133, and a light chain comprising an amino acid sequence at least 80%, 85%, 90%, 95%, 96%, 97%, 98%, 99%, or 100% identical to SEQ ID NO: 134;
f. a heavy chain comprising an amino acid sequence at least 80%, 85%, 90%, 95%, 96%, 97%, 98%, 99%, or 100% identical to SEQ ID NO: 135, and a light chain comprising an amino acid sequence at least 80%, 85%, 90%, 95%, 96%, 97%, 98%, 99%, or 100% identical to SEQ ID NO: 136; and
g. a heavy chain comprising an amino acid sequence at least 80%, 85%, 90%, 95%, 96%, 97%, 98%, 99%, or 100% identical to SEQ ID NO: 137, and a light chain comprising an amino acid sequence at least 80%, 85%, 90%, 95%, 96%, 97%, 98%, 99%, or 100% identical to SEQ ID NO: 138.

In certain aspects, the bispecific antibody capable of binding a severe acute respiratory syndrome coronavirus 2 (SARS-COV-2) spike protein receptor binding domain (RBD) comprises (i) a first heavy chain comprising a first heavy chain variable region (VH1), (ii) a first light chain comprising a first light chain variable region (VL1), (iii) a second heavy chain comprising a second heavy chain variable region (VH2), and (iv) a second light chain comprising a second light chain variable region (VL2); wherein the VH1 and the VH2 each comprises three heavy chain complementarity determining regions (HCDR1, HCDR2, and HCDR3) and the VL1 and the VL2 each comprises three light chain complementarity determining regions (LCDR1, LCDR2, and LCDR3); wherein the complementarity determining regions are selected from the group consisting of:
a. a VH1 HCDR1 comprising SEQ ID NO: 41, a VH1 HCDR2 comprising SEQ ID NO: 42, a VH1 HCDR3 comprising SEQ ID NO: 43, a VL1 LCDR1 comprising SEQ ID NO: 47, a VL1 LCDR2 comprising the amino acid sequence GSS, a VL1 LCDR3 comprising SEQ ID NO: 49, a VH2 HCDR1 comprising SEQ ID NO: 44, a VH2 HCDR2 comprising SEQ ID NO: 45, a VH2 HCDR3 comprising SEQ ID NO: 46, a VL2 LCDR1 comprising SEQ ID NO: 50, a VL2 LCDR2 comprising the amino acid sequence AAS, and a VL2 LCDR3 comprising SEQ ID NO: 52;
b. a VH1 HCDR1 comprising SEQ ID NO: 151, a VH1 HCDR2 comprising SEQ ID NO: 152, a VH1 HCDR3 comprising SEQ ID NO: 153, a VL1 LCDR1 comprising SEQ ID NO: 165, a VL1 LCDR2 comprising the amino acid sequence GAS, a VL1 LCDR3 comprising SEQ ID NO: 166, a VH2 HCDR1 comprising SEQ ID NO: 158, a VH2 HCDR2 comprising SEQ ID NO: 159, a VH2 HCDR3 comprising SEQ ID NO: 160, a VL2 LCDR1 comprising SEQ ID NO: 171, a VL2 LCDR2 comprising the amino acid sequence WAS, and a VL2 LCDR3 comprising SEQ ID NO: 172;
c. a VH1 HCDR1 comprising SEQ ID NO: 177, a VH1 HCDR2 comprising SEQ ID NO: 178, a VH1 HCDR3 comprising SEQ ID NO: 179, a VL1 LCDR1 comprising SEQ ID NO: 191, a VL1 LCDR2 comprising the amino acid sequence DAS, a VL1 LCDR3 comprising SEQ ID NO: 192, a VH2 HCDR1 comprising SEQ ID NO: 184, a VH2 HCDR2 comprising SEQ ID NO: 185, a VH2 HCDR3 comprising SEQ ID NO: 186, a VL2 LCDR1 comprising SEQ ID NO: 197, a VL2 LCDR2 comprising the amino acid sequence GAS, and a VL2 LCDR3 comprising SEQ ID NO: 198;
d. a VH1 HCDR1 comprising SEQ ID NO: 203, a VH1 HCDR2 comprising SEQ ID NO: 204, a VH1 HCDR3 comprising SEQ ID NO: 205, a VL1 LCDR1 comprising SEQ ID NO: 217, a VL1 LCDR2 comprising the amino acid sequence DAS, a VL1 LCDR3 comprising SEQ ID NO: 218, a VH2 HCDR1 comprising SEQ ID NO: 210, a VH2 HCDR2 comprising SEQ ID NO: 211, a VH2 HCDR3 comprising SEQ ID NO: 212, a VL2 LCDR1 comprising SEQ ID NO: 223, a VL2 LCDR2 comprising the amino acid sequence WAS, and a VL2 LCDR3 comprising SEQ ID NO: 224;
e. a VH1 HCDR1 comprising SEQ ID NO: 229, a VH1 HCDR2 comprising SEQ ID NO: 230, a VH1 HCDR3 comprising SEQ ID NO: 231, a VL1 LCDR1 comprising SEQ ID NO: 243, a VL1 LCDR2 comprising the amino acid sequence AAS, a VL1 LCDR3 comprising SEQ ID NO: 244, a VH2 HCDR1 comprising SEQ ID NO: 236, a VH2 HCDR2 comprising SEQ ID NO: 237, a VH2 HCDR3 comprising SEQ ID NO: 238, a VL2 LCDR1 comprising SEQ ID NO: 249, a VL2 LCDR2 comprising the amino acid sequence GAS, and a VL2 LCDR3 comprising SEQ ID NO: 250 a VH1 HCDR1 comprising SEQ ID NO: 229, a VH1 HCDR2 comprising SEQ ID NO: 230, a VH1 HCDR3 comprising SEQ ID NO: 231, a VL1 LCDR1 comprising SEQ ID NO: 243, a VL1 LCDR2 comprising the amino acid sequence AAS, a VL1 LCDR3 comprising SEQ ID NO: 244, a VH2 HCDR1 comprising SEQ ID NO: 236, a VH2 HCDR2 comprising SEQ ID NO: 237, a VH2 HCDR3 comprising SEQ ID NO: 238, a VL2 LCDR1 comprising SEQ ID NO: 249, a VL2 LCDR2 comprising the amino acid sequence GAS, and a VL2 LCDR3 comprising SEQ ID NO: 250; and
f. a VH1 HCDR1 comprising SEQ ID NO: 255, a VH1 HCDR2 comprising SEQ ID NO: 256, a VH1 HCDR3 comprising SEQ ID NO: 257, a VL1 LCDR1 comprising SEQ ID NO: 269, a VL1 LCDR2 comprising the amino acid sequence AAS, a VL1 LCDR3 comprising SEQ ID NO: 270, a VH2 HCDR1 comprising SEQ ID NO: 262, a VH2 HCDR2 comprising SEQ ID NO: 263, a VH2 HCDR3 comprising SEQ ID NO: 264, a VL2 LCDR1 comprising SEQ ID NO: 275, a VL2 LCDR2 comprising the amino acid sequence WAS, and a VL2 LCDR3 comprising SEQ ID NO: 276.

In certain embodiments, the VH1, VL1, VH2, and VL2 of the bispecific antibody are selected from the group consisting of:
a. a VH1 comprising an amino acid sequence at least 80%, 85%, 90%, 95%, 96%, 97%, 98%, 99%, or 100% identical to SEQ ID NO: 53, a VL1 comprising an amino acid sequence at least 80%, 85%, 90%, 95%, 96%, 97%, 98%, 99%, or 100% identical to SEQ ID NO: 55, a VH2 comprising an amino acid sequence at least 80%, 85%, 90%, 95%, 96%, 97%, 98%, 99%, or 100% identical to SEQ ID NO: 54, and a VL2 comprising an amino acid sequence at least 80%, 85%, 90%, 95%, 96%, 97%, 98%, 99%, or 100% identical to SEQ ID NO: 56;
b. a VH1 comprising an amino acid sequence at least 80%, 85%, 90%, 95%, 96%, 97%, 98%, 99%, or 100% identical to SEQ ID NO: 154, a VL1 comprising an amino acid sequence at least 80%, 85%, 90%, 95%, 96%, 97%, 98%, 99%, or 100% identical to SEQ ID NO: 167, a VH2 comprising an amino acid sequence at least 80%, 85%, 90%, 95%, 96%, 97%, 98%, 99%, or 100% identical to SEQ ID NO: 161, and a VL2 comprising an amino acid sequence at least 80%, 85%, 90%, 95%, 96%, 97%, 98%, 99%, or 100% identical to SEQ ID NO: 173;
c. a VH1 comprising an amino acid sequence at least 80%, 85%, 90%, 95%, 96%, 97%, 98%, 99%, or 100% identical to SEQ ID NO: 180, a VL1 comprising an amino acid sequence at least 80%, 85%, 90%, 95%, 96%, 97%, 98%, 99%, or 100% identical to SEQ ID NO: 193, a VH2 comprising an amino acid sequence at least 80%, 85%, 90%, 95%, 96%, 97%, 98%, 99%, or 100% identical to SEQ ID NO: 187, and a VL2 comprising an amino acid sequence at least 80%, 85%, 90%, 95%, 96%, 97%, 98%, 99%, or 100% identical to SEQ ID NO: 199;
d. a VH1 comprising an amino acid sequence at least 80%, 85%, 90%, 95%, 96%, 97%, 98%, 99%, or 100% identical to SEQ ID NO: 206, a VL1 comprising an amino acid sequence at least 80%, 85%, 90%, 95%, 96%, 97%, 98%, 99%, or 100% identical to SEQ ID NO: 219, a VH2 comprising an amino acid sequence at least 80%, 85%, 90%, 95%, 96%, 97%, 98%, 99%, or 100% identical to SEQ ID NO: 213, and a VL2 comprising an amino acid sequence at least 80%, 85%, 90%, 95%, 96%, 97%, 98%, 99%, or 100% identical to SEQ ID NO: 225;
e. a VH1 comprising an amino acid sequence at least 80%, 85%, 90%, 95%, 96%, 97%, 98%, 99%, or 100% identical to SEQ ID NO: 232, a VL1 comprising an amino acid sequence at least 80%, 85%, 90%, 95%, 96%, 97%, 98%, 99%, or 100% identical to SEQ ID NO: 245, a VH2 comprising an amino acid sequence at least 80%, 85%, 90%, 95%, 96%, 97%, 98%, 99%, or 100% identical to SEQ ID NO: 239, and a VL2 comprising an amino acid sequence at least 80%, 85%, 90%, 95%, 96%, 97%, 98%, 99%, or 100% identical to SEQ ID NO: 251; and
f. a VH1 comprising an amino acid sequence at least 80%, 85%, 90%, 95%, 96%, 97%, 98%, 99%, or 100% identical to SEQ ID NO: 258, a VL1 comprising an amino acid sequence at least 80%, 85%, 90%, 95%, 96%, 97%, 98%, 99%, or 100% identical to SEQ ID NO: 271, a VH2 comprising an amino acid sequence at least 80%, 85%, 90%, 95%, 96%, 97%, 98%, 99%, or 100% identical to SEQ ID NO: 265, and a VL2 comprising an amino acid sequence at least 80%, 85%, 90%, 95%, 96%, 97%, 98%, 99%, or 100% identical to SEQ ID NO: 277.

In certain embodiments, the first heavy chain, the first light chain, the second heavy chain, and the second light chain of the bispecific antibody are selected from the group consisting of:
a. a first heavy chain comprising an amino acid sequence at least 80%, 85%, 90%, 95%, 96%, 97%, 98%, 99%, or 100% identical to SEQ ID NO: 57, a first light chain comprising an amino acid sequence at least 80%, 85%, 90%, 95%, 96%, 97%, 98%, 99%, or 100% identical to SEQ ID NO: 59, a second heavy chain comprising an amino acid sequence at least 80%, 85%, 90%, 95%, 96%, 97%, 98%, 99%, or 100% identical to SEQ ID NO: 58, and a second light chain comprising an amino acid sequence at least 80%, 85%, 90%, 95%, 96%, 97%, 98%, 99%, or 100% identical to SEQ ID NO: 60;
b. a first heavy chain comprising an amino acid sequence at least 80%, 85%, 90%, 95%, 96%, 97%, 98%, 99%, or 100% identical to SEQ ID NO: 155, a first light chain comprising an amino acid sequence at least 80%, 85%, 90%, 95%, 96%, 97%, 98%, 99%, or 100% identical to SEQ ID NO: 168, a second heavy chain comprising an amino acid sequence at least 80%, 85%, 90%, 95%, 96%, 97%, 98%, 99%, or 100% identical to SEQ ID NO: 162, and a second light chain comprising an amino acid sequence at least 80%, 85%, 90%, 95%, 96%, 97%, 98%, 99%, or 100% identical to SEQ ID NO: 174;
c. a first heavy chain comprising an amino acid sequence at least 80%, 85%, 90%, 95%, 96%, 97%, 98%, 99%, or 100% identical to SEQ ID NO: 181, a first light chain comprising an amino acid sequence at least 80%, 85%, 90%, 95%, 96%, 97%, 98%, 99%, or 100% identical to SEQ ID NO: 194, a second heavy chain comprising an amino acid sequence at least 80%, 85%, 90%, 95%, 96%, 97%, 98%, 99%, or 100% identical to SEQ ID NO: 188, and a second light chain comprising an amino acid sequence at least 80%, 85%, 90%, 95%, 96%, 97%, 98%, 99%, or 100% identical to SEQ ID NO: 200;
d. a first heavy chain comprising an amino acid sequence at least 80%, 85%, 90%, 95%, 96%, 97%, 98%, 99%, or 100% identical to SEQ ID NO: 207, a first light chain comprising an amino acid sequence at least 80%, 85%, 90%, 95%, 96%, 97%, 98%, 99%, or 100% identical to SEQ ID NO: 220, a second heavy chain comprising an amino acid sequence at least 80%, 85%, 90%, 95%, 96%, 97%, 98%, 99%, or 100% identical to SEQ ID NO: 214, and a second light chain comprising an amino acid sequence at least 80%, 85%, 90%, 95%, 96%, 97%, 98%, 99%, or 100% identical to SEQ ID NO: 226;
e. a first heavy chain comprising an amino acid sequence at least 80%, 85%, 90%, 95%, 96%, 97%, 98%, 99%, or 100% identical to SEQ ID NO: 233, a first light chain comprising an amino acid sequence at least 80%, 85%, 90%, 95%, 96%, 97%, 98%, 99%, or 100% identical to SEQ ID NO: 246, a second heavy chain comprising an amino acid sequence at least 80%, 85%, 90%, 95%, 96%, 97%, 98%, 99%, or 100% identical to SEQ ID NO: 240, and a second light chain comprising an amino acid sequence at least 80%, 85%, 90%, 95%, 96%, 97%, 98%, 99%, or 100% identical to SEQ ID NO: 252; and
f. a first heavy chain comprising an amino acid sequence at least 80%, 85%, 90%, 95%, 96%, 97%, 98%, 99%, or 100% identical to SEQ ID NO: 259, a first light chain comprising an amino acid sequence at least 80%, 85%, 90%, 95%, 96%, 97%, 98%, 99%, or 100% identical to SEQ ID NO: 272, a second heavy chain comprising an amino acid sequence at least 80%, 85%, 90%, 95%, 96%, 97%, 98%, 99%, or 100% identical to SEQ ID NO: 266, and a second light chain comprising an amino acid sequence at least 80%, 85%, 90%, 95%, 96%, 97%, 98%, 99%, or 100% identical to SEQ ID NO: 278.

Amino acid sequences of exemplary monoclonal antibodies/antigen-binding fragments and bispecific antibodies are provided in Table 1 and additional sequences of the present invention are provided in Table 2.

TABLE 1
Amino acid sequences of exemplary monoclonal antibodies/
antigen-binding fragments and bispecific antibodies

| | | | | |
|---|---|---|---|---|
| Clone 2 | Heavy Chain | Light Chain | VH | VL |
| | SEQ ID NO: 9 | SEQ ID NO: 10 | SEQ ID NO: 7 | SEQ ID NO: 8 |
| | HCDR1 | HCDR2 | HCDR3 | |
| | SEQ ID NO: 1 | SEQ ID NO: 2 | SEQ ID NO: 3 | |
| | GYSFTGYS | INPSTGGT | ARSRGDY | |
| | LCDR1 | LCDR2 | LCDR3 | |
| | SEQ ID NO: 4 | AAS | SEQ ID NO: 6 | |
| | ESVEYYGTSL | | QQSRKVPWT | |
| Clone 6 | Heavy Chain | Light Chain | VH | VL |
| | SEQ ID NO: 19 | SEQ ID NO: 20 | SEQ ID NO: 17 | SEQ ID NO: 18 |
| | HCDR1 | HCDR2 | HCDR3 | |
| | SEQ ID NO: 11 | SEQ ID NO: 12 | SEQ ID NO: 13 | |
| | GYTFTSYT | INPTSGYT | AREGHRVGPAY | |
| | LCDR1 | LCDR2 | LCDR3 | |
| | SEQ ID NO: 14 | GSS | SEQ ID NO: 16 | |
| | ESVDNYGISF | | QQSKEVPYT | |
| Clone 12 | Heavy Chain | Light Chain | VH | VL |
| | SEQ ID NO: 29 | SEQ ID NO: 30 | SEQ ID NO: 27 | SEQ ID NO: 28 |
| | HCDR1 | HCDR2 | HCDR3 | |
| | SEQ ID NO: 21 | SEQ ID NO: 22 | SEQ ID NO: 23 | |
| | GYSFTGYS | INPSTGGT | ARSRGDY | |
| | LCDR1 | LCDR2 | LCDR3 | |
| | SEQ ID NO: 24 | AAS | SEQ ID NO: 26 | |
| | ESVEYYGTSL | | QQSRKVPWT | |
| Clone 13 | Heavy Chain | Light Chain | VH | VL |
| | SEQ ID NO: 39 | SEQ ID NO: 40 | SEQ ID NO: 37 | SEQ ID NO: 38 |
| | HCDR1 | HCDR2 | HCDR3 | |
| | SEQ ID NO: 31 | SEQ ID NO: 32 | SEQ ID NO: 33 | |
| | GYSFTGYS | INPSTGGT | ARSRGDY | |
| | LCDR1 | LCDR2 | LCDR3 | |
| | SEQ ID NO: 34 | AAS | SEQ ID NO: 36 | |
| | ESVEYYGTSL | | QQSRKVPWT | |
| Clone 16 | Heavy Chain 1 | Light Chain 1 | VH1 | VL1 |
| | SEQ ID NO: 57 | SEQ ID NO: 59 | SEQ ID NO: 53 | SEQ ID NO: 55 |
| | H1CDR1 | H1CDR2 | H1CDR3 | |
| | SEQ ID NO: 41 | SEQ ID NO: 42 | SEQ ID NO: 43 | |
| | GYTFTSYT | INPTSGYT | AREGHRVGPAY | |
| | L1CDR1 | L1CDR2 | L1CDR3 | |
| | SEQ ID NO: 47 | GSS | SEQ ID NO: 49 | |
| | ESVDNYGISF | | QQSKEVPYT | |
| | Heavy Chain 2 | Light Chain 2 | VH2 | VL2 |
| | SEQ ID NO: 58 | SEQ ID NO: 60 | SEQ ID NO: 54 | SEQ ID NO: 56 |
| | H2CDR1 | H2CDR2 | H2CDR3 | |
| | SEQ ID NO: 44 | SEQ ID NO: 45 | SEQ ID NO: 46 | |
| | GYSFTGYS | INPSTGGT | ARSRGDY | |
| | L2CDR1 | L2CDR2 | L2CDR3 | |
| | SEQ ID NO: 50 | AAS | SEQ ID NO: 52 | |
| | ESVEYYGTSL | | QQSRKVPWT | |
| MB.02 | Heavy Chain | Light Chain | VH | VL |
| | SEQ ID NO: 133 | SEQ ID NO: 134 | SEQ ID NO: 101 | SEQ ID NO: 104 |
| | HCDR1 | HCDR2 | HCDR3 | |
| | SEQ ID NO: 107 | SEQ ID NO: 108 | SEQ ID NO: 109 | |
| | GFSFTSYW | IYPGDSDT | CARGGYEDVFDIW | |
| | LCDR1 | LCDR2 | LCDR3 | |
| | SEQ ID NO: 116 | GAS | SEQ ID NO: 118 | |
| | QSVSSSY | | CQQYGSSPPTF | |
| MB.08 | Heavy Chain | Light Chain | VH | VL |
| | SEQ ID NO: 135 | SEQ ID NO: 136 | SEQ ID NO: 102 | SEQ ID NO: 105 |
| | HCDR1 | HCDR2 | HCDR3 | |
| | SEQ ID NO: 110 | SEQ ID NO: 111 | SEQ ID NO: 112 | |
| | GGSISSYY | IYYSGST | CARGLTGGDAFDIW | |
| | LCDR1 | LCDR2 | LCDR3 | |
| | SEQ ID NO: 119 | WAS | SEQ ID NO: 121 | |
| | QSVLYSSNNENY | | CQQYYSTPLTF | |
| PB.03 | Heavy Chain | Light Chain | VH | VL |
| | SEQ ID NO: 137 | SEQ ID NO: 138 | SEQ ID NO: 103 | SEQ ID NO: 106 |
| | HCDR1 | HCDR2 | HCDR3 | |
| | SEQ ID NO: 113 | SEQ ID NO: 114 | SEQ ID NO: 115 | |
| | GFTFSSYA | ISGSGGST | CAKDKAVEAFDIW | |
| | LCDR1 | LCDR2 | LCDR3 | |
| | SEQ ID NO: 122 | DAS | SEQ ID NO: 124 | |
| | QDISNY | | CQQYDNLPLTF | |
| COV2-0208 | Heavy Chain 1 | Light Chain 1 | VH1 | VL1 |
| | SEQ ID NO: 155 | SEQ ID NO: 168 | SEQ ID NO: 154 | SEQ ID NO: 167 |
| | H1CDR1 | H1CDR2 | H1CDR3 | |
| | SEQ ID NO: 151 | SEQ ID NO: 152 | SEQ ID NO: 153 | |
| | GGSISSYY | IYYSGST | ARGLTGGDAFDI | |
| | L1CDR1 | L1CDR2 | L1CDR3 | |
| | SEQ ID NO: 165 | GAS | SEQ ID NO: 166 | |
| | QSVSSSY | | CQQYGSSPPTF | |
| | Heavy Chain 2 | Light Chain 2 | VH2 | VL2 |
| | SEQ ID NO: 162 | SEQ ID NO: 174 | SEQ ID NO: 161 | SEQ ID NO: 173 |
| | H2CDR1 | H2CDR2 | H2CDR3 | |
| | SEQ ID NO: 158 | SEQ ID NO: 159 | SEQ ID NO: 160 | |
| | GFSFTSYW | IYPGDSDT | ARGGYEDVFDI | |
| | L2CDR1 | L2CDR2 | L2CDR3 | |
| | SEQ ID NO: 171 | WAS | SEQ ID NO: 172 | |
| | QSVLYSSNNENY | | CQQYYSTPLTF | |
| COV2-0203 | Heavy Chain 1 | Light Chain 1 | VH1 | VL1 |
| | SEQ ID NO: 181 | SEQ ID NO: 194 | SEQ ID NO: 180 | SEQ ID NO: 193 |
| | H1CDR1 | H1CDR2 | H1CDR3 | |
| | SEQ ID NO: 177 | SEQ ID NO: 178 | SEQ ID NO: 179 | |
| | GFSFTSYW | IYPGDSDT | ARGGYEDVFDI | |
| | L1CDR1 | L1CDR2 | L1CDR3 | |
| | SEQ ID NO: 191 | DAS | SEQ ID NO: 192 | |
| | QDISNY | | CQQYDNLPLTF | |
| | Heavy Chain 2 | Light Chain 2 | VH2 | VL2 |
| | SEQ ID NO: 188 | SEQ ID NO: 200 | SEQ ID NO: 187 | SEQ ID NO: 199 |
| | H2CDR1 | H2CDR2 | H2CDR3 | |
| | SEQ ID NO: 184 | SEQ ID NO: 185 | SEQ ID NO: 186 | |
| | GFTFSSYA | ISGSGGST | AKDKAVEAFDI | |
| | L2CDR1 | L2CDR2 | L2CDR3 | |
| | SEQ ID NO: 197 | GAS | SEQ ID NO: 198 | |
| | QSVSSSY | | CQQYGSSPPTF | |
| COV2-0803 | Heavy Chain 1 | Light Chain 1 | VH1 | VL1 |
| | SEQ ID NO: 207 | SEQ ID NO: 220 | SEQ ID NO: 206 | SEQ ID NO: 219 |
| | H1CDR1 | H1CDR2 | H1CDR3 | |
| | SEQ ID NO: 203 | SEQ ID NO: 204 | SEQ ID NO: 205 | |
| | GGSISSYY | IYYSGST | ARGLTGGDAFDI | |
| | L1CDR1 | L1CDR2 | L1CDR3 | |
| | SEQ ID NO: 217 | DAS | SEQ ID NO: 218 | |
| | QDISNY | | CQQYDNLPLTF | |
| | Heavy Chain 2 | Light Chain 2 | VH2 | VL2 |
| | SEQ ID NO: 214 | SEQ ID NO: 226 | SEQ ID NO: 213 | SEQ ID NO: 225 |
| | H2CDR1 | H2CDR2 | H2CDR3 | |
| | SEQ ID NO: 210 | SEQ ID NO: 211 | SEQ ID NO: 212 | |
| | GFTFSSYA | ISGSGGST | AKDKAVEAFDI | |
| | L2CDR1 | L2CDR2 | L2CDR3 | |
| | SEQ ID NO: 223 | WAS | SEQ ID NO: 224 | |
| | QSVLYSSNNENY | | CQQYYSTPLTF | |
| COV2-0213 | Heavy Chain 1 | Light Chain 1 | VH1 | VL1 |
| | SEQ ID NO: 233 | SEQ ID NO: 246 | SEQ ID NO: 232 | SEQ ID NO: 245 |
| | H1CDR1 | H1CDR2 | H1CDR3 | |
| | SEQ ID NO: 229 | SEQ ID NO: 230 | SEQ ID NO: 231 | |
| | GFSFTSYW | IYPGDSDT | ARGGYEDVFDI | |
| | L1CDR1 | L1CDR2 | L1CDR3 | |
| | SEQ ID NO: 243 | AAS | SEQ ID NO: 244 | |
| | SVEYYGTS | | QQSRKVPWT | |
| | Heavy Chain 2 | Light Chain 2 | VH2 | VL2 |
| | SEQ ID NO: 240 | SEQ ID NO: 252 | SEQ ID NO: 239 | SEQ ID NO: 251 |
| | H2CDR1 | H2CDR2 | H2CDR3 | |
| | SEQ ID NO: 236 | SEQ ID NO: 237 | SEQ ID NO: 238 | |
| | GFTFTGYS | INPSTGGT | ARSRGDY | |
| | L2CDR1 | L2CDR2 | L2CDR3 | |
| | SEQ ID NO: 249 | GAS | SEQ ID NO: 250 | |
| | QSVSSSY | | CQQYGSSPPTF | |
| COV2-0813 | Heavy Chain 1 | Light Chain 1 | VH1 | VL1 |
| | SEQ ID NO: 259 | SEQ ID NO: 272 | SEQ ID NO: 258 | SEQ ID NO: 271 |
| | H1CDR1 | H1CDR2 | H1CDR3 | |
| | SEQ ID NO: 255 | SEQ ID NO: 256 | SEQ ID NO: 257 | |
| | GGSISSYY | IYYSGST | ARGLTGGDAFDI | |
| | L1CDR1 | L1CDR2 | L1CDR3 | |
| | SEQ ID NO: 269 | AAS | SEQ ID NO: 270 | |
| | SVEYYGTS | | QQSRKVPWT | |
| | Heavy Chain 2 | Light Chain 2 | VH2 | VL2 |
| | SEQ ID NO: 266 | SEQ ID NO: 278 | SEQ ID NO: 265 | SEQ ID NO: 277 |
| | H2CDR1 | H2CDR2 | H2CDR3 | |
| | SEQ ID NO: 262 | SEQ ID NO: 263 | SEQ ID NO: 264 | |
| | GFTFTGYS | INPSTGGT | ARSRGDY | |
| | L2CDR1 | L2CDR2 | L2CDR3 | |
| | SEQ ID NO: 275 | WAS | SEQ ID NO: 276 | |
| | QSVLYSSNNENY | | CQQYYSTPLTF | |

TABLE 2
Additional Sequences

| SEQ ID | | SEQ ID | |
|---|---|---|---|
| NO | | NO | |
| 61 | pPR23 | 156 | COV2-0208 IGG1-KNOB-1 VH DNA |
| 62 | pPR24 | 157 | COV2-0208 IGG1-KNOB-1 HEAVY CHAIN |
| | | | DNA |
| 63 | oPR81-F primer | 163 | COV2-0208 IGG1-HOLE-2 VH DNA |
| 64 | oPR82-R primer | 164 | COV2-0208 IGG1-HOLE-2 HEAVY CHAIN |
| | | | DNA |
| 65 | Clone 1-VH-gBlock | 169 | COV2-0208 LIGHT CHAIN-1 VL DNA |
| 66 | Clone 2-VH-gBlock | 170 | COV2-0208 LIGHT CHAIN-1 DNA |
| 67 | Clone 3-VH-gBlock | 175 | COV2-0208 LIGHT CHAIN-2 VL DNA |
| 68 | Clone 4-VH-gBlock | 176 | COV2-0208 LIGHT CHAIN-2 DNA |
| 69 | Clone 5-VH-gBlock | 182 | COV2-0203 IGG1-KNOB-1 VH DNA |
| 70 | Clone 6-VH-gBlock | 183 | COV2-0203 IGG1-KNOB-1 HEAVY CHAIN |
| | | | DNA |
| 71 | Clone 7-VH-gBlock | 189 | COV2-0203 IGG1-HOLE-2 VH DNA |
| 72 | Clone 8-VH-gBlock | 190 | COV2-0203 IGG1-HOLE-2 HEAVY CHAIN |
| | | | DNA |
| 73 | Clone 9-VH-gBlock | 195 | COV2-0203 LIGHT CHAIN-1 VL DNA |
| 74 | Clone 10-VH-gBlock | 196 | COV2-0203 LIGHT CHAIN-1 DNA |
| 75 | Clone 11-VH-gBlock | 201 | COV2-0203 LIGHT CHAIN-2 VL DNA |
| 76 | Clone 12-VH-gBlock | 202 | COV2-0203 LIGHT CHAIN-2 DNA |
| 77 | Clone 13-VH-gBlock | 208 | COV2-0803 IGG1-KNOB-1 VH DNA |
| 78 | Clone 1-VK-gBlock | 209 | COV2-0803 IGG1-KNOB-1 HEAVY CHAIN |
| | | | DNA |
| 79 | Clone 2-VK-gBlock | 215 | COV2-0803 IGG1-HOLE-2 VH DNA |
| 80 | Clone 3-VK-gBlock | 216 | COV2-0803 IGG1-HOLE-2 HEAVY CHAIN |
| | | | DNA |
| 81 | Clone 4-VK-gBlock | 156 | COV2-0208 IGG1-KNOB-1 VH DNA |
| 82 | Clone 5-VK-gBlock | 157 | COV2-0208 IGG1-KNOB-1 HEAVY CHAIN |
| | | | DNA |
| 83 | Clone 6-VK-gBlock | 163 | COV2-0208 IGG1-HOLE-2 VH DNA |
| 84 | Clone 7-VK-gBlock | 164 | COV2-0208 IGG1-HOLE-2 HEAVY CHAIN |
| | | | DNA |
| 85 | Clone 8-VK-gBlock | 169 | COV2-0208 LIGHT CHAIN-1 VL DNA |
| 86 | Clone 9-VK-gBlock | 170 | COV2-0208 LIGHT CHAIN-1 DNA |
| 87 | Clone 10-VK-gBlock | 175 | COV2-0208 LIGHT CHAIN-2 VL DNA |
| 88 | Clone 11-VK-gBlock | 176 | COV2-0208 LIGHT CHAIN-2 DNA |
| 89 | Clone 12-VK-gBlock | 182 | COV2-0203 IGG1-KNOB-1 VH DNA |
| 90 | Clone 13-VK-gBlock | 183 | COV2-0203 IGG1-KNOB-1 HEAVY CHAIN |
| | | | DNA |
| 91 | UK variant gBlock1 | 189 | COV2-0203 IGG1-HOLE-2 VH DNA |
| 92 | UK variant gBlock2 | 190 | COV2-0203 IGG1-HOLE-2 HEAVY CHAIN |
| | | | DNA |
| 93 | UK variant gBlock3 | 195 | COV2-0203 LIGHT CHAIN-1 VL DNA |
| 94 | UK variant gBlock4 | 196 | COV2-0203 LIGHT CHAIN-1 DNA |
| 95 | South African variant-gBlock1 | 201 | COV2-0203 LIGHT CHAIN-2 VL DNA |
| 96 | South African variant-gBlock2 | 202 | COV2-0203 LIGHT CHAIN-2 DNA |
| 97 | del19R1 primer | 208 | COV2-0803 IGG1-KNOB-1 VH DNA |
| 98 | del19F1 primer | 209 | COV2-0803 IGG1-KNOB-1 HEAVY CHAIN |
| | | | DNA |
| 125 | MB.02 VH DNA | 215 | COV2-0803 IGG1-HOLE-2 VH DNA |
| 126 | MB.08 VH DNA | 216 | COV2-0803 IGG1-HOLE-2 HEAVY CHAIN |
| | | | DNA |
| 127 | PB.03 VH DNA | 221 | COV2-0803 LIGHT CHAIN-1 VL DNA |
| 128 | MB.02 VL (VK) DNA | 222 | COV2-0803 LIGHT CHAIN-1 DNA |
| 129 | MB.08 VL (VK) DNA | 227 | COV2-0803 LIGHT CHAIN-2 VL DNA |
| 130 | PB.03 VL (VK) DNA | 228 | COV2-0803 LIGHT CHAIN-2 DNA |
| 131 | hIgG1 Constant Heavy | 234 | COV2-0213 IGG1-KNOB-1 VH DNA |
| 132 | hIgGkappa Constant | 235 | COV2-0213 IGG1-KNOB-1 HEAVY CHAIN |
| | | | DNA |
| 139 | MB.02 Heavy chain DNA | 241 | COV2-0213 IGG1-HOLE-2 VH DNA |
| 140 | MB.02 light chain DNA | 242 | COV2-0213 IGG1-HOLE-2 HEAVY CHAIN |
| | | | DNA |
| 141 | MB.08 heavy chain DNA | 247 | COV2-0213 LIGHT CHAIN-1 VH DNA |
| 142 | MB.08 light chain DNA | 248 | COV2-0213 LIGHT CHAIN-1 DNA |
| 143 | PB.03 heavy chain DNA | 253 | COV2-0213 LIGHT CHAIN-2 VL DNA |
| 144 | PB.03 light chain DNA | 254 | COV2-0213 LIGHT CHAIN-2 DNA |
| 145 | MB_VH2 Heavy Chain Vector | 260 | COV2-0813 IGG1-KNOB-1 VH DNA |
| 146 | MB_VK2 Light Chain Vector | 261 | COV2-0813 IGG1-KNOB-1 HEAVY CHAIN |
| | | | DNA |
| 147 | MB_MH8 Heavy Chain Vector | 267 | COV2-0813 IGG1-HOLE-2 VH DNA |
| 148 | MB_VK8 Light Chain Vector | 268 | COV2-0813 IGG1-HOLE-2 HEAVY CHAIN |
| | | | DNA |
| 149 | PB_VH3 Heavy Chain Vector | 273 | COV2-0813 LIGHT CHAIN-1 VL DNA |
| 150 | PB_VK3 Light Chain Vector | 274 | COV2-0813 LIGHT CHAIN-1 DNA |
| | | 279 | COV2-0813 LIGHT CHAIN-2 VL DNA |
| | | 280 | COV2-0813 LIGHT CHAIN-2 DNA |
| 281 | Omicr-MB_VK2_Vector | | |
| 282 | Omicr-MB_VK8_Vector | | |
| 283 | pPR146 pfuse-IgG1-Knob-MB.08- | | |
| | VH-CH1-CH2-CH3 Vector | | |
| 284 | pPR148 pFUSE-IgG-Clone13A-VL- | | |
| | CH1 Vector | | |
| 285 | pPR149 pfuse-IgG1-Hole-Clone13A- | | |
| | VH-CKappa-CH2-CH3 Vector | | |
| 286 | pPR150 pFUSE-IgG-MB2-VL-CH1 | | |
| | Vector | | |
| 287 | pPR151 pFUSE-IgG-PB3-VL-CH1 | | |
| | Vector | | |
| 288 | pPR152 pfuse-IgG1-Hole-MB2-VH- | | |
| | CKappa-CH2-CH3 Vector | | |
| 289 | pPR153 pfuse-IgG1-Hole-PB3-VH- | | |
| | CKappa-CH2-CH3 Vector | | |
| 290 | pPR154 pfuse-IgG1-Knob-MB.02- | | |
| | VH-CH1-CH2-CH3 Vector | | |

In certain embodiments, the anti-RBD antibody or antigen-binding fragment or bispecific antibody disclosed herein is conjugated or recombinantly fused to at least one other moiety or molecule. Examples of such moieties include, but are not limited to, diagnostic, detectable, or therapeutic agents or any other molecules such as a purification tag (e.g., a His tag). The conjugated or recombinantly fused antibodies or antigen-binding fragments or bispecific antibodies can be useful, e.g., for monitoring or prognosing the onset, development, progression and/or severity of a condition or disease, for example, as part of a clinical testing procedure, such as determining the efficacy of a particular therapy. The conjugated or recombinantly fused antibodies or antigen-binding fragments or bispecific antibodies can be useful, e.g., for treating a condition or disorder described herein.

Antibodies or antigen-binding fragments or bispecific antibodies described herein can also be conjugated to a molecule (e.g., polyethylene glycol) which can affect one or more biological and/or molecular properties of the antibodies, for example, stability (e.g., in serum), half-life, solubility, and antigenicity.

In a particular aspect, provided herein is a conjugate comprising an agent (e.g., therapeutic agent) linked to an antibody or an antigen-binding fragment or bispecific antibody described herein.

Diagnosis and detection can be accomplished, for example, by coupling an antibody or antigen-binding fragment or bispecific antibody described herein to detectable molecules or substances including, but not limited to, various enzymes, prosthetic groups (such as, but not limited to, streptavidin/biotin and avidin/biotin), fluorescent molecules, bioluminescent molecules, radioactive molecules, and positron emitting metals using various positron emission tomographies, and non-radioactive paramagnetic metal ions.

Antibodies and antigen-binding fragments or bispecific antibodies described herein can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Pharmaceutical Compositions and Formulations

Also provided are compositions, such as pharmaceutical compositions and formulations suitable for administration to a subject, comprising the anti-RBD antibodies or antigen-binding fragments or bispecific antibodies disclosed herein. In certain embodiments, the pharmaceutical composition further comprises at least one pharmaceutical carrier, vehicle or diluent. In some embodiments, the composition includes at least one additional therapeutic agent.

The terms “pharmaceutical formulation” and “pharmaceutical composition” refer to a preparation which is in such form as to permit the biological activity of an active ingredient contained therein to be effective, and which contains no additional components which are unacceptably toxic to a subject to which the formulation would be administered. A “pharmaceutically acceptable carrier, vehicle or diluent” refers to an ingredient in a pharmaceutical formulation, other than an active ingredient, which is nontoxic to a subject. A pharmaceutically acceptable carrier or vehicle or diluent includes, but is not limited to, a buffer, excipient, stabilizer, or preservative. In some aspects, the choice of carrier is determined in part by the particular method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol: 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the antibodies, preferably those with activities complementary to the antibodies, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the antibodies in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the antibodies, by multiple bolus administrations of the antibodies, or by continuous infusion administration of the antibodies.

Formulations include those for oral, intravenous, intraperitoneal, subcutaneous, pulmonary, transdermal, intramuscular, intranasal, buccal, sublingual, or suppository administration. In some embodiments, the antibodies are administered parenterally. The term “parenteral,” as used herein, includes intravenous, intramuscular, subcutaneous, rectal, vaginal, and intraperitoneal administration. In some embodiments, the antibodies are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the antibodies in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

Also provided herein are kits comprising one or more antibodies or antigen-binding fragments or bispecific antibodies described herein, or conjugates thereof. In a specific embodiment, provided herein is a pharmaceutical pack or kit comprising one or more containers filled with one or more of the ingredients of the pharmaceutical compositions described herein, such as one or more antibodies or antigen-binding fragments or bispecific antibodies provided herein, or a conjugate thereof. Optionally associated with such container(s) can be a notice in the form prescribed by a governmental agency regulating the manufacture, use or sale of pharmaceuticals or biological products, which notice reflects approval by the agency of manufacture, use or sale for human administration.

### Nucleic Acids, Vectors, and Host Cells

In certain aspects, the invention provides an isolated nucleic acid comprising at least one polynucleotide sequence encoding a monoclonal antibody or antigen-binding fragment or bispecific antibody described herein (i.e., an antibody or antigen-binding fragment or bispecific antibody which is capable of binding a severe acute respiratory syndrome coronavirus 2 (SARS-CoV-2) spike protein receptor binding domain (RBD)).

Nucleic acids of the invention may be DNA or RNA. In certain embodiments, the isolated nucleic acid is DNA. In certain embodiments, the isolated nucleic acid is RNA or mRNA.

Also provided are vectors comprising the isolated nucleic acid. In certain embodiments, the vector is a cloning vector. In some embodiments, the vector is an expression vector.

In some embodiments, a nucleic acid of the present disclosure may be operably linked to a transcriptional control element, e.g., a promoter, and enhancer, etc. Suitable promoter and enhancer elements are known to those of skill in the art. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

In some embodiments, an expression control DNA sequence can be operably linked to immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the light chains, heavy chains, light/heavy chain dimers or intact antibodies, antigen-binding fragments or any other immunoglobulin forms may follow (see, Beychok, Cells of Immunoglobulin Synthesis, Academic Press, New York, 1979, which is incorporated herein by reference).

In certain aspects, the invention provides a host cell comprising the isolated nucleic acid and/or a vector comprising the isolated nucleic acid. A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA, or cosmid DNA expression vectors containing antibody coding sequences; yeast (e.g., Saccharomyces or Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, MDCK, HEK293, NS0, PER.C6, VERO, CRL 7030, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, the expression of nucleotide sequences encoding antibodies described herein is regulated by a constitutive promoter, an inducible promoter, or a tissue specific promoter. In certain embodiments, antibodies described herein are produced in mammalian cells, such as HEK 293 cells.

### Methods of Use

In one aspect, a method of detecting a severe acute respiratory syndrome coronavirus 2 (SARS-COV-2) is provided, wherein the method comprises contacting a sample with a monoclonal antibody or antigen-binding fragment or bispecific antibody, as described herein, which is capable of binding a spike protein receptor binding domain (RBD) of the SARS-COV-2.

In one aspect, a method of diagnosing a severe acute respiratory syndrome coronavirus 2 (SARS-COV-2) infection in a subject is provided, wherein the method comprises contacting a sample obtained from the subject with a monoclonal antibody or antigen-binding fragment or bispecific antibody, as described herein, which is capable of binding a spike protein receptor binding domain (RBD) of the SARS-COV-2.

In one aspect, a method of neutralizing a severe acute respiratory syndrome coronavirus 2 (SARS-COV-2) in a subject is provided, wherein the method comprises administering to the subject a monoclonal antibody or antigen-binding fragment or bispecific antibody, as described herein, which is capable of binding a spike protein receptor binding domain (RBD) of the SARS-CoV-2.

In one aspect, a method of treating at least one sign or symptom of a severe acute respiratory syndrome coronavirus 2 (SARS-COV-2) infection in a subject is provided, wherein the method comprises administering to the subject a monoclonal antibody or antigen-binding fragment or bispecific antibody, as described herein, which is capable of binding a spike protein receptor binding domain (RBD) of the SARS-COV-2.

Signs and symptoms of SARS-COV-2 infection vary widely among infected individuals. For example, an infected individual may have no noticeable symptoms, but may present at least one sign of SARS-COV-2 infection such as identification of SARS-COV-2 particles in, e.g., a sample of mucus from the subject's nose or throat, or from a sample of saliva from the subject. Signs and symptoms of SARS-COV-2 infection may include, but are not limited to, fever, chills, cough, shortness of breath, difficulty breathing, fatigue, pink eye, rash, muscle aches, body aches, headache, new loss of taste, new loss of smell, sore throat, congestion, runny nose, nausea, vomiting, diarrhea, persistent chest pain and/or pressure, new confusion, inability to wake or stay awake, or any combination thereof. Symptoms may also include pale, gray, or blue-colored skin, lips, or nail beds, depending on the subject's skin tone. Signs and symptoms of SARS-COV-2 infection also include COVD-19 and complications thereof including pneumonia, trouble breathing, organ failure, heart problems, acute respiratory distress syndrome, blood clots, acute kidney injury, additional viral and/or bacterial infection(s), cytokine storm, and any combination thereof.

Conjugated versions of the anti-RBD antibodies or antigen-binding fragments or bispecific antibodies disclosed herein are particularly useful for the methods of use described herein. For example, the antibody or antigen-binding fragment may be conjugated or recombinantly fused to at least one other moiety or molecule, such as a diagnostic, detectable, or therapeutic agent or any other molecule such as a purification tag (e.g., a His tag). The conjugated or recombinantly fused antibodies or antigen-binding fragments can be useful, e.g., for monitoring, diagnosing, or prognosing the onset, development, progression and/or severity of a condition or disease, such as COVID-19, for example, as part of a clinical testing procedure, such as determining the efficacy of a particular therapy. The conjugated or recombinantly fused antibodies or antigen-binding fragments can be useful, e.g., for treating a condition or disorder described herein, including SARS-COV-2 infection and COVID-19.

Antibodies or antigen-binding fragments or bispecific antibodies described herein for use in any of the methods disclosed herein can also be conjugated to a molecule (e.g., polyethylene glycol) which can affect one or more biological and/or molecular properties of the antibodies, for example, stability (e.g., in serum), half-life, solubility, and antigenicity.

In a particular aspect, provided herein is a conjugate comprising an agent (e.g., therapeutic agent) linked to an antibody or an antigen-binding fragment or bispecific antibody described herein.

Diagnosis and detection can be accomplished, for example, by coupling an antibody or antigen-binding fragment or bispecific antibody described herein to detectable molecules or substances including, but not limited to, various enzymes, prosthetic groups (such as, but not limited to, streptavidin/biotin and avidin/biotin), fluorescent molecules, bioluminescent molecules, radioactive molecules, and positron emitting metals using various positron emission tomographies, and non-radioactive paramagnetic metal ions.

Antibodies and antigen-binding fragments or bispecific antibodies described herein can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### EXPERIMENTAL EXAMPLES

Various embodiments of the present application can be better understood by reference to the following Examples which are offered by way of illustration. The scope of the present application is not limited to the Examples given herein.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Example 1: Generation and Characterization of High Affinity mAbs Targeting SARS-CoV-2

### Materials and Methods

### Animal Immunization

Standard 28-day repetitive immunization protocol was utilized to immunize C57BL/6J and BALB/C mice. First, all mice were ear-marked and around 200 μl blood was taken as pre-immunization sample, where serum was collected from the blood by centrifugation (1,000 g for 10 min). Two days later (day 0), for each mouse, 20 μg SARS-COV-2 RBD-his tag protein (Sino Biological) in 100 μl PBS was mixed with 100 μl Complete Freund's Adjuvant (CFA) with 3-way stopcock. The fully emulsified mixture was subcutaneously injected into the back of each mouse. On day 7, a second immunization was performed, where each mouse was injected subcutaneously with 20 μg RBD-his tag protein fully emulsified with Incomplete Freund's Adjuvant (IFA). On day 13, around 50 μl of blood from each mouse was obtained for serum preparation as first bleeds. On day 14, a third immunization was performed, where all the procedures were similar to the second immunization. On day 20, second bleeds were taken. On day 21, fourth immunization was performed, where all the procedures were similar to the second immunization. On day 24, each mouse received 20 μg RBD-his tag protein in 200 μl PBS intraperitoneally as final immunization. On day 28, mice with strong serum conversion detected by ELISA were sacrificed. Spleen, lymph nodes, and bone marrow were collected for B cells isolation and purification for single cell BCR sequencing. Serums from pre, first and second bleeds were subjected to ELISA for anti-RBD tilter determination.

### Mouse B Cell Isolation and Purification

Primary B cells from spleen, draining lymph nodes, and bone marrow of RBD-his tag protein immunized mice were isolated and purified with mouse CD138 MicroBeads (Miltenyi Biotec, 130-098-257) following standard protocol provided by the manufacturer. Spleens and draining lymph nodes were homogenized gently. Bone marrows were fragmented, rinsed with PBS containing 2% FBS, and filtered with a 100 μm cell strainer (BD Falcon, Heidelberg, Germany). The cell suspension was centrifuged for 5 min with 400 g at 4° C. Erythrocytes were lysed briefly using ACK lysis buffer (Lonza) with 1 mL per spleen for 1˜2 mins before adding 10 mL PBS containing 2% FBS to restore iso-osmolarity. The single-cell suspensions were filtered through a 40 μm cell strainer (BD Falcon, Heidelberg, Germany). CD138 positive B cells were isolated using magnetic cell sorting by positive selection according to the manufacturer's instructions. Cell samples post-magnetic selection were counted and prepared for single cell BCR sequencing.

### Single Cell BCR Sequencing

The enriched CD138″ plasma cells and progenitor B cells were loaded on a 10× Chromium Next GEM Chip G. The target cell number was 10,000 cells per sample. Single-cell lysis and RNA first-strand synthesis were performed using Chromium Next GEM Single Cell 5′ Gel Bead V3.1 according to the manufacturer's protocol. The following RNA and V(D)J library preparation was performed according to the manufacturer's protocol (Chromium Next GEM Single Cell V(D)J reagent kit, mouse BCR). The resulting VDJ-enriched libraries were sequenced following the reading mode recommended by 10× Genomics. Sequencing was performed on a NovaSeq targeted for 10,000 reads/cell, with a total of 100 million reads.

### Single Cell VDJ Sequencing Data Analysis

Raw sequencing data were processed using Cell Ranger v3.1.0 with default settings, aligning the reads to the GRCm38 mouse VDJ reference. Outputs from Cell Ranger were then visualized using the Loupe V(D)J Browser for quality control assessment and to identify the top enriched clonotypes. The consensus amino acid sequences for the top-ranked heavy/light chain pairs in each sample were then extracted and codon-optimized for human expression.

### Plasmid Construction

The cDNA sequences of the paired variable heavy and light chain region of anti-RBD antibody clones were synthesized as gBlocks (IDT) (see Table 2) and cloned by the Gibson assembly (NEB) into human IgG1 heavy chain and light chain expression plasmids, pFUSEss-CHIg-hG1 (InvivoGen, pfusess-hchg1) and pFUSE2ss-CLIg-hK (InvivoGen, pfuse2ss-hclk), respectively. pFUSEss-CHIg-hG1 plasmid is a cloning plasmid that expresses the constant region of the human IgG1 heavy chain and includes multiple cloning sites to enable cloning of the heavy chain (CH) variable region. Parallelly, pFUSE2ss-CLIg-hK is a cloning plasmid that expresses the constant region of the human kappa light chain and contains multiple cloning sites to enable cloning of the light chain variable region. For plasmid cloning of the anti-RBD antibody clones' heavy chain, so-called “gBlocks” were ordered from IDT. Each gBlock contains a cDNA sequence of a variable region of heavy chain of anti-RBD antibody clones and the regions overlapping with the corresponding flanking sequences of EcoRI and NheI restriction sites of pFUSEss-CHIg-hG1. pFUSEss-CHIg-hG1 were digested with EcoRI and NheI restriction enzyme (Thermo fisher). These synthesized gBlocks were cloned into gel-purified restriction enzyme digested backbone by the Gibson assembly (NEB). For plasmid cloning of anti-RBD antibody clones' light chain, gBlocks, containing cDNA sequence of variable region of light chain of anti-RBD antibody clones and the regions overlapping with corresponding flanking sequences of EcoRI and BsiWI restriction sites of pFUSE2ss-CLIg-hK, were ordered from IDT. The gBlocks were then cloned into the pFUSE2ss-CLIg-hK backbone, which was digested with EcoRI and BsiWI restriction enzyme (Thermo fisher). Sequences for selected monoclonal antibodies, namely Clone 2, Clone 6, Clone 12, and Clone 13, are provided (see Table 1 and Table 2).

The bi-specific antibody with the same Fab regions of Clone 2 and Clone 6 (termed Clone 16, see Table 1 and Table 2) was generated by using the CrossMab-KiH bi-specific constructs (Schaefer et al). The CrossMab-KiH bi-specific constructs were designed and generated basing on pFUSEss-CHIg-hG1 and pFUSE2ss-CLIg-hK. The bi-specific antibody consists of two hetero-half IgG1, one is knob IgG1 and the other is hole IgG1 (Knob-in-Hole conformation). Four plasmids were employed: pFUSE2ss-knobLight-hK, pFUSE2ss-knobheavy-hG1, pFUSE2ss-HoleLight-hK, pFUSE2ss-HoleHeavy-hG1. The pFUSE2ss-knobLight-hK is pFUSE2ss-CLIg-hK with no further editing. The pFUSE2ss-knobheavy-hG1 contains two knob mutations (T366W, S354C) in the CH3 region when compared with pFUSEss-CHIg-hG1. The gBlock (pPR024), containing constant region of heavy chain with two knob mutations and the regions overlapping with corresponding flanking sequences of NsiI and NheI restriction sites in pFUSEss-CHIg-hG1 was ordered from IDT, and then cloned into NsiI and NheI restriction enzymes digested pFUSEss-CHIg-hG1 backbone by the Gibson assembly (NEB). The pFUSE2ss-HoleLight-hK was generated by replacing the constant region of Light chain (CL) in pFUSE2ss-CLIg-hK with CH1 region of heavy chain in pFUSEss-CHIg-hG1 vector. The CH1 region were PCR amplified from pFUSEss-CHIg-hG1 vectors with a forward primer (oPR81-F) and a reverse primer (oPR82-R) containing regions overlapping with corresponding flanking sequences of the NcoI and NheI restriction sites in the pFUSE2ss-CLIg-hK. CH1 PCR amplified fragments were gel-purified and cloned into restriction enzyme digested pFUSE2ss-CLIg-hK by the Gibson assembly (NEB). The pFUSE2ss-HoleHeavy-hG1 possesses three “hole” mutations (T366S, L368A, Y407V) in the CH3 region and a Y349C on the “hole” side to form a stabilizing disulfide bridge. In addition, to get the correct association of the light chain and the cognate heavy chain, the CH1 region in the pFUSE2ss-HoleHeavy-hG1 was exchanged with constant region of light chain (CrossMab conformation). The gBlock (pPR023), containing cDNA sequence of constant region of light chain, CH2 and CH3 with “hole” mutations, and regions overlapping with corresponding flanking sequences of NsiI and NheI restriction sites in pFUSEss-CHIg-hG1 was ordered from IDT, and cloned into NsiI and NheI restriction enzymes digested pFUSEss-CHIg-hG1 backbone through Gibson assembly (NEB). The sequence information of gBlocks and primers sequences are provided (see Table 2). All plasmids were sequenced and Maxiprepped for subsequent experiments.

### Cloning of SARS-Cov-2 Mutant Spike Variants

Two recently emerged SARS-COV-2 mutant spike variants were generated: one is B.1.1.7 variant (Wang et al, 2021), which also named as the UK mutant; the other is the South African (SA) mutant, B.1.351 (Wang et al, 2021). pVP18-UK variant contains 9 mutations in the spike: 69del, 70 del, Y144 del, N501Y, A570D, P681H, T716I, S982A and D1118H. pVP21-SA variant includes four mutants in the N-terminal domain (L18F, D80A and D215G, R246I), three mutants at key residues in the RBD (N501Y, E484K and K417N), and one is in loop 2 (A701V). Both pVP18-UK variant and pVP21-SA variant were generated basing on pcDNA3.1-pSARS-CoV-2-S, which was derived by insertion of a synthetic human codon-optimized cDNA (Geneart) encoding a wildtype (WT) SARS-COV-2 S protein. For pVP18-UK-variant, four gBlocks, that containing cDNA sequence of UK variant's mutations and regions overlapping with corresponding flanking sequences of NheI and BamHI restriction sites pcDNA3.1-pSARA-CoV-2, were ordered from IDT. The gBlocks were then cloned into pcDNA3.1-pSARA-COV-2 backbone, digested with NheI and BamHI restriction enzyme (Thermo fisher). For pVP21-SA-variant, two gBlocks, contains mutations in SA variant regions overlapping with corresponding flanking sequences of NheI and BsrGI restriction sites pcDNA3.1-pSARA-COV-2. The gBlocks were then cloned into pcDNA3.1-pSARA-COV-2 backbone, digested with NheI and BsrGI restriction enzyme (Thermo Fisher) through Gibson assembly. For the HIV-based SARS-COV-2 spike pseudotyped virus generation, WT pcDNA3.1-pSARS-COV-2-S, pVP18-UK-variant and PVP21-SA-variant lacking the C-terminal 19 codons were employed. A pair of forward and reverse primers were utilized to amplify fragments lacking the C-terminal 19 codons with pVP18-UK variant and pVP21-SAvariant as template. The amplified fragments were gel-purified and cloned into pVP18-UK variant and pVP21-SA variant backbone, digested with BbvCI and BamHI.

### Cell Culture

HEK293FT (Thermo Fisher) and 293T-hACE2 cell lines were cultured in complete growth medium, Dulbecco's modified Eagle's medium (DMEM; Thermo Fisher) supplemented with 10% Fetal bovine serum (FBS, Hyclone), 1% penicillin-streptomycin (Gibco) (D10 media for short). Cells were typically passaged every 1-2 days at a split ratio of 1:2 or 1:4 when the confluency reached at 80%. Expi293F™ (Thermo Fisher) cells were cultured in Expi293™ Expression Medium (Thermo Fisher) in 125-mL shaker flask set on an orbital shaker with 125 rpm shake speed. For routine maintenance, Expi293F™ cells were grown to 3-5×10⁶ cells/mL, then split to 0.3-0.5×10⁶ cells/mL every 3 days.

### Recombinant Antibody Generation

The top-ranked enriched IgG clones were selected and cDNAs of relative variable region of paired heavy- and light-chain were codon-optimized and cloned separately into human IgG1 heavy chain and light chain expression vectors, containing the human IgG1 constant regions (pFuse plasmids). IgG1 antibodies were expressed in Expi293F™ cells. ExpiFectamine 293 transfection kit (Thermo Fisher) was utilized for heavy and light chain plasmids transfection following the manufacturer's instruction. After 5 days, the antibody containing supernatants were collected. Suitable amount of rProtein A Sepharose® Fast Flow beads (Cytiva) was pre-washed and added into supernatants. After overnight incubation at 4° C., antibody bound protein A beads were collected with Poly-Prep® Chromatography Columns (BIO-RAD). After 3 times wash with DPBS, mAbs were eluted with Fab elution buffer, then neutralized with Tris-HCl. Buffer exchange was performed with Amicon Ultra-4 Centrifugal Filter (Millipore Sigma) to keep mAbs in PBS for following assays. The numbering of mAbs was based on the order of mouse immunization and cloning. Clones 1˜4 were mAbs chosen from enriched clones from RBD-his tag protein immunized C57BL/6J mice. Clone 5-11 were mAbs chosen from RBD-his tag protein immunized BALB/c mice. The antibodies recombinantly produced in this study are summarized in Table 3.

TABLE 3
List of Antibody Clones

| | | Original Clonal freq % from |
|---|---|---|
| Clone ID | Origin | single cell BCR-seq |
| Clone 1 | C57BL/6-20200506 | 3.17% |
| Clone 2 | C57BL/6-20200506 | 1.61% |
| Clone 3 | C57BL/6-20200506 | 1.58% |
| Clone 4 | C57BL/6-20200506 | 1.58% |
| Clone 5 | Balb/c-20200527 | 0.95% |
| Clone 6 | Balb/c-20200527 | 0.84% |
| Clone 7 | Balb/c-20200527 | 0.64% |
| Clone 8 | Balb/c-20200527 | 0.64% |
| Clone 9 | Balb/c-20200709 | 1.07% |
| Clone 10 | Balb/c-20200709 | 0.86% |
| Clone 11 | Balb/c-20200709 | 0.64% |
| Clone 12 | Humanized clone 2 | |
| Clone 13 | Humanized clone 2 | |
| Clone 16 | Bispecific antibody of clone 2 | |
| | and clone 6 | |

### Bi-Specific Antibody Generation

Clone 16 (Clone6-KiH-Clone2) bispecific antibody is a human IgG1-like bispecific antibody, generated based on CrossMab-KiH bispecific constructs, including pFUSE2ss-knobLight-hK, pFUSE2ss-knobheavy-hG1, pFUSE2ss-HoleLight-hK, pFUSE2ss-HoleHeavy-hG1. The design and generation of CrossMab-KiH bi-specific constructs was described in the above plasmid constructs sections. The variable region of Clone 6 heavy chain was cloned into pFUSE2ss-knobheavy-hG1vector. The variable region of Clone 6 light chain was cloned into pFUSE2ss-knobLight-hK vector. Clone6-KiH-Clone2 bispecific antibody was expressed in vitro in Expi293F™ cells by co-transfecting four plasmids (clone 6 knob heavy chain plasmid, clone 6 knob light chain plasmid, clone 2 hole heavy chain plasmid, and clone 2 hole light chain plasmid) with ExpiFectamine 293 transfection kit (Thermo Fisher). The expression and antibody purification protocol was similar to recombinant antibody expression described as above.

### ELISA for Anti-Serum Titer Determination.

The antibody tilters in sera from pre, first and second bleeds were determined using direct coating ELISA. The 384-well ELISA plates (Corning) were coated with 3 μg/mL SARS-COV-2 RBD-his tag protein (Sino Biological) in PBS at 4° C. overnight. After standard washing with PBS-T washing buffer (phosphate-buffered saline containing 0.05% Tween 20), ELISA plates were blocked with blocking buffer (2% bovine serum albumin dissolved in PBS-T and filtered) for 1 h at room temperature. Serial dilutions of pre-immune, first and second immune anti-sera in blocking buffer were added into plates and for 1 h at room temperature. Plates were washed and incubated with relative goat anti-mouse IgG(H+L)/HRP (Thermo Fisher, 1:5000) for 1 h at room temperature. Plates were washed and developed using TMB reagents as substrates (Biolegend) following the manufacturer's recommended protocol. Reaction was stopped with stop solution (1 M H₃PO₄) and absorbance at 450 nm was recorded by a microplate reader (Perkin Elmer).

### ELISA for Anti-RBD Antibody Clones Binding.

SARS-COV-2 RBD protein with 6× Histidine (Sino Biological) was coated at 3 μg/ml in PBS on a 384-well microtiter plate overnight at 4° ° C. After standard washing with PBS-T and blocked with 2% (w/v) solution of BSA in PBS-T to remove nonspecific binding, purified anti-RBD antibodies were diluted proportionally in PBS-T+2% BSA and transferred to the washed and blocked microtiter plates. After one hour of incubation at RT, plates were washed, and RBD-his tag protein-bound antibody was detected with goat-anti-human IgG1 (H+L) with horseradish peroxidase (HRP) conjugated (Invitrogen, 1:1000) The plates were washed and developed using TMB substrate solution (Biolegend) according to manufacturer's recommendation and absorbance at 450 nm was measured on a microplate reader after the reaction was stopped by stop solution (1M H₃PO₄).

### Affinity Determination Via Bio-Layer Interferometry (BLI)

Antibody binding kinetics for anti-spike mAbs were evaluated by BLI on an Octet RED96e instrument (FortéBio) at room temperature. HIS1K biosensors (ForteBio) were first loaded with his-tagged SARS-COV-2 RBD protein to a response of about 1 nm, followed by a 60 s baseline step in the kinetic buffer (PBS, 0.02% Tween, pH7.4). After that, the biosensors were associated with indicated concentrations of the antibodies (from 50 nM to 0.78125 nM with 2-fold dilutions, where the kinetic buffer was served as the negative control) for 200 s, then dissociated in the kinetic buffer for 1000 s. The dissociation constants K_{D}, kinetic constants Kₒₙ and K_{off}, were calculated by using a 1:1 Langmuir binding model with FortéBio data analysis software.

### Affinity Measurement by Surface Plasmon Resonance (SPR)

Kinetics binding measurement for anti-spike mAbs in this study was performed using a Biacore T200 instrument (GE Healthcare). The system was flushed with filtered 1×HBS-P+ running buffer (0.01M HEPES, 0.15M NaCl and 0.05% v/v Surfactant P20, pH 7.4) and all steps were performed at 25° C. chip temperature.

### Kinetics Binding Measurement on CM5 Chip (Series S Sensor Chip CM5)

For kinetic binding measurements, CM5 chip surface was activated by injecting a solution of EDC/NHS (GE Healthcare). Mouse anti-human IgG (Fc) mAb (25 μg/ml) was immobilized on the sensor chip by amine coupling, followed by deactivation using 1M ethanolamine. Afterward, anti-spike mAbs (0.1 μg/ml) were then flowed over and captured on anti-human IgG (Fc) mAb-coated surface. Subsequently, gradient diluted his-tagged SARS-CoV-2 RBD solutions (1.875 nM-30 nM, two-fold serial dilution) were injected individually in single-cycle kinetic format without regeneration (30 μl/min, association: 180 s, dissociation: 60 s). The binding data were double referenced by blank cycle and reference flow cell subtraction. Processed data were fitted by 1:1 interaction model using Biacore T200 Evaluation Software 3.1.

### Kinetics Binding Measurement on NTA Chip

For kinetic binding measurements, NTA chip was activated manually by loading a solution of NiCl2. Histidine-labelled SARS-COV-2 RBD protein (0.075 μg/ml) was then flowed over the chip and captured on nickel-coated surface. Subsequently, gradient diluted anti-spike mAbs solutions (0.9875 nM-15 nM, two-fold serial dilution) were injected individually in single-cycle kinetic format without regeneration (30 μl/min, association: 240 s, dissociation: 90 s). The binding data were double referenced by blank cycle and reference flow cell subtraction. Processed data were fitted by 1:1 interaction model using Biacore T200 Evaluation Software 3.1.

SARS-Cov-2 Pseudovirus Reporter and Neutralization Assays HIV-1 based SARS-COV-2 S pseudotyped virions were generated according to a previous study (Fabian Schmidt, 2020). Two plasmids are adopted to generate HIV-1 based SARS-COV-2 S pseudotyped virions. HIV-1 dual reporter vector expressing mCherry and luciferase (NL4-3 mCherry Luciferase, plasmid #44965) was purchased from Addgene. In order to generate HIV-1 based SARS-COV-2 S pseudotyped virions, 15×10⁶ 293FT cells were seeded in a 150 mm plates one day before in 20 ml D10 media. The following day, after the cell density reaches 90%, medium was discarded and replaced with 13 mL serum-free Opti-MEM medium. μg NL4-3 mCherry Luciferase reporter plasmids and 15 μg SARS-COV-2 (pSARS-COV-2419) plasmids were mixed thoroughly in 225 μl serum-free Opti-MEM medium. Then 100 μl Lipofectamine 2000 (Invitrogen) were diluted in 225 μl serum-free Opti-MEM medium. Then the diluted plasmid mixture and Lipofectamine 2000 were mixed thoroughly and incubated for mins at RT before adding into cells. After 6 hr, the culture medium was changed back to the completed growth medium, 20 mL for one 150 mm plate. At 48 h after transfection, the 20 mL supernatant was harvested and filtered through a 0.45-μm filter, aliquoted and frozen in −80° C.

Parallelly, the three plasmids-based HIV-1 pseudotyped virus system were utilized to generate (HIV-1/NanoLuc2AEGFP)-SARS-COV-2 particles and (HIV-1/NanoLuc2AEGFP)-SARS-COV-2-South African variant particles. Briefly, 293T cells were seeded in 150 mm plates, and transfected with 21 μg pHIVNLGagPol, 21 μg pCCNanoLuc2AEGFP, and 7.5 μg of a SARS-COV-2 SΔ19 or SARS-COV-2 South African SΔ19 plasmid utilizing 198 μl PEI. At 48 h after transfection, the 20-ml supernatant was harvested and filtered through a 0.45-μm filter, and concentrated before aliquoted and frozen in −80° C.

The pseudovirus neutralization assays were performed on 293T-hACE2 cell line. One day before, 293T-hACE2 cells were plated in a 96 well plate, 0.02×10⁶ cells per well. The following day, serial dilution of monoclonal IgG from 40 μg/mL (4-fold serial dilution using complete growth medium, 55 μL aliquots) were mixed with the same volume of SARS-COV-2 pseudovirus. The mixture was incubated for 1 hr at 37° C. incubator, supplied with 5% CO2. Then 100 μL of the mixtures were added into 96-well plates with 293T-hACE2 cells. Plates were incubated at 37° ° C. supplied with 5% CO₂. 48 hr later, 1 μL D-luciferin reagent (Perkin Elmer, 33.3 mg/ml) was added to each well and incubated for 5 mins. Luciferase activity was measured with using a microplate spectrophotometer (Perkin Elmer). The inhibition rate was calculated by comparing the OD value to relative negative and positive control wells. For the three plasmids-based HIV-1 pseudotyped virus system, 293T cells were collected and the GFP+ cells were analyzed with Attune NxT Acoustic Focusing Cytometer (Thermo Fisher). The 50% inhibitory concentration (IC50) was calculated with a four-parameter logistic regression using GraphPad Prism 8.0 (GraphPad Software Inc.).

### In Vitro Neutralization Against Authentic SARS-Cov-2 in BI.3 Setting

SARS-COV-1 (USA-WA1/2020) was produced in Vero-E6 cells and tittered. SARS-CoV-2 neutralization was assessed by measuring cytotoxicity. 5×10⁵ Vero-E6 cells were plated per well of a 96-well plate. The following day, serial dilutions of antibodies were incubated with 2.5×10³ plaque forming units (PFU) SARS-COV-2 for 1 hour at room temperature. SARS-COV-2 neutralization was assessed by measuring cytotoxicity. 5×10⁵ Vero-E6 cells were plated per well of a 96-well plate. The following day, serial dilutions of antibodies were incubated with 2.5×10³ PFU SARS-COV-2 for 1 hour at room temperature. The medium was then aspirated from the cells and replaced with 100 μl of the antibody/virus mixture. After 72 hours at 37° C., 10 μl of CellTiter-Glo (Promega) was added per well to measure cellular ATP concentrations. Relative luminescence units were detected on Cytation5 plate reader. All conditions were normalized to an uninfected control. Each condition was done in triplicate in each of three independent experiments.

### In Vivo Efficacy Testing Against Authentic SARS-Cov-2 in BL3 Setting

The efficacy of mAbs against replication-competent SARS-COV-2 virus was evaluated in vivo, using both a prophylactic setting where the animals were treated with mAb prior to viral infection, and a therapeutic setting where the animals were treated post infection. These experiments were performed in an animal BSL3 (ABSL3) facility. The replication-competent SARS-COV-2 (USA-WA1/2020) virus was produced in VeroE6 cells, and the titer was determined by plaque assay using WT VeroE6.

The K18-hACE2 mice (B6.Cg-Tg(K18-ACE2)₂Prlmn/J) were purchased from the Jackson Laboratory and bred in house using a trio breeding scheme. Mice were sedated with isoflurane, and infected via intranasal inoculation of 2,000 PFU (20×LD50) SARS-COV-2 (USA-WA1/2020) virus administered in 50 μL of DPBS. Six to eight-week-old K18-hACE2 littermate-controlled mice, mixed gender (male/female) mice were divided randomly into three groups, and administered with 20 mg/kg (of mice body weight) Clone 2, Clone 6 or placebo/control, via intraperitoneal (IP) injection. For prophylactic experiment, the mAb drug/placebo treatment was 24 h prior to infection; for therapeutic experiment, the treatment was 18 h post-infection. The control for the prophylactic experiment was DPBS, and the control for the therapeutic experiment was isotype control hIgG1, where both controls are similar (no effect on disease progression). Survival, body conditions and weights of mice were monitored daily for 10 consecutive days.

### Fab Generation

The Fab fragments of Clone 2 and Clone 6 were generated from full length IgGs of Clone 2 and Clone 6 using a commercial Pierce™ Fab Preparation Kit (Thermo Fisher). All procedures were performed following the manufacturer's instructions. Briefly, 2 mg the whole IgGs of Clone 2 and Clone 6 were digested with immobilized papain at 37° C. for 4 hr with rotation. Then protein A beads were applied to bind the Fc fragments and undigested IgG. Then Fab fragments were recovered in the flow-through fraction. The concentration of Fab fragments were measured with a NanoDrop device (Thermo Fisher) and used for subsequent 3D structure analysis.

### Cryo-EM Sample Preparation, Data Collection and Processing

The purified SARS-COV-2 spike trimer at a final concentration of 0.3 mg/mL (after mixture) was mixed with Clone 2 or Clone 6 Fab at a molar ratio of 1:2 at 4° C. for 30 mins. Then 3 μl of the protein mixture was applied to a Quantifoil-Cu-2/1-3C grid (Quantifoil) pretreated by glow-discharging at 15 mA for 1 min. The grid was blotted at 4° C. with 100% humidity and plunge-frozen in liquid ethane using FEI Vitrobot Mark IV (Thermo Fisher). The grids were stored in liquid nitrogen until data collection.

Images were acquired on an FEI Titan Krios electron microscope (Thermo Fisher) equipped with a Gatan K3 Summit direct detector in super-resolution mode, at a calibrated magnification of 81,000× with the physical pixel size corresponding to 1.068 Å. Detailed data collection statistics for the Fab-spike trimer complexes are shown in a supplemental table. Automated data collection was performed using SerialEM (Mastronarde, 2005).

### Model Building and Refinement

Model building and refinement of CryoEM data were performed similar to our previous work (Yuan et al. 2020). The structures were refined with phenix.real_space_refine module in PHENIX (Adams et al., 2010). All structural figures were generated using Py Mol (Schrodinger, 2015) and Chimera (Pettersen et al., 2004).

Single Cell BCR Sequencing of RBD-Immunized Mice Identified Enriched BCRs Encoding Strong mAbs Against SARS-COV-2

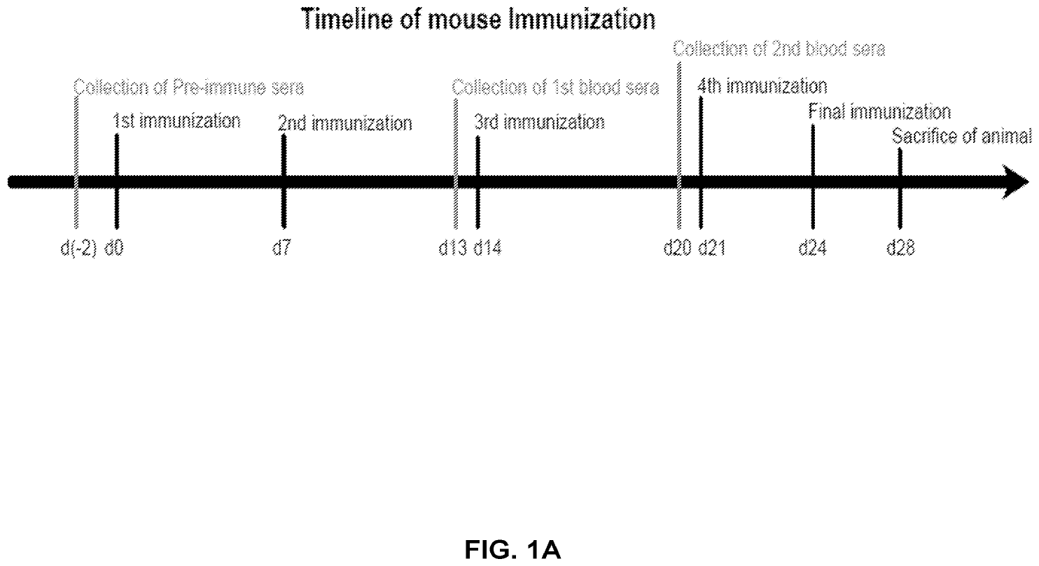

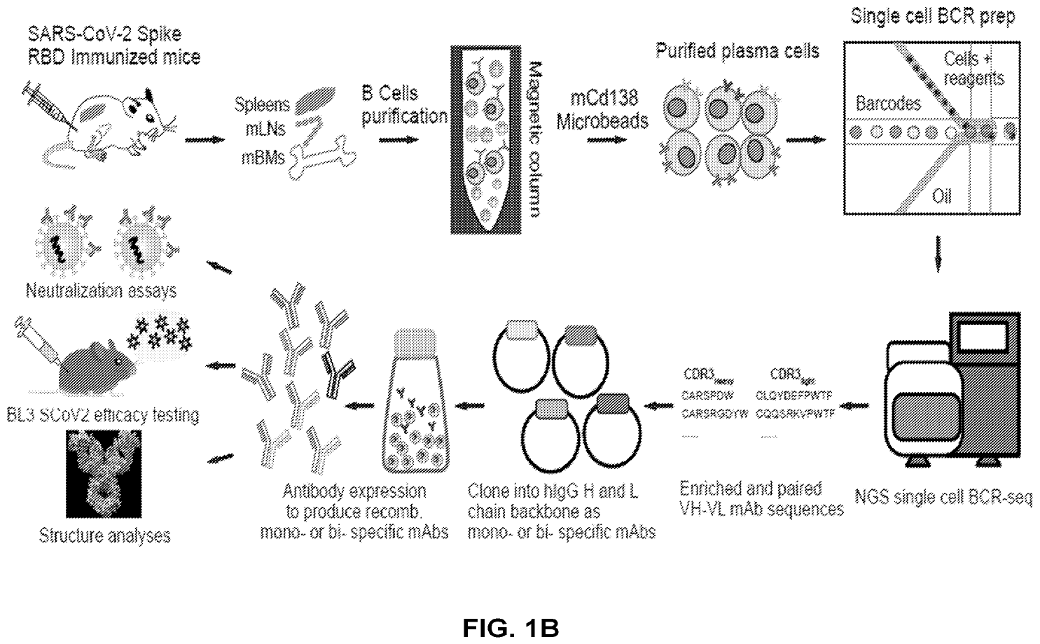

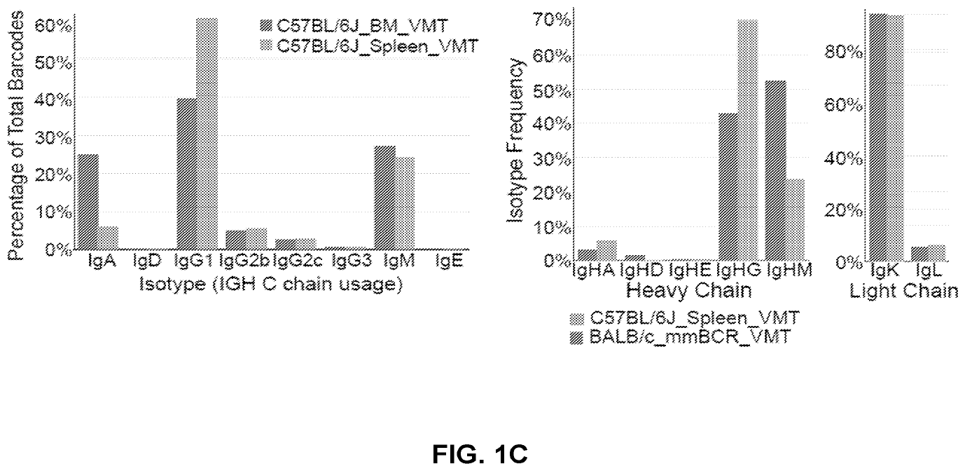

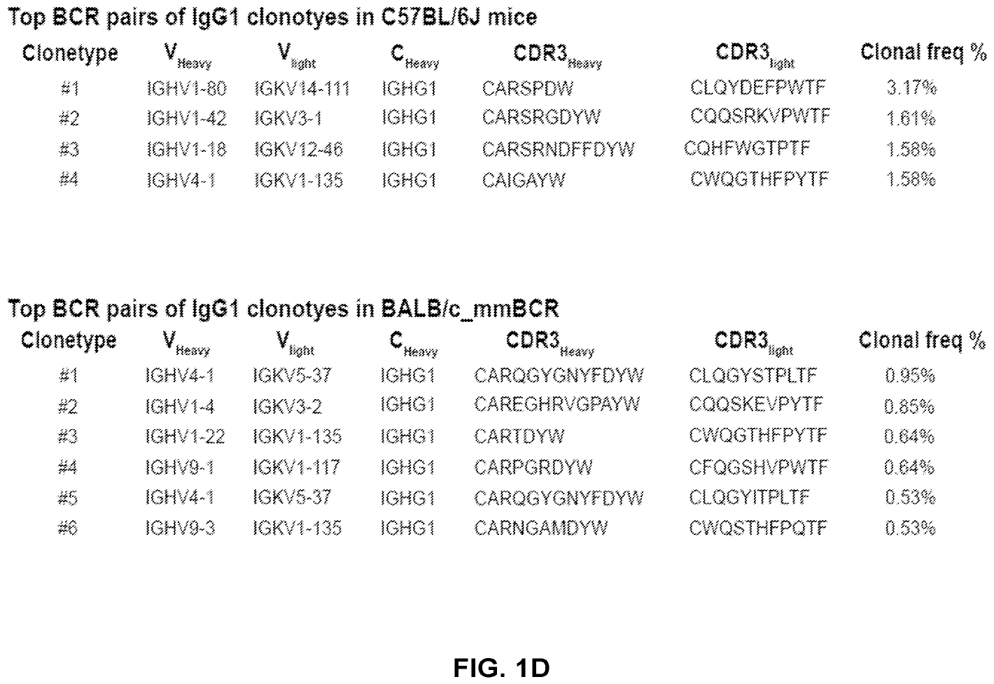

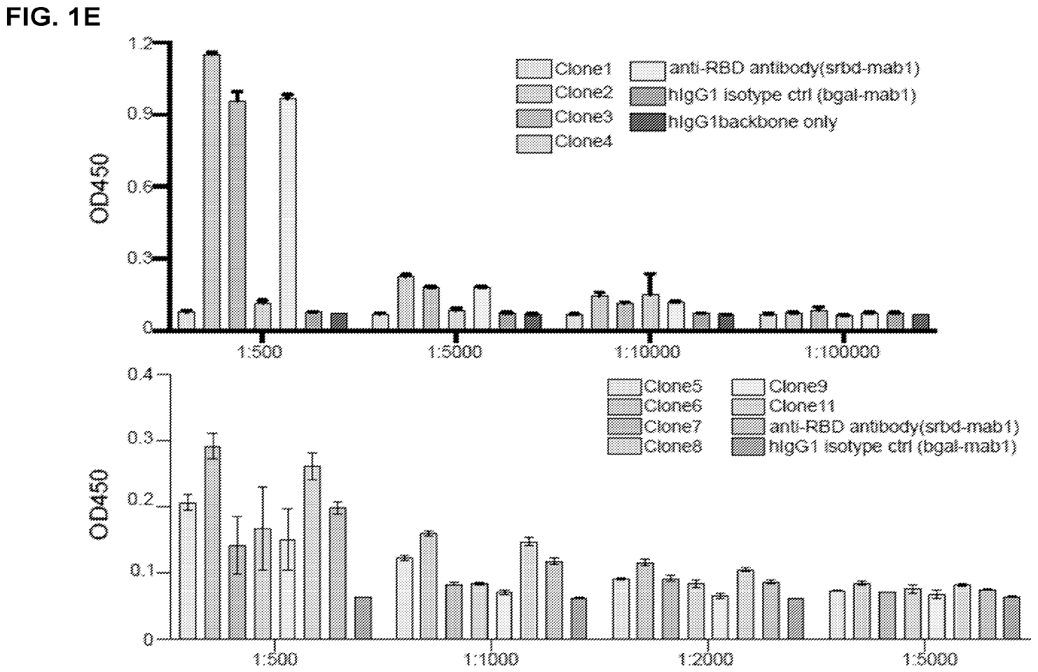

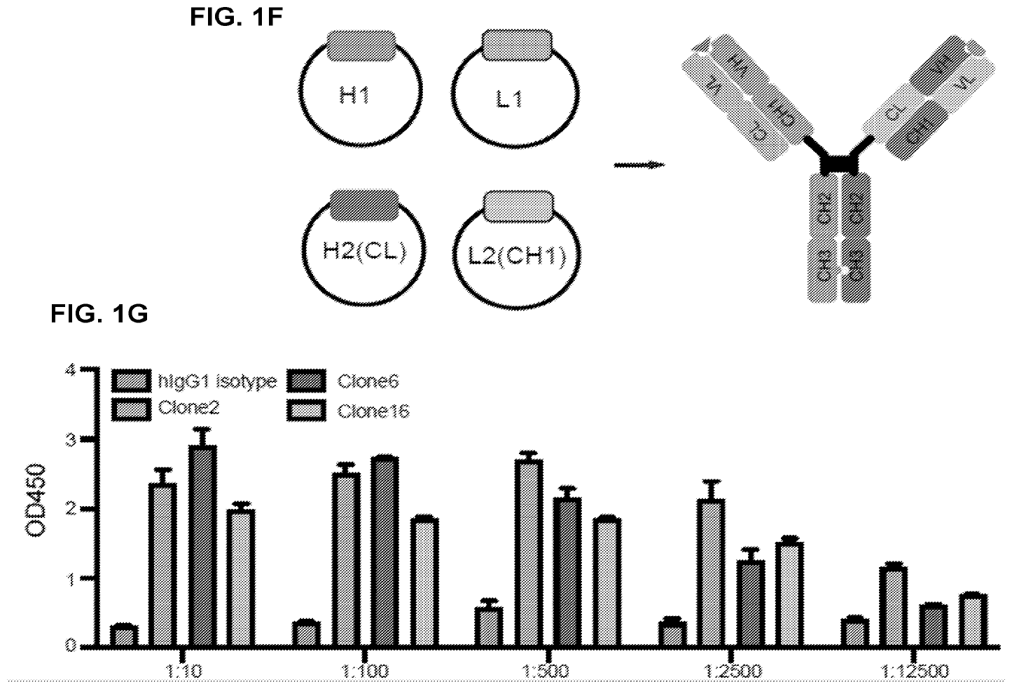

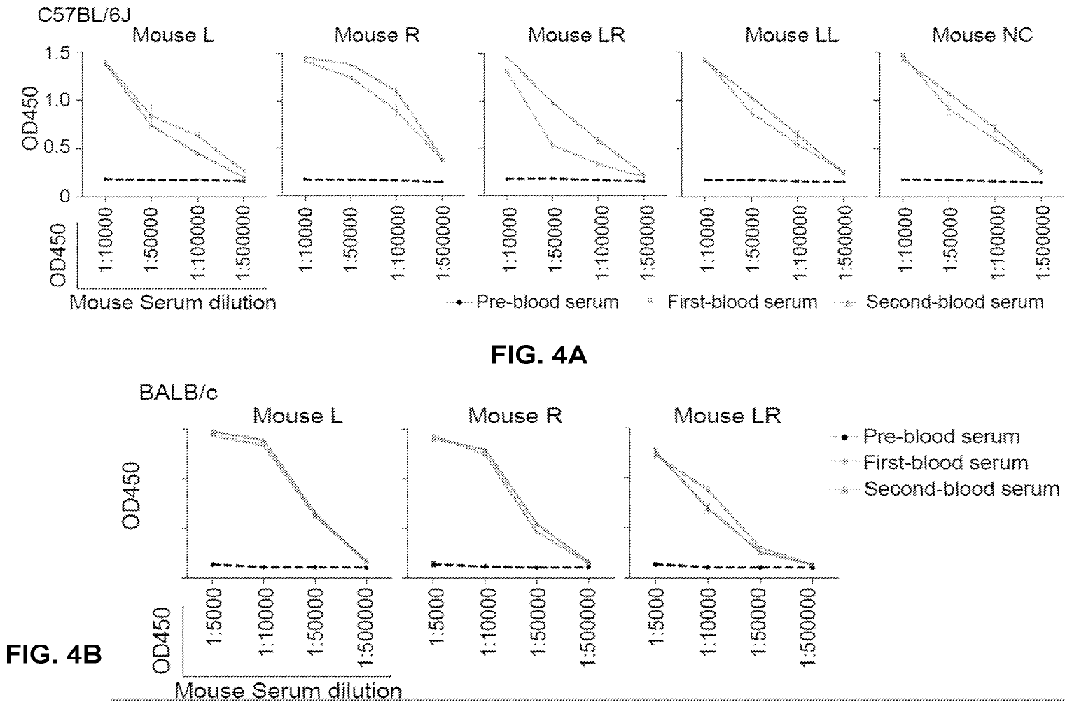

To generate potent and specific mAbs against SARS-COV-2, two different mouse strains were immunized: C57BL/6J and BALB/c with RBD-his tag protein following a standard 28-day immunized protocol (FIG. 1A). Progenitor B cells and plasma B cells were isolated from spleen, lymph node, and bone marrows of selected immunized mice with strong anti-serum titer (FIG. 4A and FIG. 4B) using anti-mouse CD138 beads. Single cell VDJ (scVDJ) sequencing was performed on the freshly isolated B cells (FIG. 1B). The scVDJ data revealed the landscape of immunoglobulin clonotypes (summarized in FIG. 1C), and identified enriched IgG1 clones (FIG. 1D). The VDJ sequences from the 11 top-ranked IgG1+ clones were selected, and the paired variable region of heavy and light chain were cloned into human IgG1 (hIgG1) heavy and light chain backbone vectors separately for antibody reconstruction utilizing the Expi293F mammalian expression system.

Anti-Spike RBD Monoclonal Antibodies have Single-Digit Nanomolar Affinity and Low-Picomolar Avidity

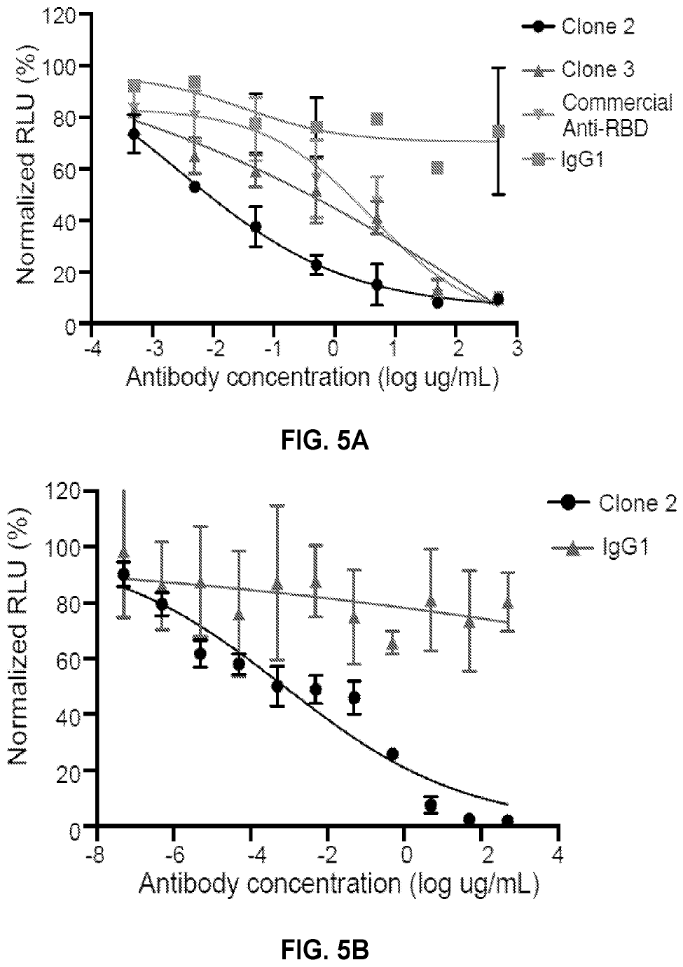

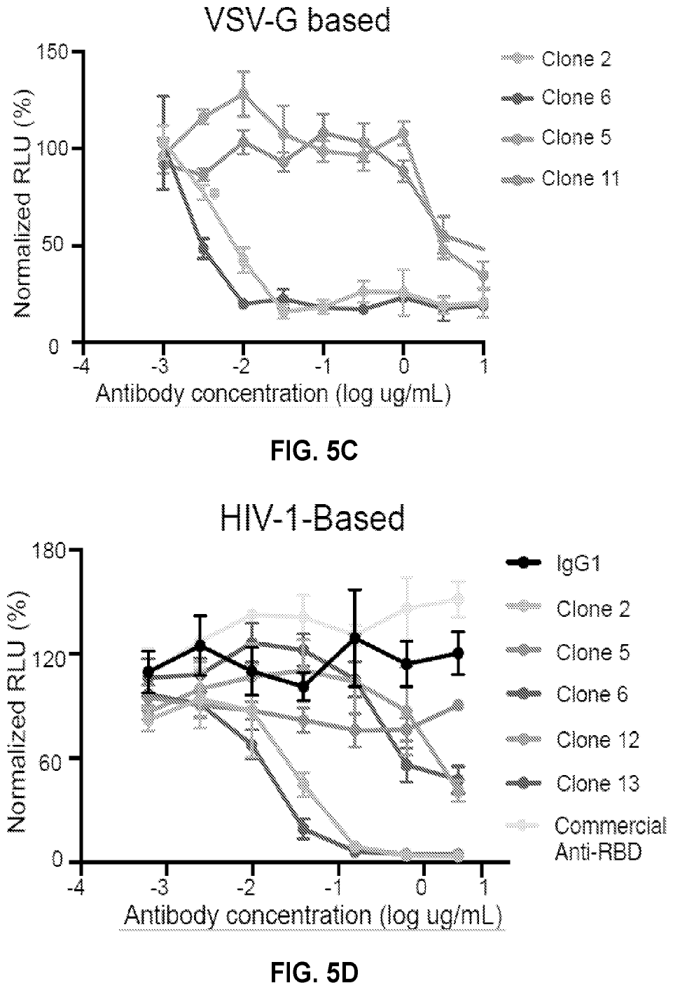

After successful expression and purification of hIgG1 antibody clones, the reactivity of the generated mAbs against SARS-COV-2 Spike RBD was tested by ELISA. Eight of the eleven mAbs showed positive RBD-binding in ELISA assay (FIG. 1E). Monoclonal antibody clones that showed high RBD ELISA positive rate for neutralizing ability were further screened using an HIV1-based SARS-COV-2 pseudovirus system and a VSV-based SARS-COV-2 pseudovirus system. Among these, two mAbs, Clone 2 and Clone 6 showed strongest ELISA activity and consistent neutralization effects (FIG. 5A-FIG. 5D).

As SARS-COV-2 continues to mutate and evolved emerging variants, it is critical to prevent viral escape from antibody recognition. Utilization of antibody cocktails from two or more mAbs has been utilized (Regeneron). An alternative approach would be multi-specific antibodies, which retain advantages of monoclonal antibodies within a single molecule. Accordingly, a bispecific antibody was generated using the antigen-specific variable regions of both Clone 2 and Clone 6 (named as Clone 16). To achieve effective reconstitution of the bispecific antibody, the following features were utilized: (i) a CrossMAb-KiH bispecific backbone, (ii) a “knobs into holes” (KiH) methodology to ensure the correct heterodimerization of the two different heavy chains, and (iii) a CH1 and CL exchange in heavy and light chain of one-half IgG to ensure correct pairing of heavy and light chains of each variable region (FIG. 1F). The antigen-specificity of the bispecific antibody (Clone 16) along with its two parent monospecific mAbs (Clone 2 and Clone 6) was validated using ELISA (FIG. 1G).

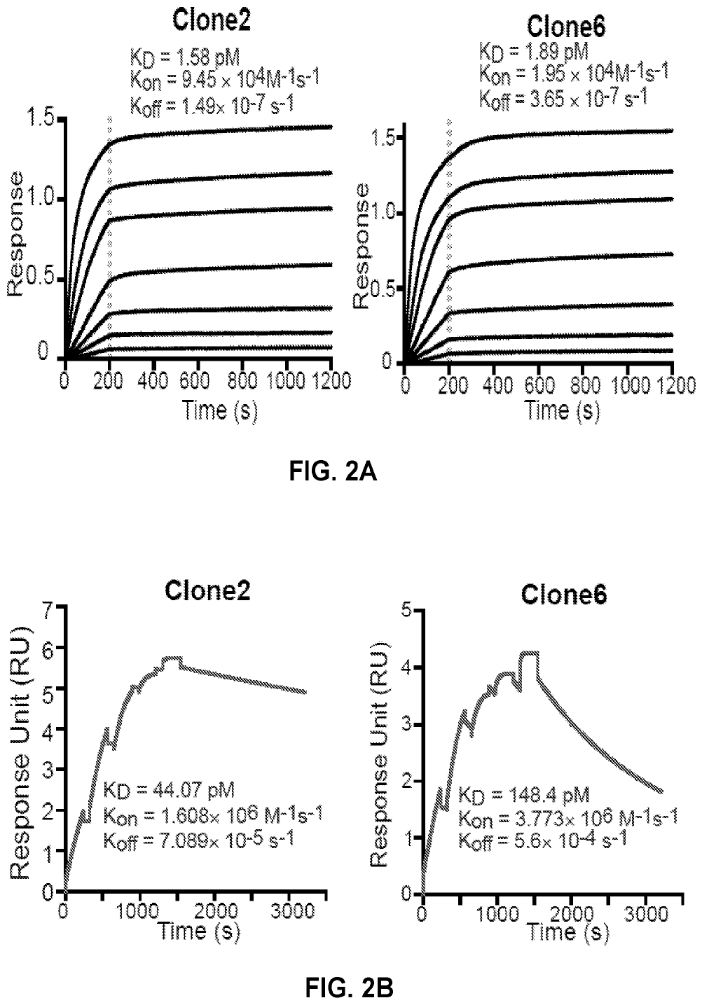

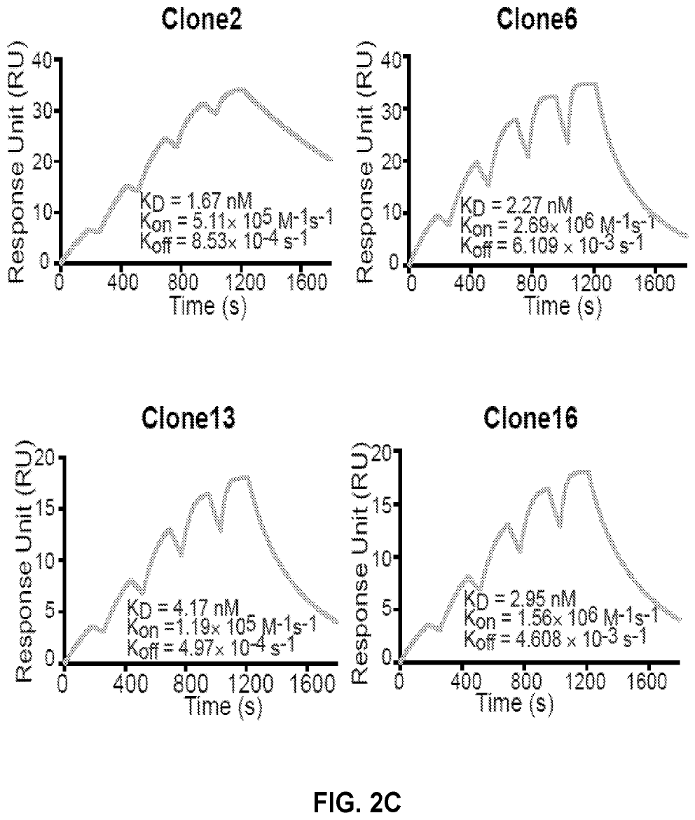

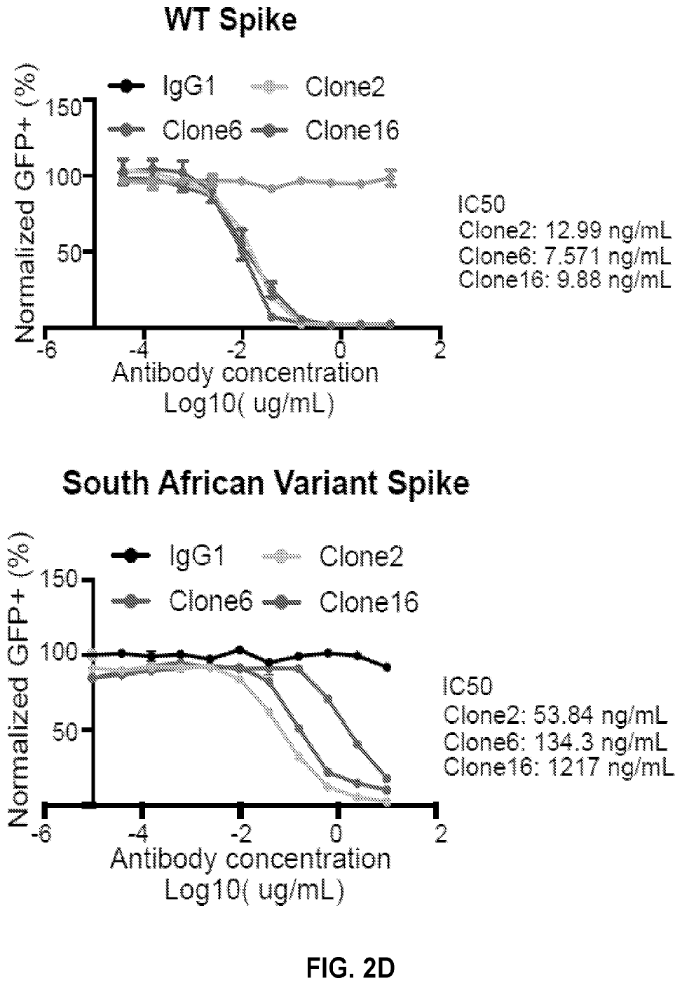

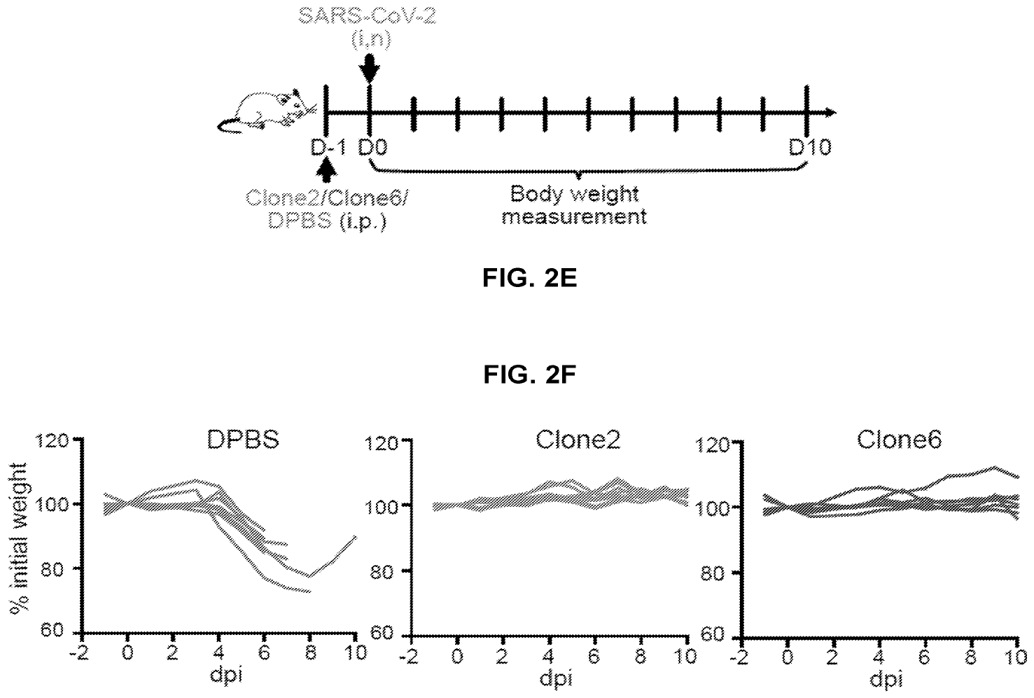

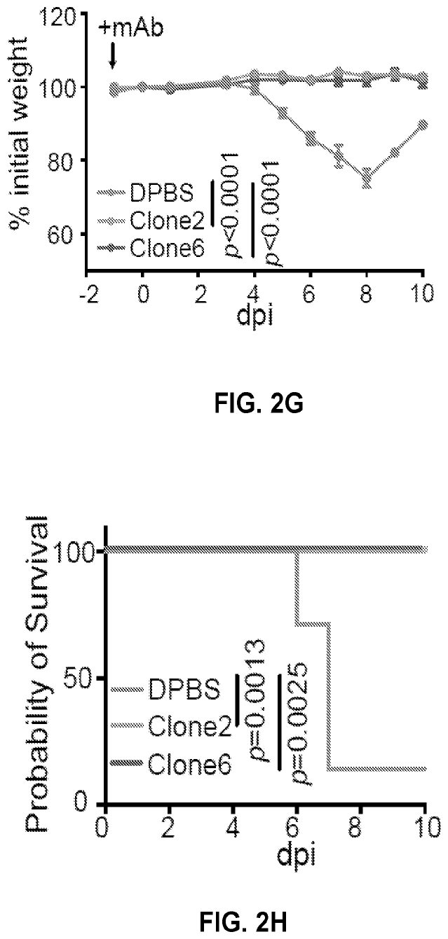

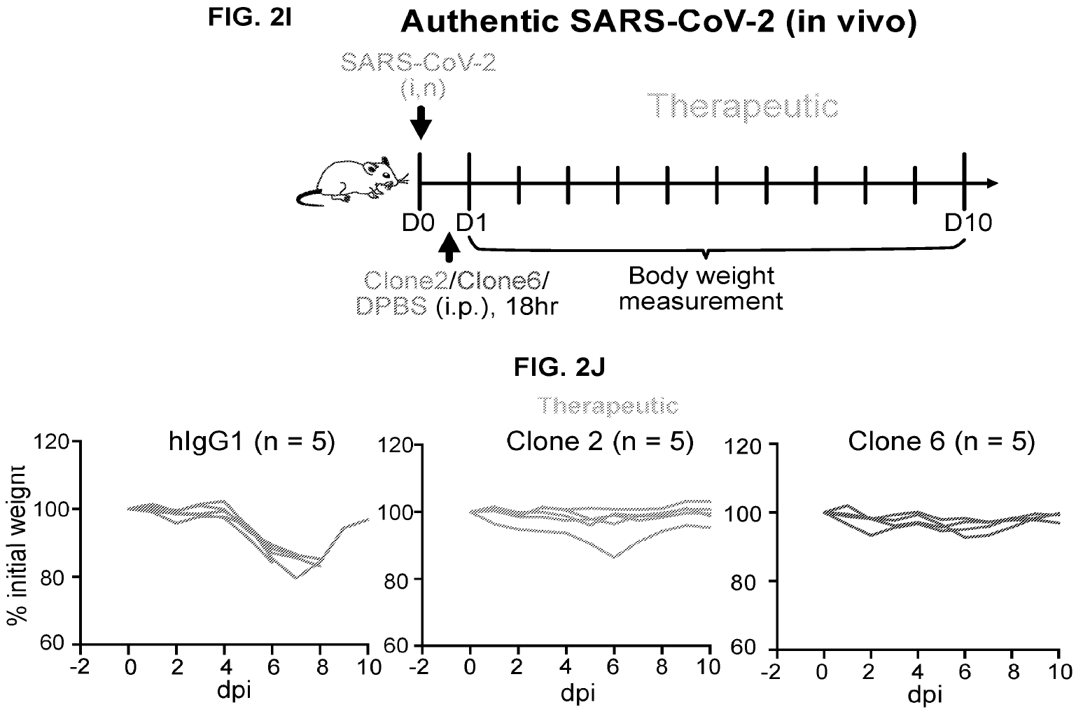

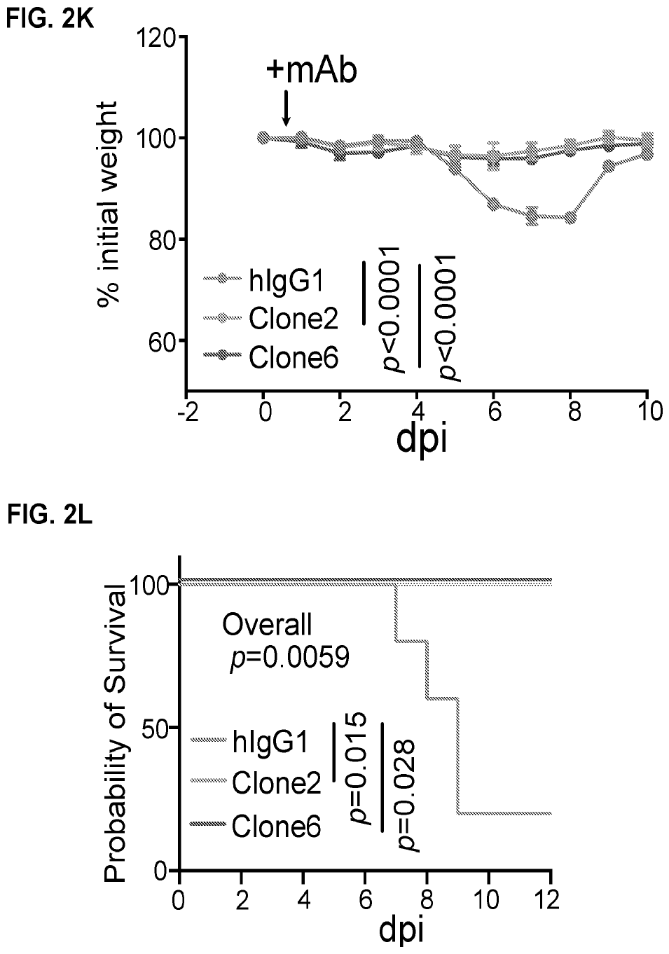

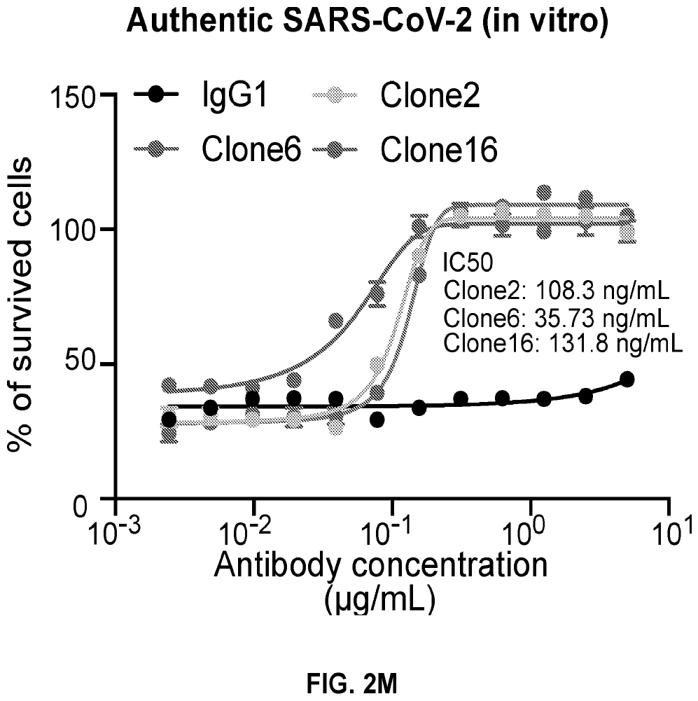

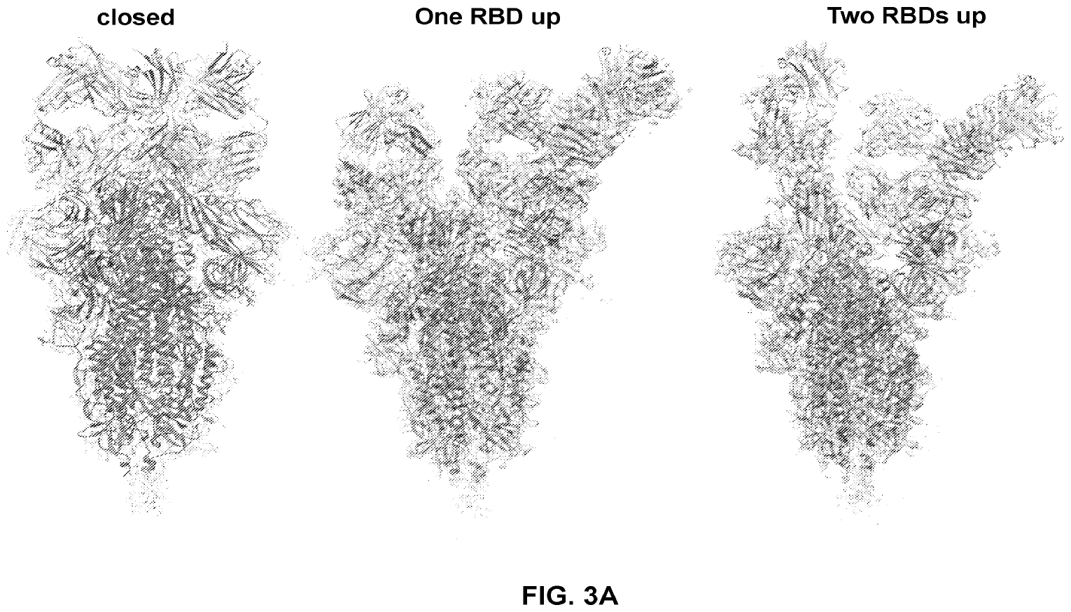

Next, bio-layer interferometry (BLI) using an Octet instrument and surface plasmon resonance (SPR) using BiaCore were used to precisely characterize the biophysical nature of the RBD reactivity of lead clones. BLI results showed that Clone 2 and Clone 6 bound to the RBD with picomolar level dissociation constant (Kd) (FIG. 2A). SPR using an NTA-CHIP based assay with the RBD-antigen immobilized also revealed picomolar level dissociation constant (Kd) for Clone 2 and Clone 6 (FIG. 2B). In the NTA-CHIP where the RBD-antigen immobilized, multiple binding can occur, leading to potential trimer formation of RBD protein and thereby rendering the measurement of avidity. In parallel, SPR was performed with a Fc-CHIP based assay with antibodies pre-coated, which measures single valent binding between RBD and the antibody. Results from Fc-CHIP SPR assay revealed single-digit nanomolar affinity of Clone 2 and Clone 6 (FIG. 2C). A humanized Clone 2 (named Clone 13) maintained strong binding affinity to RBD (FIG. 2C). The bispecific Clone 16 also displayed high affinity to RBD (FIG. 2C).

### Lead Antibody Clones Showed High Neutralizing Potency Against SARS-COV-2 and its Mutant Variant

Next, the neutralization ability of the monospecific and bispecific clones was tested using an HIV-1-based pseudovirus system pseudotyped by SARS-COV-2-WT and SARS-COV-2-South African variant spikes. As a result, all three antibodies (Clone 2, Clone 6 and the bispecific Clone 16) showed highly potent inhibitory ability against SARS-COV-2-WT pseudovirus with low ng/ml level IC₅₀ values (FIG. 2D). Interestingly, all three antibodies (Clone 2, Clone 6 and the bispecific Clone 16) also showed strong inhibitory ability against the South African variant SARS-COV-2-pseudovirus, although with a relatively higher level of IC₅₀ values (mid ng/ml for Clones 2/6, and high ng/mL for Clone 16) (FIG. 2D).

Authentic virus neutralization was performed in a biosafety level 3 (BSL3) setting, and showed that all three antibodies (Clone 2, Clone 6 and the bispecific Clone 16) inhibited the viral propagation of the fully infectious SARS-COV-2 WA1 (low-mid ng/ml level IC50 s) (FIG. 2M).

As Clone 2 and Clone 6 exhibit high potency against both pseudotyped SARS-COV-2 and its South African mutant variants in neutralization assays in vitro, their efficacy was evaluated against replication-competent SARS-COV-2 in vivo. The neutralization efficacy of Clone 2 and Clone 6 was tested in a mouse model of lethal SARS-COV-2 infection using K18-hACE2 transgenic mice. The hACE2 transgenic mice were divided into three group and injected with 20 mg/kg of mice body weight for Clone 2 and Clone 6 along with placebo control. These mice were challenged with lethal dose of replication-competent SARS-COV-2 virus in a biosafety level 3 (BL3) facility (FIG. 2E). All (8/8, 100%) mice in the placebo group developed severe disease due to viral challenge, and most (7/8, 87.5%) of them consistently decreased body weight and eventually succumbed from the disease (FIG. 2F—FIG. 2H). In sharp contrast, all mice receiving treatment of either Clone 2 (8/8, 100%) or Clone 6 (7/7, 100%) were free of severe disease symptoms, maintained their body weight throughout the duration of the study, and survived from SARS-COV-2 infection.

In the therapeutic setting, all (5/5, 100%) mice in the placebo group developed severe disease, and most (4/5, 80%) of them consistently decreased body weight, with one mouse with body weight recovered (FIG. 2I-FIG. 2L). In contrast to control group, all mice receiving therapeutic treatment of either Clone 2 (5/5, 100%) or Clone 6 (5/5, 100%) were healthy, maintained their body weight throughout the duration of the study, and survived the viral infection (FIG. 2I-FIG. 2L). These data suggested that these mAbs can protect the animals from lethal doses of SARS-COV-2 infection in both prophylactic and therapeutic settings.

### Cryo-EM Structure of Lead Antibody Clones Bound to the Trimeric Spike Revealed Epitopes and Conformations

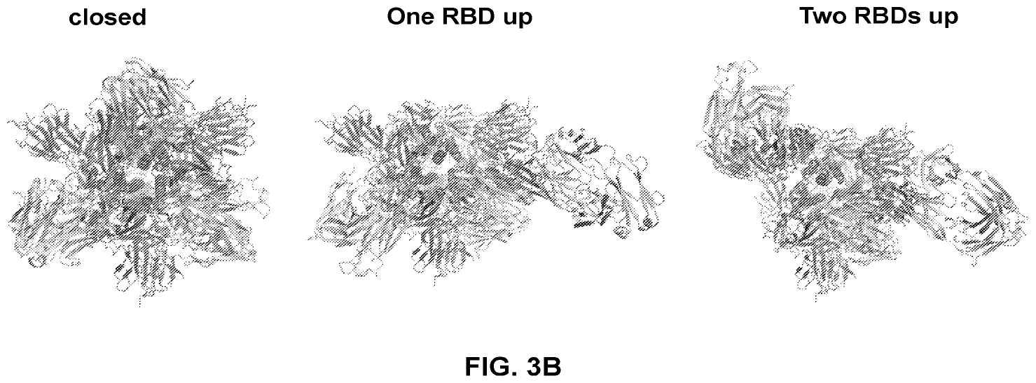

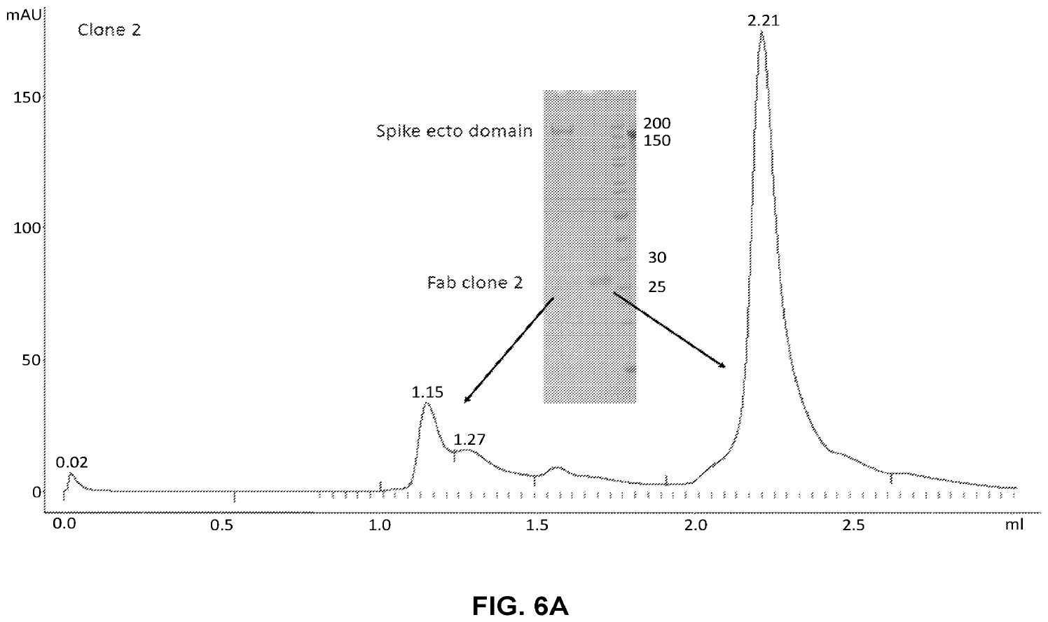

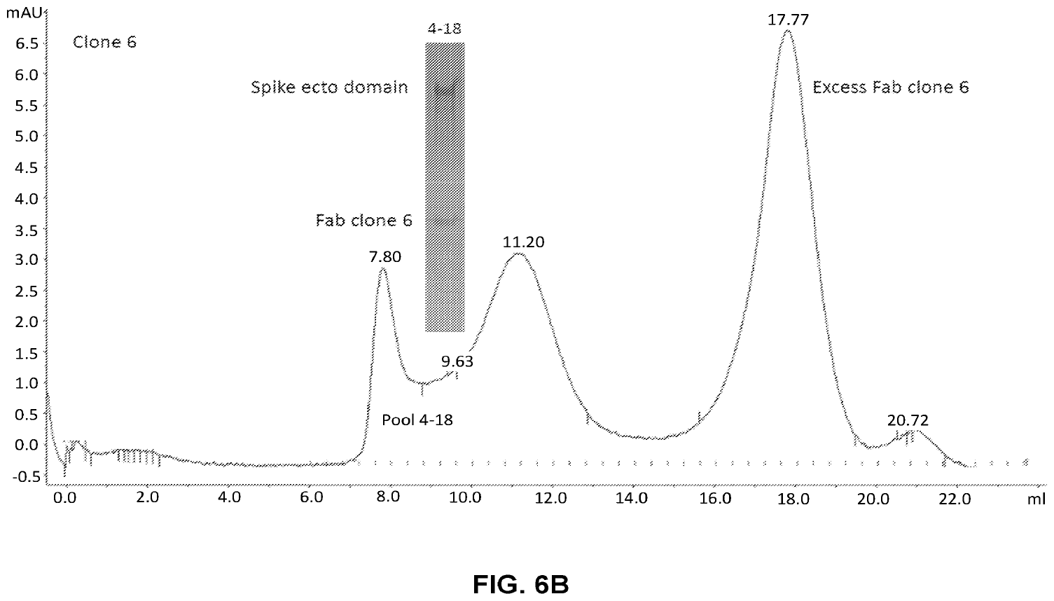

The cryo-EM structures of SARS-COV-2 trimeric spike complex with lead mAb clones were determined to elucidate the mechanism of antibody inhibition by the lead mAb clones. Fab fragments of Clone 2 and Clone 6 were generated, purified, and complexed with SARS-COV-2 spike protein for CryoEM studies. The chromatography results showed successful purification and formation of antibody-spike protein complexes (FIG. 6A and FIG. 6B) which were used for the CryoEM structure analyses. A series of high-resolution CryoEM image data were collected and processed. The CryoEM structures of lead antibodies reveal clear and distinct epitopes of Fab binding to the RBD of trimeric spike (FIG. 3A and FIG. 3B). In addition, the CryoEM structures reveal combinations of open and closed conformations of trimeric spike, which are indicators of effective binding (FIG. 3A and FIG. 3B). The structures showed that the binding sites of all clones overlap with ACE2 and due to much higher affinity of these mAbs, these antibodies can outcompete spike-ACE2 receptor binding, which aligns with the neutralization and in vivo efficacy results described herein.

The ongoing COVID-19 pandemic and the rapidly emerging new mutant variants urges for rapid development of prophylactic and therapeutic interventions. Discovery and development of new neutralization antibodies with expanding collections of epitopes that cover various binding sites are critical to provide countermeasures against viral escape. SARS-COV-2 Spike RBD protein immunization was combined herein with high-throughput single cell BCR sequencing technology to establish a platform to rapidly develop neutralizing antibody candidates. Two highly potent and specific SARS-COV-2 neutralizing mAb clones were identified (Clone 2 and Clone 6) that have single-digit nanomolar affinity and low-picomolar avidity. A bispecific antibody (Clone 16) was also generated from these two lead clones. The lead monospecific and/or bispecific antibodies showed strong neutralization ability against SARS-COV-2 and its highly contagious South African mutant that also poses a risk of reducing the efficacy of currently available therapeutic antibodies and vaccines. CryoEM structure studies of mAb-trimeric spike complexes mapped distinct epitopes of these lead antibodies that are also different from various previous mAbs. The CryoEM structural analyses also revealed combinations of open and closed conformations of trimeric spike. These antibodies expand the repertoire of the toolbox of COVID-19 countermeasures against the SARS-COV-2 pathogen and its emerging mutant variants.

### Example 2: Monoclonal Antibodies By Rapid mRNA Immunization

### Materials and Methods

Rapid mRNA Immunization of Humanized Mice.

The full-length Omicron spike sequence used in mRNA immunization was based on two North America patients identified on Nov. 23, 2021. The LNP-mRNA was generated as previously described. Humanized mice with human IgG and IgK transgene knock-ins (ATX-GK, Alloy Therapeutics) were used for rapid mRNA immunization, according to an accelerated (two-week) vaccination schedule. Pre-immune sera were collected from the mice prior to the initiation of immunization. The mice were primed with intramuscular injection of 10 μg Omicron LNP-mRNA and boosted on days 2, 4, 7 with the same dose as prime. On day 11, three days prior to sacrifice, mice received a final boost with 20 μg Omicron LNP-mRNA. All mice were retro-orbital bled on days 7, 14 and anti-plasma titers were evaluated using an immunoassay as described below.

ELISA Analysis for Plasma and mAbs Supernatant Binding to Omicron RBD Protein.

Plasma was extracted from surface layer by using SepMate-15 tubes with Lymphoprep gradient medium (StemCell Technologies) after centrifugation at 1200 g for 20 minutes. Afterwards, antibody titers in plasma against Omicron RBD were evaluated using a direct coating ELISA. 384-well microtiter plate (Corning) were coated with 3 μg/ml of Omicron RBD recombinant protein (Sino Biological 40592-V08H121) in PBS at 4° C. for overnight. Plate was washed with standard wash buffer PBS-T (PBS containing 0.05% Tween 20) and blocked with blocking buffer (PBS containing 0.5% BSA) for 1 hour at room temperature (RT). Either serially diluted plasma samples or mAbs supernatant were added to plate and incubated for 1 hour at RT. Wells were then washed and incubated with secondary goat anti-mouse IgG labeled with HRP (Fisher, Cat #A-10677) at 1:2500 dilution in a blocking buffer for 1 h at RT. Thereafter, wells were developed using TMB substrate (Biolegend, 421101) according to the manufacturer's protocol. The reactions were terminated with IM H₃PO₄ after 20 minutes incubation at RT and optical density (OD) was measured by a spectrophotometer at 450 nm (PerkinElmer EnVision 2105).

### Humanized Mice B Cell Isolation and Purification.

Three sets of single B cells were collected: PBMC sample, Omicron RBD-specific memory B cell sample and CD138+ plasma B cell sample. PBMC cells were isolated from fresh whole blood by using SepMate-15 tubes with Lymphoprep gradient medium (StemCell Technologies) after centrifugation at 1200 g for 20 minutes. Poured top layer solution that contained PBMCs from SepMate tubes to a new falcon tube and washed once with PBS+2% FBS, resuspended with PBS and stored on ice until use.

Omicron RBD-specific memory B cells were isolated from pre-enriched memory B cells by magnetic positive selection according to the manufacturer's protocol (Miltenyi Biotec, 130-095-838). Briefly, spleen and lymph nodes were gently homogenized and red blood cells were lysed in ACK lysis buffer (Lonza). The remaining cells were washed by PBS with 2% FBS and filtered through with a 50 ml falcon tube. Thereafter, memory B cells were labeled with memory B cell biotin-antibody cocktail combined with anti-biotin microbeads and isolated using a magnetic rack. Enriched memory B cells were eluted and mixed with 25 μg of Omicron RBD recombinant protein with his tag and incubated for 30 mins on ice. After incubation, the complex was washed and respectively incubated with anti-his-APC antibody and anti-APC microbeads. The final antigen-enrichment B cells were eluted in PBS and stored on ice until use.

Plasma B cells were collected by fragmenting and rinsing bone marrows with PBS containing 2% FBS. Non-plasma cells were labeled with a biotin-conjugated antibody cocktail combined with anti-biotin microbeads and separated using a magnetic rack according to the manufacturer's protocol (Miltenyi Biotec 130-092-530). Purified plasma B cells were eluted and sequentially incubated with CD138 microbeads for an additional 15 minutes at 4° C. The final CD138+ plasma B cells were eluted in PBS and stored on ice until use.

### Single Cell VDJ Sequencing and Data Analysis.

10,000 of cells per each above collection were loaded on Chromium Next GEM Chip K Single Cell Kit. Single-cell lysis and cDNA first strand synthesis were performed using Chromium Next GEM Single Cell 5′ Kit v2 according to the manufacturer's protocol. The barcoded single strand cDNA was isolated via a Dynabeads MyOne SILANE bead cleanup mixture. The cDNA was amplified by 14 PCR cycles and purified via SPRI bead cleanup (X0.6) according to the manufacturer's protocol. For BCR repertoire libraries, 2 μL of amplified cDNA underwent two rounds of Target Enrichment using nested custom primer pairs specific for BCR constant regions. The target's enrichments for heavy chain and light chain were performed in separate reactions. After each PCR reaction, the PCR products were subjected to double-sided size selection with SPRI bead cleanup (X0.6 followed by X0.8) The primers were designed by Alloy biotechnologies and synthesized by KECK.

Twenty-five nanograms of each target enrichment PCR product was combined, and used for library preparation, consisting of fragmentation, end repair, A-tailing, adaptor ligation (Library Construction Kit) and sample index PCR (Dual Index Kit TT Set A) according to the manufacturer's instructions. The final library was profiled and quantified using the D1000 ScreenTape assay (Agilent) for TapeStation system. Libraries were sequenced by paired-end sequencing (26× 91 bp) on an Illumina Miseq. All libraries were targeted for sequencing depth of 5,000 raw read pairs per cell.

For bioinformatic analysis, BCL data were converted to demultiplexed FASTQ files using Illumina Miseq controller and processed by using Cell Ranger v6.0.1 with default settings to align the reads to customized germline V and J gene references. The custom references were created by combining mouse constant genes along with human V(D)J genes. The consensus amino acid sequences of top-enriched clonotypes from each collection were selected by using the Loupe V(D)J Browser and cDNA sequences were synthesized for further molecular cloning and recombinant antibody expression.

In Vitro Generation of Recombinant mAbs.

The cDNA of paired heavy- and light-chains from top-enriched IgG clonotypes were codon-optimized and respectively subcloned into human IgG1 expression vectors, based on Gibson assembly, to generate recombinant mAbs (See Table 3). mAbs were produced by transient transfection into Expi293F™ cells with equal amounts of paired heavy- and light-chain expression vectors using ExpiFectamine 293 transfection kit according to the manufacturer's protocol (Thermo fisher). Five days post antibody expression, the secreted mAbs from cultured cells were collected and purified by affinity chromatography using rProtein A Sepharose Fast Flow beads according to the manufacturer's instruction (Cytiva). Eluted mAbs were eventually kept in PBS for long-term storage after buffer exchange using Amicon Ultra-4 Centrifugal Filter (MilliporeSigma). The purified mAbs were examined by running SDS-PAGE and kept in −80° C. for further usage.

### Omicron Pseudovirus Generation and Neutralization Assay.

Omicron pseudovirus was generated by using a modified method from a previously described study. Briefly, full length Omicron spike gene was constructed into GFP encoding (pCCNanoLuc2AEGFP) human immunodeficiency vector backbone, then Omicron spike protein expression vectors were combined with HIV-1 structural corresponding plasmids and co-transfected into HEK-293T cells with PEI (1 mg/ml, PEI MAX, Polyscience). Two-day post-transfection, viral supernatants were harvested, collected, filtered and aliquoted to use in assays.

Neutralization assays were performed by incubating pseudovirus with serial dilutions of mAbs. 10,000 cells/well of HEK-293T-hACE2 cells were seeded in a 96-well plate, 24 hours prior to assay. mAbs supernatant/purified mAbs were serially diluted in DMEM media with 10% FBS and incubated with an equal volume of purified Omicron pseudovirus at 37° C. for 1 hour. Thereafter, the virus-antibody mixture was added triplicate onto HEK-293T-hACE2 cells and incubated at 37° C. for additional 24 hours. Then, infected cells were counted and determined by evaluating GFP expression after 24 hours exposure to virus-antibody mixture using Attune NxT Acoustic Focusing Cytometer (Thermo Fisher). Half-maximal inhibitory concentration (IC50) for mAbs was calculated with a four-parameter logistic regression using GraphPad Prism (GraphPad Software Inc.).

### Antibody Binding Kinetics, Epitope Mapping by Bio-Layer Interferometry (BLI).

Antibody binding kinetics for anti-Omicron RBD mAbs were evaluated by BLI on an Octet RED96e instrument (ForteBio) at RT. 25 ng/ul of purified mAbs were captured on a AHC biosensor (Sartorius, 18-5060). The baseline was recorded for 60 s in a running buffer (PBS, 0.02% Tween-20, and 0.05% BSA, pH 7.4). Followed by sensors were subjected to an association phase for 300 s in wells containing Omicron RBD with his tag protein diluted in the buffer. In the dissociation phase, the sensors were immersed in the running buffer for 500 s. The dissociation constants K_{D}, kinetic constants Kₒₙ and K_{off} were calculated by FortéBio data analysis software.

For epitope mapping, two different antibodies were sequentially injected and monitored for binding activity to determine whether the two mAbs recognized separate or closely-situated epitopes by in-tandem approach on OCTET RED. Briefly, SARS-COV-2 RBD-His recombinant protein (Sino Biological 40592-V08H121) was diluted with PBS to 20 μg/mL, and was captured by anti-Penta-His (HIS1K) sensors (Sartorius, 18-5120). The primary antibody was diluted to 150 nM with a running buffer in wells, and then sensors were firstly subjected to an association phase for 500 s, the response value was recorded. Followed by sensors were subjected to the secondary antibody mixture, and the response value was recorded again. Competition tolerance was calculated by the percentage increase of response after the secondary antibody was added. The column indicates the primary antibody, and the row indicates secondary antibodies. Competition tolerance less than 25% indicates a high possibility of closely-situated epitope.

### ACE2 Competition Assay.

3 μg/ml of Omicron RBD recombinant protein (Sino Biological 40592-V08H121) was coated in a 384-well ELISA plate (Corning) at 4° C. for overnight incubation. Plate was washed with standard wash buffer PBS-T (PBS containing 0.05% Tween 20) and blocked with a blocking buffer (PBS containing 0.5% BSA) for 1 hour at room temperature (RT). 50 ng/mL his-tagged hACE2 protein and PBS were firstly added to plate and incubated for 1 hour at RT. Wells were washed and incubated with serially diluted purified mAbs were sequentially added and incubated for 1 hour at RT. Thereafter, wells were incubated with secondary goat anti-mouse IgG labeled with HRP (Fisher, Cat #A-10677) at 1:2500 dilution in blocking buffer for 1 h at RT after washed. Finally, wells were developed using TMB substrate (Biolegend, 421101) according to the manufacturer's protocol. The reactions were terminated with 1M H3PO4 after 20 minutes incubation at RT and optical density (OD) was measured by a spectrophotometer at 450 nm (PerkinElmer En Vision 2105).

### Standard Statistics.

Standard statistical methods were applied to non-high-throughput experimental data. The statistical methods are described in figure legends and/or supplementary Excel tables. The statistical significance was labeled as follows: n.s., not significant; *p<0.05; **p<0.01; ***p<0.001; ****p<0.0001. Prism (GraphPad Software) and RStudio were used for these analyses.

### Replication, Randomization, Blinding and Reagent Validations.

Sample size: Sample size determination was performed according to similar work in the field. Replicate experiments have been performed for key data shown in this study.

Replication: Biological or technical replicate samples were randomized where appropriate. In animal experiments, mice were randomized by cage, sex and littermates.

Binding: Experiments were not blinded. It is unnecessary for animal immunization for antibody production to be blinded.

Antibodies and dilutions: Commercial antibodies used for various experiments are described in methods, with typical dilutions noted. For custom Antibodies generated in this study, dilutions were often serial titrations (i.e. a number of dilutions as specified in each figure). Commercial antibodies were validated by the vendors, and re-validated in house as appropriate. Custom antibodies were validated by specific antibody-antigen interaction assays, such as ELISA. Isotype controls were used for antibody validations.

Eukaryotic cell lines: Cell lines are from various sources as described in methods. Cell lines were authenticated by original vendors, and re-validated in lab as appropriate. All cell lines tested negative for mycoplasma. No commonly misidentified lines involved.

Animals and other organisms: Laboratory animals: M. musculus, ATX strain (Alloy Tx).

Development of RApid mRNA Immunization of Humanized Mice (RAMIHM), a Highly Efficient Strategy to Identify Fully Human Monoclonal Antibodies.

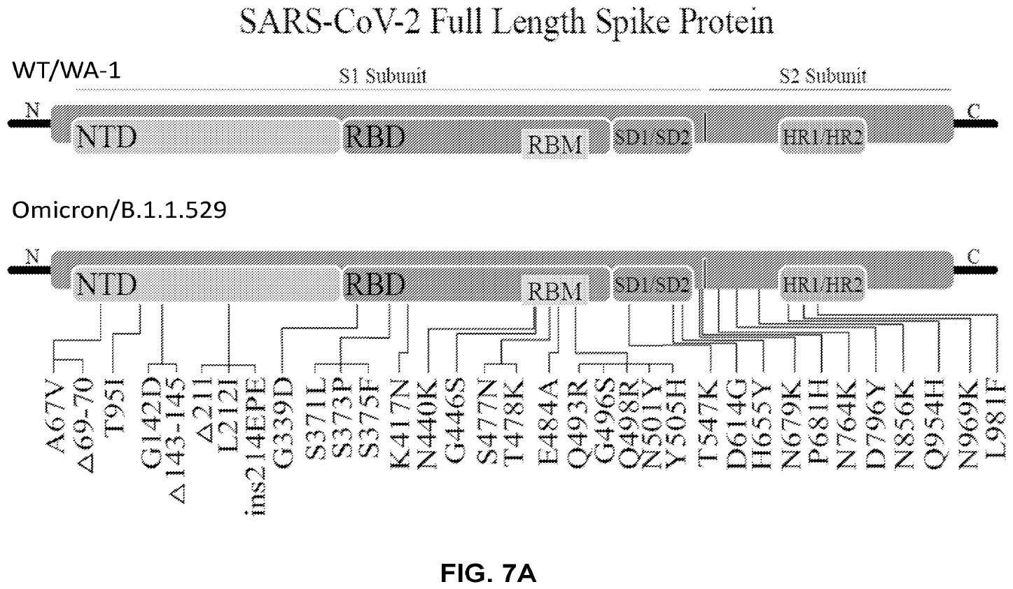

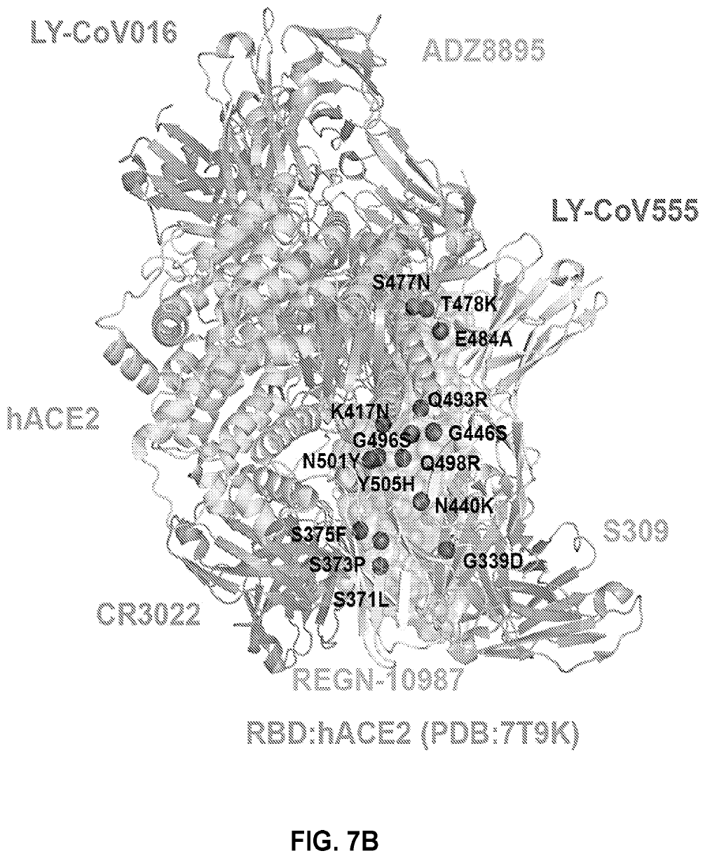

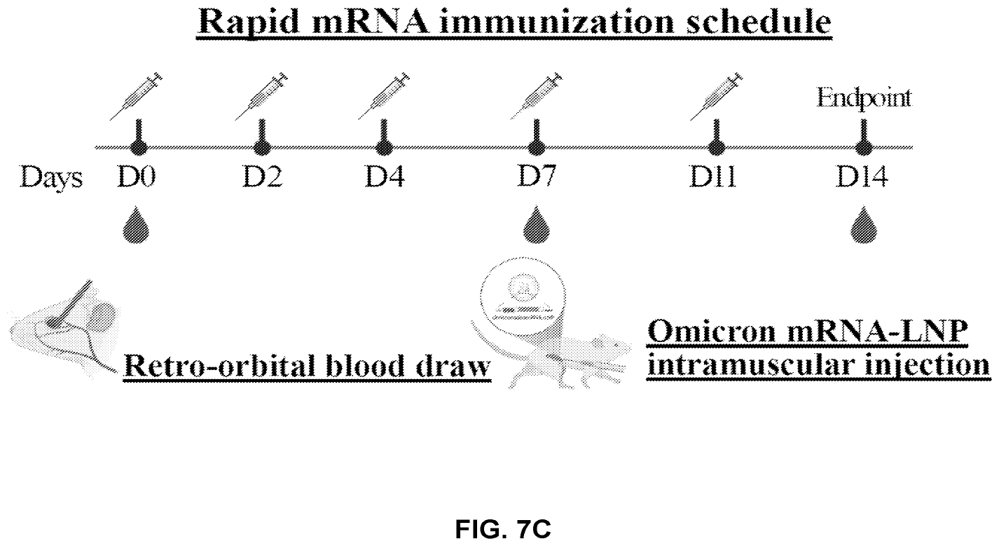

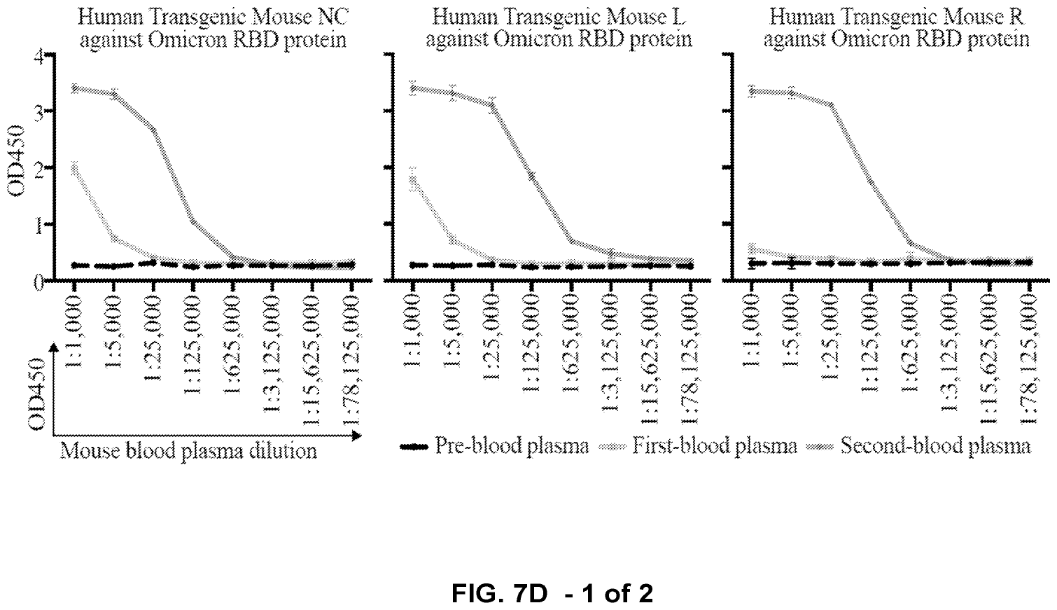

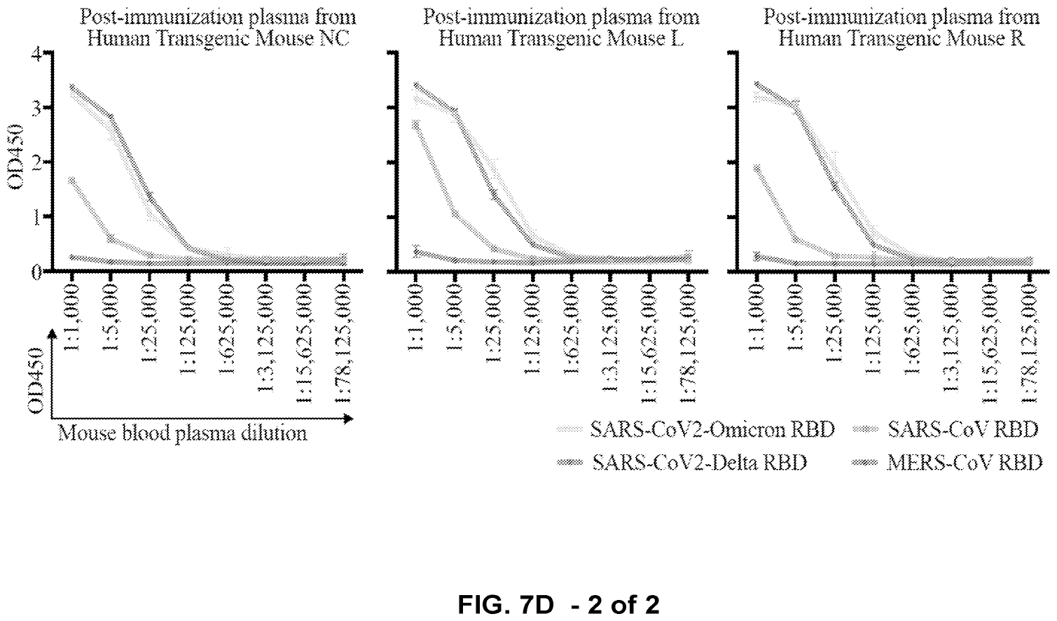

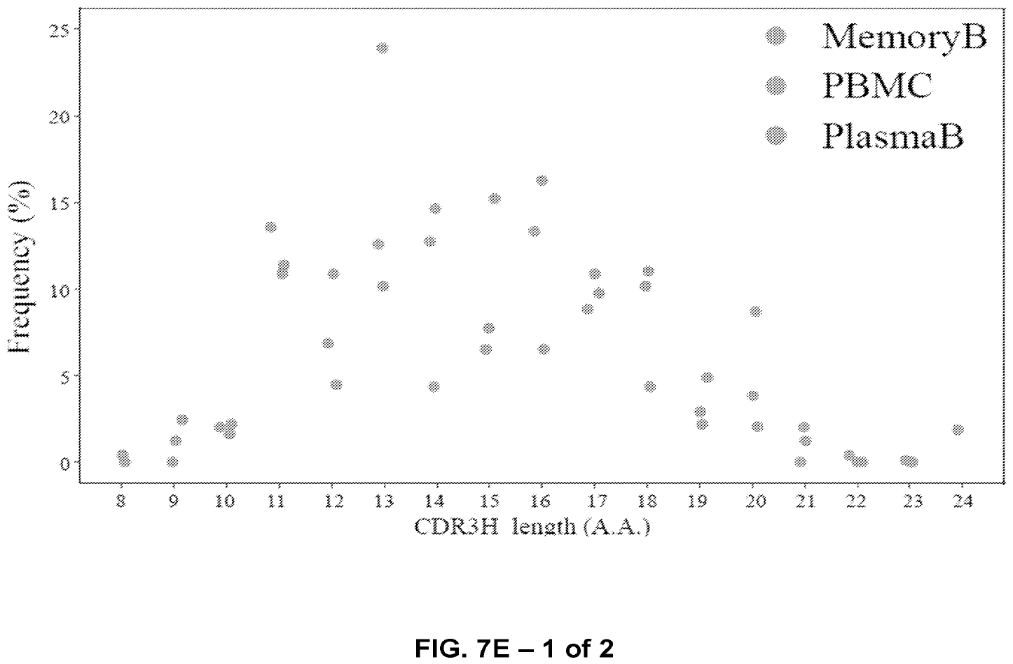

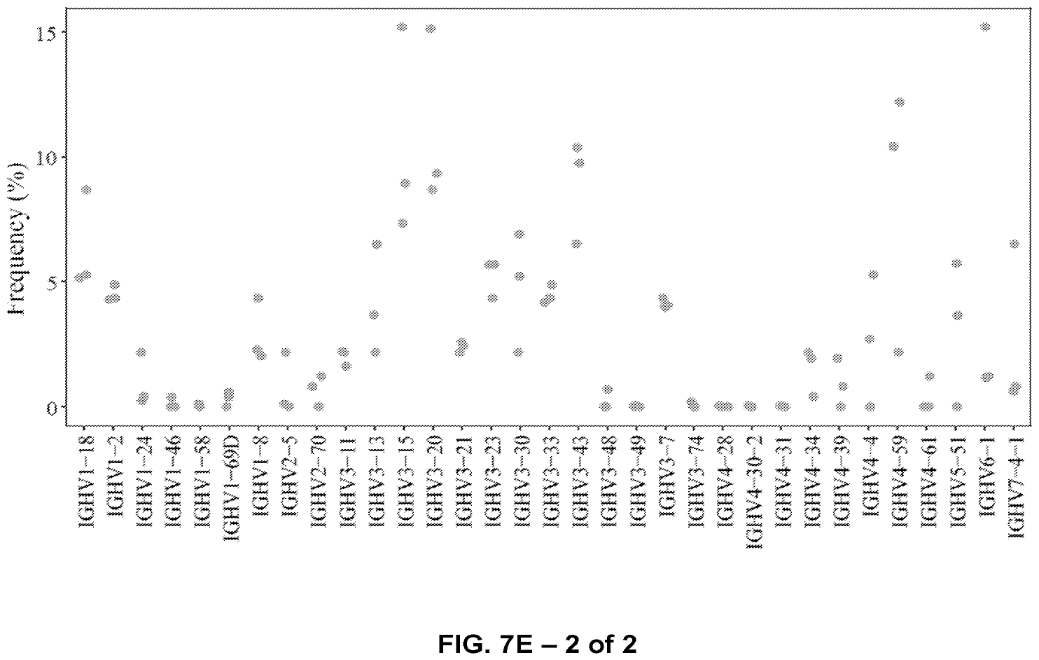

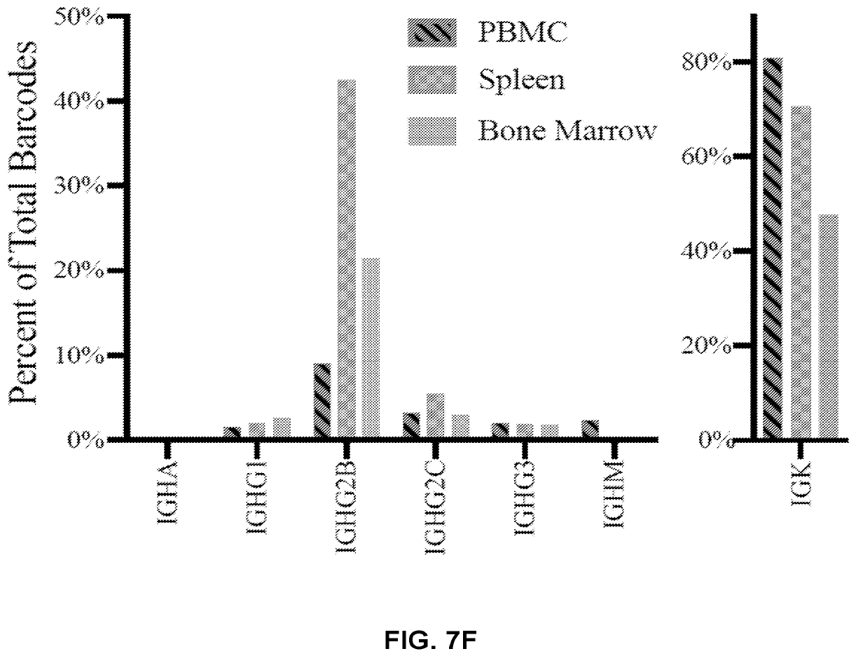

To date, two-dose SARS-COV-2 mRNA-based vaccination strategy has been demonstrated to effectively induce humoral and cellular immunity to SARS-COV-2, including the ancestral virus (ancestral, reference, wildtype (WT), Wuhan-1, or WA-1, identical sequences), and its VoCs such as Delta variant. However, a number of recent studies demonstrated that the SARS-COV-2 Omicron variant has substantial changes in its genome, especially the spike protein (FIG. 7A), and illustrated dramatically decreased neutralizing titers in convalescent or vaccinated recipients, causing waning immunity and massive breakthrough infections. Importantly, nearly all antibodies initially developed against the ancestral virus have substantially dropped, or completely lost, the neutralization ability against Omicron. For example, several of the currently approved or emergency authorized mAbs have their binding interfaces impacted by the Omicron mutations (FIG. 7B). Therefore, next-generation neutralizing antibodies are needed in speed. To combat the rapidly evolving VoCs, for example the current resurgence of the Omicron pandemic, it is important to have the ability to rapidly develop translatable human neutralizing mAbs to quickly react to the needs of new therapeutics. Thus the examples presented in the current disclosure describe the development of a novel antibody discovery approach named RAMIHM, with repetitive intramuscular injections using high doses of LNP-mRNA, followed by B cell isolation, antigen enrichment and single B cell sequencing (FIG. 7C). This method was applied directly with Omicron-spike-encoding LNP-mRNA to induce Omicron-specific immune responses for isolation of Omicron-targeting mAbs.

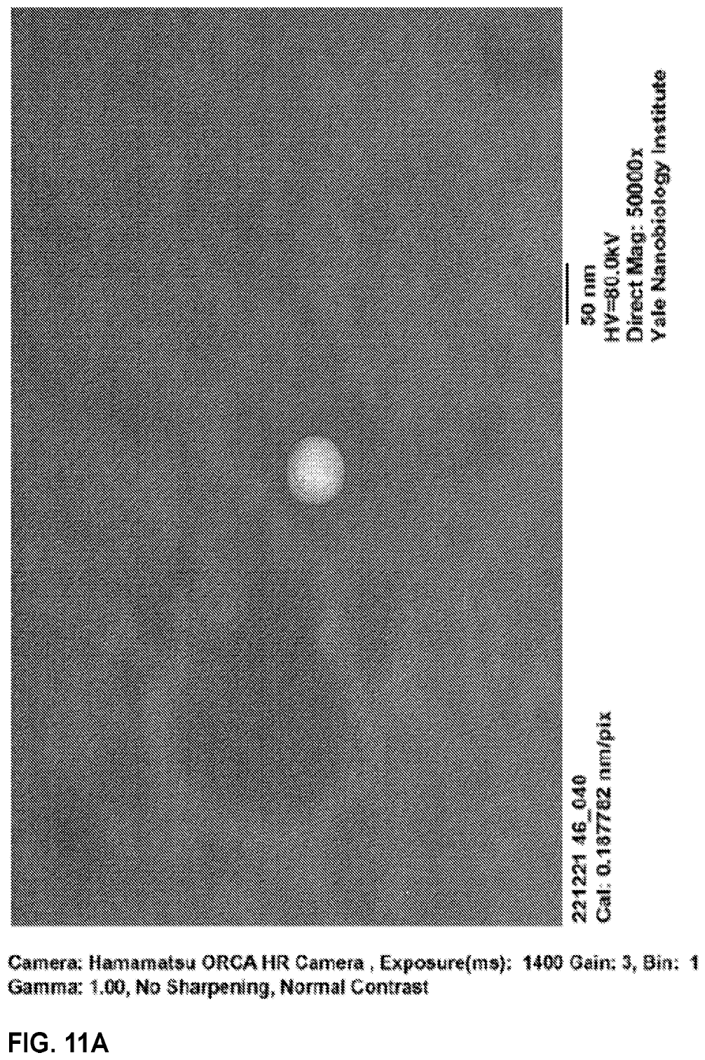

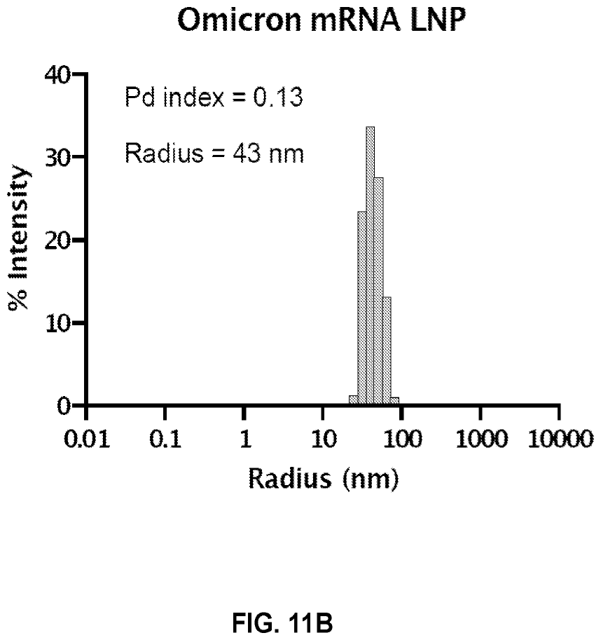

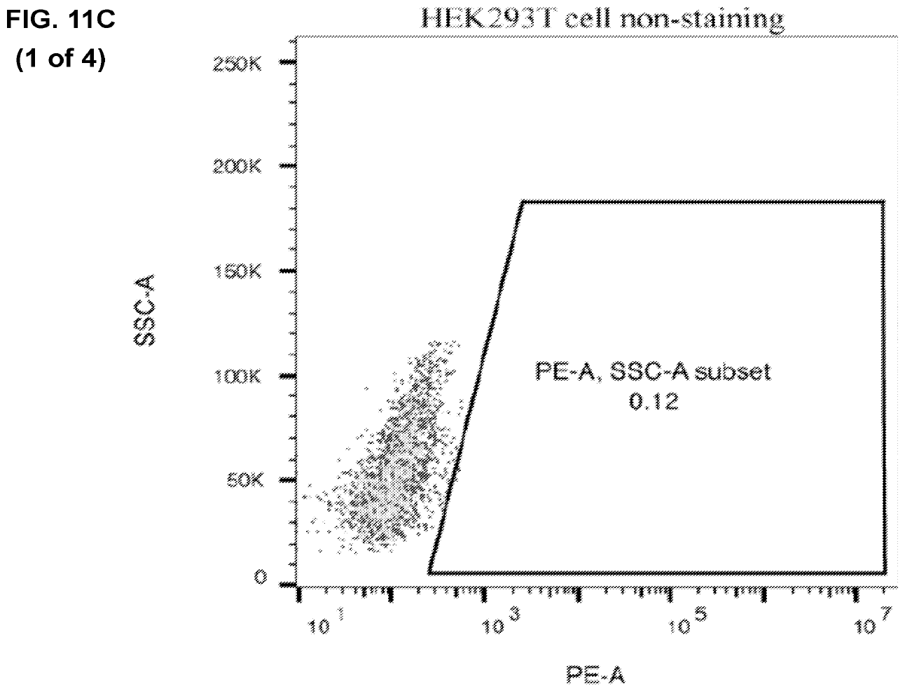

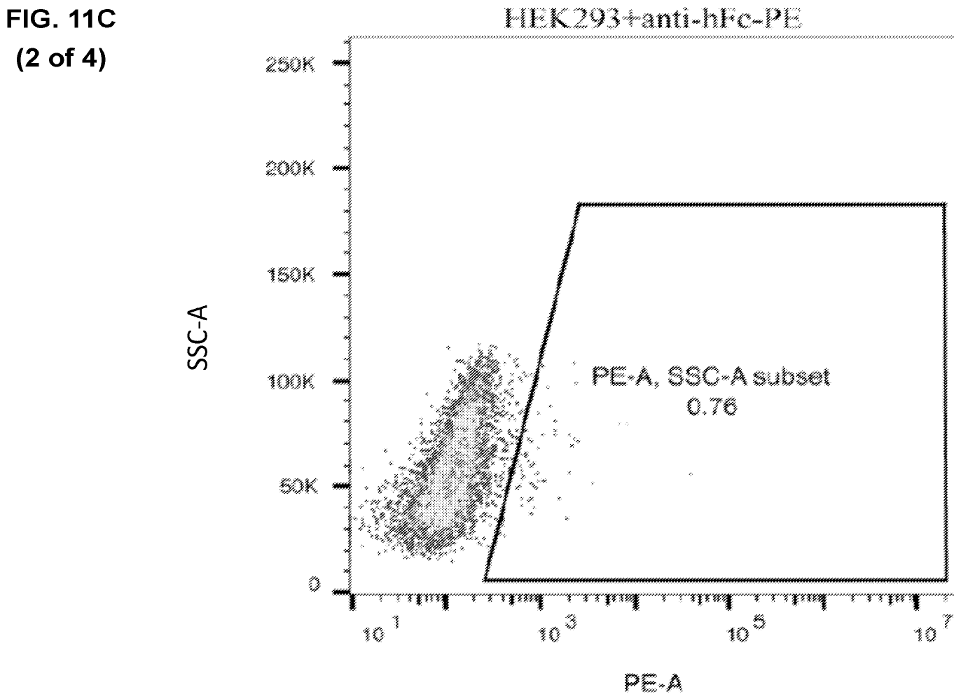

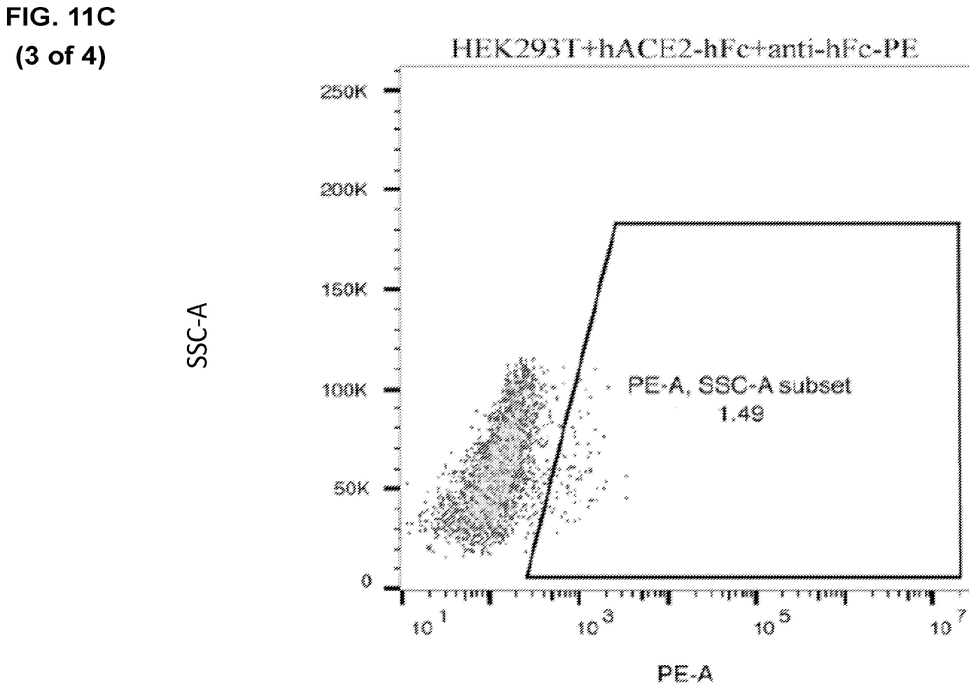

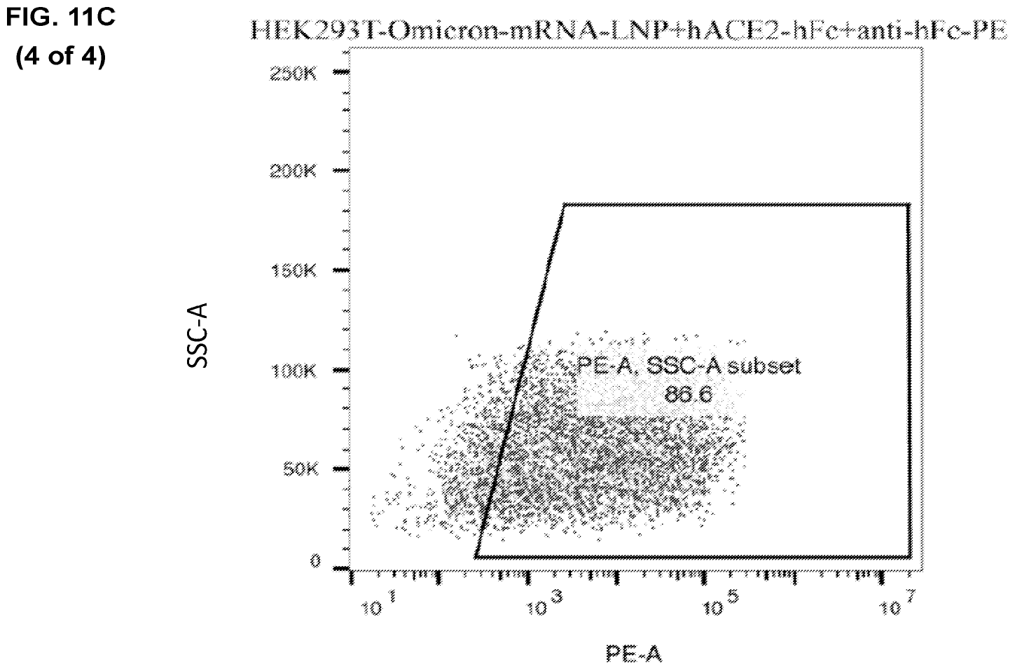

Using Omicron-specific LNP-mRNA that contains lipid nanoparticle formulated mRNA encoding the HexaPro engineered full length of Omicron spike glycoprotein (Methods), the biophysical integrity of these LNP-mRNAs was first characterized (FIG. 11A, FIG. 11B), and validated the expression of functional Omicron spike protein surface expression via human ACE2 (hACE2) staining of LNP-mRNA transfected HEK293 cells (FIG. 11C). Next, administration of four 10 μg doses and one 20 μg dose of Omicron specific-mRNA LNP was performed in 3 IgG-humanized mice, collected retro-orbital blood samples from each humanized mouse before and after booster immunization. Blood samples were labeled as pre-, 1st or 2nd immunization draw depend on collection sequence (FIG. 7C). Antibody titers were measured using serial plasma dilutions on ELISA plates coated with recombinant Omicron RBD protein. Binding activity was visualized using anti-mouse IgG antibodies at 450 nm optical density (OD). Three sequential plasma samples showed increasing vaccine-elicited antibody responses during each blood collection (FIG. 7D). All post-immunized plasma samples (2nd blood) showed strong reactivity to the recombinant SARS-COV-2 Omicron RBD protein antigen (FIG. 7D). In addition, all these samples also showed strong cross-reactivity to recombinant SARS-COV-2 Delta RBD protein, and intermediately cross-reactivity to recombinant SARS-COV RBD protein, but no cross-binding to recombinant MERS-COV RBD protein (FIG. 7D). Together, these results demonstrated that Omicron-specific rapid mRNA immunization (Omicron-RAMIHM) elicited strong anti-Omicron plasma in IgG humanized mice in two weeks, which also contains broader reactive antibodies against other variant and coronavirus species such as SARS-COV-2 Delta and SARS-COV.

Customized Single Cell BCR Sequencing (scBCRseq) Mapped the IgG Clonal Repertoires of Omicron-RAMIHM Animals.

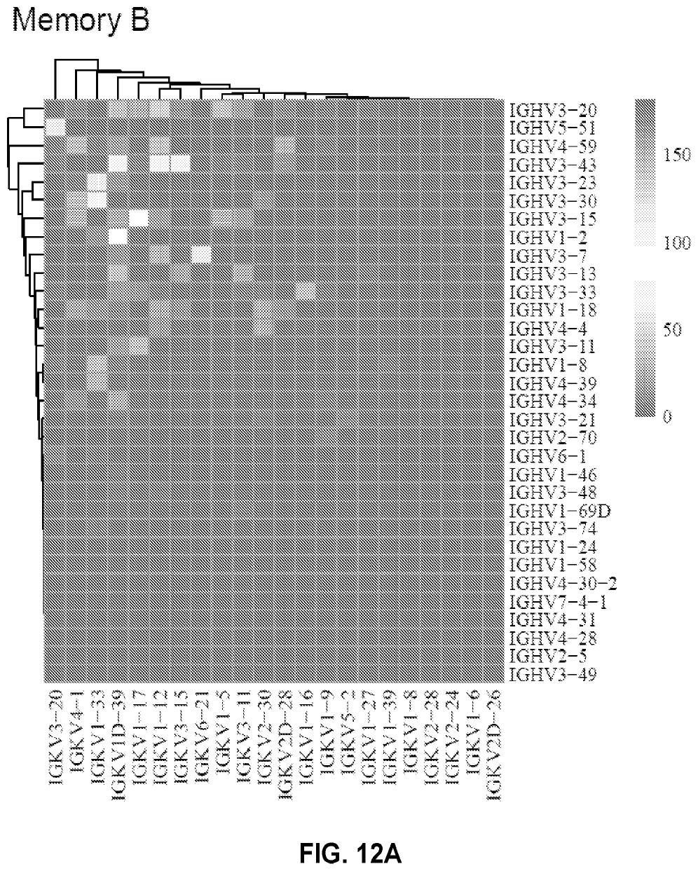

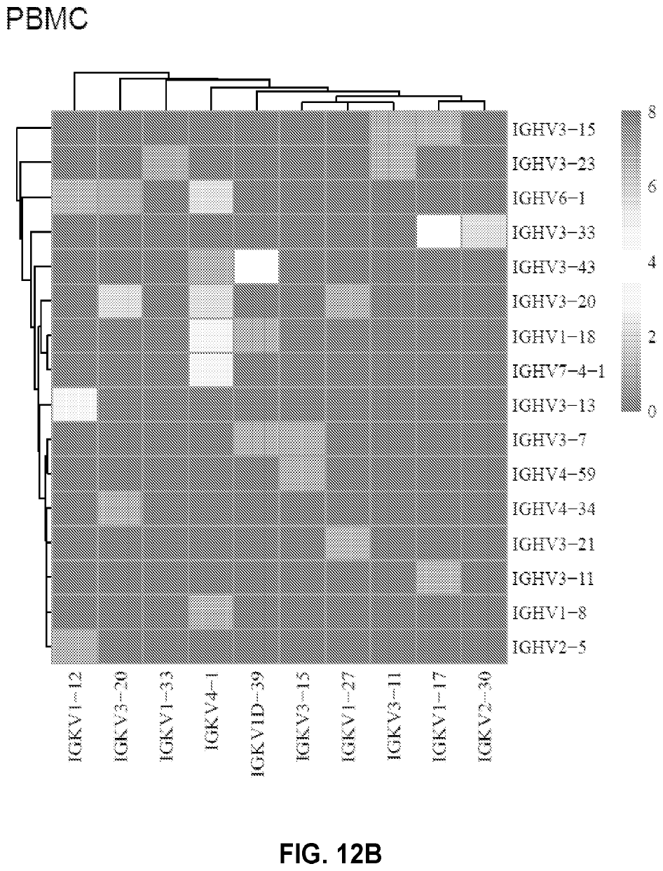

To obtain SARS-COV-2 Omicron RBD-reactive B cells, spleen, lymph nodes, bone marrow and whole blood were isolated from Omicron-RAMIHM mouse, and collected three different types of B cells (memory B cell, plasma B cells, and peripheral blood mononuclear cells) by using different isolation procedures (Methods), for B cell repertoire mapping and reactive BCR identification via scBCR-seq. To prepare memory B cells enriched library, mouse memory B cell isolation kits were used to obtain total memory B cells from fresh spleen and lymph nodes, and baited SARS-COV-2 Omicron RBD specific memory B cells by enrichment using recombinant Omicron-RBD proteins from isolated memory B cell subsets (Memory B library). To generate a plasma B cell enriched library, anti-mouse CD138+ plasma cell isolation kits were used to isolate CD138+ plasma B cells from freshly isolated raw bone marrow cells (Plasma B library). To generate peripheral blood mononuclear cells library, peripheral blood mononuclear cells (PBMCs) were isolated by centrifugation using PBMC isolation method from whole blood (PBMC/Peripheral B library). Each single cell BCR sequencing library was subjected to input of approximately 10,000 fresh cells from above. After sequencing, a total of 3,502 single B cells were analyzed, and obtained 2,558 paired heavy- and light-chain variable regions of antibody sequences (FIG. 7E). To examine the IgG clonal repertoires from the scBCRseq data, B cell clonotypes were first examined by calculating the frequencies of cells observed for the clonotype and distributions of identical CDR3 region for both heavy and light chains in pairs. By analyzing the BCR repertoires, the landscape of BCR populations in the Memory B, Plasma B and Peripheral B/PBMC in Omicron-RAMIHM immunized mouse were mapped (FIG. 12A-FIG. 12C).

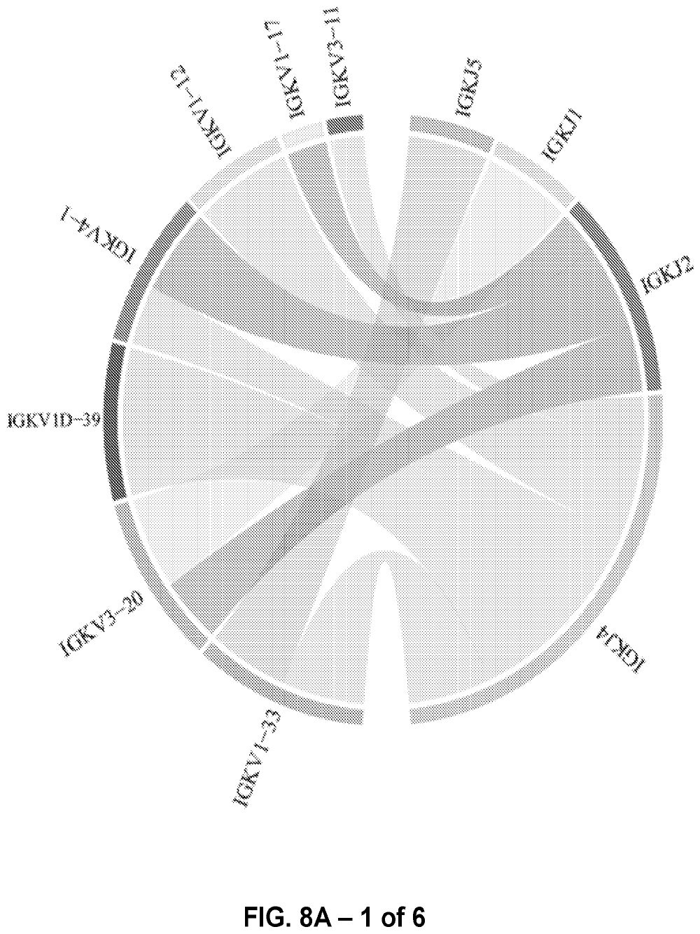

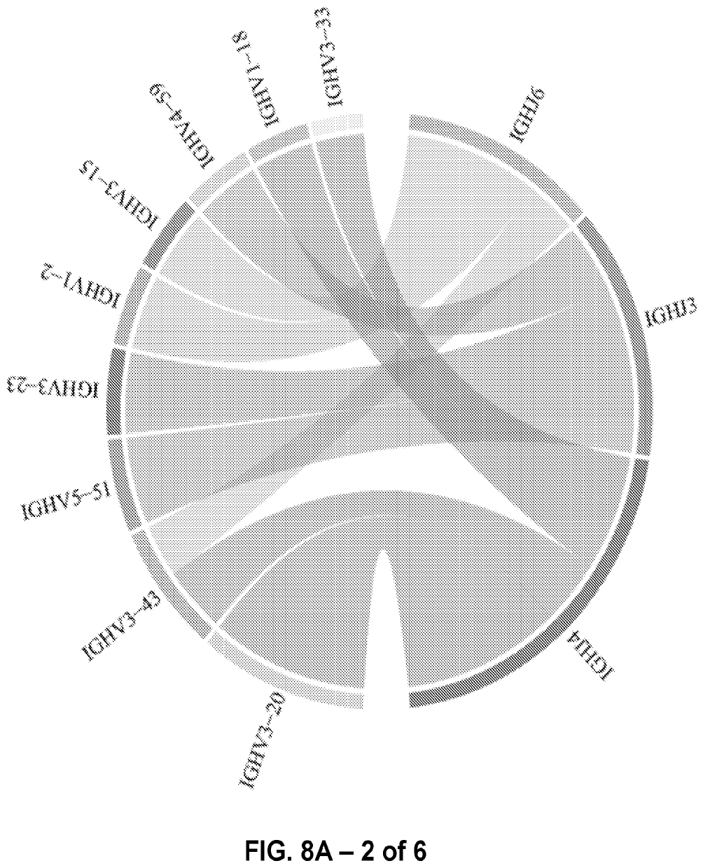

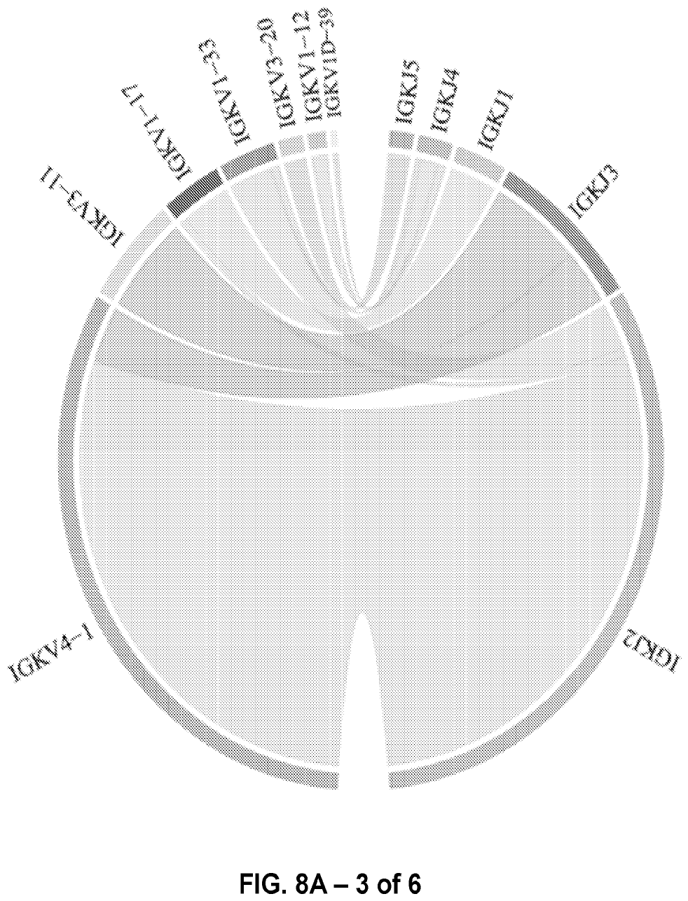

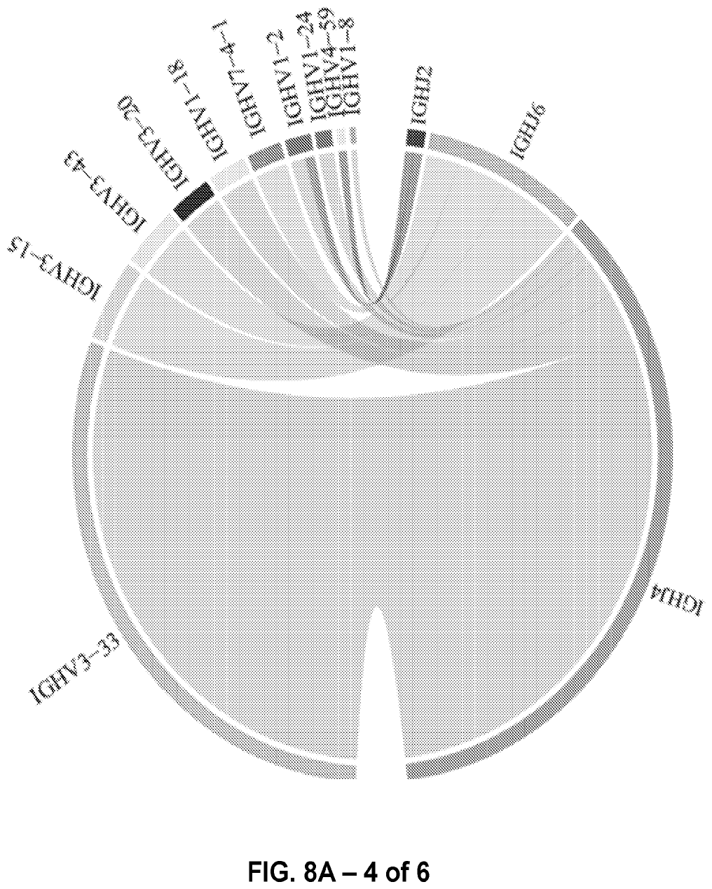

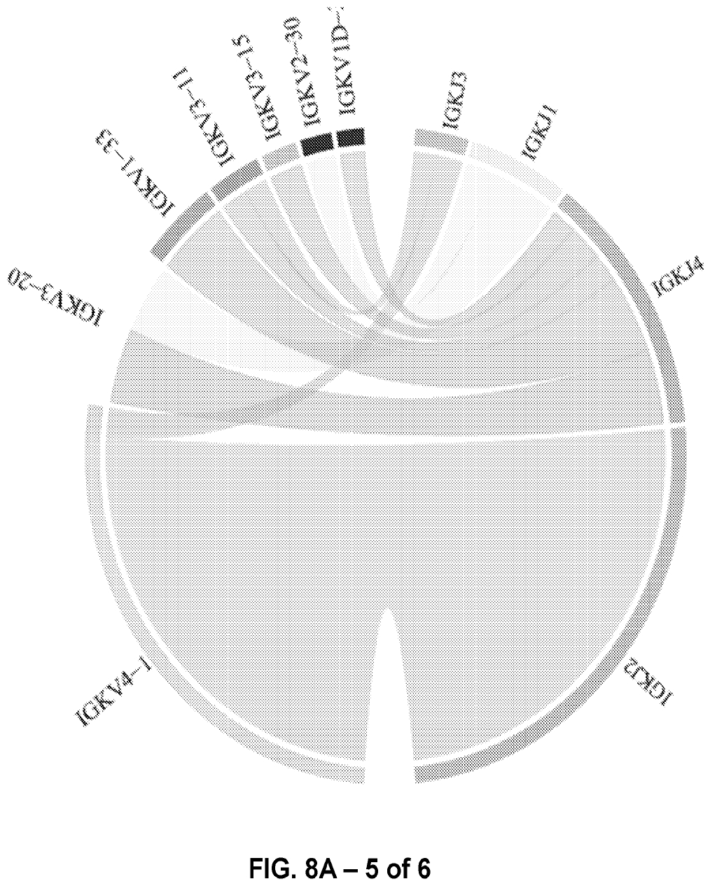

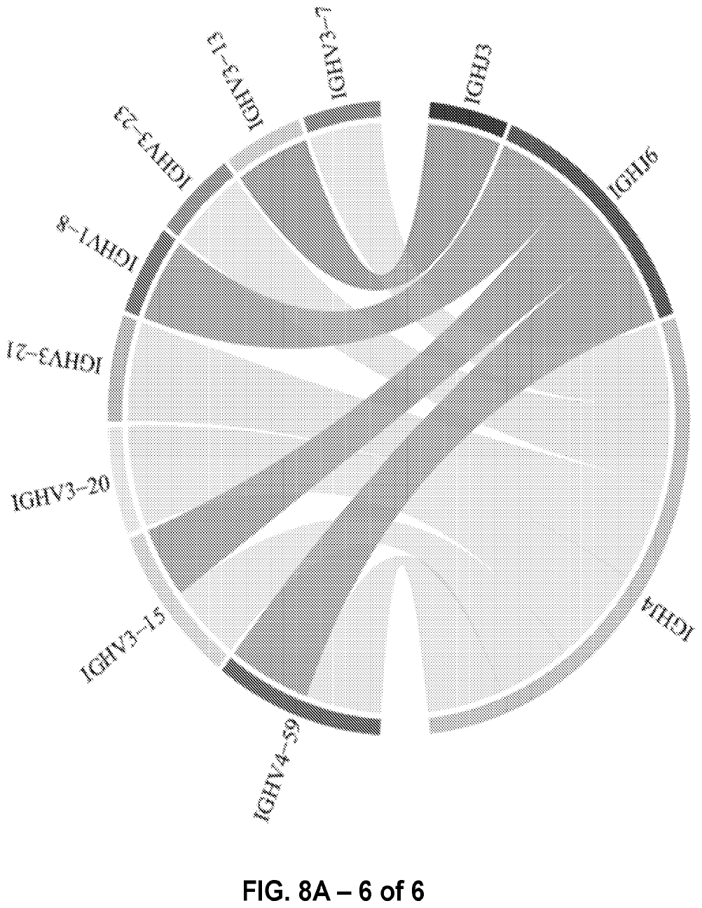

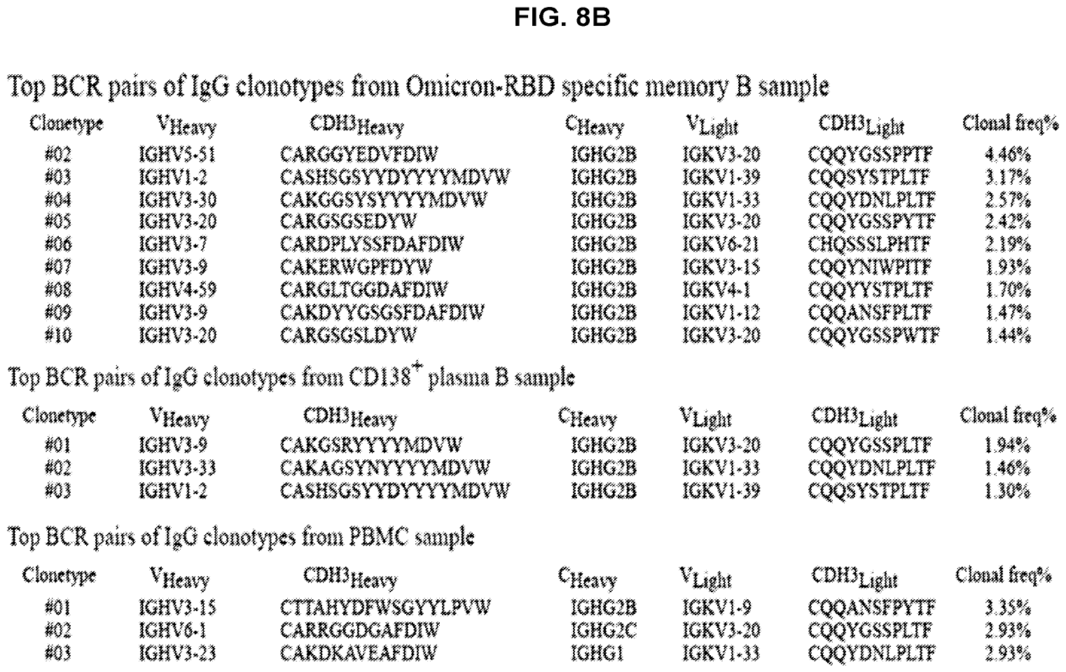

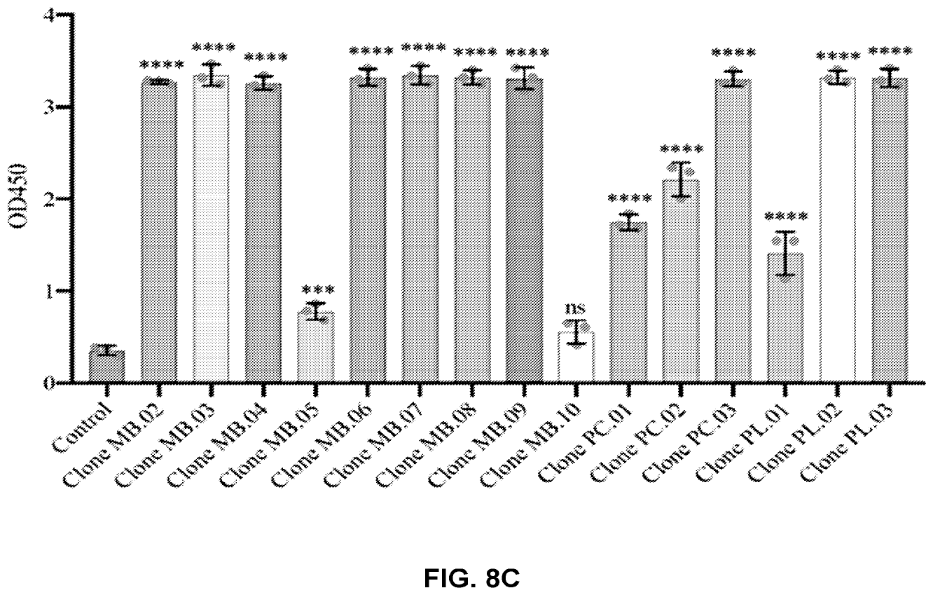

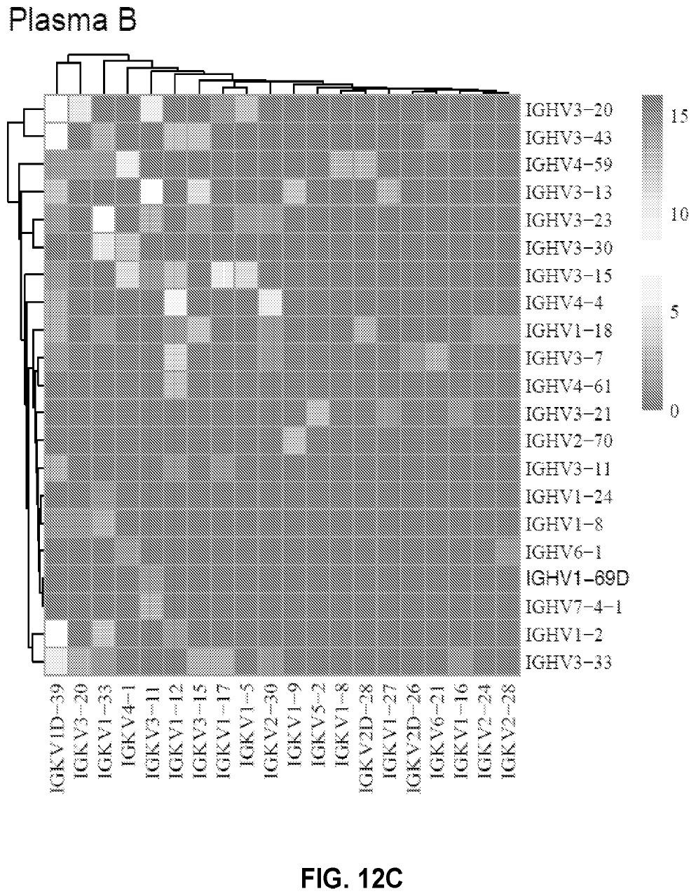

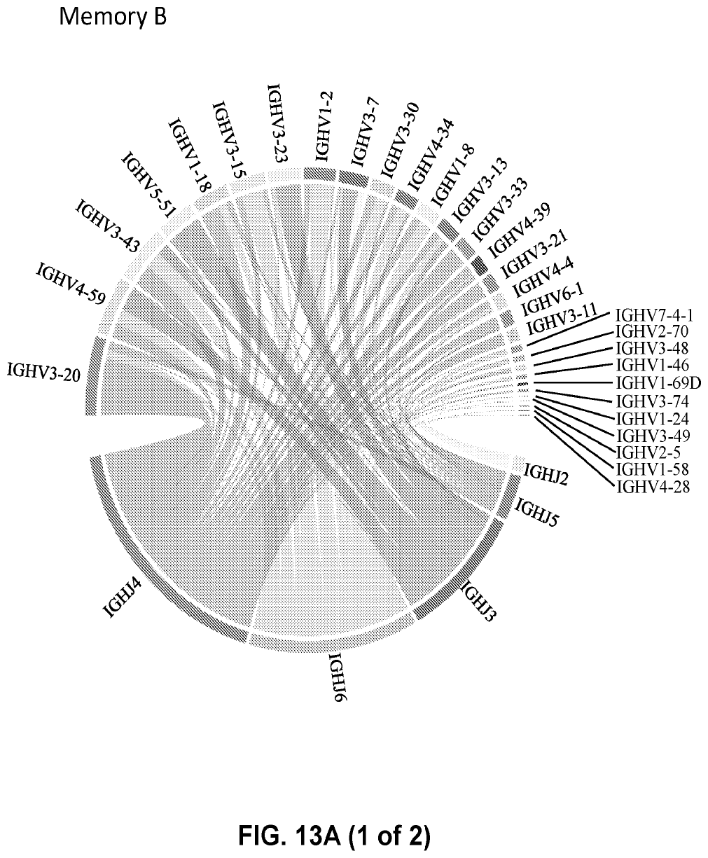

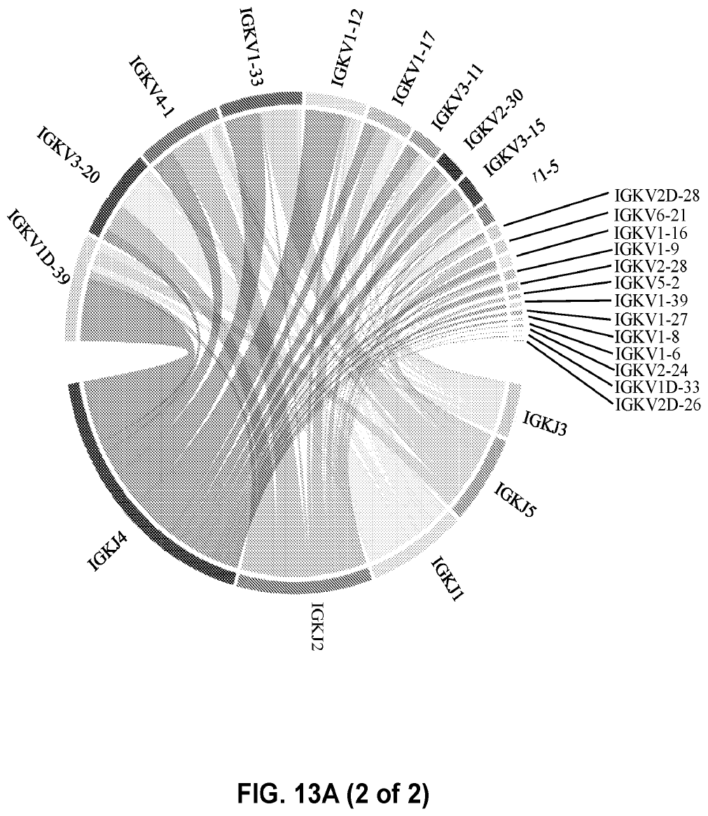

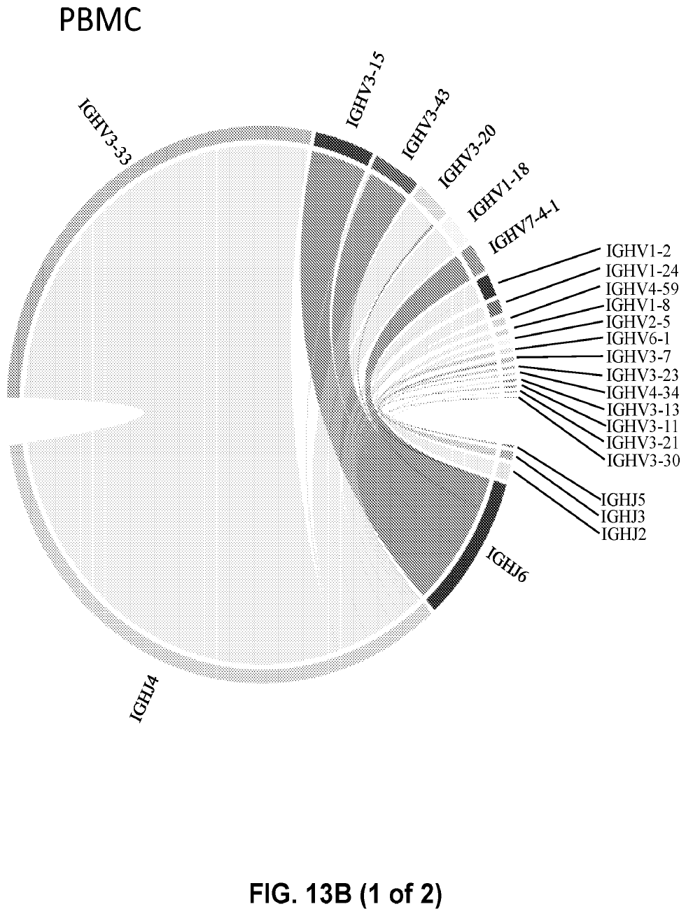

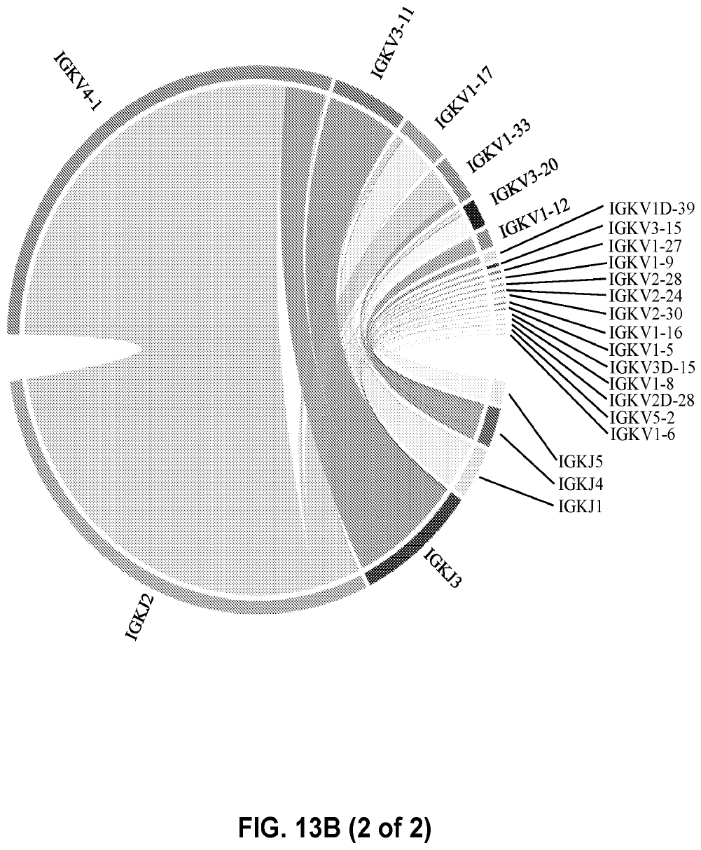

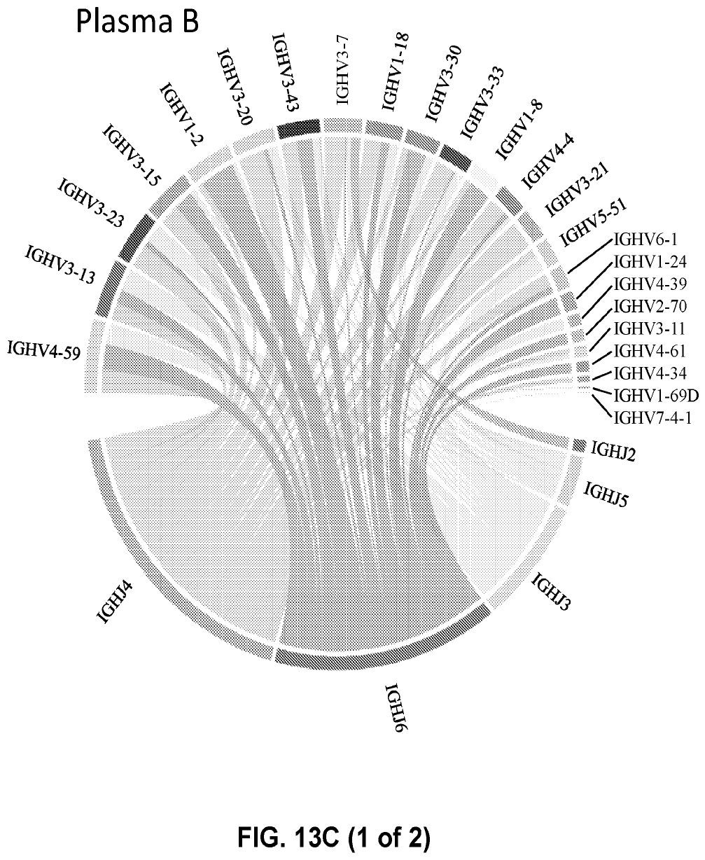

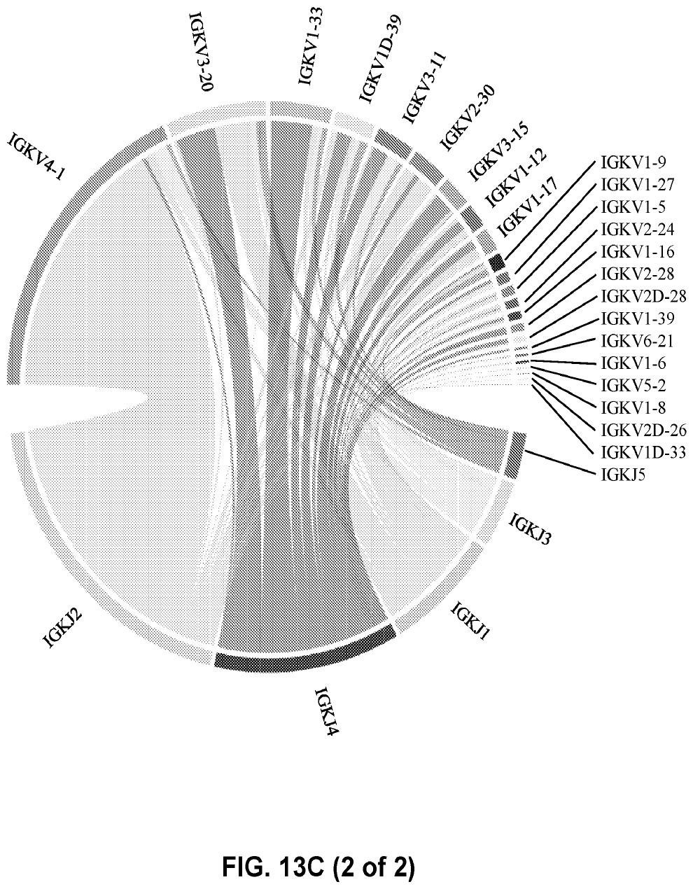

The SARS-COV-2 Omicron RBD-specific antibodies had a relative enrichment for IGVH3-7, IGVH3-15, IGVH3-20, IGVH3-23, IGVH3-30, IGVH3-33, IGHV3-43, and IGVH4-59, analyzed from 3 individual BCR libraries (FIG. 7E). A range of lengths between 8-24 aa was observed for these BCR CDRH3s (FIG. 7E). Interestingly, a large portion of IgG2B-expressing B cells were identified from three B cell type isolations (FIG. 7F), a signature of potential involvement of Th2 cells in B cells maturation and class switch in these mice undergoing the Omicron-RAMIHM procedure. By analyzing the Ig heavy chain (IGH) and light chain (IGK) paring, the overall, enriched and the top 10 heavy- and light-chain V/J segment recombination in these B cell populations was also mapped out (FIG. 8A, FIG. 8B, and FIG. 13A-FIG. 13C). In summary, scBCRseq data mapped the clonal repertoires and revealed enriched IgG clonotypes in the peripheral blood, plasma B cell and memory B cell populations in Omicron-RAMIHM humanized mouse.

Identification of Omicron-Specific Functional mAb Clones from Top-Ranked Paired Human Ig Chains of Omicron-RAMIHM Animals.

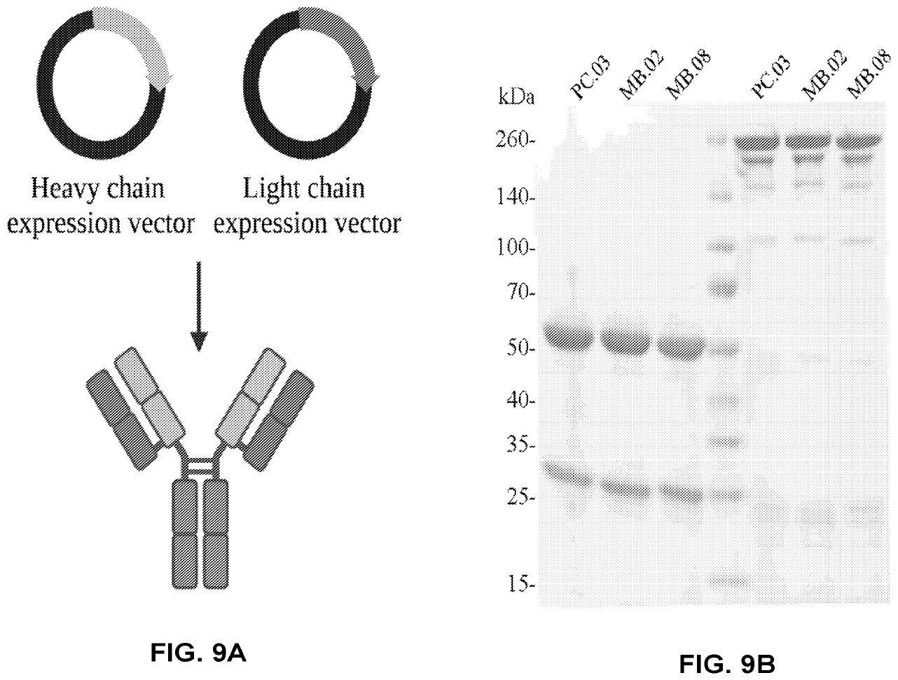

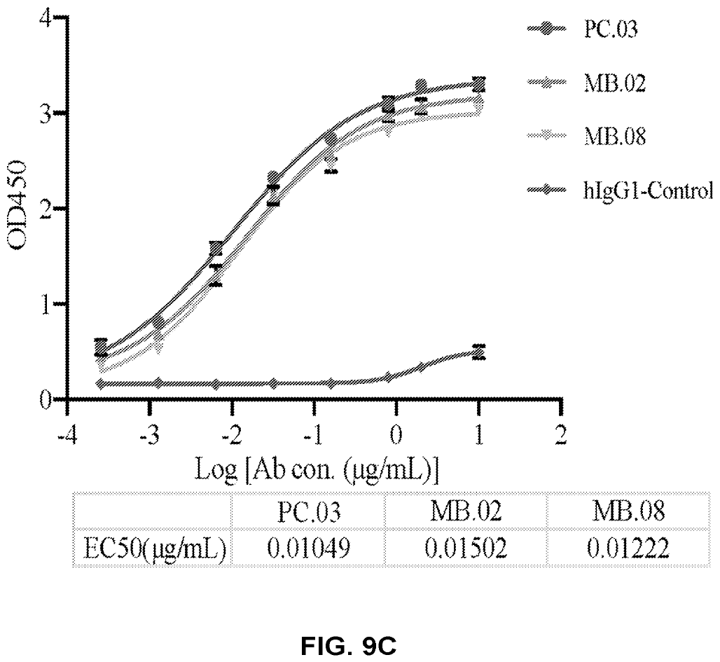

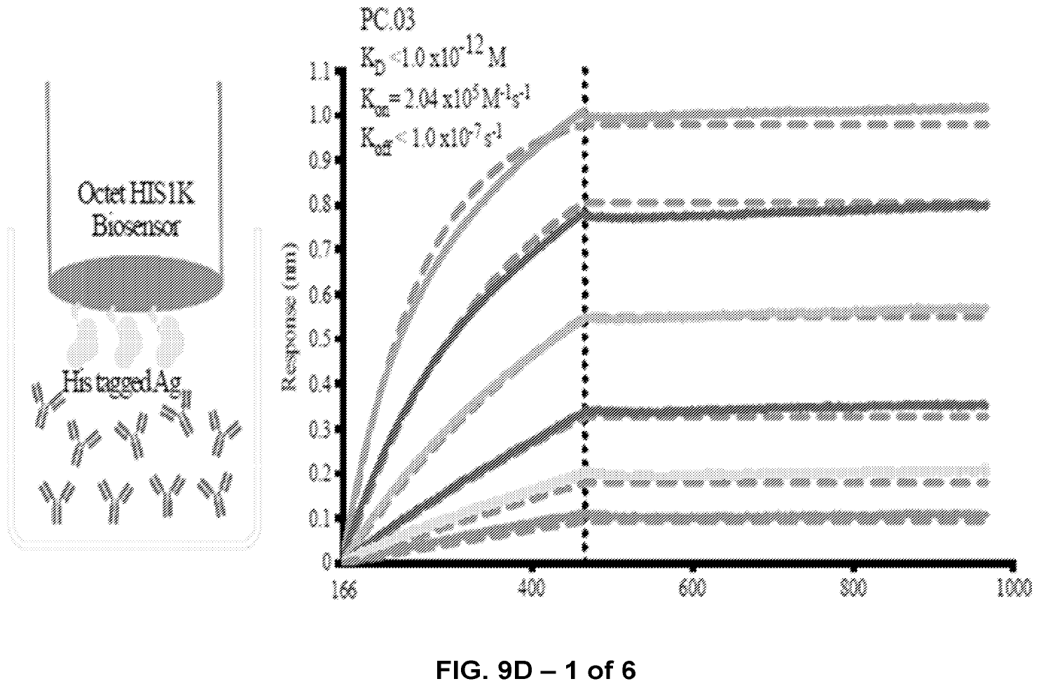

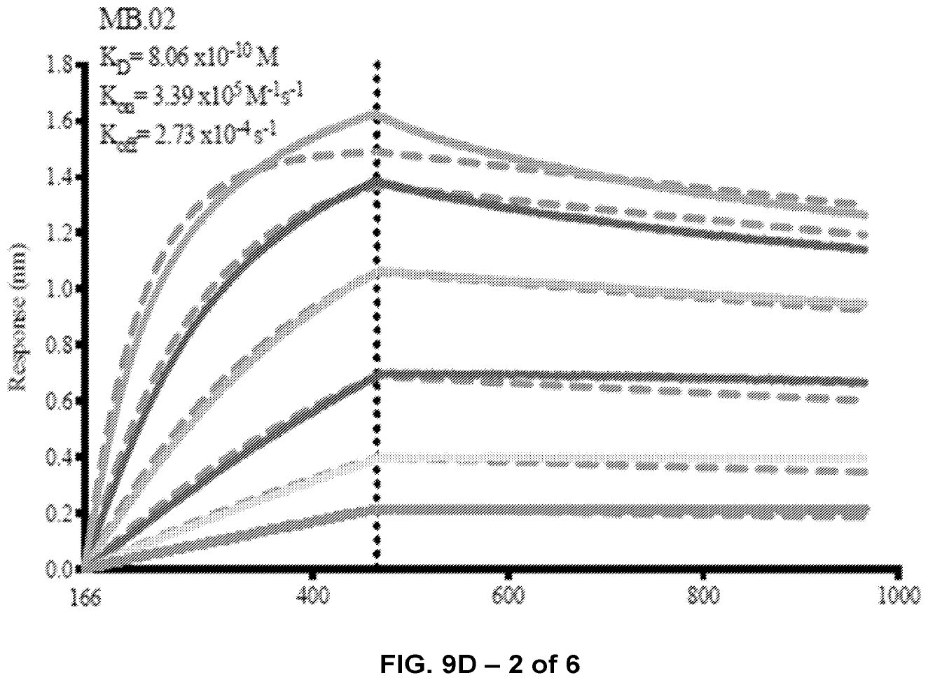

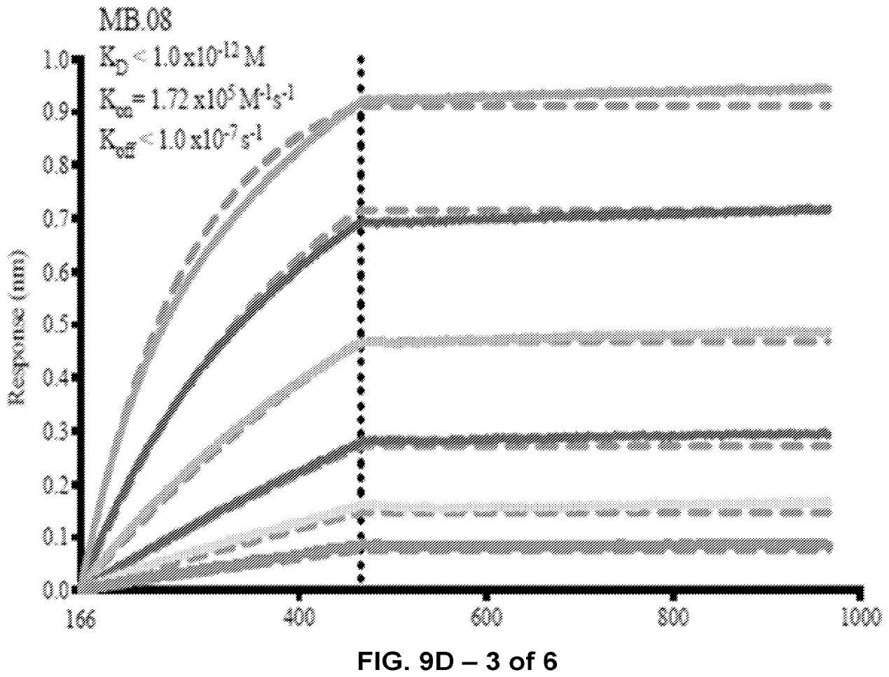

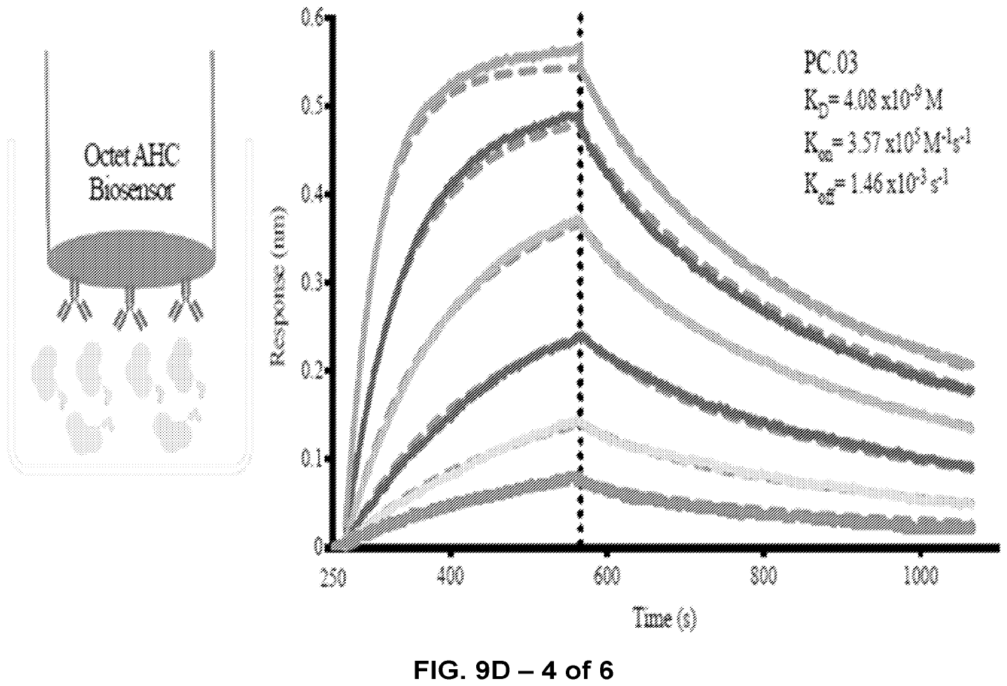

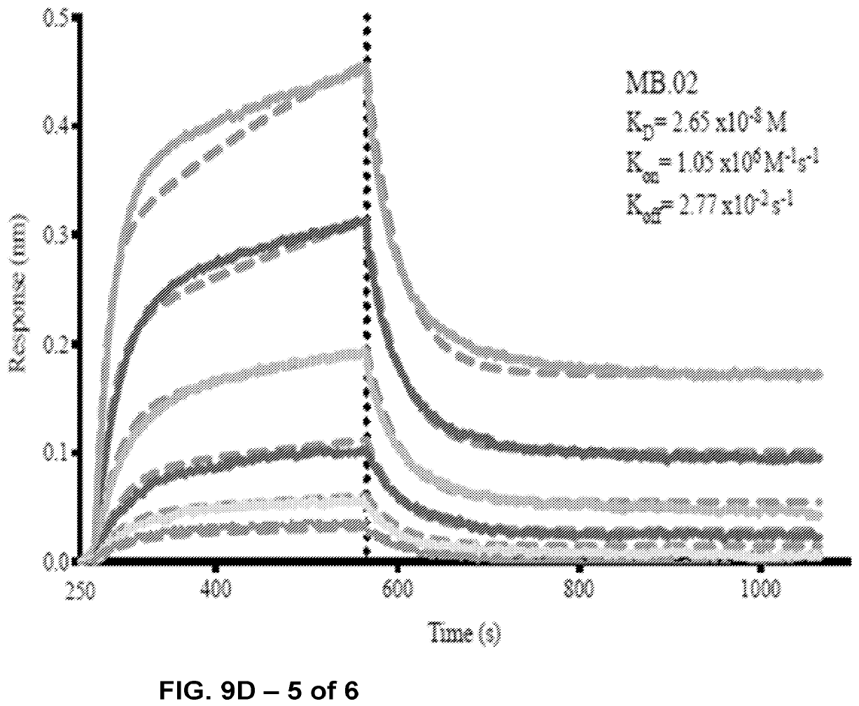

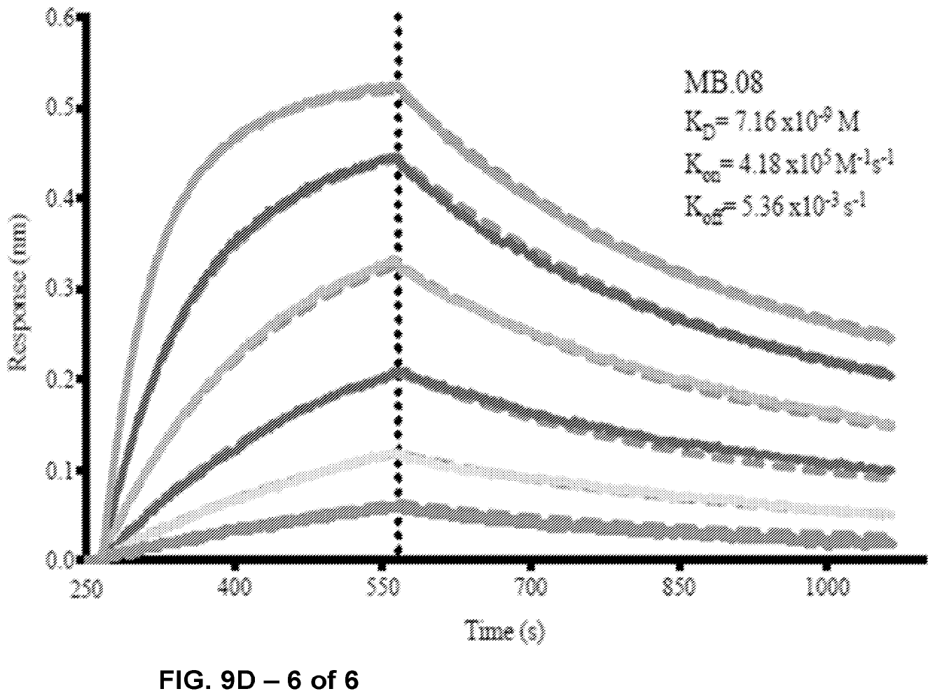

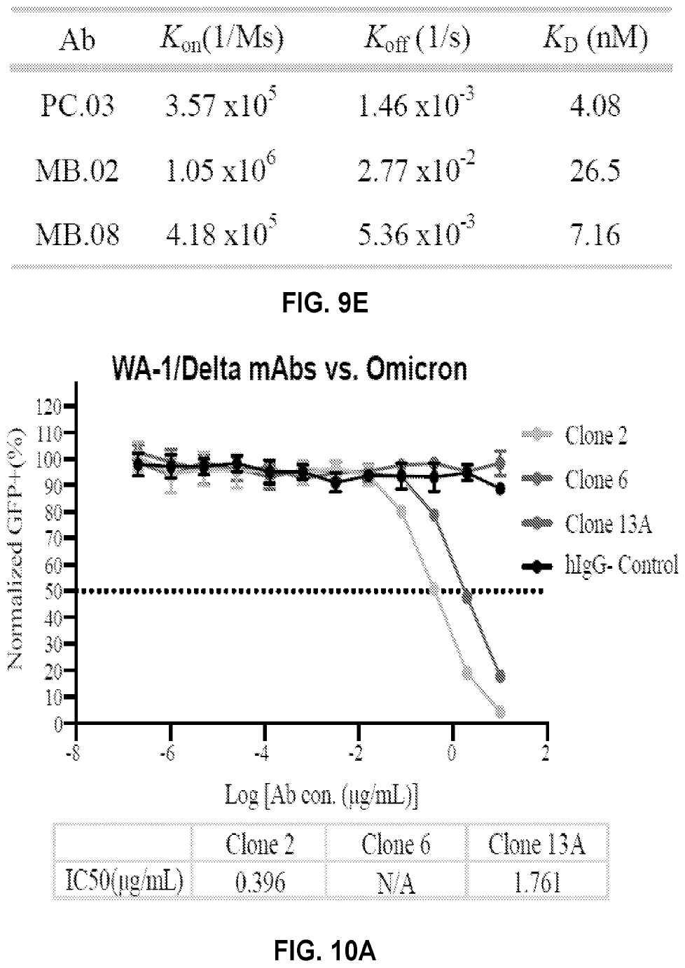

To test whether the most enriched BCRs in these B cell populations are Omicron-reactive, a panel of BCRs was selected for recombinant mAb expression, including 3 from peripheral blood, 3 from plasma B and 9 from memory B cell populations (FIG. 8C). In order to functionally analyze the antibody response to SARS-COV-2 Omicron RBD, paired heavy- and light-variable segments were cloned into human IgG1 expression vectors (FIG. 9A), and used the Expi293F mammalian expression system to produce selected mAbs. Thereafter, SARS-COV-2 Omicron RBD-specific ELISA was used to determine antibody binding by using transfected culture supernatants that contain secreted antibodies. As a result, almost all of the top-enriched antibody clones collectively from peripheral blood, plasma B cell and memory B cell populations are reactive to Omicron RBD (14/15 reactive, 1/15 slightly reactive), showing a high rate of antigen-specificity (14/15, 93.3%) from Omicron-RAMIHM-scBCRseq (FIG. 8C). Ten out of fifteen (10/15) selected clones showed potent binding capacity against recombinant SARS-COV-2 Omicron RBD proteins, 4/15 showed moderate binding, an 1/15 showed relatively weak binding (FIG. 8C). These results indicated that RAMIHM is a highly effective approach for generating and isolating antigen-specific mAbs.

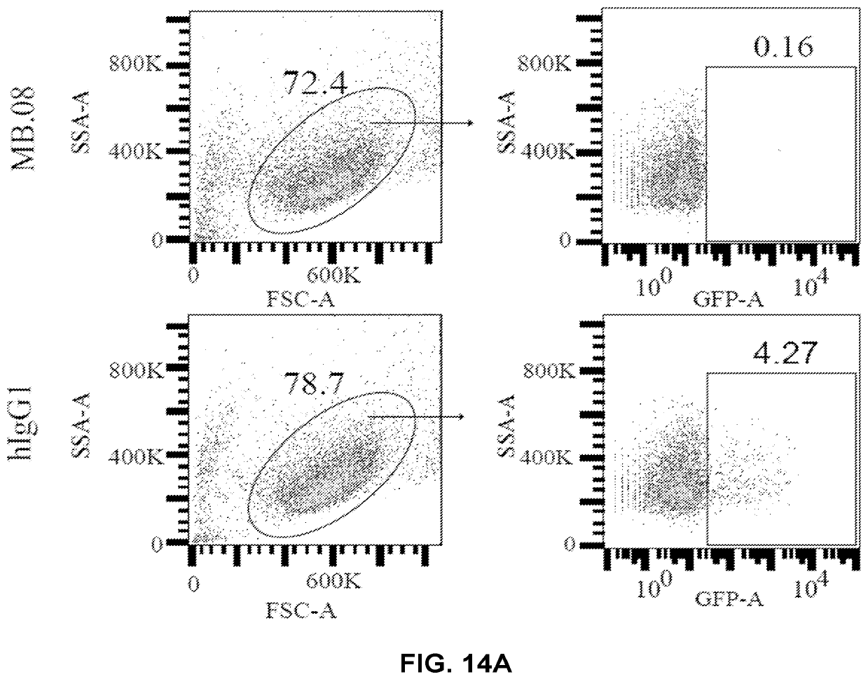

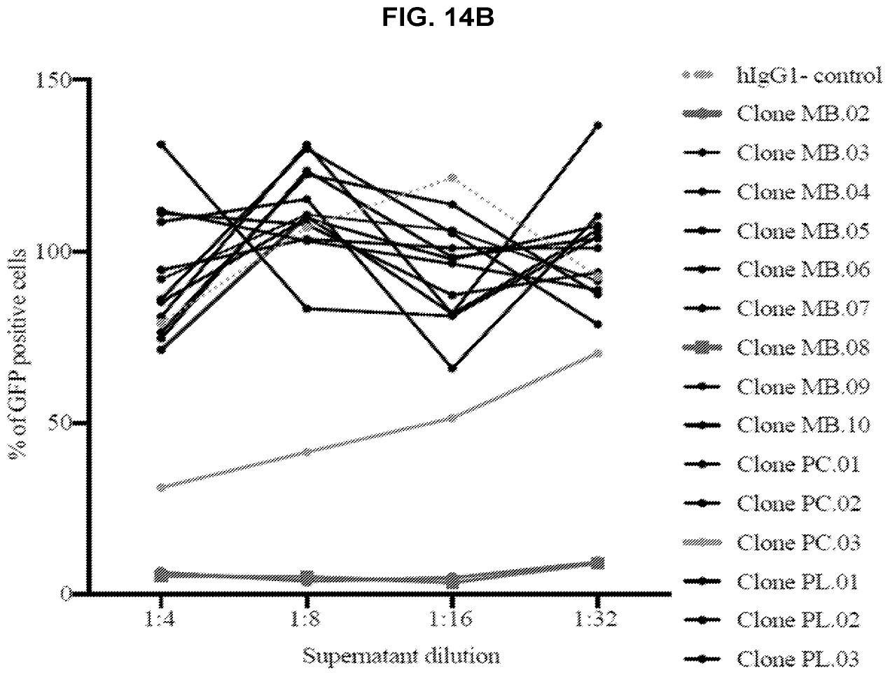

To further screen for highly potent functional mAbs, 15 mAb candidate clones were recombinantly expressed in a mammalian system and tested for neutralization ability against the Omicron variant. By screening the mAbs from culture supernatants by neutralizing assay using a spike-based SARS-COV-2 Omicron pseudovirus system, 3 clones were found with obvious neutralization activity against Omicron pseudovirus (FIGS. 14A, 14B). The top 3 clones (named as PC.03, MB.02, and MB.08) were selected for further development and characterization.

Characterization of Fully Human Lead Clones with Strong Binding to Omicron RBD.

The three leading clones, PC.03, MB.02, and MB.08 (see Table 1 and Table 2), were purified by affinity chromatography using Protein A beads and examined antibody purity by SDS-PAGE (FIG. 9B). Thereafter, purified leading mAbs were tested for SARS-COV-2 Omicron RBD reactivity by ELISA and monitored real-time association and dissociation to recombinant SARS-COV-2 Omicron RBD proteins using the Octet system. The ELISA titration result of lead mAb clones vs. recombinant SARS-COV-2 Omicron RBD proteins showed that these three mAb clones have EC50s at the level of ˜0.01 μg/mL, suggesting that these mAbs can indeed tightly bind to Omicron RBD (EC50<16 ng/ml for all 3 clones) (FIG. 9C). Octet results with his-tag Omicron RBD antigen immobilization showed ultra-strong binding (K_{D} at 0.8 nM for MB.02, and K_{D}<1 pM for PC.03 and MB.08) (FIG. 9D). Noted that this might be contributed by avidity effect due to multi-valent binding, reverse Octet assay was also performed with antibody immobilization, which measured the single-mAb binding affinity (FIG. 9D), and showed that the affinity between these clones to Omicron RBD are at the level of low nanomolar range (FIG. 9D). These K_{D} values (FIG. 9E) showed that the binding strengths of the 3 lead mAbs are stronger than that of hACE2 with Omicron RBD (31.4±11.62 nM). Noted that most of approved or EUA mAbs have much weaker binding with Omicron RBD (summarized in Table 4).

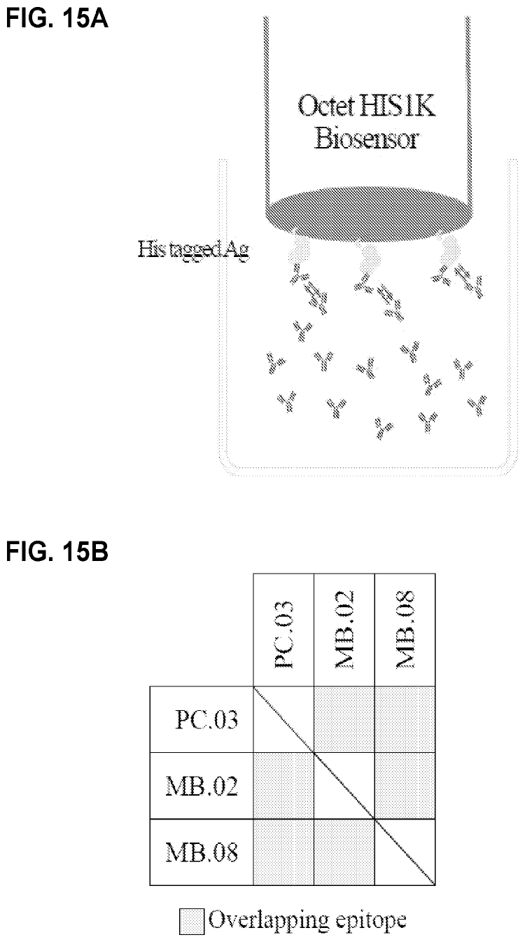

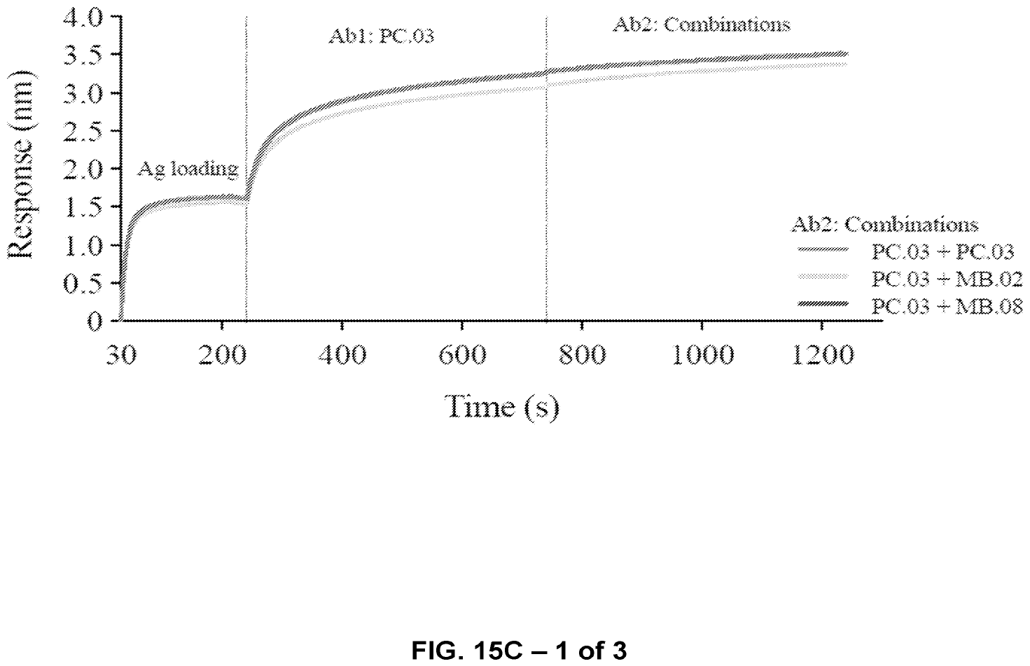

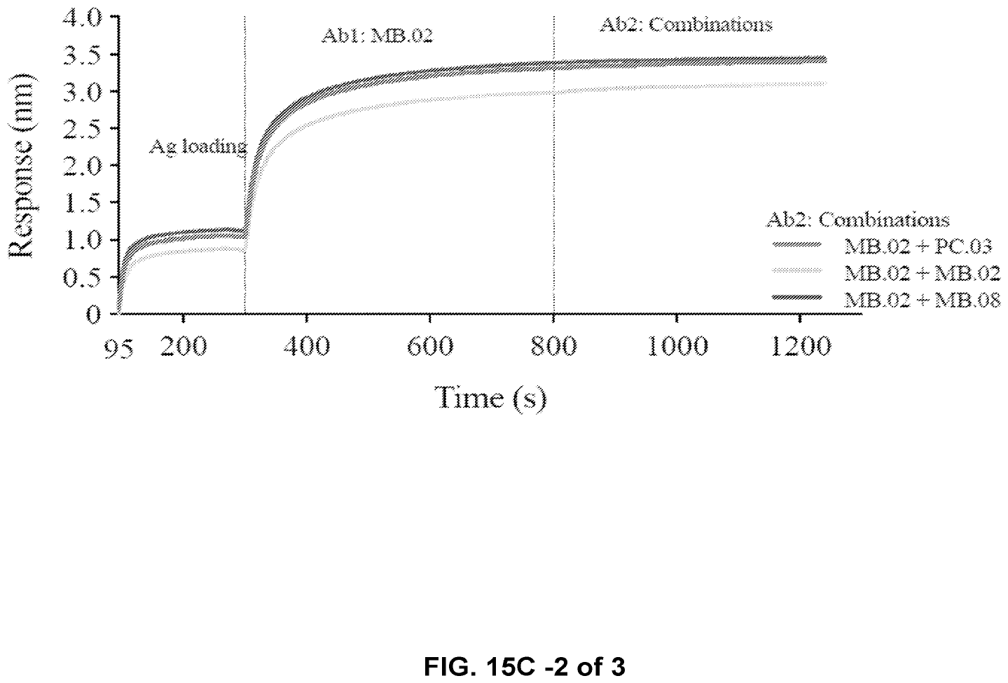

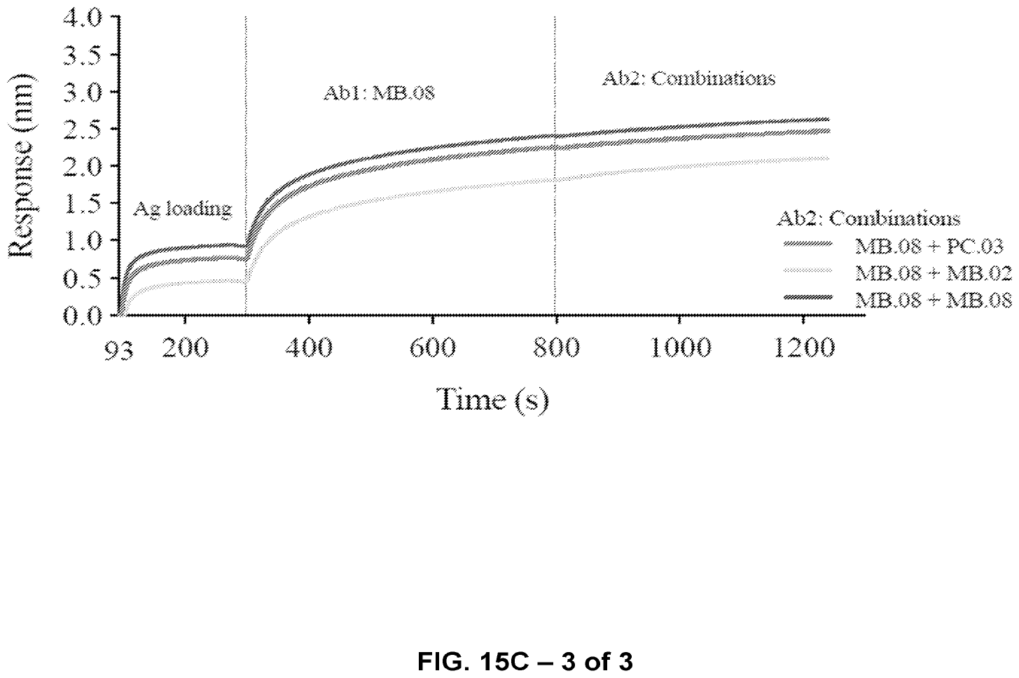

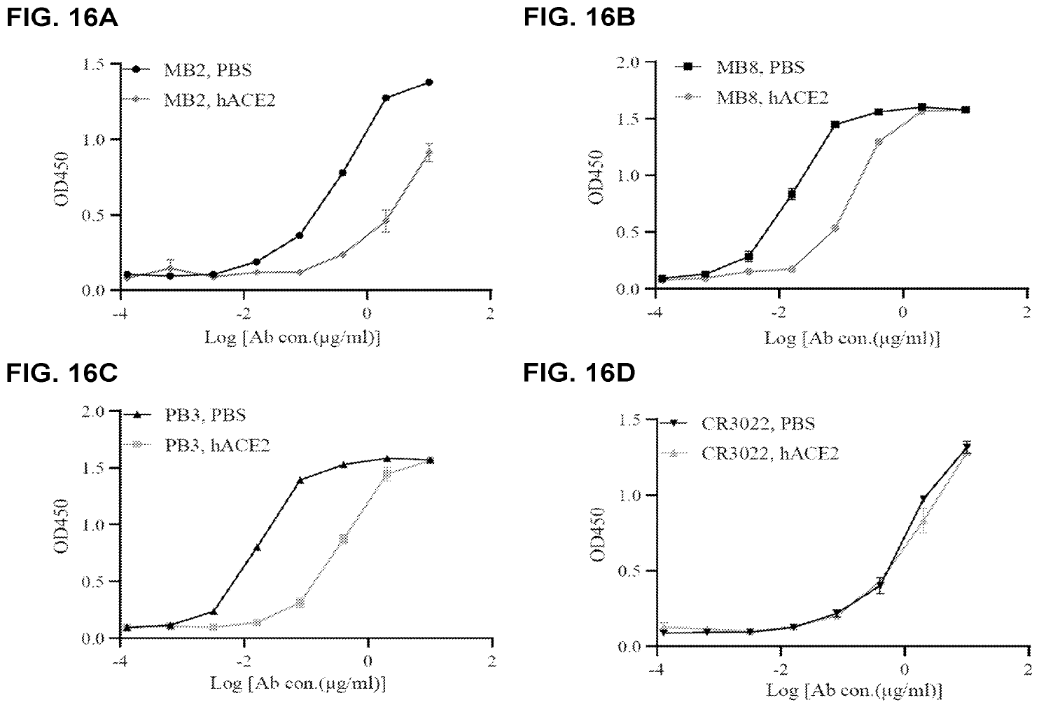

To further determine whether these leading mAbs compete for similar epitopes, epitope binning experiments were performed by Octet using an in-tandem assay (FIG. 15A). The results have exhibited that PC.03, MB.02, and MB.08 likely share overlapping epitopes (FIGS. 15B, 15C). Studies next measured antibody competition with ACE2, which was quantified as reduction in ACE2 and RBD binding. Consistent with binding affinity findings, these three leading clones showed competitive binding with ACE2 against Omicron RBD (FIGS. 16A-16D).

TABLE 4
Summary of mAb efficacy vs Omicron

| | Kd | | IC50(Psedotyped |
|---|---|---|---|
| Monoclonal | (SARS-CoV-2 | | SARS-CoV-2 |
| antibody | Omicron-RBD) | | Omicron) ng/mL |
| MB.02 | 26.5 | nM | 93.91 |
| MB.08 | 7.16 | nM | 44.29 |
| PC.03 | 4.08 | nM | 153.77 |

| | | |
|---|---|---|
| MB.02 plus | N/A | 30.41 |
| MB.08 | | |
| MB.02 plus | N/A | 78.39 |
| PC.03 | | |
| MB.08 plus | N/A | 56.61 |
| PC.03 | | |
| LY-CoV016, | No binding (Mccallum M. | >10000 (Cameroni E. |
| etesevimab | et. al, Science, 2022) | et. al, Nature, 2021) |
| LY-CoV555, | No binding (Mccallum M. | >10000 (Cameroni E. |
| bamlanivimab | et. al, Science, 2022) | et. al, Nature, 2021) |
| LY-CoV016 | N/A | >10000 (Cameroni E. |
| plus | | et. al, Nature, 2021) |
| LY-CoV555 | | |
| REGN10987, | No binding (Mccallum M. | >10000 (Cameroni E. |
| imdevimab | et. al, Science, 2022) | et. al, Nature, 2021) |
| REGN10933, | 824 nM (Mccallum M. | >10000 (Cameroni E. |
| casirivimab | et. al, Science, 2022) | et. al, Nature, 2021) |
| REGN10987 | N/A | >10000 (Cameroni E. |
| plus | | et. al, Nature, 2021) |
| REGN10933 | | |
| COV2-2196, | 488 nM (Mccallum M. | >10000 (Cameroni E. |
| tixagevimab | et. al, Science, 2022) | et. al, Nature, 2021) |
| COV2-2130, | 63.4 nM (Mccallum M. | 2772 (Cameroni E. |
| cilgavimab | et. al, Science, 2022) | et. al, Nature, 2021) |
| COV2-2196 | N/A | 418 (Cameroni E. |
| plus | | et. al, Nature, 2021) |
| COV2-2130 | | |
| S309, | 2.67 nM (Mccallum M. | 260 (Cameroni E. |
| sotrovimab | et. al, Science, 2022) | et. al, Nature, 2021) |

### Further Characterization of Fully Human Lead Neutralization mAb Clones Against Omicron.

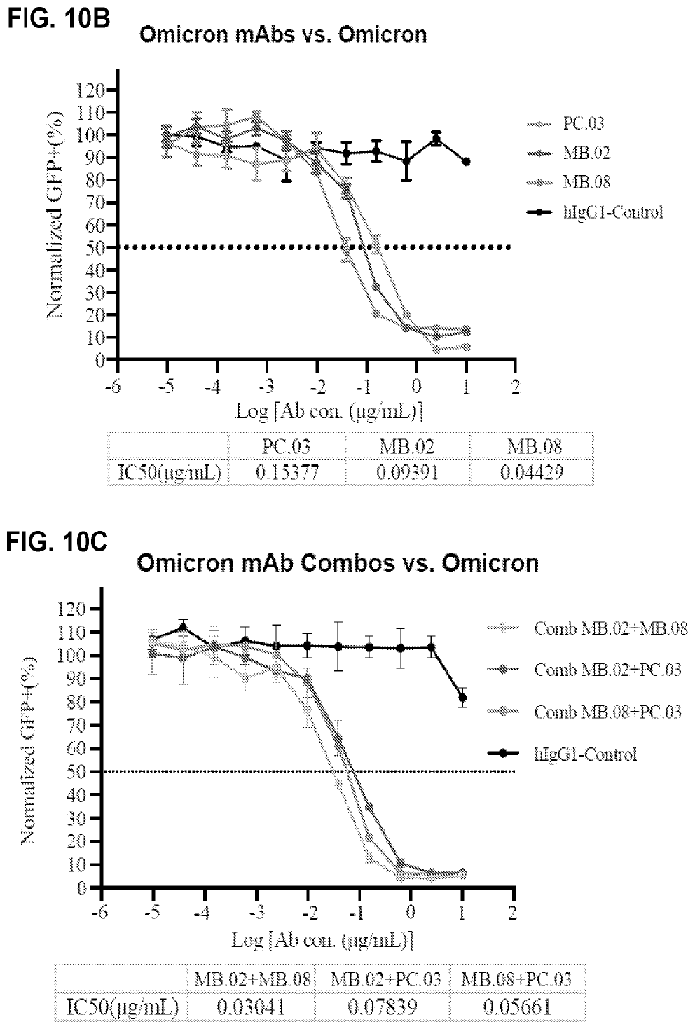

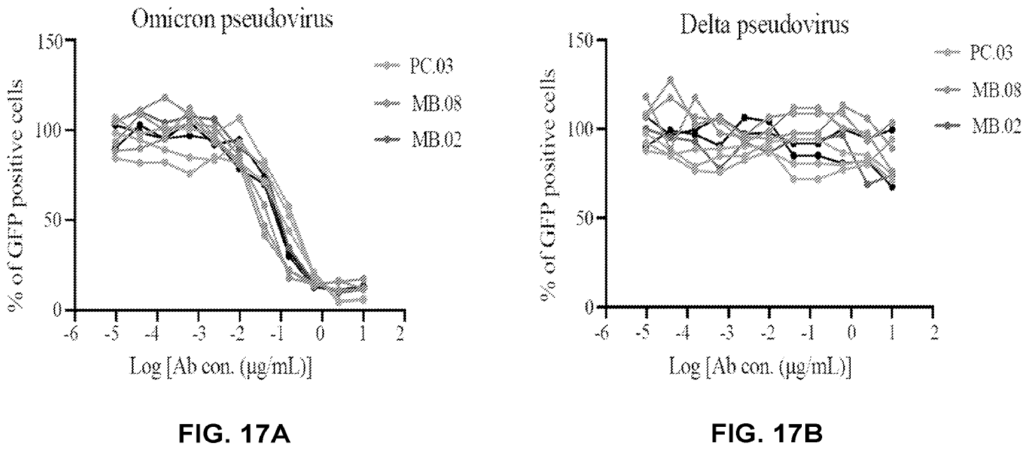

Neutralization assays were then performed for the 3 lead mAbs in purified form, along with other mAbs. Several potent and specific mAbs were previously identified and developed against the ancestral virus and the Delta variant, namely clones 2, 6 and 13A. In a pseudovirus neutralization assay, it was found that while clones 2 and 13A can still neutralize Omicron variant, the potency is significantly reduced (by 1-2 orders of magnitude in terms of IC50 values, at 0.396 and 1.761 μg/mL for clone 2 and 13A, respectively) (FIG. 10A), a phenomenon similar 10 to other mAbs developed against the ancestral spike. In contrast, all three clones, PC.03, MB.02, and MB.08, potently neutralized the Omicron variant, with IC50 values at 0.15 μg/mL (PC.03), 0.09 μg/mL (MB.02), and 0.04 μg/mL (MB.08) (FIG. 10B; FIG. 17A). The neutralization potency of the three lead Omicron-specific mAb clones are much stronger than those of clone 2, clone 6, clone 13A, and those under prior regulatory approval or EUAs (Table 4). These three mAbs however showed no neutralization against the Delta variant (FIG. 17B), further suggesting that they are Omicron-specific.

In order to test if these clones can be used in combination, neutralization assays were again performed by combining two clones. Interestingly, despite epitope overlap, these mAb clones can still enhance each other's neutralization capacity, with the best combination being an antibody cocktail of MB.02+MB.08 (IC50=0.03 μg/mL) against pseudotyped SARS-COV-2 Omicron variant (FIG. 10C). In summary, these lead neutralizing mAbs showed that they have high affinity vs Omicron RBD, and strong potency in pseudovirus neutralization, which are at least 2 orders of magnitude more potent than existing clinically approved or authorized SARS-CoV-2 mAbs, where their cocktail combinations can also further enhance the neutralization potency (Table 4).

To date, the COVID-19 pandemic has entered into a next stage since the emergence of SARS-COV-2 Omicron variant, which spread globally in recent months due to higher transmission rates and immune escape. The Omicron variant harbors 15 mutations were reported in the RBD domain compared with the ancestral Wuhan-1/WA-1 virus, with 9 of these mutations overlap with ACE2 binding footprint, the mediator of host cell entry. In addition, currently approved vaccines (such as BNT162b2, mRNA-1273, and Ad26.COV2.S) are all designed against the original wild-type SARS-COV-2. However, neutralizing antibody responses of sera from convalescent or vaccinated individuals was dramatically decreased with increased time post vaccination to against the emerging variant.

The highly mutated Omicron variant has the potential for evasion of binding and neutralization by the majority of clinically neutralizing. To experimentally validate this assumption 3 high potency neutralizing mAbs were developed and validated against authentic SARS-COV-2 ancestral virus and Delta variant. The studies of the present disclosure found that the Omicron variant, harboring substantially more mutation that prior variant, indeed could completely or partially escape neutralization by existing potent SARS-COV-2 mAbs including approved or emergency authorized clinical antibodies.

To provide countermeasures quickly to new VoCs such as the Omicron variant, the present disclosure provides methods of developing a highly effective animal immunization approach (RAMIHM) with high-throughput customized single cell BCR sequencing. RAMIHM enables us to obtain potent antigen-specific neutralizing mAbs within 3 weeks, offering the opportunity to rapidly respond the potential risks of emerging new viruses or variants. Compared to other approaches, RAMIHM does not rely on human samples and is fully controllable in the laboratory. Compared to traditional antibody development approaches, RAMIHM is faster than regular immunization, and generates fully human mAbs without the need for humanization from traditional animal immunization. Thus, the resulted mAbs developed by RAMIHM are fully human and ready for downstream IND-enabling and/or translational studies. In this study, 3 potent and specific anti-Omicron neutralizing mAbs were identified from Ig humanized mice by RAMIHM. Among those mAbs, MB.08 showed the high binding capacity (K_{D}=7 nM) and strong neutralizing ability against pseudotyped SARS-COV-2 Omicron RBD (IC₅₀=44 ng/mL). All three clones are more potent than the majority of currently approved or authorized clinical RBD-targeting mAbs. Results of epitope binning experiment suggested that MB.08 might bind to sites in Omicron spike RBD with overlapping epitope(s) to PC.03 and MB.02. Nevertheless, an antibody cocktail combining MB.08 with MB.02 exhibited enhanced SARS-COV-2 Omicron neutralization potency (IC₅₀=30 ng/mL) compared to individual clones. These antibodies or their cocktail combinations are worthy of further development, such as downstream IND-enabling and/or translational studies. In general, RAMIHM can also serve as a versatile platform broadly applicable in antibody discovery against emerging pathogens or other therapeutic targets.

### Example 3: Broadly Potent Bispecific Antibodies Against SARS-COV-2 Omicron Sublineages BA.1 and BA.2

### Materials and Methods

### Cloning and Expression of SARS-Cov 2-Specific Human Bispecific Antibodies

Bispecific antibodies termed COV2-0208, COV2-0203, COV2-0803, COV2-0213, and COV2-0813 (see Table 1 and Table 2) were cloned and expressed using methods as described herein at Example 2. In brief, indicated mAb variable regions for each bispecific antibody were amplified and subcloned into separate mammalian expression vectors using Gibson assembly. To express recombinant bispecific antibodies, four expression vectors were transiently transfected into Expi293 cells using ExpiFectamine 293 transfection kit according to the manufacturer's protocol (ThermoFisher). Antibody containing cell culture supernatants were harvested after 5 days of cultivation in shake flasks, then secreted bispecific antibodies were collected and purified by affinity chromatography using rProtein A Sepharose Fast Flow beads (Cytiva, Cat: #17127901). Purified bispecific antibodies were inspected using SDS-PAGE and stored at −80° C. after further usage.

### SARS-Cov-2 Pseudovirus Generation and Neutralization Assay

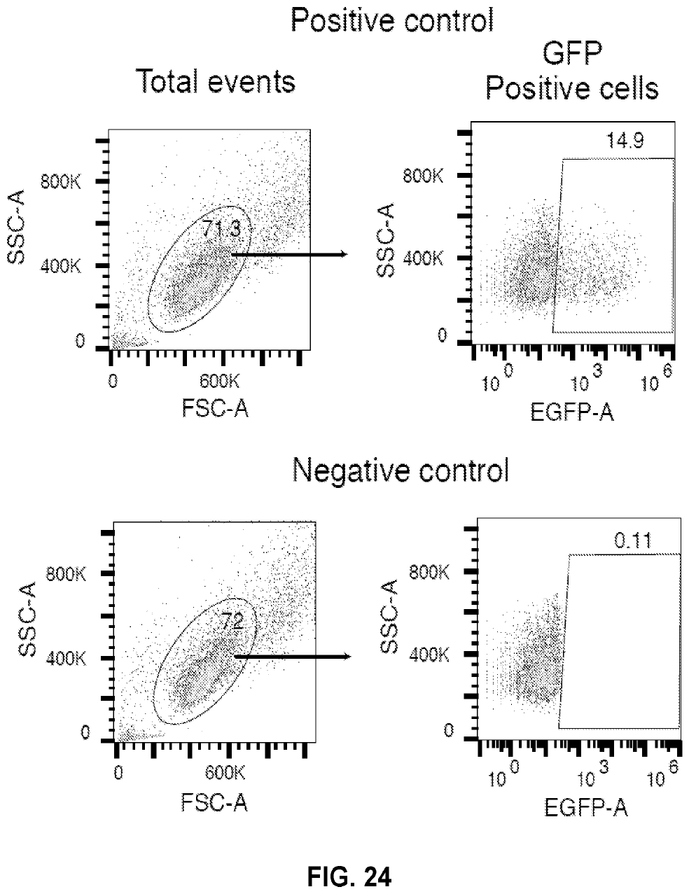

The SARS-COV-2 pseudovirus was produced as described herein at Example 2. Briefly, pseudovirus containing cell culture supernatant was harvested after 2 days of co-transfection of HEK-293T cells with a spike-expressing plasmid and env-deficient HIV-1 backbone vectors, then clarified by centrifugation and stored at −80° C. after further usage. To determine the neutralizing activity of bispecific antibody, serial diluted antibodies were incubated with pseudovirus at 37° ° C. for 1 hour, then co-cultured with HEK-293T-hACE2 cells for overnight. Finally, signal was evaluated after 24 hours by detection of GFP expression in the HEK-293T-hACE2 cells using Attune NxT Acoustic Focusing Cytometer (ThermoFisher) or BD Symphony Flow Cytometry. FIG. 24 shows representative flow gating plots.

### Antibody Binding Quantification and ACE2 Competition Assay

The binding of bispecific antibodies were quantified by ELISA and ACE2 competition assays, as described herein at Example 2. The recombinant SARS-COV-2 RBD wild type (WA-1) (Cat. No. 40592-V08B), SARS-COV-2 Delta RBD (Cat. No. 40592-V08H90), SARS-COV-2 Omicron BA.1 RBD (Cat. No. 40592-V08H121) and SARS-COV-2 Omicron BA.2 RBD (Cat. No.SPD-C522g-100 μg) used in ELISA quantification were purchased from Sino Biological and AcroBiosystems, respectively. The ACE2 (Cat. No. 10108-H08H) used in ACE2 competition assay was purchased from Sino Biological.

### Bispecific Antibody Binding Affinity Measurement

The binding affinity of antibodies with RBD was performed previously by Octet RED96e (ForteBio) using bio-layer interferometry (BLI). RBD his-tagged proteins were immobilized onto HIS1K biosensors (ForteBio) with bispecific IgGs acting as the analyte with serial dilutions. Kd values were calculated using Data Analysis HT 10 (ForteBio) with a 1:1 Langmuir binding model.

### Standard Statistics

Standard statistical methods were applied to non-high-throughput experimental data. The statistical methods are described in figure legends and/or supplementary Excel tables. Prism (GraphPad Software) and RStudio were used for data analyses and source plot generation. Additional information can be found in the supplemental excel tables.

### Replication, Randomization, Blinding and Reagent Validations

Biological or technical replicate samples were randomized where appropriate. Experiments were not blinded. Commercial antibodies were validated by the vendors, and re-validated in house as appropriate. Custom antibodies were validated by specific antibody-antigen interaction assays, such as ELISA. Isotype controls were used for antibody validations. Cell lines were authenticated by original vendors, and re-validated in lab as appropriate. All cell lines tested negative for mycoplasma. No commonly misidentified cell line.

### SARS-COV-2 Omicron Variant and Sublineages

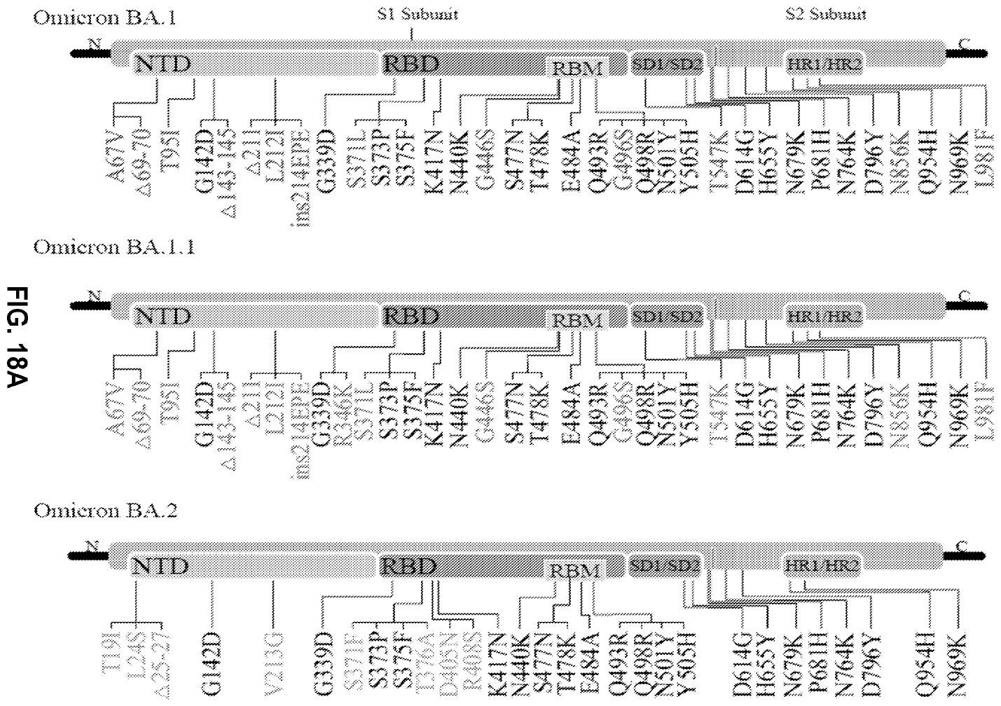

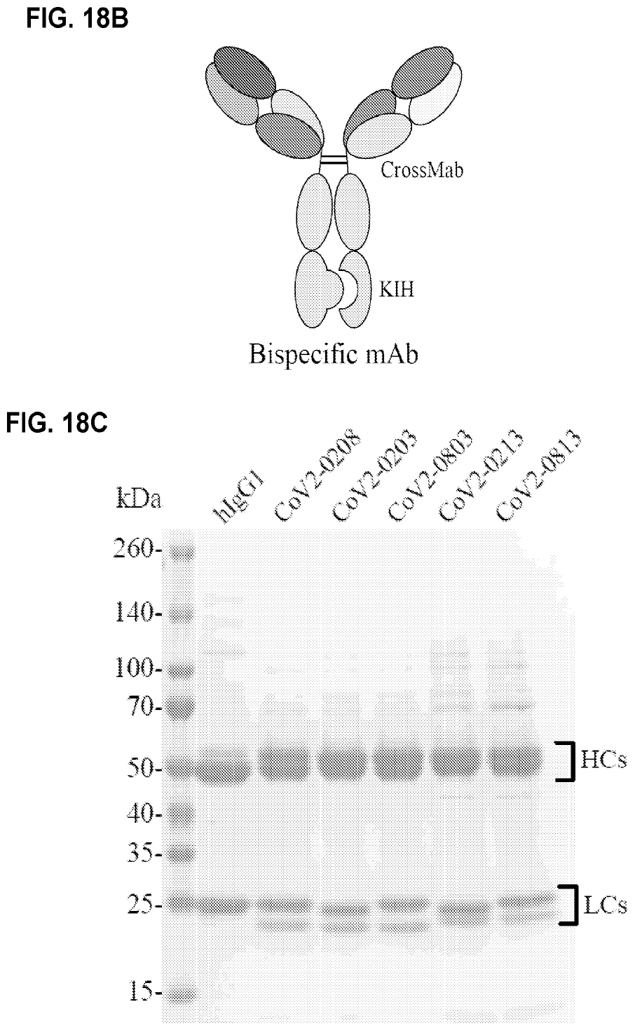

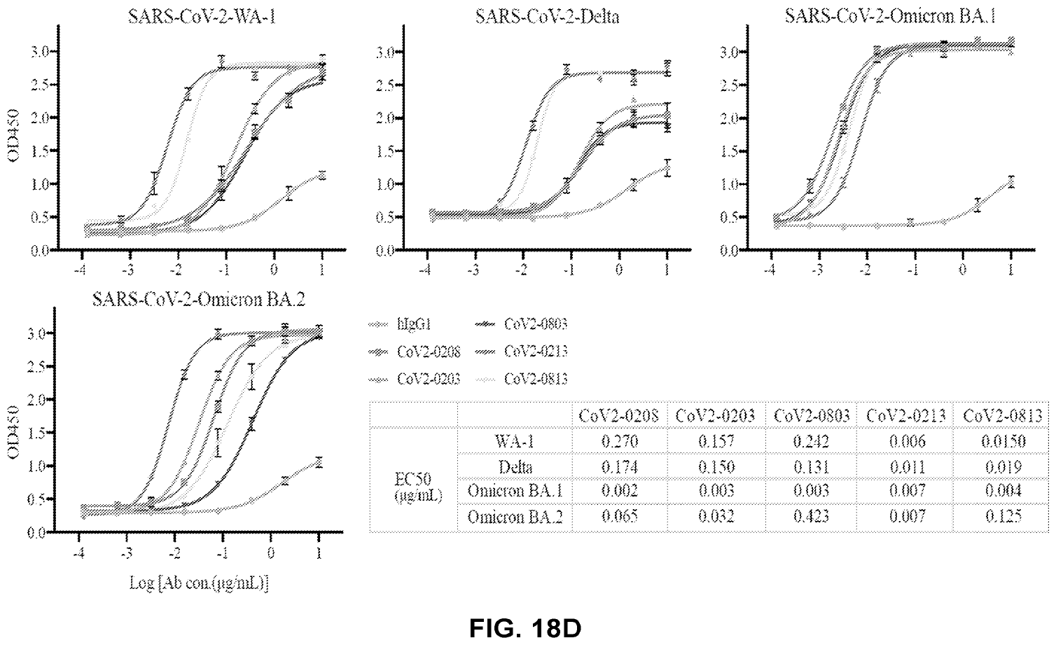

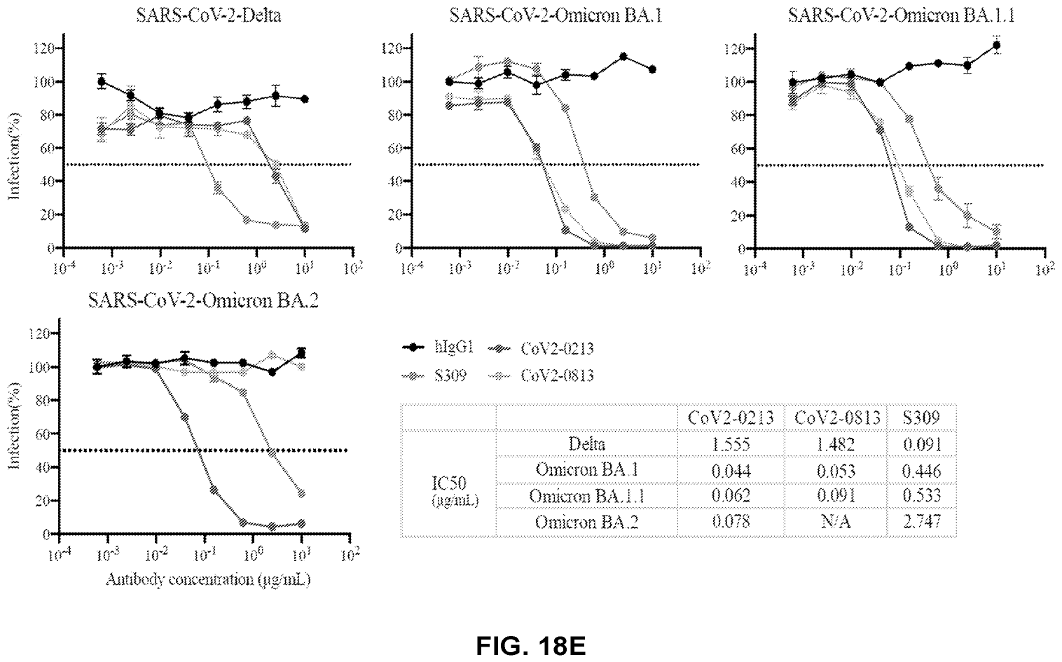

A new SARS-COV-2 lineage, Omicron (B.1.1.529), initially reported in South Africa in late 2021, rapidly became the predominant variant circulating in many countries and led to the fourth pandemic wave globally. As compared with the genome sequences of previous variants of concerns (VOCs), the Omicron B.1.1.529 variant has harbored a high number of genomic mutations, especially in the spike (S) glycoprotein and clustered in the receptor-binding domain (RBD) (FIG. 18A). These mutations drastically decrease the efficacy of currently available vaccines and FDA-approved or emergency use authorized (EUA) monoclonal antibody-based therapies. As of February 2022, several Omicron sublineages have been reported and termed according to WHO weekly epidemiological update, including sublineages BA.1, BA.2 and BA.3. BA.1.1 is a subvariant of BA.1, which has the R346K mutation in the S protein (FIG. 18A) and currently account for ˜5% of Omicron sequences globally based on GISAID update. BA.2 variant shares 12 common RBD mutations to BA.1 variant. However, BA.2 contains four unique mutations in its RBD domain (S371F, T376A, D405N and R408S) (FIG. 18A). These mutations in the Omicron BA.2 variant are consistent with the fact that it is contagious and has more severe antigenic escape as compared to BA.1. As of today, only three clinical stage mAbs (S309/Sotrovimab, LY-CoV1404/Bebtelovimab and BRII-198/Romlusevimab) show substantial neutralization activities against all three major circulating Omicron subvariants, BA.1, BA. 1.1 and BA.2. Therefore, it is critical to rapidly develop countermeasures such as new antibodies against these sublineages.

Previously reported cocktail strategies (i.e., administration of two human monoclonal antibodies), demonstrated that the cocktail approach could increase efficacy and prevent viral escape by SARS-COV-2 mutational variants. However, disadvantages of the cocktail strategy are that it requires production of multiple different m Ab molecules and thereby increases manufacturing costs and volumes. In contrast, a bispecific antibody (bsAb) strategy can simultaneously target two different antigens or antigenic sites according to its structural design, thereby simplifying manufacturing and saving costs. In addition, bsAbs have the potential for synergistic effects of the two binder Fab arms. These features suggest that bsAb strategy may have advantages in COVID-19 therapeutics. Further, because the Omicron sublineages are drastically different from the variants in the ancestral lineage (wildtype, WT, Wuhan-1, WA-1) and earlier variants (such as Delta variant, B.1.617.2), it would be beneficial to combine an Omicron-targeting mAb and a WT/Delta-targeting mAb into a bispecific form.

### Generation and Characterization of Bispecific Antibodies Targeting SARS-COV-2 Variants

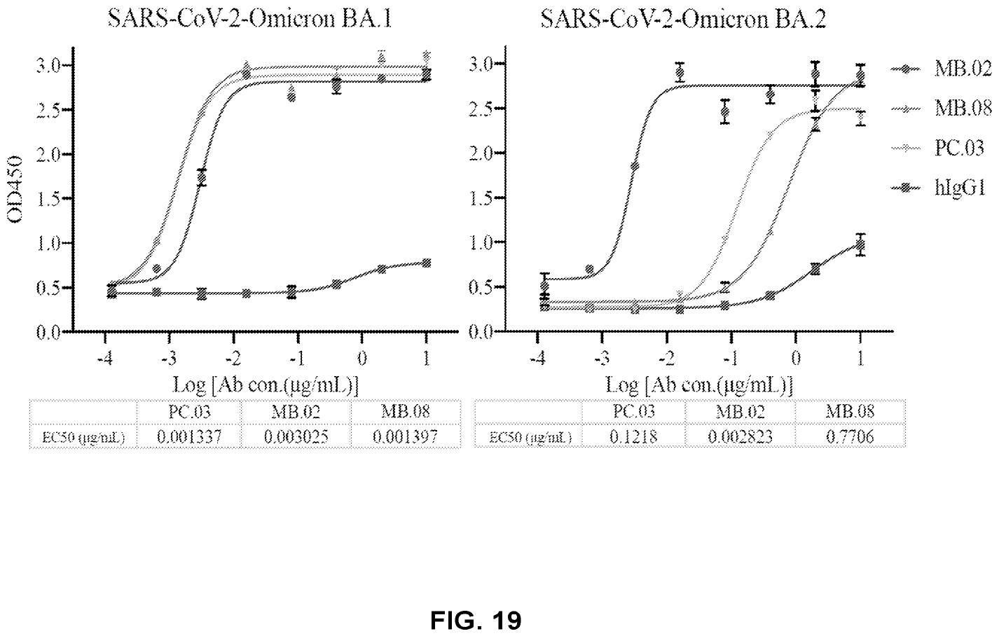

Five broad spectrum SARS-COV-2-specific functional bispecific antibodies were engineered herein by using an IgG1 knob-into-hole bispecific CrossMab antibody technique (FIG. 18B). First, new antibodies that can counter Omicron BA.1 and BA.2 were generated. Antigenic differences in the spike protein of Omicron BA.1 and BA.2 were examined by using Omicron BA.1 RBD-directed neutralizing monoclonal antibodies described herein (see Example 2). This analysis suggested two of three Omicron BA.1 RBD mAbs exhibited dramatically reduced binding activity (100-fold drop) to target on Omicron BA.2, indicating that there may be differences in the effectiveness of mAbs against these different omicron sublineages (FIG. 19). Yet, one of these mAbs, MB.02, maintains similarly high potency against BA.2 (0.003 μg/mL, low single digit ng/mL EC50) (FIG. 19).

Bispecific antibodies were generated utilizing the Fab regions from four previously developed fully human or largely humanized monoclonal antibodies, Clones MB.02, MB.08, PC.03 (see Example 2) and Clone 13A (Peng, et al., Nature Comm., 13, Article number: 1638 (2022)). The resultant bsAb clones were named as CoV2-0208, CoV2-0203, CoV2-0803, CoV2-0213 and CoV2-0813, respectively, based on the compositions of their respective parental monospecific mAb clones. To determine the purity of the bispecific antibodies, the molecular weight of separated modules was analyzed by using reduced SDS-PAGE after Protein A beads purification. The findings indicated that all five bispecific antibodies were successfully expressed with expected size with high purity after protein A purification (FIG. 18C).

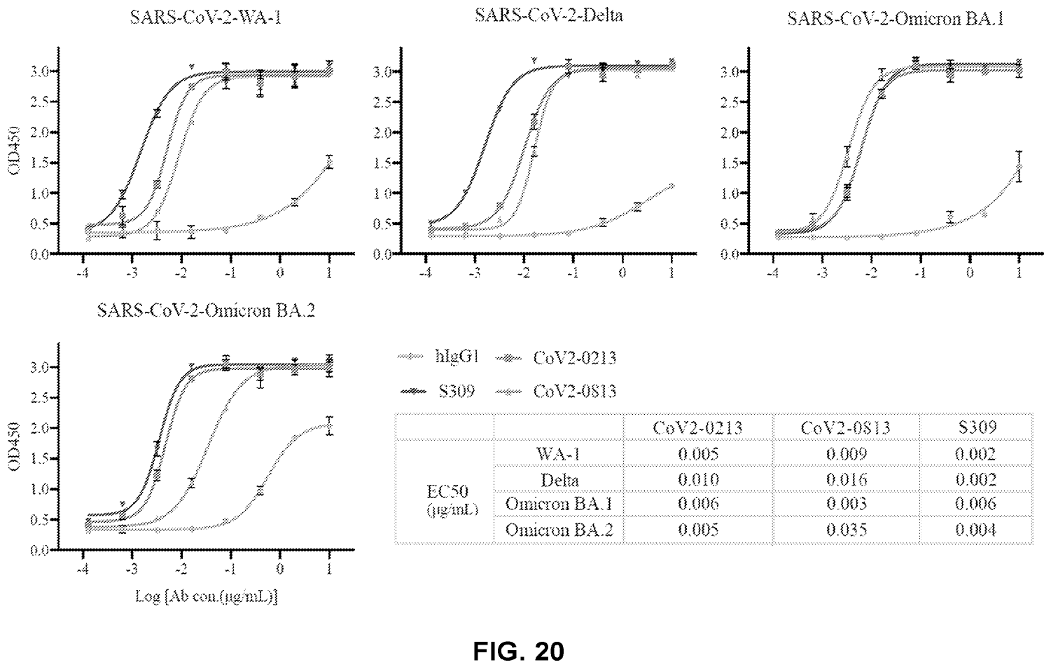

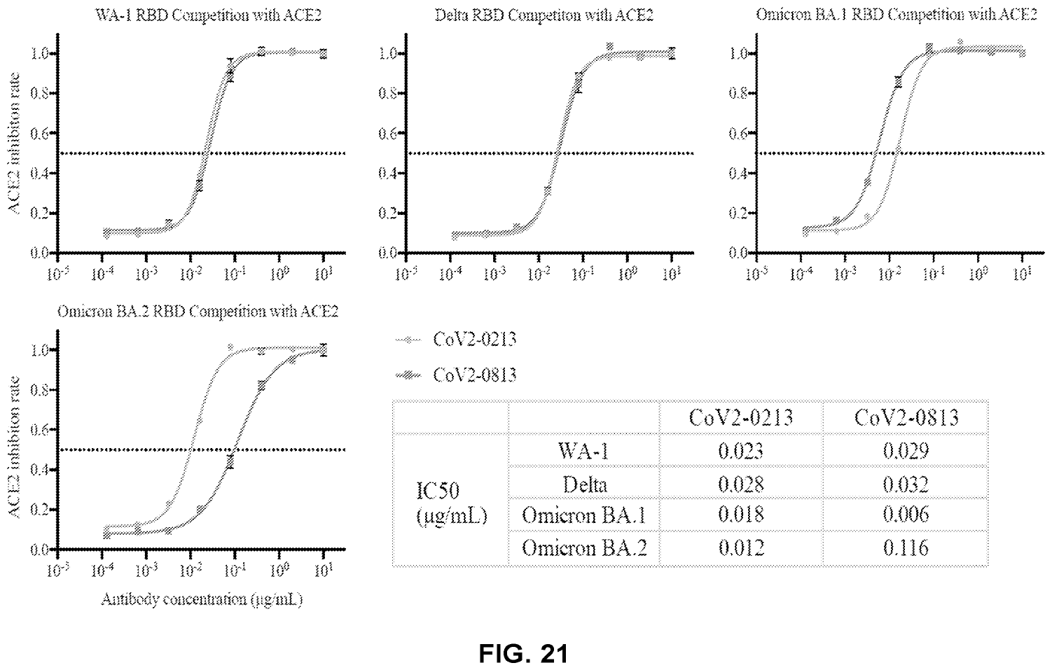

To identify the binding properties of the five purified bispecific antibodies, antibody titration assays were performed by ELISA with four SARS-COV-2 RBD recombinant proteins, which included SARS-COV-2-WA-1 RBD, SARS-COV-2-Delta RBD, SARS-COV-2-Omicron BA.1 RBD and SARS-COV-2-Omicron BA.2 RBD. The experiments were also performed with a clinical mAb S309 (Sotrovimab), which has been in human use under Food and Drug Administration (FDA) issued emergency use authorization (EUA) in the US, and similarly in Europe, UK, Japan and Australia. Among the bsAbs, only CoV2-0213 and CoV2-0813 exhibited substantially lower half-maximum effective concentration (EC₅₀) value to all four SARS-COV-2-RBDs, similar to S309. The remaining bsAbs only showed substantially lower EC₅₀(s) to one or two of SARS-COV-2-RBD(s) (FIG. 18D and FIG. 20). Meanwhile, the lead bsAbs, CoV2-0213 and CoV2-0813, also exhibited strong competition with angiotensin-converting enzyme 2 (ACE2) for binding to a range of SARS-COV-2 RBDs (FIG. 21).

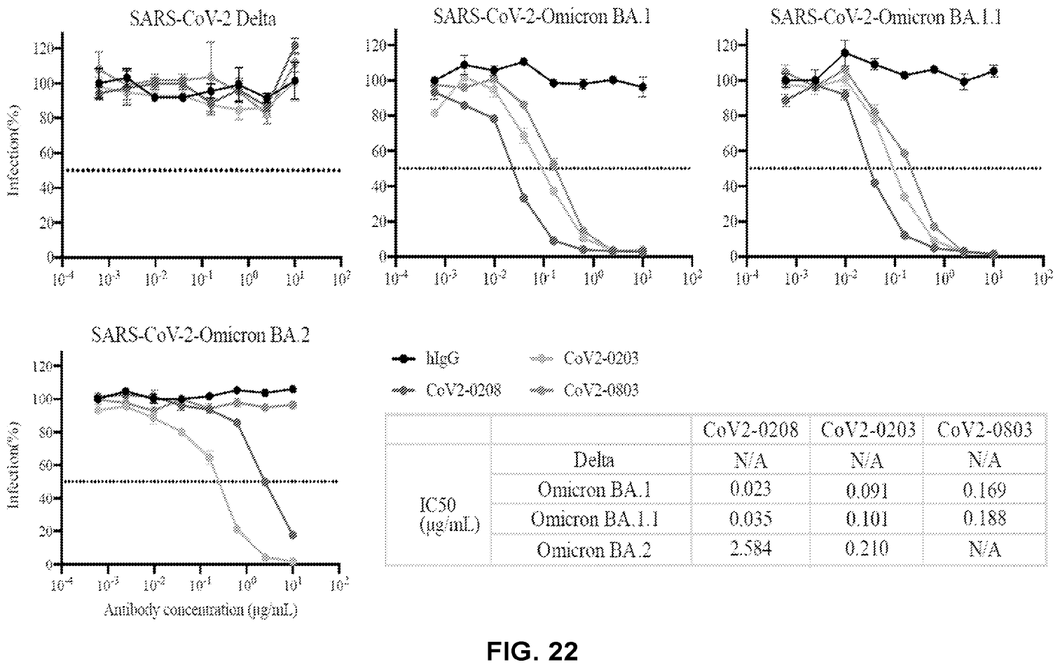

To further assess the functional properties of the five bispecific antibodies, neutralization assay with HIV-1 based pseudoviruses were performed. The control mAb S309/Sotrovimab retained neutralization activity against both Omicron BA.1 and BA.1.1 (BA.1+R346 mutation) with half-maximum inhibitory concentration (IC₅₀) values of 0.446 and 0.533 μg/mL, respectively (FIG. 18E). However, its neutralization activity against Omicron BA.2 dropped 30-fold to an IC₅₀ value of 2.75 μg/mL (FIG. 18E). Of note, the EUA of S309/Sotrovimab was recently withdrawn by FDA in March 2022 due to the high prevalence of BA.2 variant in many states in the US. In contrast, one of the lead bispecific antibodies described herein, CoV2-0213, displayed broad spectrum neutralization ability to a range of SARS-COV-2 VOCs (FIG. 18E). Specifically, CoV2-0213 remained potent in neutralizing three of Omicron sublineages, BA.1, BA. 1.1 and BA.2, with ICso values of 0.044, 0.062 and 0.078 μg/mL, respectively (FIG. 18E). The bsAb CoV2-0213 is an order-of-magnitude more effective than S309/Sotrovimab in neutralization against BA.1 and BA.1.1, and ˜78× more effective than S309/Sotrovimab against BA.2. Meanwhile, two other bispecific antibodies also exhibit potent Omicron-specific neutralization. CoV2-0203 showed high potency in neutralizing the three of Omicron sublineages, although it showed somewhat weaker neutralization ability (1.5-fold weaker) in comparison to CoV2-0213 (FIG. 22). CoV2-0208 showed high potency against BA.1 and BA.1.1, but its activity against BA.2 is on par with S309 (FIG. 22). Both CoV2-0208 and CoV2-0203 lost neutralization activity against Delta. Thus, the lead bsAb is CoV2-0213, which has strong neutralization potency against Omicron BA.1, BA.1.1 and BA.2 and maintains reasonable activity against Delta.

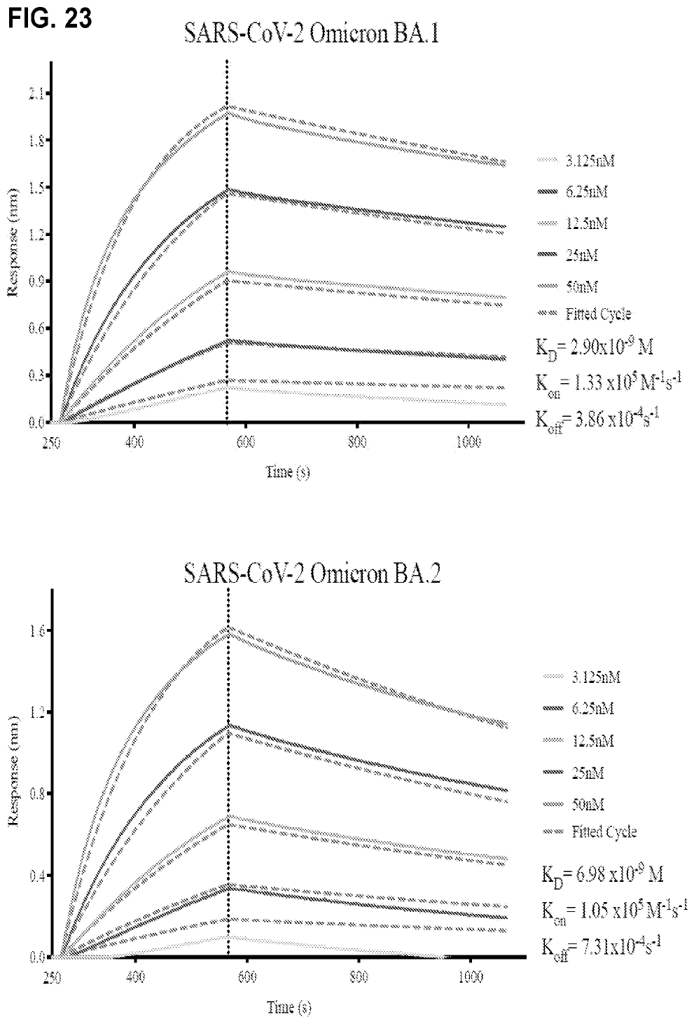

Next, the binding affinity of CoV2-0213 with Omicron BA. 1 and BA.2 RBD recombinant proteins was analyzed by biolayer interferometry (BLI). The BLI results revealed that the lead bispecific antibody, CoV2-0213, displays high single molecule affinity, at single-digit nanomolar Kd against Omicron BA.1 (Kd=2.9 nM) and BA.2 (Kd=6.98 nM) RBD, respectively (FIG. 23). Interestingly, the Kd values of CoV2-0213 against Omicron BA.1 is significantly lower than that of its parental monoclonal antibody (MB.02, Kd=26.5 nM) (see Example 2), suggesting enhanced affinity.

### Enumerated Embodiments

The following exemplary embodiments are provided, the numbering of which is not to be construed as designating levels of importance:

Embodiment 1: A method of identifying antigen-specific antibodies, the method comprising:
a. vaccinating a subject with an effective amount of a lipid nanoparticle comprising at least one mRNA encoding an antigen;
b. boosting the subject with at least one additional vaccination with an effective amount of the lipid nanoparticle comprising at least one mRNA encoding the antigen;
c. isolating B cells from the subject after the vaccination of step a;
d. identifying and cloning BCR heavy and light chain pairs expressed by enriched B cells; and
e. screening the BCR heavy and light chains for antigen-specificity thereby identifying antigen-specific antibodies.

Embodiment 2: The method of embodiment 1, wherein isolating B cells from the subject is performed at least about 14 days after the vaccination of step a.

Embodiment 3: The method of embodiment 1 or embodiment 2, wherein boosting the subject comprises four additional vaccinations.

Embodiment 4: The method of embodiment 3, wherein the additional vaccinations occur about two days, four days, seven days, and eleven days after the initial vaccination.

Embodiment 5: The method of any one of embodiments 1-4, wherein identifying BCR heavy and light chain pairs is performed by single-cell BCR sequencing (scBCRseq).

Embodiment 6: The method of any one of embodiments 1-5, wherein the subject is a mammal.

Embodiment 7: The method of any one of embodiments 1-6, wherein the subject is a mouse.

Embodiment 8: The method of any one of embodiments 1-7, wherein the subject is a transgenic mouse.

Embodiment 9: The method of any one of embodiments 1-8, wherein the subject is a humanized mouse.

Embodiment 10: The method of embodiment 9, wherein the humanized mouse comprises human IgG and IgK transgenes.

Embodiment 11: The method of any one of embodiments 1-10, wherein the vaccination is administered intramuscularly.

Embodiment 12: The method of any one of embodiments 1-11, wherein the antigen is a severe acute respiratory syndrome coronavirus 2 (SARS-COV-2) antigen.

Embodiment 13: The method of embodiment 12, wherein the SARS-COV-2 antigen is a SARS-COV-2 spike protein receptor binding domain (RBD).

Embodiment 14: An antibody produced by the method of any one of embodiments 1-13.

Embodiment 15: A method for rapid identification of a monoclonal antibody or antigen-binding fragment which is capable of binding a spike protein receptor binding domain (RBD) of a severe acute respiratory syndrome coronavirus 2 (SARS-COV-2), the method comprising:
a. immunizing an animal with a polypeptide comprising the spike protein RBD or with a nucleic acid comprising a nucleotide sequence encoding the spike protein RBD;
b. sequencing fully recombined VDJ sequences of B cell receptor (BCR) repertoires from multiple individual B cells of the animal, thereby generating sequencing data;
c. identifying candidate antibody clones from the sequencing data;
d. producing a recombinant monoclonal antibody or antigen-binding fragment comprising paired heavy and light chain variable regions from one or more candidate antibody clones; and
e. testing the produced antibody or antigen-binding fragment for an ability to bind the spike protein RBD.

Embodiment 16: The method of embodiment 15, wherein the sequencing is high-throughput single-cell B-cell receptor sequencing (scBCR-seq).

Embodiment 17: The method of embodiment 15 or embodiment 16, wherein the animal is a mouse.

Embodiment 18: The method of any one of embodiments 15-17, wherein the animal is a C57BL/6J mouse or a BALB/c mouse.

Embodiment 19: The method of any one of embodiments 15-18, wherein the multiple individual B cells are isolated progenitor B cells and/or plasma B cells from spleen, lymph node, and/or bone marrow of the animal.

Embodiment 20: The method of any one of embodiments 15-19, wherein the SARS-CoV-2 is wild-type SARS-COV-2 or a variant SARS-COV-2.

Embodiment 21: The method of any one of embodiments 15-20, wherein the produced antibody or antigen-binding fragment binds the RBD with nanomolar affinity.

Embodiment 22: The method of any one of embodiments 15-21, wherein the produced antibody or antigen-binding fragment binds the RBD with picomolar avidity.

Embodiment 23: The method of any one of embodiments 15-22, wherein the produced antibody or antigen-binding fragment has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 pM, less than 20 pM, less than 10 pM, less than 5 pM, or less than 2.5 pM.

Embodiment 24: The method of any one of embodiments 15-23, wherein the produced antibody or antigen-binding fragment specifically binds the SARS-COV-2 spike protein RBD.

Embodiment 25: The method of any one of embodiments 15-24, wherein the antibody or antigen-binding fragment binds an epitope of the RBD which partially or completely overlaps with the angiotensin converting enzyme II (ACE2) binding site of RBD, thereby preventing RBD binding to ACE2.

Embodiment 26: The method of any one of embodiments 15-25, wherein the produced antibody or antigen-binding fragment is a neutralizing antibody or antigen-binding fragment.

Embodiment 27: The method of any one of embodiments 15-26, wherein the produced antibody or antigen-binding fragment is effective against lethal challenge of SARS-COV-2 in vivo.

Embodiment 28: The method of any one of embodiments 15-27, wherein the produced antibody or antigen-binding fragment is selected from the group consisting of a monospecific antibody, a bispecific antibody, a Fab, an Fab′, an F(ab′)₂, an Fv, an scFv, a linear antibody, and a single domain antibody (sdAb).

Embodiment 29: The method of embodiment 28, wherein the produced antibody or antigen-binding fragment is a bispecific antibody selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb).

Embodiment 30: The method of embodiment 29, wherein the bispecific antibody is an asymmetric IgG-like bispecific antibody which binds two distinct epitopes of the RBD.

Embodiment 31: The method of any one of embodiments 15-30, wherein the produced antibody or antigen-binding fragment is conjugated or recombinantly fused to at least one other moiety or molecule selected from the group consisting of a diagnostic agent, a detectable agent, a therapeutic agent, a purification tag, a molecule affecting one or more biological or molecular properties of the antibody or antigen-binding domain, and any combination thereof, wherein the biological or molecular properties comprise serum stability, half-life, solubility, and antigenicity.

Embodiment 32: An antibody produced by the method of any one of embodiments 15-31.

Embodiment 33: A monoclonal antibody or antigen-binding fragment, wherein the antibody or antigen-binding fragment is capable of binding a severe acute respiratory syndrome coronavirus 2 (SARS-COV-2) spike protein receptor binding domain (RBD), wherein the antibody or antigen-binding fragment comprises three heavy chain complementarity determining regions (HCDR1, HCDR2, and HCDR3) and three light chain complementarity determining regions (LCDR1, LCDR2, and LCDR3) selected from the group consisting of:
a. an HCDR1 comprising SEQ ID NO: 1, an HCDR2 comprising SEQ ID NO: 2, an HCDR3 comprising SEQ ID NO: 3, an LCDR1 comprising SEQ ID NO: 4, an LCDR2 comprising the amino acid sequence AAS, and an LCDR3 comprising SEQ ID NO: 6;
b. an HCDR1 comprising SEQ ID NO: 11, an HCDR2 comprising SEQ ID NO: 12, an HCDR3 comprising SEQ ID NO: 13, an LCDR1 comprising SEQ ID NO: 14, an LCDR2 comprising the amino acid sequence GSS, and an LCDR3 comprising SEQ ID NO: 16;
c. an HCDR1 comprising SEQ ID NO: 21, an HCDR2 comprising SEQ ID NO: 22, an HCDR3 comprising SEQ ID NO: 23, an LCDR1 comprising SEQ ID NO: 24, an LCDR2 comprising the amino acid sequence AAS, and an LCDR3 comprising SEQ ID NO: 26;
d. an HCDR1 comprising SEQ ID NO: 31, an HCDR2 comprising SEQ ID NO: 32, an HCDR3 comprising SEQ ID NO: 33, an LCDR1 comprising SEQ ID NO: 34, an LCDR2 comprising the amino acid sequence AAS, and an LCDR3 comprising SEQ ID NO: 36;
e. an HCDR1 comprising SEQ ID NO: 107, an HCDR2 comprising SEQ ID NO: 108, an HCDR3 comprising SEQ ID NO: 109, an LCDR1 comprising SEQ ID NO: 116, an LCDR2 comprising the amino acid sequence GAS, and an LCDR3 comprising SEQ ID NO: 118;
f. an HCDR1 comprising SEQ ID NO: 110, an HCDR2 comprising SEQ ID NO: 111, an HCDR3 comprising SEQ ID NO: 112, an LCDR1 comprising SEQ ID NO: 119, an LCDR2 comprising the amino acid sequence WAS, and an LCDR3 comprising SEQ ID NO: 121; and
g. an HCDR1 comprising SEQ ID NO: 113, an HCDR2 comprising SEQ ID NO: 114, an HCDR3 comprising SEQ ID NO: 115, an LCDR1 comprising SEQ ID NO: 122, an LCDR2 comprising the amino acid sequence DAS, and an LCDR3 comprising SEQ ID NO: 124.

Embodiment 34: The antibody or antigen-binding fragment of embodiment 33, wherein the SARS—Co-V-2 is wild-type SARS-COV-2 or a variant SARS-COV-2.

Embodiment 35: The antibody or antigen-binding fragment of embodiment 33 or embodiment 34, wherein the antibody or antigen-binding fragment binds the RBD with nanomolar affinity.

Embodiment 36: The antibody or antigen-binding fragment of any one of embodiments 33-35, wherein the antibody or antigen-binding fragment binds the RBD with picomolar avidity.

Embodiment 37: The antibody or antigen-binding fragment of any one of embodiments 33-36, wherein the antibody or antigen-binding fragment has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 pM, less than 20 pM, less than 10 pM, less than 5 pM, or less than 2.5 pM.

Embodiment 38: The antibody or antigen-binding fragment of any one of embodiments 33-37, wherein the antibody or antigen-binding fragment specifically binds the SARS-COV-2 spike protein RBD.

Embodiment 39: The antibody or antigen-binding fragment of any one of embodiments 33-38, wherein the antibody or antigen-binding fragment binds an epitope of the RBD which partially or completely overlaps with the angiotensin converting enzyme II (ACE2) binding site of RBD, thereby preventing RBD binding to ACE2.

Embodiment 40: The antibody or antigen-binding fragment of any one of embodiments 33-39, wherein the antibody or antigen-binding fragment is a neutralizing antibody or antigen-binding fragment.

Embodiment 41: The antibody or antigen-binding fragment of any one of embodiments 33-40, wherein the antibody or antigen-binding fragment is effective against lethal challenge of SARS-COV-2 in vivo.

Embodiment 42: The antibody or antigen-binding fragment of any one of embodiments 33-41, wherein the antibody or antigen-binding fragment is selected from the group consisting of a monospecific antibody, a bispecific antibody, an Fab, an Fab′, an F(ab′)₂, an Fv, an scFv, a linear antibody, and a single domain antibody (sdAb).

Embodiment 43: The antibody or antigen-binding fragment of embodiment 42, wherein the antibody or antigen-binding fragment is a bispecific antibody selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb).

Embodiment 44: The antibody or antigen-binding fragment of embodiment 43, wherein the bispecific antibody is an asymmetric IgG-like bispecific antibody which binds two distinct epitopes of the RBD.

Embodiment 45: The antibody or antigen-binding fragment of any one of embodiments 33-44, wherein the antibody or antigen-binding fragment comprises at least one heavy chain variable region (VH) and at least one light chain variable region (VL) selected from the group consisting of:
a. a VH comprising an amino acid sequence at least 80%, 85%, 90%, 95%, 96%, 97%, 98%, 99%, or 100% identical to SEQ ID NO: 7, and a VL comprising an amino acid sequence at least 80%, 85%, 90%, 95%, 96%, 97%, 98%, 99%, or 100% identical to SEQ ID NO: 8;
b. a VH comprising an amino acid sequence at least 80%, 85%, 90%, 95%, 96%, 97%, 98%, 99%, or 100% identical to SEQ ID NO: 17, and a VL comprising an amino acid sequence at least 80%, 85%, 90%, 95%, 96%, 97%, 98%, 99%, or
c. a VH comprising an amino acid sequence at least 80%, 85%, 90%, 95%, 96%, 97%, 98%, 99%, or 100% identical to SEQ ID NO: 27, and a VL comprising an amino acid sequence at least 80%, 85%, 90%, 95%, 96%, 97%, 98%, 99%, or 100% identical to SEQ ID NO: 28;
d. a VH comprising an amino acid sequence at least 80%, 85%, 90%, 95%, 96%, 97%, 98%, 99%, or 100% identical to SEQ ID NO: 37, and a VL comprising an amino acid sequence at least 80%, 85%, 90%, 95%, 96%, 97%, 98%, 99%, or 100% identical to SEQ ID NO: 38;
e a VH comprising an amino acid sequence at least 80%, 85%, 90%, 95%, 96%, 97%, 98%, 99%, or 100% identical to SEQ ID NO: 101, and a VL comprising an amino acid sequence at least 80%, 85%, 90%, 95%, 96%, 97%, 98%, 99%, or 100% identical to SEQ ID NO: 104;
f. a VH comprising an amino acid sequence at least 80%, 85%, 90%, 95%, 96%, 97%, 98%, 99%, or 100% identical to SEQ ID NO: 102, and a VL comprising an amino acid sequence at least 80%, 85%, 90%, 95%, 96%, 97%, 98%, 99%, or 100% identical to SEQ ID NO: 105; and
g. a VH comprising an amino acid sequence at least 80%, 85%, 90%, 95%, 96%, 97%, 98%, 99%, or 100% identical to SEQ ID NO: 103, and a VL comprising an amino acid sequence at least 80%, 85%, 90%, 95%, 96%, 97%, 98%, 99%, or 100% identical to SEQ ID NO: 106.

Embodiment 46: The antibody or antigen-binding fragment of embodiment 45, wherein the antibody or antigen-binding fragment comprises at least one heavy chain and at least one light chain selected from the group consisting of:
a. a heavy chain comprising an amino acid sequence at least 80%, 85%, 90%, 95%, 96%, 97%, 98%, 99%, or 100% identical to SEQ ID NO: 9, and a light chain comprising an amino acid sequence at least 80%, 85%, 90%, 95%, 96%, 97%, 98%, 99%, or 100% identical to SEQ ID NO: 10;
b. a heavy chain comprising an amino acid sequence at least 80%, 85%, 90%, 95%, 96%, 97%, 98%, 99%, or 100% identical to SEQ ID NO: 19, and a light chain comprising an amino acid sequence at least 80%, 85%, 90%, 95%, 96%, 97%, 98%, 99%, or 100% identical to SEQ ID NO: 20;
c. a heavy chain comprising an amino acid sequence at least 80%, 85%, 90%, 95%, 96%, 97%, 98%, 99%, or 100% identical to SEQ ID NO: 29, and a light chain comprising an amino acid sequence at least 80%, 85%, 90%, 95%, 96%, 97%, 98%, 99%, or 100% identical to SEQ ID NO: 30;
d. a heavy chain comprising an amino acid sequence at least 80%, 85%, 90%, 95%, 96%, 97%, 98%, 99%, or 100% identical to SEQ ID NO: 39, and a light chain comprising an amino acid sequence at least 80%, 85%, 90%, 95%, 96%, 97%, 98%, 99%, or 100% identical to SEQ ID NO: 40;
e. a heavy chain comprising an amino acid sequence at least 80%, 85%, 90%, 95%, 96%, 97%, 98%, 99%, or 100% identical to SEQ ID NO: 133, and a light chain comprising an amino acid sequence at least 80%, 85%, 90%, 95%, 96%, 97%, 98%, 99%, or 100% identical to SEQ ID NO: 134;
f. a heavy chain comprising an amino acid sequence at least 80%, 85%, 90%, 95%, 96%, 97%, 98%, 99%, or 100% identical to SEQ ID NO: 135, and a light chain comprising an amino acid sequence at least 80%, 85%, 90%, 95%, 96%, 97%, 98%, 99%, or 100% identical to SEQ ID NO: 136; and
g. a heavy chain comprising an amino acid sequence at least 80%, 85%, 90%, 95%, 96%, 97%, 98%, 99%, or 100% identical to SEQ ID NO: 137, and a light chain comprising an amino acid sequence at least 80%, 85%, 90%, 95%, 96%, 97%, 98%, 99%, or 100% identical to SEQ ID NO: 138.

Embodiment 47: The antibody or antigen-biding fragment of any one of embodiments 33-46, wherein the antibody or antigen-binding fragment is conjugated or recombinantly fused to at least one other moiety or molecule selected from the group consisting of a diagnostic agent, a detectable agent, a therapeutic agent, a purification tag, a molecule affecting one or more biological or molecular properties of the antibody or antigen-binding domain, or any combination thereof, wherein the biological or molecular properties comprise serum stability, half-life, solubility, and antigenicity.

Embodiment 48: A bispecific antibody capable of binding a severe acute respiratory syndrome coronavirus 2 (SARS-COV-2) spike protein receptor binding domain (RBD), wherein the bispecific antibody comprises (i) a first heavy chain comprising a first heavy chain variable region (VH1), (ii) a first light chain comprising a first light chain variable region (VL1), (iii) a second heavy chain comprising a second heavy chain variable region (VH2), and (iv) a second light chain comprising a second light chain variable region (VL2); wherein the VH1 and the VH2 each comprises three heavy chain complementarity determining regions (HCDR1, HCDR2, and HCDR3) and the VL1 and the VL2 each comprises three light chain complementarity determining regions (LCDR1, LCDR2, and LCDR3); wherein the complementarity determining regions are selected from the group consisting of:
a. a VH1 HCDR1 comprising SEQ ID NO: 41, a VH1 HCDR2 comprising SEQ ID NO: 42, a VH1 HCDR3 comprising SEQ ID NO: 43, a VL1 LCDR1 comprising SEQ ID NO: 47, a VL1 LCDR2 comprising the amino acid sequence GSS, a VL1 LCDR3 comprising SEQ ID NO: 49, a VH2 HCDR1 comprising SEQ ID NO: 44, a VH2 HCDR2 comprising SEQ ID NO: 45, a VH2 HCDR3 comprising SEQ ID NO: 46, a VL2 LCDR1 comprising SEQ ID NO: 50, a VL2 LCDR2 comprising the amino acid sequence AAS, and a VL2 LCDR3 comprising SEQ ID NO: 52;
b. a VH1 HCDR1 comprising SEQ ID NO: 151, a VH1 HCDR2 comprising SEQ ID NO: 152, a VH1 HCDR3 comprising SEQ ID NO: 153, a VL1 LCDR1 comprising SEQ ID NO: 165, a VL1 LCDR2 comprising the amino acid sequence GAS, a VL1 LCDR3 comprising SEQ ID NO: 166, a VH2 HCDR1 comprising SEQ ID NO: 158, a VH2 HCDR2 comprising SEQ ID NO: 159, a VH2 HCDR3 comprising SEQ ID NO: 160, a VL2 LCDR1 comprising SEQ ID NO: 171, a VL2 LCDR2 comprising the amino acid sequence WAS, and a VL2 LCDR3 comprising SEQ ID NO: 172;
c. a VH1 HCDR1 comprising SEQ ID NO: 177, a VH1 HCDR2 comprising SEQ ID NO: 178, a VH1 HCDR3 comprising SEQ ID NO: 179, a VL1 LCDR1 comprising SEQ ID NO: 191, a VL1 LCDR2 comprising the amino acid sequence DAS, a VL1 LCDR3 comprising SEQ ID NO: 192, a VH2 HCDR1 comprising SEQ ID NO: 184, a VH2 HCDR2 comprising SEQ ID NO: 185, a VH2 HCDR3 comprising SEQ ID NO: 186, a VL2 LCDR1 comprising SEQ ID NO: 197, a VL2 LCDR2 comprising the amino acid sequence GAS, and a VL2 LCDR3 comprising SEQ ID NO: 198;
d. a VH1 HCDR1 comprising SEQ ID NO: 203, a VH1 HCDR2 comprising SEQ ID NO: 204, a VH1 HCDR3 comprising SEQ ID NO: 205, a VL1 LCDR1 comprising SEQ ID NO: 217, a VL1 LCDR2 comprising the amino acid sequence DAS, a VL1 LCDR3 comprising SEQ ID NO: 218, a VH2 HCDR1 comprising SEQ ID NO: 210, a VH2 HCDR2 comprising SEQ ID NO: 211, a VH2 HCDR3 comprising SEQ ID NO: 212, a VL2 LCDR1 comprising SEQ ID NO: 223, a VL2 LCDR2 comprising the amino acid sequence WAS, and a VL2 LCDR3 comprising SEQ ID NO: 224;
e. a VH1 HCDR1 comprising SEQ ID NO: 229, a VH1 HCDR2 comprising SEQ ID NO: 230, a VH1 HCDR3 comprising SEQ ID NO: 231, a VL1 LCDR1 comprising SEQ ID NO: 243, a VL1 LCDR2 comprising the amino acid sequence AAS, a VL1 LCDR3 comprising SEQ ID NO: 244, a VH2 HCDR1 comprising SEQ ID NO: 236, a VH2 HCDR2 comprising SEQ ID NO: 237, a VH2 HCDR3 comprising SEQ ID NO: 238, a VL2 LCDR1 comprising SEQ ID NO: 249, a VL2 LCDR2 comprising the amino acid sequence GAS, and a VL2 LCDR3 comprising SEQ ID NO: 250; and
f. a VH1 HCDR1 comprising SEQ ID NO: 255, a VH1 HCDR2 comprising SEQ ID NO: 256, a VH1 HCDR3 comprising SEQ ID NO: 257, a VL1 LCDR1 comprising SEQ ID NO: 269, a VL1 LCDR2 comprising the amino acid sequence AAS, a VL1 LCDR3 comprising SEQ ID NO: 270, a VH2 HCDR1 comprising SEQ ID NO: 262, a VH2 HCDR2 comprising SEQ ID NO: 263, a VH2 HCDR3 comprising SEQ ID NO: 264, a VL2 LCDR1 comprising SEQ ID NO: 275, a VL2 LCDR2 comprising the amino acid sequence WAS, and a VL2 LCDR3 comprising SEQ ID NO: 276.

Embodiment 49: The bispecific antibody of embodiment 48, wherein the SARS—Co-V-2 is wild-type SARS-COV-2 or a variant SARS-COV-2.

Embodiment 50: The bispecific antibody of embodiment 48 or embodiment 49, wherein the bispecific antibody binds the RBD with nanomolar affinity.

Embodiment 51: The bispecific antibody of any one of embodiments 48-50, wherein the bispecific antibody binds the RBD with picomolar avidity.

Embodiment 52: The bispecific antibody of any one of embodiments 48-51, wherein the bispecific antibody has a dissociation constant (K_{D}) for the RBD of less than 100 nM, less than 50 nM, less than 40 nM, less than 30 nM, less than 20 nM, less than 10 nM, less than 5 nM, less than 1 nM, less than 500 pM, less than 250 pM, less than 100 pM, less than 50 pM, less than 40 pM, less than 30 pM, less than 20 pM, less than 10 pM, less than 5 pM, or less than 2.5 pM.

Embodiment 53: The bispecific antibody of any one of embodiments 48-52, wherein the bispecific antibody specifically binds the SARS-COV-2 spike protein RBD.

Embodiment 54: The bispecific antibody of any one of embodiments 48-53, wherein the bispecific antibody binds an epitope of the RBD which partially or completely overlaps with the angiotensin converting enzyme II (ACE2) binding site of RBD, thereby preventing RBD binding to ACE2.

Embodiment 55: The bispecific antibody of any one of embodiments 48-54, wherein the bispecific antibody is a neutralizing antibody or antigen-binding fragment.

Embodiment 56: The bispecific antibody of any one of embodiments 48-55, wherein the bispecific antibody is effective against lethal challenge of SARS-COV-2 in vivo.

Embodiment 57: The bispecific antibody of any one of embodiments 48-56, wherein the bispecific antibody is selected from the group consisting of an asymmetric IgG-like bispecific antibody, a bispecific T-cell engager (BiTE), a BITE-Fc, a dual-affinity re-targeting protein (DART), a DART-Fc, and a tandem diabody (TandAb).

Embodiment 58: The bispecific antibody of any one of embodiments 48-57, wherein the bispecific antibody is an asymmetric IgG-like bispecific antibody which binds two distinct epitopes of the RBD.

Embodiment 59: The bispecific antibody of any one of embodiments 48-58, wherein the VH1, VL1, VH2, and VL2 are selected from the group consisting of:
a. a VH1 comprising an amino acid sequence at least 80%, 85%, 90%, 95%, 96%, 97%, 98%, 99%, or 100% identical to SEQ ID NO: 53, a VL1 comprising an amino acid sequence at least 80%, 85%, 90%, 95%, 96%, 97%, 98%, 99%, or 100% identical to SEQ ID NO: 55, a VH2 comprising an amino acid sequence at least 80%, 85%, 90%, 95%, 96%, 97%, 98%, 99%, or 100% identical to SEQ ID NO: 54, and a VL2 comprising an amino acid sequence at least 80%, 85%, 90%, 95%, 96%, 97%, 98%, 99%, or 100% identical to SEQ ID NO: 56;
b. a VH1 comprising an amino acid sequence at least 80%, 85%, 90%, 95%, 96%, 97%, 98%, 99%, or 100% identical to SEQ ID NO: 154, a VL1 comprising an amino acid sequence at least 80%, 85%, 90%, 95%, 96%, 97%, 98%, 99%, or 100% identical to SEQ ID NO: 167, a VH2 comprising an amino acid sequence at least 80%, 85%, 90%, 95%, 96%, 97%, 98%, 99%, or 100% identical to SEQ ID NO: 161, and a VL2 comprising an amino acid sequence at least 80%, 85%, 90%, 95%, 96%, 97%, 98%, 99%, or 100% identical to SEQ ID NO: 173;
c. a VH1 comprising an amino acid sequence at least 80%, 85%, 90%, 95%, 96%, 97%, 98%, 99%, or 100% identical to SEQ ID NO: 180, a VL1 comprising an amino acid sequence at least 80%, 85%, 90%, 95%, 96%, 97%, 98%, 99%, or 100% identical to SEQ ID NO: 193, a VH2 comprising an amino acid sequence at least 80%, 85%, 90%, 95%, 96%, 97%, 98%, 99%, or 100% identical to SEQ ID NO: 187, and a VL2 comprising an amino acid sequence at least 80%, 85%, 90%, 95%, 96%, 97%, 98%, 99%, or 100% identical to SEQ ID NO: 199;
d. a VH1 comprising an amino acid sequence at least 80%, 85%, 90%, 95%, 96%, 97%, 98%, 99%, or 100% identical to SEQ ID NO: 206, a VL1 comprising an amino acid sequence at least 80%, 85%, 90%, 95%, 96%, 97%, 98%, 99%, or 100% identical to SEQ ID NO: 219, a VH2 comprising an amino acid sequence at least 80%, 85%, 90%, 95%, 96%, 97%, 98%, 99%, or 100% identical to SEQ ID NO: 213, and a VL2 comprising an amino acid sequence at least 80%, 85%, 90%, 95%, 96%, 97%, 98%, 99%, or 100% identical to SEQ ID NO: 225;
e. a VH1 comprising an amino acid sequence at least 80%, 85%, 90%, 95%, 96%, 97%, 98%, 99%, or 100% identical to SEQ ID NO: 232, a VL1 comprising an amino acid sequence at least 80%, 85%, 90%, 95%, 96%, 97%, 98%, 99%, or 100% identical to SEQ ID NO: 245, a VH2 comprising an amino acid sequence at least 80%, 85%, 90%, 95%, 96%, 97%, 98%, 99%, or 100% identical to SEQ ID NO: 239, and a VL2 comprising an amino acid sequence at least 80%, 85%, 90%, 95%, 96%, 97%, 98%, 99%, or 100% identical to SEQ ID NO: 251; and
f. a VH1 comprising an amino acid sequence at least 80%, 85%, 90%, 95%, 96%, 97%, 98%, 99%, or 100% identical to SEQ ID NO: 258, a VL1 comprising an amino acid sequence at least 80%, 85%, 90%, 95%, 96%, 97%, 98%, 99%, or 100% identical to SEQ ID NO: 271, a VH2 comprising an amino acid sequence at least 80%, 85%, 90%, 95%, 96%, 97%, 98%, 99%, or 100% identical to SEQ ID NO: 265, and a VL2 comprising an amino acid sequence at least 80%, 85%, 90%, 95%, 96%, 97%, 98%, 99%, or 100% identical to SEQ ID NO: 277.

Embodiment 60: The bispecific antibody of any one of embodiments 48-59, wherein the first heavy chain, the first light chain, the second heavy chain, and the second light chain are selected from the group consisting of:
a. a first heavy chain comprising an amino acid sequence at least 80%, 85%, 90%, 95%, 96%, 97%, 98%, 99%, or 100% identical to SEQ ID NO: 57, a first light chain comprising an amino acid sequence at least 80%, 85%, 90%, 95%, 96%, 97%, 98%, 99%, or 100% identical to SEQ ID NO: 59, a second heavy chain comprising an amino acid sequence at least 80%, 85%, 90%, 95%, 96%, 97%, 98%, 99%, or 100% identical to SEQ ID NO: 58, and a second light chain comprising an amino acid sequence at least 80%, 85%, 90%, 95%, 96%, 97%, 98%, 99%, or 100% identical to SEQ ID NO: 60;
b. a first heavy chain comprising an amino acid sequence at least 80%, 85%, 90%, 95%, 96%, 97%, 98%, 99%, or 100% identical to SEQ ID NO: 155, a first light chain comprising an amino acid sequence at least 80%, 85%, 90%, 95%, 96%, 97%, 98%, 99%, or 100% identical to SEQ ID NO: 168, a second heavy chain comprising an amino acid sequence at least 80%, 85%, 90%, 95%, 96%, 97%, 98%, 99%, or 100% identical to SEQ ID NO: 162, and a second light chain comprising an amino acid sequence at least 80%, 85%, 90%, 95%, 96%, 97%, 98%, 99%, or 100% identical to SEQ ID NO: 174;
c. a first heavy chain comprising an amino acid sequence at least 80%, 85%, 90%, 95%, 96%, 97%, 98%, 99%, or 100% identical to SEQ ID NO: 181, a first light chain comprising an amino acid sequence at least 80%, 85%, 90%, 95%, 96%, 97%, 98%, 99%, or 100% identical to SEQ ID NO: 194, a second heavy chain comprising an amino acid sequence at least 80%, 85%, 90%, 95%, 96%, 97%, 98%, 99%, or 100% identical to SEQ ID NO: 188, and a second light chain comprising an amino acid sequence at least 80%, 85%, 90%, 95%, 96%, 97%, 98%, 99%, or 100% identical to SEQ ID NO: 200;
d. a first heavy chain comprising an amino acid sequence at least 80%, 85%, 90%, 95%, 96%, 97%, 98%, 99%, or 100% identical to SEQ ID NO: 207, a first light chain comprising an amino acid sequence at least 80%, 85%, 90%, 95%, 96%, 97%, 98%, 99%, or 100% identical to SEQ ID NO: 220, a second heavy chain comprising an amino acid sequence at least 80%, 85%, 90%, 95%, 96%, 97%, 98%, 99%, or 100% identical to SEQ ID NO: 214, and a second light chain comprising an amino acid sequence at least 80%, 85%, 90%, 95%, 96%, 97%, 98%, 99%, or 100% identical to SEQ ID NO: 226;
e. a first heavy chain comprising an amino acid sequence at least 80%, 85%, 90%, 95%, 96%, 97%, 98%, 99%, or 100% identical to SEQ ID NO: 233, a first light chain comprising an amino acid sequence at least 80%, 85%, 90%, 95%, 96%, 97%, 98%, 99%, or 100% identical to SEQ ID NO: 246, a second heavy chain comprising an amino acid sequence at least 80%, 85%, 90%, 95%, 96%, 97%, 98%, 99%, or 100% identical to SEQ ID NO: 240, and a second light chain comprising an amino acid sequence at least 80%, 85%, 90%, 95%, 96%, 97%, 98%, 99%, or 100% identical to SEQ ID NO: 252; and
f. a first heavy chain comprising an amino acid sequence at least 80%, 85%, 90%, 95%, 96%, 97%, 98%, 99%, or 100% identical to SEQ ID NO: 259, a first light chain comprising an amino acid sequence at least 80%, 85%, 90%, 95%, 96%, 97%, 98%, 99%, or 100% identical to SEQ ID NO: 272, a second heavy chain comprising an amino acid sequence at least 80%, 85%, 90%, 95%, 96%, 97%, 98%, 99%, or 100% identical to SEQ ID NO: 266, and a second light chain comprising an amino acid sequence at least 80%, 85%, 90%, 95%, 96%, 97%, 98%, 99%, or 100% identical to SEQ ID NO: 278.

Embodiment 61: The bispecific antibody of any one of embodiments 48-60, wherein the bispecific antibody is conjugated or recombinantly fused to at least one other moiety or molecule selected from the group consisting of a diagnostic agent, a detectable agent, a therapeutic agent, a purification tag, a molecule affecting one or more biological or molecular properties of the antibody or antigen-binding domain, or any combination thereof, wherein the biological or molecular properties comprise serum stability, half-life, solubility, and antigenicity.

Embodiment 62: An isolated nucleic acid comprising at least one polynucleotide sequence encoding the antibody or antigen-binding fragment of any one of embodiments 33-47 or the bispecific antibody of any one of embodiments 48-61.

Embodiment 63: The isolated nucleic acid embodiment 62, wherein the nucleic acid is DNA.

Embodiment 64: A vector comprising the isolated nucleic acid of embodiment 62 or embodiment 63.

Embodiment 65: The vector of embodiment 64, wherein the vector is a cloning vector or an expression vector.

Embodiment 66: A host cell comprising the isolated nucleic acid of embodiment 62 or embodiment 63 and/or the vector of embodiment 64 or embodiment 65.

Embodiment 67: A pharmaceutical composition comprising the monoclonal antibody or antigen-binding fragment of any one of embodiments 33-47 or the bispecific antibody of any one of embodiments 48-61, and at least one pharmaceutical carrier, vehicle or diluent.

Embodiment 68: A method of detecting a severe acute respiratory syndrome coronavirus 2 (SARS-COV-2), wherein the method comprises contacting a sample with the monoclonal antibody or antigen-binding fragment of any one of embodiments 33-47 or the bispecific antibody of any one of embodiments 48-61.

Embodiment 69: A method of diagnosing a severe acute respiratory syndrome coronavirus 2 (SARS-COV-2) infection in a subject, wherein the method comprises contacting a sample obtained from the subject with the monoclonal antibody or antigen-binding fragment of any one of embodiments 33-47 or the bispecific antibody of any one of embodiments 48-61.

Embodiment 70: A method of neutralizing a severe acute respiratory syndrome coronavirus 2 (SARS-COV-2) in a subject, wherein the method comprises administering to the subject the monoclonal antibody or antigen-binding fragment of any one of embodiments 33-47 or the bispecific antibody of any one of embodiments 48-61.

Embodiment 71: A method of treating at least one sign or symptom of a severe acute respiratory syndrome coronavirus 2 (SARS-COV-2) infection in a subject, wherein the method comprises administering to the subject the monoclonal antibody or antigen-binding fragment of any one of embodiments 33-47 or the bispecific antibody of any one of embodiments 48-61.

1. A method of identifying antigen-specific antibodies, the method comprising:
a. vaccinating a subject with an effective amount of a lipid nanoparticle comprising at least one mRNA encoding an antigen;
b boosting the subject with at least one additional vaccination with an effective amount of the lipid nanoparticle comprising at least one mRNA encoding the antigen;
c. isolating B cells from the subject after the vaccination of step a;
d. identifying and cloning BCR heavy and light chain pairs expressed by enriched B cells; and
e. screening the BCR heavy and light chains for antigen-specificity, thereby identifying antigen-specific antibodies.

2. The method of claim 1, wherein isolating B cells from the subject is performed at least about 14 days after the vaccination of step a.

3. The method of claim 1, wherein boosting the subject comprises four additional vaccinations.

4. (canceled)

5. The method of claim 1, wherein identifying BCR heavy and light chain pairs is performed by single-cell BCR sequencing (scBCRseq).

6-11. (canceled)

12. The method of claim 1, wherein the antigen is a severe acute respiratory syndrome coronavirus 2 (SARS-COV-2) antigen.

13. The method of claim 12, wherein the SARS-COV-2 antigen is a SARS-COV-2 spike protein receptor binding domain (RBD).

14. An antibody produced by the method of claim 1.

15. A method for rapid identification of a monoclonal antibody or antigen-binding fragment which is capable of binding a spike protein receptor binding domain (RBD) of a severe acute respiratory syndrome coronavirus 2 (SARS-COV-2), the method comprising:
a. immunizing an animal with a polypeptide comprising the spike protein RBD or with a nucleic acid comprising a nucleotide sequence encoding the spike protein RBD;
b sequencing fully recombined VDJ sequences of B cell receptor (BCR) repertoires from multiple individual B cells of the animal, thereby generating sequencing data;
c. identifying candidate antibody clones from the sequencing data;
d. producing a recombinant monoclonal antibody or antigen-binding fragment comprising paired heavy and light chain variable regions from one or more candidate antibody clones; and
e. testing the produced antibody or antigen-binding fragment for an ability to bind the spike protein RBD.

16. The method of claim 15, wherein the sequencing is high-throughput single-cell B-cell receptor sequencing (scBCR-seq).

17-31. (canceled)

32. An antibody produced by the method of claim 15.

33. A monoclonal antibody or antigen-binding fragment, or a bispecific antibody, wherein the antibody or antigen-binding fragment, or the bispecific antibody is capable of binding a severe acute respiratory syndrome coronavirus 2 (SARS-CoV-2) spike protein receptor binding domain (RBD),
wherein the antibody or antigen-binding fragment comprises three heavy chain complementarity determining regions (HCDR1, HCDR2, and HCDR3) and three light chain complementarity determining regions (LCDR1, LCDR2, and LCDR3) selected from the group consisting of:
a. an HCDR1 comprising SEQ ID NO: 1, an HCDR2 comprising SEQ ID NO: 2, an HCDR3 comprising SEQ ID NO: 3, an LCDR1 comprising SEQ ID NO: 4, an LCDR2 comprising the amino acid sequence AAS, and an LCDR3 comprising SEQ ID NO: 6;
b. an HCDR1 comprising SEQ ID NO: 11, an HCDR2 comprising SEQ ID NO: 12, an HCDR3 comprising SEQ ID NO: 13, an LCDR1 comprising SEQ ID NO: 14, an LCDR2 comprising the amino acid sequence GSS, and an LCDR3 comprising SEQ ID NO: 16;
c. an HCDR1 comprising SEQ ID NO: 21, an HCDR2 comprising SEQ ID NO: 22, an HCDR3 comprising SEQ ID NO: 23, an LCDR1 comprising SEQ ID NO: 24, an LCDR2 comprising the amino acid sequence AAS, and an LCDR3 comprising SEQ ID NO: 26;
d. an HCDR1 comprising SEQ ID NO: 31, an HCDR2 comprising SEQ ID NO: 32, an HCDR3 comprising SEQ ID NO: 33, an LCDR1 comprising SEQ ID NO: 34, an LCDR2 comprising the amino acid sequence AAS, and an LCDR3 comprising SEQ ID NO: 36;
e. an HCDR1 comprising SEQ ID NO: 107, an HCDR2 comprising SEQ ID NO: 108, an HCDR3 comprising SEQ ID NO: 109, an LCDR1 comprising SEQ ID NO: 116, an LCDR2 comprising the amino acid sequence GAS, and an LCDR3 comprising SEQ ID NO: 118;
f. an HCDR1 comprising SEQ ID NO: 110, an HCDR2 comprising SEQ ID NO: 111, an HCDR3 comprising SEQ ID NO: 112, an LCDR1 comprising SEQ ID NO: 119, an LCDR2 comprising the amino acid sequence WAS, and an LCDR3 comprising SEQ ID NO: 121; and
g. an HCDR1 comprising SEQ ID NO: 113, an HCDR2 comprising SEQ ID NO: 114, an HCDR3 comprising SEQ ID NO: 115, an LCDR1 comprising SEQ ID NO: 122, an LCDR2 comprising the amino acid sequence DAS, and an LCDR3 comprising SEQ ID NO: 124; and
wherein the bispecific antibody comprises a first heavy chain comprising a first heavy chain variable region (VH1), a first light chain comprising a first light chain variable region (VL1), a second heavy chain comprising a second heavy chain variable region (VH2), and a second light chain comprising a second light chain variable region (VL2); wherein the VH1 and the VH2 each comprises three heavy chain complementarity determining regions (HCDR1, HCDR2, and HCDR3) and the VL1 and the VL2 each comprises three light chain complementarity determining regions (LCDR1, LCDR2, and LCDR3); wherein the complementarity determining regions are selected from the group consisting of:
h. a VH1 HCDR1 comprising SEQ ID NO: 41, a VH1 HCDR2 comprising SEQ ID NO: 42, a VH1 HCDR3 comprising SEQ ID NO: 43, a VL1 LCDR1 comprising SEQ ID NO: 47, a VL1 LCDR2 comprising the amino acid sequence GSS, a VL1 LCDR3 comprising SEQ ID NO: 49, a VH2 HCDR1 comprising SEQ ID NO: 44, a VH2 HCDR2 comprising SEQ ID NO: 45, a VH2 HCDR3 comprising SEQ ID NO: 46, a VL2 LCDR1 comprising SEQ ID NO: 50, a VL2 LCDR2 comprising the amino acid sequence AAS, and a VL2 LCDR3 comprising SEQ ID NO: 52;
i. a VH1 HCDR1 comprising SEQ ID NO: 151, a VH1 HCDR2 comprising SEQ ID NO: 152, a VH1 HCDR3 comprising SEQ ID NO: 153, a VL1 LCDR1 comprising SEQ ID NO: 165, a VL1 LCDR2 comprising the amino acid sequence GAS, a VL1 LCDR3 comprising SEQ ID NO: 166, a VH2 HCDR1 comprising SEQ ID NO: 158, a VH2 HCDR2 comprising SEQ ID NO: 159, a VH2 HCDR3 comprising SEQ ID NO: 160, a VL2 LCDR1 comprising SEQ ID NO: 171, a VL2 LCDR2 comprising the amino acid sequence WAS, and a VL2 LCDR3 comprising SEQ ID NO: 172;
j. a VH1 HCDR1 comprising SEQ ID NO: 177, a VH1 HCDR2 comprising SEQ ID NO: 178, a VH1 HCDR3 comprising SEQ ID NO: 179, a VL1 LCDR1 comprising SEQ ID NO: 191, a VL1 LCDR2 comprising the amino acid sequence DAS, a VL1 LCDR3 comprising SEQ ID NO: 192, a VH2 HCDR1 comprising SEQ ID NO: 184, a VH2 HCDR2 comprising SEQ ID NO: 185, a VH2 HCDR3 comprising SEQ ID NO: 186, a VL2 LCDR1 comprising SEQ ID NO: 197, a VL2 LCDR2 comprising the amino acid sequence GAS, and a VL2 LCDR3 comprising SEQ ID NO: 198;
k. a VH1 HCDR1 comprising SEQ ID NO: 203, a VH1 HCDR2 comprising SEQ ID NO: 204, a VH1 HCDR3 comprising SEQ ID NO: 205, a VL1 LCDR1 comprising SEQ ID NO: 217, a VL1 LCDR2 comprising the amino acid sequence DAS, a VL1 LCDR3 comprising SEQ ID NO: 218, a VH2 HCDR1 comprising SEQ ID NO: 210, a VH2 HCDR2 comprising SEQ ID NO: 211, a VH2 HCDR3 comprising SEQ ID NO: 212, a VL2 LCDR1 comprising SEQ ID NO: 223, a VL2 LCDR2 comprising the amino acid sequence WAS, and a VL2 LCDR3 comprising SEQ ID NO: 224;
l. a VH1 HCDR1 comprising SEQ ID NO: 229, a VH1 HCDR2 comprising SEQ ID NO: 230, a VH1 HCDR3 comprising SEQ ID NO: 231, a VL1 LCDR1 comprising SEQ ID NO: 243, a VL1 LCDR2 comprising the amino acid sequence AAS, a VL1 LCDR3 comprising SEQ ID NO: 244, a VH2 HCDR1 comprising SEQ ID NO: 236, a VH2 HCDR2 comprising SEQ ID NO: 237, a VH2 HCDR3 comprising SEQ ID NO: 238, a VL2 LCDR1 comprising SEQ ID NO: 249, a VL2 LCDR2 comprising the amino acid sequence GAS, and a VL2 LCDR3 comprising SEQ ID NO: 250; and
m. a VH1 HCDR1 comprising SEQ ID NO: 255, a VH1 HCDR2 comprising SEQ ID NO: 256, a VH1 HCDR3 comprising SEQ ID NO: 257, a VL1 LCDR1 comprising SEQ ID NO: 269, a VL1 LCDR2 comprising the amino acid sequence AAS, a VL1 LCDR3 comprising SEQ ID NO: 270, a VH2 HCDR1 comprising SEQ ID NO: 262, a VH2 HCDR2 comprising SEQ ID NO: 263, a VH2 HCDR3 comprising SEQ ID NO: 264, a VL2 LCDR1 comprising SEQ ID NO: 275, a VL2 LCDR2 comprising the amino acid sequence WAS, and a VL2 LCDR3 comprising SEQ ID NO: 276.

34-61. (canceled)

62. An isolated nucleic acid comprising at least one polynucleotide sequence encoding the antibody or antigen-binding fragment, or the bispecific antibody, of claim 33.

63. (canceled)

64. A vector comprising the isolated nucleic acid of claim 62.

65. (canceled)

66. A host cell comprising the vector of claim 64.

67. A pharmaceutical composition comprising the monoclonal antibody or antigen-binding fragment, or the bispecific antibody, of claim 33, and at least one pharmaceutical carrier, vehicle or diluent.

68. A method of detecting a severe acute respiratory syndrome coronavirus 2 (SARS-COV-2), wherein the method comprises contacting a sample with the monoclonal antibody or antigen-binding fragment, or the bispecific antibody, of claim 33.

69. A method of diagnosing a severe acute respiratory syndrome coronavirus 2 (SARS-COV-2) infection in a subject, wherein the method comprises contacting a sample obtained from the subject with the monoclonal antibody or antigen-binding fragment, or the bispecific antibody, of claim 33.

70. A method of neutralizing a severe acute respiratory syndrome coronavirus 2 (SARS-COV-2) in a subject, wherein the method comprises administering to the subject the monoclonal antibody or antigen-binding fragment, or the bispecific antibody, of claim 33.

71. A method of treating at least one sign or symptom of a severe acute respiratory syndrome coronavirus 2 (SARS-COV-2) infection in a subject, wherein the method comprises administering to the subject the monoclonal antibody or antigen-binding fragment, or the bispecific antibody, of claim 33.


SEQUENCE LISTING (290 sequences)

SEQ ID NO: 1 - protein, 8 aa, Artificial Sequence
  Clone 2 HCDR1
GYSFTGYS

SEQ ID NO: 2 - protein, 8 aa, Artificial Sequence
  Clone 2 HCDR2
INPSTGGT

SEQ ID NO: 3 - protein, 7 aa, Artificial Sequence
  Clone 2 HCDR3
ARSRGDY

SEQ ID NO: 4 - protein, 10 aa, Artificial Sequence
  Clone 2 LCDR1
ESVEYYGTSL

SEQ ID NO: 5 - skipped

SEQ ID NO: 6 - protein, 9 aa, Artificial Sequence
  Clone 2 LCDR3
QQSRKVPWT

SEQ ID NO: 7 - protein, 133 aa, Artificial Sequence
  Clone 2 Heavy Chain Variable Region
MGWNWIFILILSVTTGVHSEVQLQQSGPELVKPGASVKISCKASGYSFTGYSMNWMKQSPEKSLEWIGEINPSTGGTTDNQKFKAKATLTVDKSSSTAYMQLKSLTSEDSAVYYCARSRGDYWGQGTSVTVSS

SEQ ID NO: 8 - protein, 132 aa, Artificial Sequence
  Clone 2 Light Chain Variable Region
MESDTLLLWVLLLWVPGSTGDIVLTQSPASLAVSLGQRATISCRASESVEYYGTSLMQWYQQKPGQPPKVLIYAASNVESGVPARFSGSGSGTDFSLNIHPVEEDDIAMYFCQQSRKVPWTFGGGTKLEIKR

SEQ ID NO: 9 - protein, 463 aa, Artificial Sequence
  Clone 2 Heavy Chain
MGWNWIFILILSVTTGVHSEVQLQQSGPELVKPGASVKISCKASGYSFTGYSMNWMKQSPEKSLEWIGEINPSTGGTTDNQKFKAKATLTVDKSSSTAYMQLKSLTSEDSAVYYCARSRGDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 238 aa, Artificial Sequence
  Clone 2 Light Chain
MESDTLLLWVLLLWVPGSTGDIVLTQSPASLAVSLGQRATISCRASESVEYYGTSLMQWYQQKPGQPPKVLIYAASNVESGVPARFSGSGSGTDFSLNIHPVEEDDIAMYFCQQSRKVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, Artificial Sequence
  Clone 6 HCDR1
GYTFTSYT

SEQ ID NO: 12 - protein, 8 aa, Artificial Sequence
  Clone 6 HCDR2
INPTSGYT

SEQ ID NO: 13 - protein, 11 aa, Artificial Sequence
  Clone 6 HCDR3
AREGHRVGPAY

SEQ ID NO: 14 - protein, 10 aa, Artificial Sequence
  Clone 6 LCDR1
ESVDNYGISF

SEQ ID NO: 15 - skipped

SEQ ID NO: 16 - protein, 9 aa, Artificial Sequence
  Clone 6 LCDR3
QQSKEVPYT

SEQ ID NO: 17 - protein, 137 aa, Artificial Sequence
  Clone 6 Heavy Chain Variable Region
MERHWIFLFLLSVTAGVHSQVQLQQSAAELARPGASVKMSCKASGYTFTSYTMHWVKQRPGQGLEWIGYINPTSGYTEYNQNFKDKTTLTADKSSSTAYMQLNSLTSEDSAVYYCAREGHRVGPAYWGQGTLVTVSA

SEQ ID NO: 18 - protein, 132 aa, Artificial Sequence
  Clone 6 Light Chain Variable Region
MEKDTLLLWVLLLWVPGSTGDIVLTQSPASLAVSLGQRATISCRASESVDNYGISFMNWFQQTPGQPPKLLIYGSSNQGSGVPARFSGSGSGTDFSLNIHPMEEDDTAMYFCQQSKEVPYTFGGGTKLEIKR

SEQ ID NO: 19 - protein, 467 aa, Artificial Sequence
  Clone 6 Heavy Chain
MERHWIFLFLLSVTAGVHSQVQLQQSAAELARPGASVKMSCKASGYTFTSYTMHWVKQRPGQGLEWIGYINPTSGYTEYNQNFKDKTTLTADKSSSTAYMQLNSLTSEDSAVYYCAREGHRVGPAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 238 aa, Artificial Sequence
  Clone 6 Light Chain
MEKDTLLLWVLLLWVPGSTGDIVLTQSPASLAVSLGQRATISCRASESVDNYGISFMNWFQQTPGQPPKLLIYGSSNQGSGVPARFSGSGSGTDFSLNIHPMEEDDTAMYFCQQSKEVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, Artificial Sequence
  Clone 12 HCDR1
GYSFTGYS

SEQ ID NO: 22 - protein, 8 aa, Artificial Sequence
  Clone 12 HCDR2
INPSTGGT

SEQ ID NO: 23 - protein, 7 aa, Artificial Sequence
  Clone 12 HCDR3
ARSRGDY

SEQ ID NO: 24 - protein, 10 aa, Artificial Sequence
  Clone 12 LCDR1
ESVEYYGTSL

SEQ ID NO: 25 - skipped

SEQ ID NO: 26 - protein, 9 aa, Artificial Sequence
  Clone 12 LCDR3
QQSRKVPWT

SEQ ID NO: 27 - protein, 133 aa, Artificial Sequence
  Clone 12 Heavy Chain Variable Region
MGWNWIFILILSVTTGVHSEVQLVQSGAEVKKPGESLKISCKGSGYSFTGYSMNWIRQPPGKGLEWIGEINPSTGGTTDNQKFKARVTITADESTSTAYMELSSLRSEDTAVYYCARSRGDYWGQGTLVTVSS

SEQ ID NO: 28 - protein, 132 aa, Artificial Sequence
  Clone 12 Light Chain Variable Region
MESDTLLLWVLLLWVPGSTGDIVMTQSPDSLAVSLGERATINCRASESVEYYGTSLMQWYQQKPGQPPKLLIYAASNVESGVPSRFSGSGSGTDFTFTISSLQPEDIATYYCQQSRKVPWTFGGGTKVEIKR

SEQ ID NO: 29 - protein, 463 aa, Artificial Sequence
  Clone 12 Heavy Chain
MGWNWIFILILSVTTGVHSEVQLVQSGAEVKKPGESLKISCKGSGYSFTGYSMNWIRQPPGKGLEWIGEINPSTGGTTDNQKFKARVTITADESTSTAYMELSSLRSEDTAVYYCARSRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 238 aa, Artificial Sequence
  Clone 12 Light Chain
MESDTLLLWVLLLWVPGSTGDIVMTQSPDSLAVSLGERATINCRASESVEYYGTSLMQWYQQKPGQPPKLLIYAASNVESGVPSRFSGSGSGTDFTFTISSLQPEDIATYYCQQSRKVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 8 aa, Artificial Sequence
  Clone 13 HCDR1
GYSFTGYS

SEQ ID NO: 32 - protein, 8 aa, Artificial Sequence
  Clone 13 HCDR2
INPSTGGT

SEQ ID NO: 33 - protein, 7 aa, Artificial Sequence
  Clone 13 HCDR3
ARSRGDY

SEQ ID NO: 34 - protein, 10 aa, Artificial Sequence
  Clone 13 LCDR1
ESVEYYGTSL

SEQ ID NO: 35 - skipped

SEQ ID NO: 36 - protein, 9 aa, Artificial Sequence
  Clone 13 LCDR3
QQSRKVPWT

SEQ ID NO: 37 - protein, 133 aa, Artificial Sequence
  Clone 13 Heavy Chain Variable Region
MGWNWIFILILSVTTGVHSEVQLVESGGGLVQPGGSLRLSCAASGFTFSGYSMNWVRQAPGKGLEWVAEINPSTGGTTDNQKFKARFTISRDNSKNTLYLQMNSLRAEDTAVYYCARSRGDYWGQGTLVTVSS

SEQ ID NO: 38 - protein, 132 aa, Artificial Sequence
  Clone 13 Light Chain Variable Region
MESDTLLLWVLLLWVPGSTGDIQMTQSPSSLSASVGDRVTITCRASESVEYYGTSLMQWYQQKPGKAPKLLIYAASNVESGVPSRFSGSGSGTDFTLTISSLQPEDFATYYCQQSRKVPWTFGQGTKVEIKR

SEQ ID NO: 39 - protein, 463 aa, Artificial Sequence
  Clone 13 Heavy Chain
MGWNWIFILILSVTTGVHSEVQLVESGGGLVQPGGSLRLSCAASGFTFSGYSMNWVRQAPGKGLEWVAEINPSTGGTTDNQKFKARFTISRDNSKNTLYLQMNSLRAEDTAVYYCARSRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 238 aa, Artificial Sequence
  Clone 13 Light Chain
MESDTLLLWVLLLWVPGSTGDIQMTQSPSSLSASVGDRVTITCRASESVEYYGTSLMQWYQQKPGKAPKLLIYAASNVESGVPSRFSGSGSGTDFTLTISSLQPEDFATYYCQQSRKVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 8 aa, Artificial Sequence
  Clone 16 H1CDR1
GYTFTSYT

SEQ ID NO: 42 - protein, 8 aa, Artificial Sequence
  Clone 16 H1CDR2
INPTSGYT

SEQ ID NO: 43 - protein, 11 aa, Artificial Sequence
  Clone 16 H1CDR3
AREGHRVGPAY

SEQ ID NO: 44 - protein, 8 aa, Artificial Sequence
  Clone 16 H2CDR1
GYSFTGYS

SEQ ID NO: 45 - protein, 8 aa, Artificial Sequence
  Clone 16 H2CDR2
INPSTGGT

SEQ ID NO: 46 - protein, 7 aa, Artificial Sequence
  Clone 16 H2CDR3
ARSRGDY

SEQ ID NO: 47 - protein, 10 aa, Artificial Sequence
  Clone 16 L1CDR1
ESVDNYGISF

SEQ ID NO: 48 - skipped

SEQ ID NO: 49 - protein, 9 aa, Artificial Sequence
  Clone 16 L1CDR3
QQSKEVPYT

SEQ ID NO: 50 - protein, 10 aa, Artificial Sequence
  Clone 16 L2CDR1
ESVEYYGTSL

SEQ ID NO: 51 - skipped

SEQ ID NO: 52 - protein, 9 aa, Artificial Sequence
  Clone 16 L2CDR3
QQSRKVPWT

SEQ ID NO: 53 - protein, 137 aa, Artificial Sequence; identical to SEQ ID NO: 17
  Clone 16 Heavy Chain (H1) Variable Region

SEQ ID NO: 54 - protein, 133 aa, Artificial Sequence; identical to SEQ ID NO: 7
  Clone 16 Heavy Chain (H2) Variable Region

SEQ ID NO: 55 - protein, 132 aa, Artificial Sequence; identical to SEQ ID NO: 18
  Clone 16 Light Chain (L1) Variable Region

SEQ ID NO: 56 - protein, 132 aa, Artificial Sequence; identical to SEQ ID NO: 8
  Clone 16 Light Chain (L2) Variable Region

SEQ ID NO: 57 - protein, 467 aa, Artificial Sequence
  Clone 16 Heavy Chain (H1)
MERHWIFLFLLSVTAGVHSQVQLQQSAAELARPGASVKMSCKASGYTFTSYTMHWVKQRPGQGLEWIGYINPTSGYTEYNQNFKDKTTLTADKSSSTAYMQLNSLTSEDSAVYYCAREGHRVGPAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 467 aa, Artificial Sequence
  Clone 16 Heavy Chain (H2)
MGWNWIFILILSVTTGVHSEVQLQQSGPELVKPGASVKISCKASGYSFTGYSMNWMKQSPEKSLEWIGEINPSTGGTTDNQKFKAKATLTVDKSSSTAYMQLKSLTSEDSAVYYCARSRGDYWGQGTSVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 238 aa, Artificial Sequence; identical to SEQ ID NO: 20
  Clone 16 Light Chain (L1)

SEQ ID NO: 60 - protein, 237 aa, Artificial Sequence
  Clone 16 Light Chain (L2)
MESDTLLLWVLLLWVPGSTGDIVLTQSPASLAVSLGQRATISCRASESVEYYGTSLMQWYQQKPGQPPKVLIYAASNVESGVPARFSGSGSGTDFSLNIHPVEEDDIAMYFCQQSRKVPWTFGGGTKLEIKRSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - DNA, 991 bp, Artificial Sequence
  pPR23
cgaattcgatatctcgagtgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacg

SEQ ID NO: 62 - DNA, 983 bp, Artificial Sequence
  pPR24
gtcacgaattcgatatctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatgccgggacgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacg

SEQ ID NO: 63 - DNA, 65 bp, Artificial Sequence
  oPR81-F primer
cttgtcacgaattcaccggtcaccatggaaatcaaatcgagtgctagcaccaagggcccatcggt

SEQ ID NO: 64 - DNA, 59 bp, Artificial Sequence
  oPR82-R primer
catcaatgtatcttatcatgtcgagcttgctccctctaacaagatttgggctcaacttt

SEQ ID NO: 65 - DNA, 450 bp, Artificial Sequence
  Clone 1-VH-gBlcok
cactaagtcttgcacttgtcacgaattcgatggaatggcccctgatatttcttttcttgctcagtggaactgccggcgtgcaaagccaggtacaacttcagcaatctggtgcagagctggttagaccaggagcaagtgtgaagatttcctgtaaggcatctgggtacgccttctccacctattggatgaactgggtcaaacaacggcccggtaagggtctggagtggataggccaaaaccatccaggcgatggagaaactaactacaatggaaaatttaaagggaaggccacactgacagccgataaatcatcctcaaccgcatacatgcagctttcctcactcacttcagaggactccgctgtctacttctgcgctcggtcccccgattggggacaagggacactcgtgacagtctctgctgctagcaccaagggcccatcggtcttcc

SEQ ID NO: 66 - DNA, 456 bp, Artificial Sequence
  Clone 2-VH-gBlcok
cactaagtcttgcacttgtcacgaattcgatgggatggaactggattttcatcctcattcttagtgttactacaggcgttcactcagaagtccagcttcaacagagcggcccagagcttgtaaaaccaggcgcttccgtcaaaatttcctgcaaagcaagcggttatagcttcactggatatagtatgaattggatgaaacagtcacctgagaagtcattggaatggataggagagatcaatcctagtacaggaggtacaactgataaccaaaagtttaaagcaaaggctaccttgactgtagacaaatcttcatcaactgcctatatgcaacttaaaagtttgacttcagaagacagtgccgtttactattgcgctaggtcacgcggagattattggggacaggggacctccgtcaccgtgtcttccgctagcaccaagggcccatcggtcttcc

SEQ ID NO: 67 - DNA, 465 bp, Artificial Sequence
  Clone 3-VH-gBlcok
cactaagtcttgcacttgtcacgaattcgatgggttggtcttggatttttctcctgttccttagcggtactgccggcgtcctttctgaagtccagctccaacagtcaggccctgaactggttaagccaggagcatccgtcaaaataccatgtaaggcatccggttacactttcaccgattacaacatggactgggtaaagcagtcccacgggaagagtctggagtggatcggagatataaatccaaacaatggaggcacaatctataatcagaaatttaaagggaaggcaactctgaccgtagacaagtctagctccacagcatatatggagttgcggtctctgacaagcgaggataccgccgtctattactgcgcaaggtcaaggaacgatttcttcgattattggggacaagggactactttgaccgtttcttctgctagcaccaagggcccatcggtcttcc

SEQ ID NO: 68 - DNA, 447 bp, Artificial Sequence
  Clone 4-VH-gBlcok
cactaagtcttgcacttgtcacgaattcgatggacttcggtctcatcttctttatcgtcgcactcctcaagggcgtccaatgcgaggttaagctgctgcaatctggtggaggtcttgtacagcctggcggcagcctgaaactttcttgtgctgcaagtgggatcgacttcagtagatattggatgacctgggttcggagagcacctggaaaagggttggagtggattggcgagataaatcccgatagctccactatcgactatgctccttcattgaaagacaagtttatcatctctcgggacaacgctaagaatactctgtatttgcaaatgtcaaaagtcaggagcgaagatactgctctgtattactgcgctatcggtgcttactggggacaggggacattggtcaccgtatccgccgctagcaccaagggcccatcggtcttcc

SEQ ID NO: 69 - DNA, 465 bp, Artificial Sequence
  Clone 5-VH-gBlcok
cactaagtcttgcacttgtcacgaattcgatggacttcggcctgatcttcttcatcgtggccctgctgaagggcgtgcagtgcgaggtgaagctgctggagagcggcggcggcctggtgcagcccggcggcagcctgaagctgagctgcgccgccagcggcttcgacttcagcaggtactggatgagctgggtgaggcaggcccccggcaagggcctggagtggatcggcgagatcaaccccgacagcagcaccatcaactacacccccagcctgaaggacaagttcatcatcagcagggacaacgccaagaacaccctgtacctgcagatgagcaaggtgaggagcgaggacaccgccctgtactactgcgccaggcagggctacggcaactacttcgactactggggccagggcaccaccctgaccgtgagcagcgctagcaccaagggcccatcggtcttcc

SEQ ID NO: 70 - DNA, 468 bp, Artificial Sequence
  Clone 6-VH-gBlcok
cactaagtcttgcacttgtcacgaattcgatggagcgacattggatctttctcttccttctcagtgtgacagctggggttcactcccaggtacagctccaacagtcagccgcagaactcgctcgccccggtgcctcagttaagatgtcctgtaaagcctcagggtatacctttacatcttatactatgcattgggtgaaacagaggcccggtcaaggactggagtggatcggatacataaatcccacatcaggatacactgagtataatcaaaactttaaggacaaaactactttgaccgccgacaaatcttctagtaccgcctatatgcagcttaatagtttgacatccgaggacagcgccgtctattattgtgctcgggaggggcaccgagtcgggcctgcatattggggccaagggactcttgttactgtgagcgccgctagcaccaagggcccatcggtcttcc

SEQ ID NO: 71 - DNA, 450 bp, Artificial Sequence
  Clone7-VH-gBlcok
cactaagtcttgcacttgtcacgaattcgatgggctggtcttggatctttctgtttctcctgtcaggtaccgctggcgtactctcagaggtccagcttcaacagtccggccctgagcttgtaaagcctggtgccagtgtcaaaataagctgcaagacaagtgggtacacttttactaagtacaccatgcattgggtgaagcagagccacggcaaatctctggaatggataggcggtatcaatcctaacaatggaggtactagctacaaccaaaaattcaaggggaaagccaccttgaccgtcgataaatctagctctactgcttacatggagttgcgctcactgactagtgaggactccgctgtgtactactgtgctaggaccgactattgggggcagggtacaactttgactgtatcaagcgctagcaccaagggcccatcggtcttcc

SEQ ID NO: 72 - DNA, 456 bp, Artificial Sequence
  Clone 8-VH-gBlcok
cactaagtcttgcacttgtcacgaattcgatggcctgggtgtggaccctgctgttcctgatggccgccgcccagagcatccaggcccagatccagctggtgcagagcggccccgagctgaagaagcccggcgagaccgtgaagatcagctgcaaggccagcggctacaccttcaccgactacagcatgcactgggtgaagcaggcccccggcaagggcctgaagtggatgggctggatcaacaccgagaccggcgagcccacctacgccgacgacttcaagggcaggttcgccttcagcctggagaccagcgccagcaccgcctacctgcagatcaacaacctgaagaacgaggacaccgccacctacttctgcgccaggcccggcagggactactggggccagggcaccaccctgaccgtgagcagcgctagcaccaagggcccatcggtcttcc

SEQ ID NO: 73 - DNA, 465 bp, Artificial Sequence
  Clone 9-VH-gBlcok
cactaagtcttgcacttgtcacgaattcgatggagtggacctgggtgttcctgttcctgctgagcgtgaccgccggcgtgcacagccaggtgcaggtgcagcagagcggcgccgagctgatgaagcccggcgccagcgccaagatcagctgcaaggccaccggctacaccttcagcaactactggatcgagtgggtgaagcagaggcccggccacggcctggagtggatcggcgagatcctgcccggcagcggcgacaccacctacaacgagaggttcaagggcaaggccaccttcaccgccgacaccagcagcaacaccgcctacatccagctgagcagcctgaccagcgaggacagcgccgtgtactactgcgccggcggcccctacggcaggttcgcctactggggccagggcaccctggtgaccgtgagcgccgctagcaccaagggcccatcggtcttcc

SEQ ID NO: 74 - DNA, 468 bp, Artificial Sequence
  Clone 10-VH-gBlcok
cactaagtcttgcacttgtcacgaattcgatgggttggatctggatcttcctcttcttgctttcaggaactgctggggtacactctgaggttcagttgcagcagagtgggcctgaacttgttaaaaccggggcatctgtaaagatcagttgtactgcttcaggatactccttctcagggttcaacatgcactgggtgaagcaaagccatggaaaaagtctcgagtggattggttatagcagttgttacaatggagctactagttataatcagaagttcaaggggaaggccacttttactgtggacacttccagcagcaccgcctatatgcaatttaattcacttacatccgaggattctgccgtctactattgcgcccggagttatggtaatagtgccatggattattggggtcaaggaacatcagtaaccgtttcaagcgctagcaccaagggcccatcggtcttcc

SEQ ID NO: 75 - DNA, 528 bp, Artificial Sequence
  Clone 11-VH-gBlcok
cactaagtcttgcacttgtcacgaattcgcactaagtcttgcacttgtcacgaattcgatgaacttcggcctgagcctgatcttcctggtgctggtgctgaagggcgtgcagtgcgaggtgatgctggtggagagcggcggcggcctggtgaagcccggcggcagcctgaagctgagctgcgccgccagcggcttcaccttcagcagctacgccatgagctgggtgaggcagacccccgagaagaggctggagtgggtggccaccatcagcagcggcagcagctacacctacttccccgacaccatcatgggcaggttcaccatcagcagggacaacgccaagaacaccctgtacctgcagatgagcagcctgaggagcgaggacaccgccatgtactactgcgccaggaggctggacgacgacggctacttcgactactggggccagggcaccaccctgaccgtgagcagcgctagcaccaagggcccatcggtcttccgctagcaccaagggcccatcggtcttcc

SEQ ID NO: 76 - DNA, 456 bp, Artificial Sequence
  Clone 12-VH-gBlcok
cactaagtcttgcacttgtcacgaattcgatgggctggaattggatattcattctcatccttagcgtcacaacaggggttcatagcgaagtccaacttgttcagtcaggtgcagaggtgaaaaaaccaggcgagtctcttaagatttcctgtaagggatctggatatagcttcacggggtacagtatgaattggataagacagccaccgggtaaaggactggagtggattggagaaataaatccttccactggtggcactacagacaaccaaaaatttaaggcccgagtcacgataacggctgacgaatcaacaagtacggcctacatggaacttagcagccttcgctctgaagataccgccgtttactactgcgccagaagtagaggggactattggggccagggcactttggtcacggtgtcctccgctagcaccaagggcccatcggtcttcc

SEQ ID NO: 77 - DNA, 456 bp, Artificial Sequence
  Clone 13-VH-gBlcok
cactaagtcttgcacttgtcacgaattcgatgggctggaactggatattcatccttatcctttcagtcactactggagttcactccgaggttcagcttgtagaatcagggggtgggctggtccagcctgggggatctttgcgactgagttgtgctgcttcaggtttcacatttagtggctacagcatgaactgggttcggcaagcgccaggcaaagggcttgaatgggtggcggagatcaatccatccacaggaggcacgaccgataaccagaaatttaaggcgaggtttaccataagtagagataactctaaaaacacgctctatctccaaatgaacagtctgcgggcggaggatactgcagtctactattgcgcccgcagccgaggggactattgggggcagggcactttggttacagtgtccagcgctagcaccaagggcccatcggtcttcc

SEQ ID NO: 78 - DNA, 446 bp, Artificial Sequence
  Clone 1-VK-gBlcok
cactaagtcttgcacttgtcacgaattcaatggatatgcggacacccgctcaatttctcgggattctcctcctttggtttcctggcatcaaatgcgatattaagatgacacaaagtccttcttctatgtacgccagcctcggcgaacgggtcaccataacttgtaaggcatcacaggacataaactcataccttagttggttccaacagaagccaggcaaaagtcctaagactctgatatatcgcgctaatcgattggtggatggggtgccctctcgcttttccggcagtggctccgggcaagactactctctcacaatcagcagcttggagtatgaagacatgggaatatactactgcctccagtatgatgaatttccttggacatttggaggcgggactaaattggagataaagcgtacggtggctgcaccatctgtcttcatc

SEQ ID NO: 79 - DNA, 452 bp, Artificial Sequence
  Clone 2-VK-gBlcok
cactaagtcttgcacttgtcacgaattcaatggaatcagatacactcctcctctgggtgcttcttttgtgggtgcctgggtccaccggggacattgtccttacccagtcacccgcctccttggcagtcagtctgggacaaagagccactataagttgtcgggcctctgaatcagttgaatactacggcacctctcttatgcaatggtatcagcaaaagcccggacagcctcctaaggtactcatttacgccgcaagtaatgtagaaagtggagtgcctgccaggttctctggcagcggaagcggtacagatttctcactcaacatccatcctgttgaagaagatgacattgccatgtatttctgccagcaaagcaggaaagtgccttggactttcggtggtgggacaaagctcgaaataaagcgtacggtggctgcaccatctgtcttcatc

SEQ ID NO: 80 - DNA, 437 bp, Artificial Sequence
  Clone 3-VK-gBlcok
cactaagtcttgcacttgtcacgaattcaatgtctgtccccacacaagttctcggcctgctgctcctttggctcacagatgcccggtgtgacatacagatgacacaatcccccgccagtctctcagcttccgtaggggagaccgtaaccattacctgccgagcatccgagaatatttactcaaacttggcatggtatcagcagaagcaaggaaaaagcccccaactcctggtttacgttgccacaaatctcgcccacggggtcccctcaaggttcagcggtagtggctccgggacccaatacagcctgaaaataaatagccttcagtctgaggatttcgggtcctactattgccaacatttctgggggaccccaacctttggaagtgggaccaaactcgaaatcaagcgtacggtggctgcaccatctgtcttcatc

SEQ ID NO: 81 - DNA, 449 bp, Artificial Sequence
  Clone 4-VK-gBlcok
cactaagtcttgcacttgtcacgaattcaatgatgtcaccagctcagttcctcttcctgttggtgctctggattcgagaaactaacggagatgtcgtgatgacccaaaccccacttactctctccgtgactatcggccaacccgctagtatatcatgtaagtcatctcaaagtttgttggattccgacggcaagacatatcttaattggctgctgcaacgcccaggacagtcccctaagagactgatctatctcgtgagtaagctcgatagcggggtccctgaccgattcactggtagtggaagcggtactgattttaccctgaagatttctcgggtcgaggctgaggacctcggtgtatattattgttggcagggaactcacttcccatacaccttcggaggcggtacaaagctggaaataaaacgtacggtggctgcaccatctgtc

SEQ ID NO: 82 - DNA, 440 bp, Artificial Sequence
  Clone 5-VK-gBlcok
cactaagtcttgcacttgtcacgaattcaatggtgttcaccccccacatcctgggcctgctgctgttctggatcagcgccagcaccggcgacatcctgctgacccagagccccgccaccctgagcgtgacccccggcgagaccgtgagcctgagctgcagggccagccagagcatctacaagaacctgcactggtaccagcagaagagccacaggagccccaggctgctgatcaagtacgccagcgacagcatcagcggcatccccagcaggttcaccggcagcggcagcggcaccgactacaccctgagcatcaacagcgtgaagcccgaggacgagggcatctactactgcctgcagggctacagcacccccctgaccttcggcgccggcaccaagctggagctgaagaggacggtggctgcaccatctgtcttcatc

SEQ ID NO: 83 - DNA, 452 bp, Artificial Sequence
  Clone 6-VK-gBlcok
cactaagtcttgcacttgtcacgaattcaatggagaaagataccctcttgctctgggtactgctgctctgggtgcctgggtccaccggcgacattgtgctgacacaatcacctgcctccctggcagtaagtctgggccaacgggcaaccatctcttgccgggctagcgaaagtgtagataattacgggatctctttcatgaattggtttcaacaaacacctgggcagcctcccaaactgctcatctacggctcaagtaaccaaggtagtggtgtgcctgctaggttttcaggttccgggagtggcacagacttcagtctgaacatacacccaatggaagaagacgatactgctatgtatttctgtcaacagagtaaggaggtaccatatacattcggaggcggaaccaaactcgaaatcaagaggacggtggctgcaccatctgtcttcatc

SEQ ID NO: 84 - DNA, 455 bp, Artificial Sequence
  Clone 7-VK-gBlcok
cactaagtcttgcacttgtcacgaattcaatgatgagccccgcccagttcctgttcctgctggtgctgtggatcagggagaccaacggcgacgtggtgatgacccagacccccctgaccctgagcgtgaccatcggccagcccgccagcatcagctgcaagagcagccagagcctgctggacagcgacggcaagacctacctgaactggctgctgcagaggcccggccagagccccaagaggctgatctacctggtgagcaagctggacagcggcgtgcccgacaggttcaccggcagcggcagcggcaccgacttcaccctgaagatcagcagggtggaggccgaggacctgggcgtgtactactgctggcagggcacccacttcccctacaccttcggcggcggcaccaagctggagatcaagaggacggtggctgcaccatctgtcttcatc

SEQ ID NO: 85 - DNA, 452 bp, Artificial Sequence
  Clone 8-VK-gBlcok
cactaagtcttgcacttgtcacgaattcaatgaagctgcccgtgaggctgctggtgctgatgttctggatccccgccagcagcagcgacgtgctgatgacccagacccccctgagcctgcccgtgagcctgggcgaccaggccagcatcagctgcaggagcagccagagcatcgtgcacagcaacggcaacacctacctggactggtacctgcagaagcccggccagagccccaagctgctgatctacaaggtgagcaacaggttcagcggcgtgcccgacaggttcagcggcagcggcagcggcaccgacttcaccctgaagatcagcagggtggaggccgaggacctgggcgtgtactactgcttccagggcagccacgtgccctggaccttcggcggcggcaccaagctggagatcaagaggacggtggctgcaccatctgtcttcatc

SEQ ID NO: 86 - DNA, 440 bp, Artificial Sequence
  Clone 9-VK-gBlcok
cactaagtcttgcacttgtcacgaattcaatgatgagcagcgcccagttcctgggcctgctgctgctgtgcttccagggcaccaggtgcgacctgcagatgacccagaccaccagcagcctgagcgccagcctgggcgacagggtgaccatcaactgcagggccagccaggacatcaccaactacctgaactggtaccagcagaagcccgacggcaccctgaagctgctgatctactacaccagcaggctgcccagcggcgtgcccagcaggttcagcggcagcggcagcggcaccgactacagcctgaccatcagcaacctggagcaggaggacatcgccacctacttctgccagcagggcaagaccttcccctggaccttcggcggcggcaccaagctggagatcaagaggacggtggctgcaccatctgtcttcatc

SEQ ID NO: 87 - DNA, 440 bp, Artificial Sequence
  Clone 10-VK-gBlcok
cactaagtcttgcacttgtcacgaattcaatggagagccagatccaggccttcgtgttcgtgttcctgtggctgagcggcgtggacggcgacatcgtgatgacccagagccacaagttcatgagcaccagcgtgggcgacagggtgagcatcacctgcaaggccagccaggacgtgagcagcgccgtggcctggtaccagcagaagcccggccagagccccaaggtgctgatctactgggccagcaccaggcacaccggcgtgcccgacaggttcaccggcagcggcagcggcaccgactacaccctgaccatcaggagcgtgcaggccgaggacctggccctgtactactgccagcagcactacaacacccccaggaccttcggcggcggcaccaagctggagatcaagaggacggtggctgcaccatctgtcttcatc

SEQ ID NO: 88 - DNA, 437 bp, Artificial Sequence
  Clone 11-VK-gBlcok
cactaagtcttgcacttgtcacgaattcaatgcgcccatctatacagtttttgggacttcttctgttctggcttcatggagcacaatgcgatatacaaatgacccagtccccctcctccctcagcgccagtttgggcggcaaagtcactgttacttgcaaggcctctcaggacataaacaagtatatcgcttggtatcaacacaagcccggtaagggaccacggcttctgattcactatacatcaacacttcaaccagggattccctcaagattctccggcagcggttcagggagggattactcattcagtatcaacaatctggaacctgaggatatagccacatactactgcttgcaatacgacaatttgtacacattcggaggtgggactaagctggagatgaaacgcacggtggctgcaccatctgtcttcatc

SEQ ID NO: 89 - DNA, 452 bp, Artificial Sequence
  Clone 12-VK-gBlcok
cactaagtcttgcacttgtcacgaattcaatggaatctgatacattgcttctttgggtgctcttgctgtgggttccaggctccactggagatattgttatgacccaaagccctgatagcctcgccgtctccctgggcgagcgggcgacgattaactgcagggcatcagagagtgttgaatactatggtacaagtctcatgcagtggtatcagcaaaagcccggtcagcctccgaaattgttgatatacgcagcgtccaacgtggaatccggggtgccatcacggttctccggttccggtagtggtacggacttcaccttcaccatttcctctctgcagcccgaggatattgccacatattactgtcaacaaagtcggaaagttccatggaccttcgggggtgggactaaagtggagataaagcgaacggtggctgcaccatctgtcttcatc

SEQ ID NO: 90 - DNA, 452 bp, Artificial Sequence
  Clone 13-VK-gBlcok
cactaagtcttgcacttgtcacgaattcaatggagtccgatacccttctcctgtgggtgctgttgctttgggtcccaggcagtacgggagatattcagatgacacaatccccctctagcctgtcagcgtccgtgggcgaccgagttacgattacctgccgagcatcagaatccgtagagtattatggcacgtctcttatgcaatggtatcagcagaaaccgggtaaagcgccaaaactgctcatctacgcggcgtctaacgtggagtcaggtgtaccctccagattttctggttcaggaagcgggactgattttacattgactataagctcactgcaaccggaagatttcgctacctactattgtcaacaatcacggaaggtcccatggaccttcgggcagggtactaaagtcgagattaaaaggacggtggctgcaccatctgtcttcatc

SEQ ID NO: 91 - DNA, 998 bp, Artificial Sequence
  UK variant gBlock1
actatagggagacccaagctggctagccaccatgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatctctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagca

SEQ ID NO: 92 - DNA, 998 bp, Artificial Sequence
  UK variant gBlock2
gggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaacctacggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgacgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgatt

SEQ ID NO: 93 - DNA, 963 bp, Artificial Sequence
  UK variant gBlock3
ccagggctggctgtctgattggagcagagcatgtgaacaactcctatgagtgtgacatcccaattggagcaggcatctgtgcctcctaccagacccagaccaacagccataggagggcaaggtctgtggcaagccagagcatcattgcctacacaatgagtctgggagcagagaactctgtggcttacagcaacaacagcattgccatcccaattaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaac

SEQ ID NO: 94 - DNA, 998 bp, Artificial Sequence
  UK variant gBlock4
aacaccctggtgaagcaactttccagcaactttggagccatctcctctgtgctgaatgacatcctggctagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacacataacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctgaggatccgagctcggtaccaagcttaagtttaaaccgctgatcagcctcgactg

SEQ ID NO: 95 - DNA, 993 bp, Artificial Sequence
  South African variant-gBlock1
cactatagggagacccaagctggctagccaccatgtttgtgttcctggtgctgctgccactggtgtccagccagtgtgtgaacttcaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgccaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgaggggcctgccacagggcttctctgccttggaaccactggtggacctgccaattggcatcaacatcaccaggttccagaccctgctggctctgcacatttcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtcc

SEQ ID NO: 96 - DNA, 1299 bp, Artificial Sequence
  South African variant-gBlock2
ccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaacattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtgaagggcttcaactgttactttccactccaatcctatggcttccaaccaacctacggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagacactggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagtggagaactctgtggcttacagcaacaacagcattgccatcccaaccaacttcaccatctctgtgaccacagagattctgcctgtgagtatgaccaagacctctgtggactgtacaatgtatatctgtggagacagcacag

SEQ ID NO: 97 - DNA, 53 bp, Artificial Sequence
  del19R1 primer
cttaagcttggtaccgagctcggatcctcaacaacaggagccacaggaacaac

SEQ ID NO: 98 - DNA, 23 bp, Artificial Sequence
  del19F1 primer
gctagactggacaaggtggaggc

SEQ ID NO: 99-100 - skipped (2 entries)

SEQ ID NO: 101 - protein, 118 aa, Artificial Sequence
  MB_VH2
EVQLVQSGAEVKKPGESLKISCKGSGFSFTSYWIGWVRQMPGKGLEWMGIIYPGDSDTRYSPSFQGQVTISADKSISTAYLQWSSLKASDTAMYYCARGGYEDVFDIWGQGTMVTVSS

SEQ ID NO: 102 - protein, 118 aa, Artificial Sequence
  MB_VH8
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGLTGGDAFDIWGQGTMVTVSS

SEQ ID NO: 103 - protein, 118 aa, Artificial Sequence
  PB_VH3
EVQLVESGGGLVQPGGSLRLSCAASGFTFSSYAMSWVRQAPGKGLEWVSVISGSGGSTSYADSVKGRFTISRDNSKNTLYLQMNSLRAEDTAVYYCAKDKAVEAFDIWGQGTMVTVSS

SEQ ID NO: 104 - protein, 109 aa, Artificial Sequence
  MB_VK2
EIVLTQSPGTLSLSPGGRATLSCRASQSVSSSYLAWYQQKPGQAPRLLIYGASSRATGIPDRFSGSGSGTDFTLTISRLEPEDFAVYYCQQYGSSPPTFGQGTKVEIKR

SEQ ID NO: 105 - protein, 114 aa, Artificial Sequence
  MB_VK8
DIVMTQSPDSLAVSLGERATINCKSSQSVLYSSNNENYLAWYQQKPGQPPKLLIYWASTRESGVPDRFSGSGSGTDFTLTISSLQAEDVAVYYCQQYYSTPLTFGGGTKVEIKR

SEQ ID NO: 106 - protein, 108 aa, Artificial Sequence
  PB_VK3
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIKR

SEQ ID NO: 107 - protein, 8 aa, Artificial Sequence
  MB_VH2 CDR1
GFSFTSYW

SEQ ID NO: 108 - protein, 8 aa, Artificial Sequence
  MB_VH2 CDR2
IYPGDSDT

SEQ ID NO: 109 - protein, 13 aa, Artificial Sequence
  MB_VH2 CDR3
CARGGYEDVFDIW

SEQ ID NO: 110 - protein, 8 aa, Artificial Sequence
  MB_VH8 CDR1
GGSISSYY

SEQ ID NO: 111 - protein, 7 aa, Artificial Sequence
  MB_VH8 CDR2
IYYSGST

SEQ ID NO: 112 - protein, 14 aa, Artificial Sequence
  MB_VH8 CDR3
CARGLTGGDAFDIW

SEQ ID NO: 113 - protein, 8 aa, Artificial Sequence
  PB_VH3 CDR1
GFTFSSYA

SEQ ID NO: 114 - protein, 8 aa, Artificial Sequence
  PB_VH3 CDR2
ISGSGGST

SEQ ID NO: 115 - protein, 13 aa, Artificial Sequence
  PB_VH3 CDR3
CAKDKAVEAFDIW

SEQ ID NO: 116 - protein, 7 aa, Artificial Sequence
  MB_VK2 CDR1
QSVSSSY

SEQ ID NO: 117 - skipped

SEQ ID NO: 118 - protein, 11 aa, Artificial Sequence [sequence omitted]

SEQ ID NO: 119 - protein, 12 aa, Artificial Sequence
  MB_VK8 CDR1
QSVLYSSNNENY

SEQ ID NO: 120 - skipped

SEQ ID NO: 121 - protein, 11 aa, Artificial Sequence [sequence omitted]

SEQ ID NO: 122 - protein, 6 aa, Artificial Sequence
  PB_VK3 CDR1
QDISNY

SEQ ID NO: 123 - skipped

SEQ ID NO: 124 - protein, 11 aa, Artificial Sequence [sequence omitted]

SEQ ID NO: 125 - DNA, 354 bp, Artificial Sequence
  MB_VH2
gaagttcaacttgttcaaagcggcgcagaggtaaagaaacctggagagagtctcaaaatcagttgcaagggatcaggattctcatttacttcctactggataggttgggtccgacaaatgcctgggaagggtctcgaatggatggggatcatatatcctggagattcagacacaagatactccccatctttccaggggcaggttactattagtgcggacaaaagtatctcaacagcgtatctccagtggtccagtctcaaggcgagcgatactgcgatgtattattgtgcgaggggtggttatgaggacgtgttcgatatttgggggcagggcacgatggtcacggtatcttcc

SEQ ID NO: 126 - DNA, 354 bp, Artificial Sequence
  MB_VH8
caggtccagctgcaagagtccgggccgggcctcgtaaaaccctctgagacactgtccctgacttgcacggtgtctggtggttctatctcatcctactattggtcctggattagacagcccccagggaagggccttgagtggatcgggtacatatattattcagggagcactaactacaatccttcactcaagtcacgagtgactatatccgttgacacgagcaaaaaccagttttccctcaaattgagtagcgttacagcagcggatacagcagtctattactgtgcaagaggtctcacgggaggggacgccttcgatatatggggccagggtacgatggtgaccgttagttcc

SEQ ID NO: 127 - DNA, 354 bp, Artificial Sequence
  PB_VH3
gaggttcagcttgtagagtccggtggcggtctcgttcaacccggcgggagcctgaggctgtcttgcgcggcttcaggattcacatttagttcctacgccatgtcctgggtcagacaagcaccaggaaaagggttggagtgggtgtcagttatctcaggttccggaggctcaaccagttacgccgacagcgtcaaaggtcggttcacgatctcccgagacaattcaaagaataccctttatctccagatgaactctttgagggcggaggacaccgccgtgtactattgtgctaaggacaaagcagtagaggctttcgacatttgggggcagggaactatggtcaccgtgtccagt

SEQ ID NO: 128 - DNA, 327 bp, Artificial Sequence
  MB_VK2
gaaatagtattgacgcagtccccagggactctgtcccttagtcccggtggtcgcgctacgctgagttgccgagcctcccaatcagtgtcaagttcttatcttgcttggtatcagcaaaagcctggtcaggccccaaggcttctcatctatggcgcttcatcaagagcgactggtattccagaccgctttagtggttcaggaagtggaaccgactttacgctcactatctcaagactcgaacctgaagactttgcagtttattactgtcagcagtatgggagcagcccacctacattcggtcaaggcacgaaggtcgagattaagaga

SEQ ID NO: 129 - DNA, 342 bp, Artificial Sequence
  MB_VK8
gacattgtaatgacacagagccccgactccctggccgtgtctttgggcgagagagcaaccattaattgtaagtctagccaaagtgtcctgtattcatctaacaacgagaattatttggcctggtatcaacagaagcctggacagccaccgaaacttcttatctattgggcgtcaaccagagagagcggtgtgccagaccggtttagcggatctggttctggaactgattttactttgaccatatctagcctccaggcggaagacgtagcggtctattactgccaacagtattattccaccccgttgaccttcggaggcggaacaaaggttgaaatcaagaga

SEQ ID NO: 130 - DNA, 324 bp, Artificial Sequence
  PB_VK3
gacattcagatgacccaatccccgtcaagtctttctgcatctgttggagaccgagtcacaatcacatgtcaagcaagtcaagatataagcaactaccttaattggtatcagcagaaaccggggaaggcccccaagctgttgatttatgatgcgtctaatcttgagacgggagtcccatcccgattcagcgggtctgggtctggcacggatttcacatttacaataagctcccttcaaccagaggacattgccacatattactgtcagcaatacgataatttgccgctgacgtttggaggtgggactaaggttgagattaaacgg

SEQ ID NO: 131 - protein, 330 aa, Artificial Sequence
  hIgG1 Constant Heavy
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 106 aa, Artificial Sequence
  hIgGkappa Constant
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 330 aa, Artificial Sequence; identical to SEQ ID NO: 131
  MB.02 Heavy

SEQ ID NO: 134 - protein, 106 aa, Artificial Sequence; identical to SEQ ID NO: 132
  MB.02 Light

SEQ ID NO: 135 - protein, 330 aa, Artificial Sequence; identical to SEQ ID NO: 131
  MB.08 Heavy

SEQ ID NO: 136 - protein, 106 aa, Artificial Sequence; identical to SEQ ID NO: 132
  MB.08 Light

SEQ ID NO: 137 - protein, 330 aa, Artificial Sequence; identical to SEQ ID NO: 131
  PB.03 Heavy

SEQ ID NO: 138 - protein, 108 aa, Artificial Sequence; identical to SEQ ID NO: 106
  PB.03 Light

SEQ ID NO: 139 - DNA, 1347 bp, Artificial Sequence
  MB.02 Heavy
gaagttcaacttgttcaaagcggcgcagaggtaaagaaacctggagagagtctcaaaatcagttgcaagggatcaggattctcatttacttcctactggataggttgggtccgacaaatgcctgggaagggtctcgaatggatggggatcatatatcctggagattcagacacaagatactccccatctttccaggggcaggttactattagtgcggacaaaagtatctcaacagcgtatctccagtggtccagtctcaaggcgagcgatactgcgatgtattattgtgcgaggggtggttatgaggacgtgttcgatatttgggggcagggcacgatggtcacggtatcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 140 - DNA, 648 bp, Artificial Sequence
  MB.02 Light
gaaatagtattgacgcagtccccagggactctgtcccttagtcccggtggtcgcgctacgctgagttgccgagcctcccaatcagtgtcaagttcttatcttgcttggtatcagcaaaagcctggtcaggccccaaggcttctcatctatggcgcttcatcaagagcgactggtattccagaccgctttagtggttcaggaagtggaaccgactttacgctcactatctcaagactcgaacctgaagactttgcagtttattactgtcagcagtatgggagcagcccacctacattcggtcaaggcacgaaggtcgagattaagagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 141 - DNA, 1347 bp, Artificial Sequence
  MB.08 Heavy
caggtccagctgcaagagtccgggccgggcctcgtaaaaccctctgagacactgtccctgacttgcacggtgtctggtggttctatctcatcctactattggtcctggattagacagcccccagggaagggccttgagtggatcgggtacatatattattcagggagcactaactacaatccttcactcaagtcacgagtgactatatccgttgacacgagcaaaaaccagttttccctcaaattgagtagcgttacagcagcggatacagcagtctattactgtgcaagaggtctcacgggaggggacgccttcgatatatggggccagggtacgatggtgaccgttagttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 142 - DNA, 663 bp, Artificial Sequence
  MB.08 Light
gacattgtaatgacacagagccccgactccctggccgtgtctttgggcgagagagcaaccattaattgtaagtctagccaaagtgtcctgtattcatctaacaacgagaattatttggcctggtatcaacagaagcctggacagccaccgaaacttcttatctattgggcgtcaaccagagagagcggtgtgccagaccggtttagcggatctggttctggaactgattttactttgaccatatctagcctccaggcggaagacgtagcggtctattactgccaacagtattattccaccccgttgaccttcggaggcggaacaaaggttgaaatcaagagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 143 - DNA, 1347 bp, Artificial Sequence
  PB.03 Heavy
gaggttcagcttgtagagtccggtggcggtctcgttcaacccggcgggagcctgaggctgtcttgcgcggcttcaggattcacatttagttcctacgccatgtcctgggtcagacaagcaccaggaaaagggttggagtgggtgtcagttatctcaggttccggaggctcaaccagttacgccgacagcgtcaaaggtcggttcacgatctcccgagacaattcaaagaataccctttatctccagatgaactctttgagggcggaggacaccgccgtgtactattgtgctaaggacaaagcagtagaggctttcgacatttgggggcagggaactatggtcaccgtgtccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 144 - DNA, 645 bp, Artificial Sequence
  PB.03 Light
gacattcagatgacccaatccccgtcaagtctttctgcatctgttggagaccgagtcacaatcacatgtcaagcaagtcaagatataagcaactaccttaattggtatcagcagaaaccggggaaggcccccaagctgttgatttatgatgcgtctaatcttgagacgggagtcccatcccgattcagcgggtctgggtctggcacggatttcacatttacaataagctcccttcaaccagaggacattgccacatattactgtcagcaatacgataatttgccgctgacgtttggaggtgggactaaggttgagattaaacggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 145 - DNA, 4836 bp, Artificial Sequence
  MB_VH2 Heavy Chain Vector Sequence
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaagttcaacttgttcaaagcggcgcagaggtaaagaaacctggagagagtctcaaaatcagttgcaagggatcaggattctcatttacttcctactggataggttgggtccgacaaatgcctgggaagggtctcgaatggatggggatcatatatcctggagattcagacacaagatactccccatctttccaggggcaggttactattagtgcggacaaaagtatctcaacagcgtatctccagtggtccagtctcaaggcgagcgatactgcgatgtattattgtgcgaggggtggttatgaggacgtgttcgatatttgggggcagggcacgatggtcacggtatcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 146 - DNA, 4178 bp, Artificial Sequence
  MB_VK2 Light Chain Vector Sequence
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagaaatagtattgacgcagtccccagggactctgtcccttagtcccggtggtcgcgctacgctgagttgccgagcctcccaatcagtgtcaagttcttatcttgcttggtatcagcaaaagcctggtcaggccccaaggcttctcatctatggcgcttcatcaagagcgactggtattccagaccgctttagtggttcaggaagtggaaccgactttacgctcactatctcaagactcgaacctgaagactttgcagtttattactgtcagcagtatgggagcagcccacctacattcggtcaaggcacgaaggtcgagattaagagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 147 - DNA, 4836 bp, Artificial Sequence
  MB_MH8 Heavy Chain Vector Sequence
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgcaggtccagctgcaagagtccgggccgggcctcgtaaaaccctctgagacactgtccctgacttgcacggtgtctggtggttctatctcatcctactattggtcctggattagacagcccccagggaagggccttgagtggatcgggtacatatattattcagggagcactaactacaatccttcactcaagtcacgagtgactatatccgttgacacgagcaaaaaccagttttccctcaaattgagtagcgttacagcagcggatacagcagtctattactgtgcaagaggtctcacgggaggggacgccttcgatatatggggccagggtacgatggtgaccgttagttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 148 - DNA, 4193 bp, Artificial Sequence
  MB_VK8 Light Chain Vector Sequence
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagacattgtaatgacacagagccccgactccctggccgtgtctttgggcgagagagcaaccattaattgtaagtctagccaaagtgtcctgtattcatctaacaacgagaattatttggcctggtatcaacagaagcctggacagccaccgaaacttcttatctattgggcgtcaaccagagagagcggtgtgccagaccggtttagcggatctggttctggaactgattttactttgaccatatctagcctccaggcggaagacgtagcggtctattactgccaacagtattattccaccccgttgaccttcggaggcggaacaaaggttgaaatcaagagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 149 - DNA, 4836 bp, Artificial Sequence
  PB_VH3 Heavy Chain Vector Sequence
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggttcagcttgtagagtccggtggcggtctcgttcaacccggcgggagcctgaggctgtcttgcgcggcttcaggattcacatttagttcctacgccatgtcctgggtcagacaagcaccaggaaaagggttggagtgggtgtcagttatctcaggttccggaggctcaaccagttacgccgacagcgtcaaaggtcggttcacgatctcccgagacaattcaaagaataccctttatctccagatgaactctttgagggcggaggacaccgccgtgtactattgtgctaaggacaaagcagtagaggctttcgacatttgggggcagggaactatggtcaccgtgtccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 150 - DNA, 4175 bp, Artificial Sequence
  PB_VK3 Light Chain Vector Sequence
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagacattcagatgacccaatccccgtcaagtctttctgcatctgttggagaccgagtcacaatcacatgtcaagcaagtcaagatataagcaactaccttaattggtatcagcagaaaccggggaaggcccccaagctgttgatttatgatgcgtctaatcttgagacgggagtcccatcccgattcagcgggtctgggtctggcacggatttcacatttacaataagctcccttcaaccagaggacattgccacatattactgtcagcaatacgataatttgccgctgacgtttggaggtgggactaaggttgagattaaacggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 151 - protein, 8 aa, Artificial Sequence
  COV2-0208 IGG1-KNOB-1 HCDR1
GGSISSYY

SEQ ID NO: 152 - protein, 7 aa, Artificial Sequence
  COV2-0208 IGG1-KNOB-1 HCDR2
IYYSGST

SEQ ID NO: 153 - protein, 12 aa, Artificial Sequence
  COV2-0208 IGG1-KNOB-1 HCDR3
ARGLTGGDAFDI

SEQ ID NO: 154 - protein, 118 aa, Artificial Sequence; identical to SEQ ID NO: 102
  COV2-0208 IGG1-KNOB-1 VH

SEQ ID NO: 155 - protein, 448 aa, Artificial Sequence
  COV2-0208 IGG1-KNOB-1 HEAVY CHAIN
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGLTGGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - DNA, 354 bp, Artificial Sequence; identical to SEQ ID NO: 126
  COV2-0208 IGG1-KNOB-1 VH

SEQ ID NO: 157 - DNA, 1347 bp, Artificial Sequence
  COV2-0208 IGG1-KNOB-1 HEAVY CHAIN
caggtccagctgcaagagtccgggccgggcctcgtaaaaccctctgagacactgtccctgacttgcacggtgtctggtggttctatctcatcctactattggtcctggattagacagcccccagggaagggccttgagtggatcgggtacatatattattcagggagcactaactacaatccttcactcaagtcacgagtgactatatccgttgacacgagcaaaaaccagttttccctcaaattgagtagcgttacagcagcggatacagcagtctattactgtgcaagaggtctcacgggaggggacgccttcgatatatggggccagggtacgatggtgaccgttagttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatgccgggacgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 158 - protein, 8 aa, Artificial Sequence
  COV2-0208 IGG1-HOLE-2 HCDR1
GFSFTSYW

SEQ ID NO: 159 - protein, 8 aa, Artificial Sequence
  COV2-0208 IGG1-HOLE-2 HCDR2
IYPGDSDT

SEQ ID NO: 160 - protein, 11 aa, Artificial Sequence
  COV2-0208 IGG1-HOLE-2 HCDR3
ARGGYEDVFDI

SEQ ID NO: 161 - protein, 118 aa, Artificial Sequence; identical to SEQ ID NO: 101
  COV2-0208 IGG1-HOLE-2 VH

SEQ ID NO: 162 - protein, 452 aa, Artificial Sequence
  COV2-0208 IGG1-HOLE-2 HEAVY CHAIN
EVQLVQSGAEVKKPGESLKISCKGSGFSFTSYWIGWVRQMPGKGLEWMGIIYPGDSDTRYSPSFQGQVTISADKSISTAYLQWSSLKASDTAMYYCARGGYEDVFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 163 - DNA, 354 bp, Artificial Sequence; identical to SEQ ID NO: 125
  COV2-0208 IGG1-HOLE-2 VH

SEQ ID NO: 164 - DNA, 1359 bp, Artificial Sequence
  COV2-0208 IGG1-HOLE-2 HEAVY CHAIN
gaagttcaacttgttcaaagcggcgcagaggtaaagaaacctggagagagtctcaaaatcagttgcaagggatcaggattctcatttacttcctactggataggttgggtccgacaaatgcctgggaagggtctcgaatggatggggatcatatatcctggagattcagacacaagatactccccatctttccaggggcaggttactattagtgcggacaaaagtatctcaacagcgtatctccagtggtccagtctcaaggcgagcgatactgcgatgtattattgtgcgaggggtggttatgaggacgtgttcgatatttgggggcagggcacgatggtcacggtatcttccgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 165 - protein, 7 aa, Artificial Sequence
  COV2-0208 LIGHT CHAIN-1 LCDR1
QSVSSSY

SEQ ID NO: 166 - protein, 11 aa, Artificial Sequence [sequence omitted]

SEQ ID NO: 167 - protein, 109 aa, Artificial Sequence; identical to SEQ ID NO: 104
  COV2-0208 LIGHT CHAIN-1 VL

SEQ ID NO: 168 - protein, 214 aa, Artificial Sequence
  COV2-0208 LIGHT CHAIN-1
EIVLTQSPGTLSLSPGGRATLSCRASQSVSSSYLAWYQQKPGQAPRLLIYGASSRATGIPDRFSGSGSGTDFTLTISRLEPEDFAVYYCQQYGSSPPTFGQGTKVEIKRSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 169 - DNA, 327 bp, Artificial Sequence; identical to SEQ ID NO: 128
  COV2-0208 LIGHT CHAIN-1 VL

SEQ ID NO: 170 - DNA, 645 bp, Artificial Sequence
  COV2-0208 LIGHT CHAIN-1
gaaatagtattgacgcagtccccagggactctgtcccttagtcccggtggtcgcgctacgctgagttgccgagcctcccaatcagtgtcaagttcttatcttgcttggtatcagcaaaagcctggtcaggccccaaggcttctcatctatggcgcttcatcaagagcgactggtattccagaccgctttagtggttcaggaagtggaaccgactttacgctcactatctcaagactcgaacctgaagactttgcagtttattactgtcagcagtatgggagcagcccacctacattcggtcaaggcacgaaggtcgagattaagagatcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttag

SEQ ID NO: 171 - protein, 12 aa, Artificial Sequence
  COV2-0208 LIGHT CHAIN-2 LCDR1
QSVLYSSNNENY

SEQ ID NO: 172 - protein, 11 aa, Artificial Sequence [sequence omitted]

SEQ ID NO: 173 - protein, 114 aa, Artificial Sequence; identical to SEQ ID NO: 105
  COV2-0208 LIGHT CHAIN-2 VL

SEQ ID NO: 174 - protein, 220 aa, Artificial Sequence
  COV2-0208 LIGHT CHAIN-2
DIVMTQSPDSLAVSLGERATINCKSSQSVLYSSNNENYLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - DNA, 342 bp, Artificial Sequence; identical to SEQ ID NO: 129
  COV2-0208 LIGHT CHAIN-2 VL

SEQ ID NO: 176 - DNA, 663 bp, Artificial Sequence; identical to SEQ ID NO: 142
  COV2-0208 LIGHT CHAIN-2

SEQ ID NO: 177 - protein, 8 aa, Artificial Sequence
  COV2-0203 IGG1-KNOB-1 HCDR1
GFSFTSYW

SEQ ID NO: 178 - protein, 8 aa, Artificial Sequence
  COV2-0203 IGG1-KNOB-1 HCDR2
IYPGDSDT

SEQ ID NO: 179 - protein, 11 aa, Artificial Sequence
  COV2-0203 IGG1-KNOB-1 HCDR3
ARGGYEDVFDI

SEQ ID NO: 180 - protein, 118 aa, Artificial Sequence; identical to SEQ ID NO: 101
  COV2-0203 IGG1-KNOB-1 VH

SEQ ID NO: 181 - protein, 448 aa, Artificial Sequence
  COV2-0203 IGG1-KNOB-1 HEAVY CHAIN
EVQLVQSGAEVKKPGESLKISCKGSGFSFTSYWIGWVRQMPGKGLEWMGIIYPGDSDTRYSPSFQGQVTISADKSISTAYLQWSSLKASDTAMYYCARGGYEDVFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - DNA, 354 bp, Artificial Sequence; identical to SEQ ID NO: 125
  COV2-0203 IGG1-KNOB-1 VH

SEQ ID NO: 183 - DNA, 1347 bp, Artificial Sequence
  COV2-0203 IGG1-KNOB-1 HEAVY CHAIN
gaagttcaacttgttcaaagcggcgcagaggtaaagaaacctggagagagtctcaaaatcagttgcaagggatcaggattctcatttacttcctactggataggttgggtccgacaaatgcctgggaagggtctcgaatggatggggatcatatatcctggagattcagacacaagatactccccatctttccaggggcaggttactattagtgcggacaaaagtatctcaacagcgtatctccagtggtccagtctcaaggcgagcgatactgcgatgtattattgtgcgaggggtggttatgaggacgtgttcgatatttgggggcagggcacgatggtcacggtatcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatgccgggacgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 184 - protein, 8 aa, Artificial Sequence
  COV2-0203 IGG1-HOLE-2 HCDR1
GFTFSSYA

SEQ ID NO: 185 - protein, 8 aa, Artificial Sequence
  COV2-0203 IGG1-HOLE-2 HCDR2
ISGSGGST

SEQ ID NO: 186 - protein, 11 aa, Artificial Sequence
  COV2-0203 IGG1-HOLE-2 HCDR3
AKDKAVEAFDI

SEQ ID NO: 187 - protein, 118 aa, Artificial Sequence; identical to SEQ ID NO: 103
  COV2-0203 IGG1-HOLE-2 VH

SEQ ID NO: 188 - protein, 452 aa, Artificial Sequence
  COV2-0203 IGG1-HOLE-2 HEAVY CHAIN
EVQLVESGGGLVQPGGSLRLSCAASGFTFSSYAMSWVRQAPGKGLEWVSVISGSGGSTSYADSVKGRFTISRDNSKNTLYLQMNSLRAEDTAVYYCAKDKAVEAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 189 - DNA, 354 bp, Artificial Sequence; identical to SEQ ID NO: 127
  COV2-0203 IGG1-HOLE-2 VH

SEQ ID NO: 190 - DNA, 1359 bp, Artificial Sequence
  COV2-0203 IGG1-HOLE-2 HEAVY CHAIN
gaggttcagcttgtagagtccggtggcggtctcgttcaacccggcgggagcctgaggctgtcttgcgcggcttcaggattcacatttagttcctacgccatgtcctgggtcagacaagcaccaggaaaagggttggagtgggtgtcagttatctcaggttccggaggctcaaccagttacgccgacagcgtcaaaggtcggttcacgatctcccgagacaattcaaagaataccctttatctccagatgaactctttgagggcggaggacaccgccgtgtactattgtgctaaggacaaagcagtagaggctttcgacatttgggggcagggaactatggtcaccgtgtccagtgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 191 - protein, 6 aa, Artificial Sequence
  COV2-0203 LIGHT CHAIN-1 LCDR1
QDISNY

SEQ ID NO: 192 - protein, 11 aa, Artificial Sequence [sequence omitted]

SEQ ID NO: 193 - protein, 108 aa, Artificial Sequence; identical to SEQ ID NO: 106
  COV2-0203 LIGHT CHAIN-1 VL

SEQ ID NO: 194 - protein, 213 aa, Artificial Sequence
  COV2-0203 LIGHT CHAIN-1
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIKRSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 195 - DNA, 324 bp, Artificial Sequence; identical to SEQ ID NO: 130
  COV2-0203 LIGHT CHAIN-1 VL

SEQ ID NO: 196 - DNA, 642 bp, Artificial Sequence
  COV2-0203 LIGHT CHAIN-1
gacattcagatgacccaatccccgtcaagtctttctgcatctgttggagaccgagtcacaatcacatgtcaagcaagtcaagatataagcaactaccttaattggtatcagcagaaaccggggaaggcccccaagctgttgatttatgatgcgtctaatcttgagacgggagtcccatcccgattcagcgggtctgggtctggcacggatttcacatttacaataagctcccttcaaccagaggacattgccacatattactgtcagcaatacgataatttgccgctgacgtttggaggtgggactaaggttgagattaaacggtcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttag

SEQ ID NO: 197 - protein, 7 aa, Artificial Sequence
  COV2-0203 LIGHT CHAIN-2 LCDR1
QSVSSSY

SEQ ID NO: 198 - protein, 11 aa, Artificial Sequence [sequence omitted]

SEQ ID NO: 199 - protein, 109 aa, Artificial Sequence; identical to SEQ ID NO: 104
  COV2-0203 LIGHT CHAIN-2 VL

SEQ ID NO: 200 - protein, 215 aa, Artificial Sequence
  COV2-0203 LIGHT CHAIN-2
EIVLTQSPGTLSLSPGGRATLSCRASQSVSSSYLAWYQQKPGQAPRLLIYGASSRATGIPDRFSGSGSGTDFTLTISRLEPEDFAVYYCQQYGSSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - DNA, 327 bp, Artificial Sequence; identical to SEQ ID NO: 128
  COV2-0203 LIGHT CHAIN-2 VL

SEQ ID NO: 202 - DNA, 648 bp, Artificial Sequence; identical to SEQ ID NO: 140
  COV2-0203 LIGHT CHAIN-2

SEQ ID NO: 203 - protein, 8 aa, Artificial Sequence
  COV2-0803 IGG1-KNOB-1 HCDR1
GGSISSYY

SEQ ID NO: 204 - protein, 7 aa, Artificial Sequence
  COV2-0803 IGG1-KNOB-1 HCDR2
IYYSGST

SEQ ID NO: 205 - protein, 12 aa, Artificial Sequence
  COV2-0803 IGG1-KNOB-1 HCDR3
ARGLTGGDAFDI

SEQ ID NO: 206 - protein, 118 aa, Artificial Sequence; identical to SEQ ID NO: 102
  COV2-0803 IGG1-KNOB-1 VH

SEQ ID NO: 207 - protein, 448 aa, Artificial Sequence; identical to SEQ ID NO: 155
  COV2-0803 IGG1-KNOB-1 HEAVY CHAIN

SEQ ID NO: 208 - DNA, 354 bp, Artificial Sequence; identical to SEQ ID NO: 126
  COV2-0803 IGG1-KNOB-1 VH

SEQ ID NO: 209 - DNA, 1347 bp, Artificial Sequence; identical to SEQ ID NO: 157
  COV2-0803 IGG1-KNOB-1 HEAVY CHAIN

SEQ ID NO: 210 - protein, 8 aa, Artificial Sequence
  COV2-0803 IGG1-HOLE-2 HCDR1
GFTFSSYA

SEQ ID NO: 211 - protein, 8 aa, Artificial Sequence
  COV2-0803 IGG1-HOLE-2 HCDR2
ISGSGGST

SEQ ID NO: 212 - protein, 11 aa, Artificial Sequence
  COV2-0803 IGG1-HOLE-2 HCDR3
AKDKAVEAFDI

SEQ ID NO: 213 - protein, 118 aa, Artificial Sequence; identical to SEQ ID NO: 103
  COV2-0803 IGG1-HOLE-2 VH

SEQ ID NO: 214 - protein, 452 aa, Artificial Sequence; identical to SEQ ID NO: 188
  COV2-0803 IGG1-HOLE-2 HEAVY CHAIN

SEQ ID NO: 215 - DNA, 354 bp, Artificial Sequence; identical to SEQ ID NO: 127
  COV2-0803 IGG1-HOLE-2 VH

SEQ ID NO: 216 - DNA, 1359 bp, Artificial Sequence; identical to SEQ ID NO: 190
  COV2-0803 IGG1-HOLE-2 HEAVY CHAIN

SEQ ID NO: 217 - protein, 6 aa, Artificial Sequence
  COV2-0803 LIGHT CHAIN-1 LCDR1
QDISNY

SEQ ID NO: 218 - protein, 11 aa, Artificial Sequence [sequence omitted]

SEQ ID NO: 219 - protein, 108 aa, Artificial Sequence; identical to SEQ ID NO: 106
  COV2-0803 LIGHT CHAIN-1 VL

SEQ ID NO: 220 - protein, 213 aa, Artificial Sequence; identical to SEQ ID NO: 194
  COV2-0803 LIGHT CHAIN-1

SEQ ID NO: 221 - DNA, 324 bp, Artificial Sequence; identical to SEQ ID NO: 130
  COV2-0803 LIGHT CHAIN-1 VL

SEQ ID NO: 222 - DNA, 642 bp, Artificial Sequence; identical to SEQ ID NO: 196
  COV2-0803 LIGHT CHAIN-1

SEQ ID NO: 223 - protein, 12 aa, Artificial Sequence
  COV2-0803 LIGHT CHAIN-2 LCDR1
QSVLYSSNNENY

SEQ ID NO: 224 - protein, 11 aa, Artificial Sequence [sequence omitted]

SEQ ID NO: 225 - protein, 114 aa, Artificial Sequence; identical to SEQ ID NO: 105
  COV2-0803 LIGHT CHAIN-2 VL

SEQ ID NO: 226 - protein, 220 aa, Artificial Sequence; identical to SEQ ID NO: 174
  COV2-0803 LIGHT CHAIN-2

SEQ ID NO: 227 - DNA, 342 bp, Artificial Sequence; identical to SEQ ID NO: 129
  COV2-0803 LIGHT CHAIN-2 VL

SEQ ID NO: 228 - DNA, 663 bp, Artificial Sequence; identical to SEQ ID NO: 142
  COV2-0803 LIGHT CHAIN-2

SEQ ID NO: 229 - protein, 8 aa, Artificial Sequence
  GFSFTSYW
GFSFTSYW

SEQ ID NO: 230 - protein, 8 aa, Artificial Sequence
  COV2-0213 IGG1-KNOB-1 HCDR2
IYPGDSDT

SEQ ID NO: 231 - protein, 11 aa, Artificial Sequence
  COV2-0213 IGG1-KNOB-1 HCDR3
ARGGYEDVFDI

SEQ ID NO: 232 - protein, 118 aa, Artificial Sequence; identical to SEQ ID NO: 101
  COV2-0213 IGG1-KNOB-1 VH

SEQ ID NO: 233 - protein, 448 aa, Artificial Sequence; identical to SEQ ID NO: 181
  COV2-0213 IGG1-KNOB-1 HEAVY CHAIN

SEQ ID NO: 234 - DNA, 354 bp, Artificial Sequence; identical to SEQ ID NO: 125
  COV2-0213 IGG1-KNOB-1 VH

SEQ ID NO: 235 - DNA, 1347 bp, Artificial Sequence; identical to SEQ ID NO: 183
  COV2-0213 IGG1-KNOB-1 HEAVY CHAIN

SEQ ID NO: 236 - protein, 8 aa, Artificial Sequence
  COV2-0213 IGG1-HOLE-2 HCDR1
GFTFTGYS

SEQ ID NO: 237 - protein, 8 aa, Artificial Sequence
  COV2-0213 IGG1-HOLE-2 HCDR2
INPSTGGT

SEQ ID NO: 238 - protein, 7 aa, Artificial Sequence
  COV2-0213 IGG1-HOLE-2 HCDR3
ARSRGDY

SEQ ID NO: 239 - protein, 114 aa, Artificial Sequence
  COV2-0213 IGG1-HOLE-2 VH
EVQLVESGGGLVQPGGSLRLSCAASGFTFTGYSMNWMRQAPGKGLEWVAEINPSTGGTTDNQKFKARFTISRDNSKNTLYLQMNSLRAEDTAVYYCARSRGDYWGQGTLVTVSS

SEQ ID NO: 240 - protein, 448 aa, Artificial Sequence
  COV2-0213 IGG1-HOLE-2 HEAVY CHAIN
EVQLVESGGGLVQPGGSLRLSCAASGFTFTGYSMNWMRQAPGKGLEWVAEINPSTGGTTDNQKFKARFTISRDNSKNTLYLQMNSLRAEDTAVYYCARSRGDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 241 - DNA, 342 bp, Artificial Sequence
  COV2-0213 IGG1-HOLE-2 VH
gaggttcagcttgtggaatcagggggtgggctggtccagcctgggggatctttgcgactgagttgtgctgcttcaggtttcacatttactggctacagcatgaactggatgcggcaagcgccaggcaaagggcttgaatgggtggcggagatcaatccatccacaggaggcacgaccgataaccagaaatttaaggcgaggtttaccataagtagagataactctaaaaacacgctctatctccaaatgaacagtctgcgggcggaggatactgcagtctactattgcgcccgcagccgaggggactattgggggcagggcactttggttacagtgtccagc

SEQ ID NO: 242 - DNA, 1347 bp, Artificial Sequence
  COV2-0213 IGG1-HOLE-2 HEAVY CHAIN
gaggttcagcttgtggaatcagggggtgggctggtccagcctgggggatctttgcgactgagttgtgctgcttcaggtttcacatttactggctacagcatgaactggatgcggcaagcgccaggcaaagggcttgaatgggtggcggagatcaatccatccacaggaggcacgaccgataaccagaaatttaaggcgaggtttaccataagtagagataactctaaaaacacgctctatctccaaatgaacagtctgcgggcggaggatactgcagtctactattgcgcccgcagccgaggggactattgggggcagggcactttggttacagtgtccagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 243 - protein, 8 aa, Artificial Sequence
  COV2-0213 LIGHT CHAIN-1 LCDR1
SVEYYGTS

SEQ ID NO: 244 - protein, 9 aa, Artificial Sequence
  COV2-0213 LIGHT CHAIN-1 LCDR3
QQSRKVPWT

SEQ ID NO: 245 - protein, 112 aa, Artificial Sequence
  COV2-0213 LIGHT CHAIN-1 VH
DIQMTQSPSSLSASVGDRVTITCRASESVEYYGTSLMQWYQQKPGKAPKLLIYAASNVESGVPARFSGSGSGTDFTLTISSLQPEDFATYYCQQSRKVPWTFGQGTKVEIKR

SEQ ID NO: 246 - protein, 217 aa, Artificial Sequence
  COV2-0213 LIGHT CHAIN-1
DIQMTQSPSSLSASVGDRVTITCRASESVEYYGTSLMQWYQQKPGKAPKLLIYAASNVESGVPARFSGSGSGTDFTLTISSLQPEDFATYYCQQSRKVPWTFGQGTKVEIKRSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 247 - DNA, 336 bp, Artificial Sequence
  COV2-0213 LIGHT CHAIN-1 VH
gacattcagatgacacaatccccctctagcctgtcagcgtccgtgggcgaccgagttacgattacctgccgagcatcagaatccgtagagtattatggcacgtctcttatgcaatggtatcagcagaaaccgggtaaagcgccaaaactgctcatctacgcggcgtctaacgtggagtcaggtgtacccgccagattttctggttcaggaagcgggactgattttacattgactataagctcactgcaaccggaagatttcgctacctactattgtcaacaatcacggaaggtcccatggaccttcgggcagggtactaaagtcgagattaaaagg

SEQ ID NO: 248 - DNA, 654 bp, Artificial Sequence
  COV2-0213 LIGHT CHAIN-1
gacattcagatgacacaatccccctctagcctgtcagcgtccgtgggcgaccgagttacgattacctgccgagcatcagaatccgtagagtattatggcacgtctcttatgcaatggtatcagcagaaaccgggtaaagcgccaaaactgctcatctacgcggcgtctaacgtggagtcaggtgtacccgccagattttctggttcaggaagcgggactgattttacattgactataagctcactgcaaccggaagatttcgctacctactattgtcaacaatcacggaaggtcccatggaccttcgggcagggtactaaagtcgagattaaaaggtcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttag

SEQ ID NO: 249 - protein, 7 aa, Artificial Sequence
  COV2-0213 LIGHT CHAIN-2 LCDR1
QSVSSSY

SEQ ID NO: 250 - protein, 11 aa, Artificial Sequence [sequence omitted]

SEQ ID NO: 251 - protein, 109 aa, Artificial Sequence; identical to SEQ ID NO: 104
  COV2-0213 LIGHT CHAIN-2 VL

SEQ ID NO: 252 - protein, 215 aa, Artificial Sequence; identical to SEQ ID NO: 200
  COV2-0213 LIGHT CHAIN-2

SEQ ID NO: 253 - DNA, 327 bp, Artificial Sequence; identical to SEQ ID NO: 128
  COV2-0213 LIGHT CHAIN-2 VL

SEQ ID NO: 254 - DNA, 648 bp, Artificial Sequence; identical to SEQ ID NO: 140
  COV2-0213 LIGHT CHAIN-2

SEQ ID NO: 255 - protein, 8 aa, Artificial Sequence
  COV2-0813 IGG1-KNOB-1 HCDR1
GGSISSYY

SEQ ID NO: 256 - protein, 7 aa, Artificial Sequence
  COV2-0813 IGG1-KNOB-1 HCDR2
IYYSGST

SEQ ID NO: 257 - protein, 12 aa, Artificial Sequence
  COV2-0813 IGG1-KNOB-1 HCDR3
ARGLTGGDAFDI

SEQ ID NO: 258 - protein, 118 aa, Artificial Sequence; identical to SEQ ID NO: 102
  COV2-0813 IGG1-KNOB-1 VH

SEQ ID NO: 259 - protein, 448 aa, Artificial Sequence; identical to SEQ ID NO: 155
  COV2-0813 IGG1-KNOB-1 HEAVY CHAIN

SEQ ID NO: 260 - DNA, 354 bp, Artificial Sequence; identical to SEQ ID NO: 126
  COV2-0813 IGG1-KNOB-1 VH

SEQ ID NO: 261 - DNA, 1347 bp, Artificial Sequence; identical to SEQ ID NO: 157
  COV2-0813 IGG1-KNOB-1 HEAVY CHAIN

SEQ ID NO: 262 - protein, 8 aa, Artificial Sequence
  COV2-0813 IGG1-HOLE-2 HCDR1
GFTFTGYS

SEQ ID NO: 263 - protein, 8 aa, Artificial Sequence
  COV2-0813 IGG1-HOLE-2 HCDR2
INPSTGGT

SEQ ID NO: 264 - protein, 7 aa, Artificial Sequence
  COV2-0813 IGG1-HOLE-2 HCDR3
ARSRGDY

SEQ ID NO: 265 - protein, 114 aa, Artificial Sequence; identical to SEQ ID NO: 239
  COV2-0813 IGG1-HOLE-2 VH

SEQ ID NO: 266 - protein, 448 aa, Artificial Sequence; identical to SEQ ID NO: 240
  COV2-0813 IGG1-HOLE-2 HEAVY CHAIN

SEQ ID NO: 267 - DNA, 342 bp, Artificial Sequence; identical to SEQ ID NO: 241
  COV2-0813 IGG1-HOLE-2 VH

SEQ ID NO: 268 - DNA, 1347 bp, Artificial Sequence; identical to SEQ ID NO: 242
  COV2-0813 IGG1-HOLE-2 HEAVY CHAIN

SEQ ID NO: 269 - protein, 8 aa, Artificial Sequence
  COV2-0813 LIGHT CHAIN-1 LCDR1
SVEYYGTS

SEQ ID NO: 270 - protein, 9 aa, Artificial Sequence
  COV2-0813 LIGHT CHAIN-1 LCDR3
QQSRKVPWT

SEQ ID NO: 271 - protein, 112 aa, Artificial Sequence; identical to SEQ ID NO: 245
  COV2-0813 LIGHT CHAIN-1 VL

SEQ ID NO: 272 - protein, 217 aa, Artificial Sequence; identical to SEQ ID NO: 246
  COV2-0813 LIGHT CHAIN-1

SEQ ID NO: 273 - DNA, 336 bp, Artificial Sequence; identical to SEQ ID NO: 247
  COV2-0813 LIGHT CHAIN-1 VL

SEQ ID NO: 274 - DNA, 654 bp, Artificial Sequence; identical to SEQ ID NO: 248
  COV2-0813 LIGHT CHAIN-1

SEQ ID NO: 275 - protein, 12 aa, Artificial Sequence
  COV2-0813 LIGHT CHAIN-2 LCDR1
QSVLYSSNNENY

SEQ ID NO: 276 - protein, 11 aa, Artificial Sequence [sequence omitted]

SEQ ID NO: 277 - protein, 114 aa, Artificial Sequence; identical to SEQ ID NO: 105
  COV2-0813 LIGHT CHAIN-2 VL

SEQ ID NO: 278 - protein, 220 aa, Artificial Sequence; identical to SEQ ID NO: 174
  COV2-0813 LIGHT CHAIN-2

SEQ ID NO: 279 - DNA, 342 bp, Artificial Sequence; identical to SEQ ID NO: 129
  COV2-0813 LIGHT CHAIN-2 VL

SEQ ID NO: 280 - DNA, 663 bp, Artificial Sequence; identical to SEQ ID NO: 142
  COV2-0813 LIGHT CHAIN-2

SEQ ID NO: 281 - DNA, 4178 bp, Artificial Sequence; identical to SEQ ID NO: 146
  Omicr-MB_VK2

SEQ ID NO: 282 - DNA, 4193 bp, Artificial Sequence; identical to SEQ ID NO: 148
  Omicr-MB_VK8

SEQ ID NO: 283 - DNA, 4836 bp, Artificial Sequence
  pPR146 pfuse-IgG1-Knob-MB.08-VH-CH1-CH2-CH3
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgcaggtccagctgcaagagtccgggccgggcctcgtaaaaccctctgagacactgtccctgacttgcacggtgtctggtggttctatctcatcctactattggtcctggattagacagcccccagggaagggccttgagtggatcgggtacatatattattcagggagcactaactacaatccttcactcaagtcacgagtgactatatccgttgacacgagcaaaaaccagttttccctcaaattgagtagcgttacagcagcggatacagcagtctattactgtgcaagaggtctcacgggaggggacgccttcgatatatggggccagggtacgatggtgaccgttagttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatgccgggacgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 284 - DNA, 4184 bp, Artificial Sequence
  pPR148 pFUSE-IgG-Clone13A-VL-CH1
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagacattcagatgacacaatccccctctagcctgtcagcgtccgtgggcgaccgagttacgattacctgccgagcatcagaatccgtagagtattatggcacgtctcttatgcaatggtatcagcagaaaccgggtaaagcgccaaaactgctcatctacgcggcgtctaacgtggagtcaggtgtacccgccagattttctggttcaggaagcgggactgattttacattgactataagctcactgcaaccggaagatttcgctacctactattgtcaacaatcacggaaggtcccatggaccttcgggcagggtactaaagtcgagattaaaaggtcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttagagggagcaagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 285 - DNA, 4836 bp, Artificial Sequence
  pPR149 pfuse-IgG1-Hole-Clone13A-VH-CKappa-CH2-CH3
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggttcagcttgtggaatcagggggtgggctggtccagcctgggggatctttgcgactgagttgtgctgcttcaggtttcacatttactggctacagcatgaactggatgcggcaagcgccaggcaaagggcttgaatgggtggcggagatcaatccatccacaggaggcacgaccgataaccagaaatttaaggcgaggtttaccataagtagagataactctaaaaacacgctctatctccaaatgaacagtctgcgggcggaggatactgcagtctactattgcgcccgcagccgaggggactattgggggcagggcactttggttacagtgtccagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 286 - DNA, 4175 bp, Artificial Sequence
  pPR150 pFUSE-IgG-MB2-VL-CH1
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagaaatagtattgacgcagtccccagggactctgtcccttagtcccggtggtcgcgctacgctgagttgccgagcctcccaatcagtgtcaagttcttatcttgcttggtatcagcaaaagcctggtcaggccccaaggcttctcatctatggcgcttcatcaagagcgactggtattccagaccgctttagtggttcaggaagtggaaccgactttacgctcactatctcaagactcgaacctgaagactttgcagtttattactgtcagcagtatgggagcagcccacctacattcggtcaaggcacgaaggtcgagattaagagatcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttagagggagcaagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 287 - DNA, 4172 bp, Artificial Sequence
  pPR151 pFUSE-IgG-PB3-VL-CH1
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaacatgtacaggatgcaactcctgtcttgcattgcactaagtcttgcacttgtcacgaattcagacattcagatgacccaatccccgtcaagtctttctgcatctgttggagaccgagtcacaatcacatgtcaagcaagtcaagatataagcaactaccttaattggtatcagcagaaaccggggaaggcccccaagctgttgatttatgatgcgtctaatcttgagacgggagtcccatcccgattcagcgggtctgggtctggcacggatttcacatttacaataagctcccttcaaccagaggacattgccacatattactgtcagcaatacgataatttgccgctgacgtttggaggtgggactaaggttgagattaaacggtcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttagagggagcaagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagttcctggtgtacttgagggggatgagttcctcaatggtggttttgaccagcttgccattcatctcaatgagcacaaagcagtcaggagcatagtcagagatgagctctctgcacatgccacaggggctgaccaccctgatggatctgtccacctcatcagagtaggggtgcctgacagccacaatggtgtcaaagtccttctgcccgttgctcacagcagacccaatggcaatggcttcagcacagacagtgaccctgccaatgtaggcctcaatgtggacagcagagatgatctccccagtcttggtcctgatggccgccccgacatggtgcttgttgtcctcatagagcatggtgatcttctcagtggcgacctccaccagctccagatcctgctgagagatgttgaaggtcttcatgatggctcctcctgtcaggagaggaaagagaagaaggttagtacaattgctatagtgagttgtattatactatgcttatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 288 - DNA, 4848 bp, Artificial Sequence
  pPR152 pfuse-IgG1-Hole-MB2-VH-CKappa-CH2-CH3
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaagttcaacttgttcaaagcggcgcagaggtaaagaaacctggagagagtctcaaaatcagttgcaagggatcaggattctcatttacttcctactggataggttgggtccgacaaatgcctgggaagggtctcgaatggatggggatcatatatcctggagattcagacacaagatactccccatctttccaggggcaggttactattagtgcggacaaaagtatctcaacagcgtatctccagtggtccagtctcaaggcgagcgatactgcgatgtattattgtgcgaggggtggttatgaggacgtgttcgatatttgggggcagggcacgatggtcacggtatcttccgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 289 - DNA, 4848 bp, Artificial Sequence
  pPR153 pfuse-IgG1-Hole-PB3-VH-CKappa-CH2-CH3
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaggttcagcttgtagagtccggtggcggtctcgttcaacccggcgggagcctgaggctgtcttgcgcggcttcaggattcacatttagttcctacgccatgtcctgggtcagacaagcaccaggaaaagggttggagtgggtgtcagttatctcaggttccggaggctcaaccagttacgccgacagcgtcaaaggtcggttcacgatctcccgagacaattcaaagaataccctttatctccagatgaactctttgagggcggaggacaccgccgtgtactattgtgctaaggacaaagcagtagaggctttcgacatttgggggcagggaactatggtcaccgtgtccagtgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggacgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 290 - DNA, 4836 bp, Artificial Sequence
  pPR154 pfuse-IgG1-Knob-MB.02-VH-CH1-CH2-CH3
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcggaagttcaacttgttcaaagcggcgcagaggtaaagaaacctggagagagtctcaaaatcagttgcaagggatcaggattctcatttacttcctactggataggttgggtccgacaaatgcctgggaagggtctcgaatggatggggatcatatatcctggagattcagacacaagatactccccatctttccaggggcaggttactattagtgcggacaaaagtatctcaacagcgtatctccagtggtccagtctcaaggcgagcgatactgcgatgtattattgtgcgaggggtggttatgaggacgtgttcgatatttgggggcagggcacgatggtcacggtatcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatgccgggacgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa